SEQUENCE LISTING (495 sequences)

SEQ ID NO: 1 - protein, 5 aa, Artificial Sequence
  Description of Artificial Sequence: Synthetic peptide
SYGMH

SEQ ID NO: 2 - protein, 17 aa, Artificial Sequence
  Description of Artificial Sequence: Synthetic peptide
VIWYEGSNKYYADSVKG

SEQ ID NO: 3 - protein, 15 aa, Artificial Sequence
  Description of Artificial Sequence: Synthetic peptide
GGSMVRGDYYYGMDV

SEQ ID NO: 4 - protein, 11 aa, Artificial Sequence
  Description of Artificial Sequence: Synthetic peptide
RASQGISSALA

SEQ ID NO: 5 - protein, 7 aa, Artificial Sequence
  Description of Artificial Sequence: Synthetic peptide
DASSLES

SEQ ID NO: 6 - protein, 9 aa, Artificial Sequence
  Description of Artificial Sequence: Synthetic peptide
QQFNSYPYT

SEQ ID NO: 7 - protein, 5 aa, Artificial Sequence
  Description of Artificial Sequence: Synthetic peptide
DYYWN

SEQ ID NO: 8 - protein, 16 aa, Artificial Sequence
  Description of Artificial Sequence: Synthetic peptide
EINHRGSTNSNPSLKS

SEQ ID NO: 9 - protein, 12 aa, Artificial Sequence
  Description of Artificial Sequence: Synthetic peptide
GYSDYEYNWFDP

SEQ ID NO: 10 - protein, 11 aa, Artificial Sequence
  Description of Artificial Sequence: Synthetic peptide
RASQSISSYLA

SEQ ID NO: 11 - protein, 7 aa, Artificial Sequence
  Description of Artificial Sequence: Synthetic peptide
DASNRAT

SEQ ID NO: 12 - protein, 9 aa, Artificial Sequence
  Description of Artificial Sequence: Synthetic peptide
QQRSNWPLT

SEQ ID NO: 13 - protein, 8 aa, Artificial Sequence
  Description of Artificial Sequence: Synthetic peptide
GYSFSTYW

SEQ ID NO: 14 - protein, 8 aa, Artificial Sequence
  Description of Artificial Sequence: Synthetic peptide
IYPGDSYT

SEQ ID NO: 15 - protein, 9 aa, Artificial Sequence
  Description of Artificial Sequence: Synthetic peptide
ARGYGIFDY

SEQ ID NO: 16 - protein, 6 aa, Artificial Sequence
  Description of Artificial Sequence: Synthetic peptide
NIGDQY

SEQ ID NO: 17 - protein, 3 aa, Artificial Sequence
  Description of Artificial Sequence: Synthetic peptide
QDK

SEQ ID NO: 18 - protein, 11 aa, Artificial Sequence
  Description of Artificial Sequence: Synthetic peptide
ATYTGFGSLAV

SEQ ID NO: 19 - protein, 8 aa, Artificial Sequence
  Description of Artificial Sequence: Synthetic peptide
GFTFSSYA

SEQ ID NO: 20 - protein, 8 aa, Artificial Sequence
  Description of Artificial Sequence: Synthetic peptide
ISGSGGST

SEQ ID NO: 21 - protein, 10 aa, Artificial Sequence
  Description of Artificial Sequence: Synthetic peptide
AKGWLGNFDY

SEQ ID NO: 22 - protein, 7 aa, Artificial Sequence
  Description of Artificial Sequence: Synthetic peptide
QSVSRSY

SEQ ID NO: 23 - protein, 3 aa, Artificial Sequence
  Description of Artificial Sequence: Synthetic peptide
GAS

SEQ ID NO: 24 - protein, 9 aa, Artificial Sequence
  Description of Artificial Sequence: Synthetic peptide
QQGQVIPPT

SEQ ID NO: 25 - protein, 8 aa, Artificial Sequence
  Description of Artificial Sequence: Synthetic peptide
GFTFSSYA

SEQ ID NO: 26 - protein, 8 aa, Artificial Sequence
  Description of Artificial Sequence: Synthetic peptide
IIGSGAST

SEQ ID NO: 27 - protein, 10 aa, Artificial Sequence
  Description of Artificial Sequence: Synthetic peptide
AKGWFGGFNY

SEQ ID NO: 28 - protein, 7 aa, Artificial Sequence
  Description of Artificial Sequence: Synthetic peptide
QSVTSSY

SEQ ID NO: 29 - protein, 3 aa, Artificial Sequence
  Description of Artificial Sequence: Synthetic peptide
VGS

SEQ ID NO: 30 - protein, 9 aa, Artificial Sequence
  Description of Artificial Sequence: Synthetic peptide
QQGIMLPPT

SEQ ID NO: 31 - protein, 8 aa, Artificial Sequence
  Description of Artificial Sequence: Synthetic peptide
GFTFSSHA

SEQ ID NO: 32 - protein, 7 aa, Artificial Sequence
  Description of Artificial Sequence: Synthetic peptide
IWASGEQ

SEQ ID NO: 33 - protein, 10 aa, Artificial Sequence
  Description of Artificial Sequence: Synthetic peptide
AKGWLGNFDY

SEQ ID NO: 34 - protein, 7 aa, Artificial Sequence
  Description of Artificial Sequence: Synthetic peptide
QSVSRSY

SEQ ID NO: 35 - protein, 3 aa, Artificial Sequence
  Description of Artificial Sequence: Synthetic peptide
GAS

SEQ ID NO: 36 - protein, 9 aa, Artificial Sequence
  Description of Artificial Sequence: Synthetic peptide
QQGQVIPPT

SEQ ID NO: 37 - protein, 8 aa, Artificial Sequence
  Description of Artificial Sequence: Synthetic peptide
GYTFTDYV

SEQ ID NO: 38 - protein, 8 aa, Artificial Sequence
  Description of Artificial Sequence: Synthetic peptide
INPYDDDT

SEQ ID NO: 39 - protein, 17 aa, Artificial Sequence
  Description of Artificial Sequence: Synthetic peptide
ARRGNSYDGYFDYSMDY

SEQ ID NO: 40 - protein, 6 aa, Artificial Sequence
  Description of Artificial Sequence: Synthetic peptide
QNVGTA

SEQ ID NO: 41 - protein, 3 aa, Artificial Sequence
  Description of Artificial Sequence: Synthetic peptide
SAS

SEQ ID NO: 42 - protein, 10 aa, Artificial Sequence
  Description of Artificial Sequence: Synthetic peptide
QQYTNYPMYT

SEQ ID NO: 43 - protein, 10 aa, Artificial Sequence
  Description of Artificial Sequence: Synthetic peptide
GGSITSSSYY

SEQ ID NO: 44 - protein, 7 aa, Artificial Sequence
  Description of Artificial Sequence: Synthetic peptide
IYYRGST

SEQ ID NO: 45 - protein, 14 aa, Artificial Sequence
  Description of Artificial Sequence: Synthetic peptide
ARQNGAARPSWFDP

SEQ ID NO: 46 - protein, 7 aa, Artificial Sequence
  Description of Artificial Sequence: Synthetic peptide
QSISSIY

SEQ ID NO: 47 - protein, 3 aa, Artificial Sequence
  Description of Artificial Sequence: Synthetic peptide
GAS

SEQ ID NO: 48 - protein, 9 aa, Artificial Sequence
  Description of Artificial Sequence: Synthetic peptide
QHYGISPFT

SEQ ID NO: 49 - protein, 8 aa, Artificial Sequence
  Description of Artificial Sequence: Synthetic peptide
GYTFTSYG

SEQ ID NO: 50 - protein, 8 aa, Artificial Sequence
  Description of Artificial Sequence: Synthetic peptide
ISVYSGNT

SEQ ID NO: 51 - protein, 17 aa, Artificial Sequence
  Description of Artificial Sequence: Synthetic peptide
AREGSSSSGDYYYGMDV

SEQ ID NO: 52 - protein, 11 aa, Artificial Sequence
  Description of Artificial Sequence: Synthetic peptide
QSLLHTDGTTY

SEQ ID NO: 53 - protein, 3 aa, Artificial Sequence
  Description of Artificial Sequence: Synthetic peptide
EVS

SEQ ID NO: 54 - protein, 9 aa, Artificial Sequence
  Description of Artificial Sequence: Synthetic peptide
MQNIQLPWT

SEQ ID NO: 55 - protein, 8 aa, Artificial Sequence
  Description of Artificial Sequence: Synthetic peptide
GYTFTSYG

SEQ ID NO: 56 - protein, 8 aa, Artificial Sequence
  Description of Artificial Sequence: Synthetic peptide
ISVYSGNT

SEQ ID NO: 57 - protein, 17 aa, Artificial Sequence
  Description of Artificial Sequence: Synthetic peptide
AREGSSSSGDYYYGMDV

SEQ ID NO: 58 - protein, 11 aa, Artificial Sequence
  Description of Artificial Sequence: Synthetic peptide
QSLLYSDGKTY

SEQ ID NO: 59 - protein, 3 aa, Artificial Sequence
  Description of Artificial Sequence: Synthetic peptide
EVS

SEQ ID NO: 60 - protein, 9 aa, Artificial Sequence
  Description of Artificial Sequence: Synthetic peptide
MQSIQLPWT

SEQ ID NO: 61 - protein, 8 aa, Artificial Sequence
  Description of Artificial Sequence: Synthetic peptide
GFTFSSYG

SEQ ID NO: 62 - protein, 7 aa, Artificial Sequence
  Description of Artificial Sequence: Synthetic peptide
IGTGGGT

SEQ ID NO: 63 - protein, 14 aa, Artificial Sequence
  Description of Artificial Sequence: Synthetic peptide
ARGDYYGSGSFFDC

SEQ ID NO: 64 - protein, 6 aa, Artificial Sequence
  Description of Artificial Sequence: Synthetic peptide
QGISSW

SEQ ID NO: 65 - protein, 3 aa, Artificial Sequence
  Description of Artificial Sequence: Synthetic peptide
AAS

SEQ ID NO: 66 - protein, 9 aa, Artificial Sequence
  Description of Artificial Sequence: Synthetic peptide
QQYNSYPPT

SEQ ID NO: 67 - protein, 10 aa, Artificial Sequence
  Description of Artificial Sequence: Synthetic peptide
GYTFTSYTIH

SEQ ID NO: 68 - protein, 17 aa, Artificial Sequence
  Description of Artificial Sequence: Synthetic peptide
YINPGSGYTNYNEKFQD

SEQ ID NO: 69 - protein, 5 aa, Artificial Sequence
  Description of Artificial Sequence: Synthetic peptide
WDRGY

SEQ ID NO: 70 - protein, 16 aa, Artificial Sequence
  Description of Artificial Sequence: Synthetic peptide
RSSQTIVHSNGNTYLE

SEQ ID NO: 71 - protein, 7 aa, Artificial Sequence
  Description of Artificial Sequence: Synthetic peptide
KVSNRFS

SEQ ID NO: 72 - protein, 9 aa, Artificial Sequence
  Description of Artificial Sequence: Synthetic peptide
FQGSHVPYT

SEQ ID NO: 73 - protein, 8 aa, Artificial Sequence
  Description of Artificial Sequence: Synthetic peptide
GFTFSNYG

SEQ ID NO: 74 - protein, 8 aa, Artificial Sequence
  Description of Artificial Sequence: Synthetic peptide
ISAGGDKT

SEQ ID NO: 75 - protein, 5 aa, Artificial Sequence
  Description of Artificial Sequence: Synthetic peptide
AKTSR

SEQ ID NO: 76 - protein, 8 aa, Artificial Sequence
  Description of Artificial Sequence: Synthetic peptide
TGNIGSNY

SEQ ID NO: 77 - protein, 3 aa, Artificial Sequence
  Description of Artificial Sequence: Synthetic peptide
RDD

SEQ ID NO: 78 - protein, 8 aa, Artificial Sequence
  Description of Artificial Sequence: Synthetic peptide
HSYSSGIV

SEQ ID NO: 79 - protein, 8 aa, Artificial Sequence
  Description of Artificial Sequence: Synthetic peptide
GFNIKDTY

SEQ ID NO: 80 - protein, 8 aa, Artificial Sequence
  Description of Artificial Sequence: Synthetic peptide
IDPANGYT

SEQ ID NO: 81 - protein, 16 aa, Artificial Sequence
  Description of Artificial Sequence: Synthetic peptide
AREGYYGNYGVYAMDY

SEQ ID NO: 82 - protein, 6 aa, Artificial Sequence
  Description of Artificial Sequence: Synthetic peptide
QDINKY

SEQ ID NO: 83 - protein, 3 aa, Artificial Sequence
  Description of Artificial Sequence: Synthetic peptide
YTS

SEQ ID NO: 84 - protein, 8 aa, Artificial Sequence
  Description of Artificial Sequence: Synthetic peptide
LQYDNLWT

SEQ ID NO: 85 - protein, 8 aa, Artificial Sequence
  Description of Artificial Sequence: Synthetic peptide
GYTFTSYR

SEQ ID NO: 86 - protein, 8 aa, Artificial Sequence
  Description of Artificial Sequence: Synthetic peptide
INPSTGYT

SEQ ID NO: 87 - protein, 9 aa, Artificial Sequence
  Description of Artificial Sequence: Synthetic peptide
ARGGGVFDY

SEQ ID NO: 88 - protein, 6 aa, Artificial Sequence
  Description of Artificial Sequence: Synthetic peptide
SSSISY

SEQ ID NO: 89 - protein, 3 aa, Artificial Sequence
  Description of Artificial Sequence: Synthetic peptide
TTS

SEQ ID NO: 90 - protein, 9 aa, Artificial Sequence
  Description of Artificial Sequence: Synthetic peptide
HQRSTYPLT

SEQ ID NO: 91 - protein, 8 aa, Artificial Sequence
  Description of Artificial Sequence: Synthetic peptide
GYTFTDYN

SEQ ID NO: 92 - protein, 8 aa, Artificial Sequence
  Description of Artificial Sequence: Synthetic peptide
INPNYEST

SEQ ID NO: 93 - protein, 9 aa, Artificial Sequence
  Description of Artificial Sequence: Synthetic peptide
RDKGWYFDV

SEQ ID NO: 94 - protein, 5 aa, Artificial Sequence
  Description of Artificial Sequence: Synthetic peptide
SSVKN

SEQ ID NO: 95 - protein, 3 aa, Artificial Sequence
  Description of Artificial Sequence: Synthetic peptide
YTS

SEQ ID NO: 96 - protein, 9 aa, Artificial Sequence
  Description of Artificial Sequence: Synthetic peptide
QQFTSSPYT

SEQ ID NO: 97 - protein, 10 aa, Artificial Sequence
  Description of Artificial Sequence: Synthetic peptide
GFSLSTSGMG

SEQ ID NO: 98 - protein, 7 aa, Artificial Sequence
  Description of Artificial Sequence: Synthetic peptide
IWWDDDK

SEQ ID NO: 99 - protein, 11 aa, Artificial Sequence
  Description of Artificial Sequence: Synthetic peptide
ARLTGTRYFDY

SEQ ID NO: 100 - protein, 6 aa, Artificial Sequence
  Description of Artificial Sequence: Synthetic peptide
QDINKF

SEQ ID NO: 101 - protein, 3 aa, Artificial Sequence
  Description of Artificial Sequence: Synthetic peptide
YTS

SEQ ID NO: 102 - protein, 8 aa, Artificial Sequence
  Description of Artificial Sequence: Synthetic peptide
LQYGNLWT

SEQ ID NO: 103 - protein, 8 aa, Artificial Sequence
  Description of Artificial Sequence: Synthetic peptide
GYTFTDYS

SEQ ID NO: 104 - protein, 8 aa, Artificial Sequence
  Description of Artificial Sequence: Synthetic peptide
INTETGEP

SEQ ID NO: 105 - protein, 12 aa, Artificial Sequence
  Description of Artificial Sequence: Synthetic peptide
ATYYGSSYVPDY

SEQ ID NO: 106 - protein, 6 aa, Artificial Sequence
  Description of Artificial Sequence: Synthetic peptide
QNVGTA

SEQ ID NO: 107 - protein, 3 aa, Artificial Sequence
  Description of Artificial Sequence: Synthetic peptide
WTS

SEQ ID NO: 108 - protein, 8 aa, Artificial Sequence
  Description of Artificial Sequence: Synthetic peptide
QYSDYPYT

SEQ ID NO: 109 - protein, 7 aa, Artificial Sequence
  Description of Artificial Sequence: Synthetic peptide
GYTFTDY

SEQ ID NO: 110 - protein, 8 aa, Artificial Sequence
  Description of Artificial Sequence: Synthetic peptide
WVDPEYGS

SEQ ID NO: 111 - protein, 12 aa, Artificial Sequence
  Description of Artificial Sequence: Synthetic peptide
ARDDGSYSPFDY

SEQ ID NO: 112 - protein, 6 aa, Artificial Sequence
  Description of Artificial Sequence: Synthetic peptide
QNINKY

SEQ ID NO: 113 - protein, 3 aa, Artificial Sequence
  Description of Artificial Sequence: Synthetic peptide
YTS

SEQ ID NO: 114 - protein, 8 aa, Artificial Sequence
  Description of Artificial Sequence: Synthetic peptide
LQYVNLLT

SEQ ID NO: 115 - protein, 6 aa, Artificial Sequence
  Description of Artificial Sequence: Synthetic peptide
ENVVTY

SEQ ID NO: 116 - protein, 3 aa, Artificial Sequence
  Description of Artificial Sequence: Synthetic peptide
GAS

SEQ ID NO: 117 - protein, 8 aa, Artificial Sequence
  Description of Artificial Sequence: Synthetic peptide
QGYSYPYT

SEQ ID NO: 118 - protein, 10 aa, Artificial Sequence
  Description of Artificial Sequence: Synthetic peptide
GGSISSPGYY

SEQ ID NO: 119 - protein, 7 aa, Artificial Sequence
  Description of Artificial Sequence: Synthetic peptide
IYKSGST

SEQ ID NO: 120 - protein, 13 aa, Artificial Sequence
  Description of Artificial Sequence: Synthetic peptide
TRPVVRYFGWFDP

SEQ ID NO: 121 - protein, 6 aa, Artificial Sequence
  Description of Artificial Sequence: Synthetic peptide
QGISSA

SEQ ID NO: 122 - protein, 3 aa, Artificial Sequence
  Description of Artificial Sequence: Synthetic peptide
DAS

SEQ ID NO: 123 - protein, 8 aa, Artificial Sequence
  Description of Artificial Sequence: Synthetic peptide
QQFNSYPT

SEQ ID NO: 124 - protein, 8 aa, Artificial Sequence
  Description of Artificial Sequence: Synthetic peptide
GFTFSNYG

SEQ ID NO: 125 - protein, 8 aa, Artificial Sequence
  Description of Artificial Sequence: Synthetic peptide
IWYDGSNK

SEQ ID NO: 126 - protein, 11 aa, Artificial Sequence
  Description of Artificial Sequence: Synthetic peptide
ARDLWGWYFDY

SEQ ID NO: 127 - protein, 6 aa, Artificial Sequence
  Description of Artificial Sequence: Synthetic peptide
QSVSSY

SEQ ID NO: 128 - protein, 3 aa, Artificial Sequence
  Description of Artificial Sequence: Synthetic peptide
DAS

SEQ ID NO: 129 - protein, 9 aa, Artificial Sequence
  Description of Artificial Sequence: Synthetic peptide
QQRRNWPLT

SEQ ID NO: 130 - protein, 8 aa, Artificial Sequence
  Description of Artificial Sequence: Synthetic peptide
GDSFTTYW

SEQ ID NO: 131 - protein, 8 aa, Artificial Sequence
  Description of Artificial Sequence: Synthetic peptide
IYPGDSDT

SEQ ID NO: 132 - protein, 9 aa, Artificial Sequence
  Description of Artificial Sequence: Synthetic peptide
TRGDRGVDY

SEQ ID NO: 133 - protein, 6 aa, Artificial Sequence
  Description of Artificial Sequence: Synthetic peptide
QGISRW

SEQ ID NO: 134 - protein, 3 aa, Artificial Sequence
  Description of Artificial Sequence: Synthetic peptide
AAS

SEQ ID NO: 135 - protein, 9 aa, Artificial Sequence
  Description of Artificial Sequence: Synthetic peptide
QQYNSYPRT

SEQ ID NO: 136 - protein, 10 aa, Artificial Sequence
  Description of Artificial Sequence: Synthetic peptide
GFSLSTSGMG

SEQ ID NO: 137 - protein, 7 aa, Artificial Sequence
  Description of Artificial Sequence: Synthetic peptide
IWWDDDK

SEQ ID NO: 138 - protein, 11 aa, Artificial Sequence
  Description of Artificial Sequence: Synthetic peptide
ARITGTRYFDY

SEQ ID NO: 139 - protein, 6 aa, Artificial Sequence
  Description of Artificial Sequence: Synthetic peptide
QDINKF

SEQ ID NO: 140 - protein, 3 aa, Artificial Sequence
  Description of Artificial Sequence: Synthetic peptide
YTS

SEQ ID NO: 141 - protein, 8 aa, Artificial Sequence
  Description of Artificial Sequence: Synthetic peptide
LQYGNLWT

SEQ ID NO: 142 - protein, 8 aa, Artificial Sequence
  Description of Artificial Sequence: Synthetic peptide
GYSFTDYN

SEQ ID NO: 143 - protein, 8 aa, Artificial Sequence
  Description of Artificial Sequence: Synthetic peptide
IDPYYGGT

SEQ ID NO: 144 - protein, 18 aa, Artificial Sequence
  Description of Artificial Sequence: Synthetic peptide
ARWDYRYDDGRAYYVMDF

SEQ ID NO: 145 - protein, 12 aa, Artificial Sequence
  Description of Artificial Sequence: Synthetic peptide
QSVLYSSNQKNY

SEQ ID NO: 146 - protein, 3 aa, Artificial Sequence
  Description of Artificial Sequence: Synthetic peptide
WAS

SEQ ID NO: 147 - protein, 8 aa, Artificial Sequence
  Description of Artificial Sequence: Synthetic peptide
HQYLYSWT

SEQ ID NO: 148 - protein, 8 aa, Artificial Sequence
  Description of Artificial Sequence: Synthetic peptide
GYSFTDYN

SEQ ID NO: 149 - protein, 8 aa, Artificial Sequence
  Description of Artificial Sequence: Synthetic peptide
IDPYYGGT

SEQ ID NO: 150 - protein, 18 aa, Artificial Sequence
  Description of Artificial Sequence: Synthetic peptide
ARWDYRYDDGRAYYVMDF

SEQ ID NO: 151 - protein, 12 aa, Artificial Sequence
  Description of Artificial Sequence: Synthetic peptide
QSVLYSSNQKNY

SEQ ID NO: 152 - protein, 3 aa, Artificial Sequence
  Description of Artificial Sequence: Synthetic peptide
WAS

SEQ ID NO: 153 - protein, 8 aa, Artificial Sequence
  Description of Artificial Sequence: Synthetic peptide
HQYLYSWT

SEQ ID NO: 154 - protein, 9 aa, Artificial Sequence
  Description of Artificial Sequence: Synthetic peptide
GYTFTNYII

SEQ ID NO: 155 - protein, 8 aa, Artificial Sequence
  Description of Artificial Sequence: Synthetic peptide
FNPYNHGT

SEQ ID NO: 156 - protein, 11 aa, Artificial Sequence
  Description of Artificial Sequence: Synthetic peptide
ARSGPYAWFDT

SEQ ID NO: 157 - protein, 6 aa, Artificial Sequence
  Description of Artificial Sequence: Synthetic peptide
QNIGTS

SEQ ID NO: 158 - protein, 3 aa, Artificial Sequence
  Description of Artificial Sequence: Synthetic peptide
SSS

SEQ ID NO: 159 - protein, 9 aa, Artificial Sequence
  Description of Artificial Sequence: Synthetic peptide
QQSNTWPFT

SEQ ID NO: 160 - protein, 9 aa, Artificial Sequence
  Description of Artificial Sequence: Synthetic peptide
GYTFTNYII

SEQ ID NO: 161 - protein, 8 aa, Artificial Sequence
  Description of Artificial Sequence: Synthetic peptide
FNPYNHGT

SEQ ID NO: 162 - protein, 11 aa, Artificial Sequence
  Description of Artificial Sequence: Synthetic peptide
ARSGPYAWFDT

SEQ ID NO: 163 - protein, 6 aa, Artificial Sequence
  Description of Artificial Sequence: Synthetic peptide
QNIGTS

SEQ ID NO: 164 - protein, 3 aa, Artificial Sequence
  Description of Artificial Sequence: Synthetic peptide
SSS

SEQ ID NO: 165 - protein, 9 aa, Artificial Sequence
  Description of Artificial Sequence: Synthetic peptide
QQSNTWPFT

SEQ ID NO: 166 - protein, 8 aa, Artificial Sequence
  Description of Artificial Sequence: Synthetic peptide
GFTFSNYG

SEQ ID NO: 167 - protein, 8 aa, Artificial Sequence
  Description of Artificial Sequence: Synthetic peptide
IWYDGSKK

SEQ ID NO: 168 - protein, 9 aa, Artificial Sequence
  Description of Artificial Sequence: Synthetic peptide
ARGGGDFDF

SEQ ID NO: 169 - protein, 7 aa, Artificial Sequence
  Description of Artificial Sequence: Synthetic peptide
QSVSGNY

SEQ ID NO: 170 - protein, 3 aa, Artificial Sequence
  Description of Artificial Sequence: Synthetic peptide
GAS

SEQ ID NO: 171 - protein, 10 aa, Artificial Sequence
  Description of Artificial Sequence: Synthetic peptide
QQYGKWPPLT

SEQ ID NO: 172 - protein, 10 aa, Artificial Sequence
  Description of Artificial Sequence: Synthetic peptide
GFSLSTSGVG

SEQ ID NO: 173 - protein, 7 aa, Artificial Sequence
  Description of Artificial Sequence: Synthetic peptide
IDWDDDK

SEQ ID NO: 174 - protein, 12 aa, Artificial Sequence
  Description of Artificial Sequence: Synthetic peptide
ARSPRYRGAFDY

SEQ ID NO: 175 - protein, 8 aa, Artificial Sequence
  Description of Artificial Sequence: Synthetic peptide
ESNIGNNY

SEQ ID NO: 176 - protein, 3 aa, Artificial Sequence
  Description of Artificial Sequence: Synthetic peptide
DNN

SEQ ID NO: 177 - protein, 9 aa, Artificial Sequence
  Description of Artificial Sequence: Synthetic peptide
QSYDLIRHV

SEQ ID NO: 178 - protein, 10 aa, Artificial Sequence
  Description of Artificial Sequence: Synthetic peptide
GFSLSTSGVG

SEQ ID NO: 179 - protein, 7 aa, Artificial Sequence
  Description of Artificial Sequence: Synthetic peptide
IDWDDDK

SEQ ID NO: 180 - protein, 12 aa, Artificial Sequence
  Description of Artificial Sequence: Synthetic peptide
ARSPRYRGAFDY

SEQ ID NO: 181 - protein, 8 aa, Artificial Sequence
  Description of Artificial Sequence: Synthetic peptide
ESNIGNNY

SEQ ID NO: 182 - protein, 3 aa, Artificial Sequence
  Description of Artificial Sequence: Synthetic peptide
DNN

SEQ ID NO: 183 - protein, 8 aa, Artificial Sequence
  Description of Artificial Sequence: Synthetic peptide
QSYDMNVH

SEQ ID NO: 184 - protein, 10 aa, Artificial Sequence
  Description of Artificial Sequence: Synthetic peptide
GFSLSTSGMG

SEQ ID NO: 185 - protein, 6 aa, Artificial Sequence
  Description of Artificial Sequence: Synthetic peptide
IYWDDK

SEQ ID NO: 186 - protein, 17 aa, Artificial Sequence
  Description of Artificial Sequence: Synthetic peptide
ARSSHYYGYGYGGYFDV

SEQ ID NO: 187 - protein, 10 aa, Artificial Sequence
  Description of Artificial Sequence: Synthetic peptide
ESIHSYGNSF

SEQ ID NO: 188 - protein, 3 aa, Artificial Sequence
  Description of Artificial Sequence: Synthetic peptide
LAS

SEQ ID NO: 189 - protein, 9 aa, Artificial Sequence
  Description of Artificial Sequence: Synthetic peptide
QQNNEDPWT

SEQ ID NO: 190 - protein, 8 aa, Artificial Sequence
  Description of Artificial Sequence: Synthetic peptide
GFTFSSYD

SEQ ID NO: 191 - protein, 8 aa, Artificial Sequence
  Description of Artificial Sequence: Synthetic peptide
VSSGGGST

SEQ ID NO: 192 - protein, 10 aa, Artificial Sequence
  Description of Artificial Sequence: Synthetic peptide
ARHLHGSFAS

SEQ ID NO: 193 - protein, 7 aa, Artificial Sequence
  Description of Artificial Sequence: Synthetic peptide
QSISNFL

SEQ ID NO: 194 - protein, 3 aa, Artificial Sequence
  Description of Artificial Sequence: Synthetic peptide
YRS

SEQ ID NO: 195 - protein, 9 aa, Artificial Sequence
  Description of Artificial Sequence: Synthetic peptide
QQSGSWPLT

SEQ ID NO: 196 - protein, 8 aa, Artificial Sequence
  Description of Artificial Sequence: Synthetic peptide
GFVFSRYW

SEQ ID NO: 197 - protein, 8 aa, Artificial Sequence
  Description of Artificial Sequence: Synthetic peptide
INPDSSTI

SEQ ID NO: 198 - protein, 9 aa, Artificial Sequence
  Description of Artificial Sequence: Synthetic peptide
ASLITTEDY

SEQ ID NO: 199 - protein, 6 aa, Artificial Sequence
  Description of Artificial Sequence: Synthetic peptide
QDINSY

SEQ ID NO: 200 - protein, 3 aa, Artificial Sequence
  Description of Artificial Sequence: Synthetic peptide
YAN

SEQ ID NO: 201 - protein, 9 aa, Artificial Sequence
  Description of Artificial Sequence: Synthetic peptide
LQYDEFPYT

SEQ ID NO: 202 - protein, 8 aa, Artificial Sequence
  Description of Artificial Sequence: Synthetic peptide
GFTFSRYT

SEQ ID NO: 203 - protein, 8 aa, Artificial Sequence
  Description of Artificial Sequence: Synthetic peptide
ISFDGSNK

SEQ ID NO: 204 - protein, 12 aa, Artificial Sequence
  Description of Artificial Sequence: Synthetic peptide
AREARGSYAFDI

SEQ ID NO: 205 - protein, 6 aa, Artificial Sequence
  Description of Artificial Sequence: Synthetic peptide
QSVSSY

SEQ ID NO: 206 - protein, 3 aa, Artificial Sequence
  Description of Artificial Sequence: Synthetic peptide
DAS

SEQ ID NO: 207 - protein, 10 aa, Artificial Sequence
  Description of Artificial Sequence: Synthetic peptide
QQRSNWPPFT

SEQ ID NO: 208 - protein, 8 aa, Artificial Sequence
  Description of Artificial Sequence: Synthetic peptide
GYTFSSFW

SEQ ID NO: 209 - protein, 8 aa, Artificial Sequence
  Description of Artificial Sequence: Synthetic peptide
INPRSGYT

SEQ ID NO: 210 - protein, 11 aa, Artificial Sequence
  Description of Artificial Sequence: Synthetic peptide
ASFLGRGAMDY

SEQ ID NO: 211 - protein, 6 aa, Artificial Sequence
  Description of Artificial Sequence: Synthetic peptide
QDISNY

SEQ ID NO: 212 - protein, 3 aa, Artificial Sequence
  Description of Artificial Sequence: Synthetic peptide
YTS

SEQ ID NO: 213 - protein, 9 aa, Artificial Sequence
  Description of Artificial Sequence: Synthetic peptide
QQGNTFPYT

SEQ ID NO: 214 - protein, 10 aa, Artificial Sequence
  Description of Artificial Sequence: Synthetic peptide
GGSISSGVYY

SEQ ID NO: 215 - protein, 7 aa, Artificial Sequence
  Description of Artificial Sequence: Synthetic peptide
IYYSGST

SEQ ID NO: 216 - protein, 16 aa, Artificial Sequence
  Description of Artificial Sequence: Synthetic peptide
AREGPLRGDYYYGLDV

SEQ ID NO: 217 - protein, 7 aa, Artificial Sequence
  Description of Artificial Sequence: Synthetic peptide
QTISSRY

SEQ ID NO: 218 - protein, 3 aa, Artificial Sequence
  Description of Artificial Sequence: Synthetic peptide
GAS

SEQ ID NO: 219 - protein, 9 aa, Artificial Sequence
  Description of Artificial Sequence: Synthetic peptide
QQYGSSPRT

SEQ ID NO: 220 - protein, 8 aa, Artificial Sequence
  Description of Artificial Sequence: Synthetic peptide
GFTFSSYA

SEQ ID NO: 221 - protein, 8 aa, Artificial Sequence
  Description of Artificial Sequence: Synthetic peptide
ISGSGGRT

SEQ ID NO: 222 - protein, 10 aa, Artificial Sequence
  Description of Artificial Sequence: Synthetic peptide
ARLGYGRVDE

SEQ ID NO: 223 - protein, 8 aa, Artificial Sequence
  Description of Artificial Sequence: Synthetic peptide
LSNIGRNP

SEQ ID NO: 224 - protein, 3 aa, Artificial Sequence
  Description of Artificial Sequence: Synthetic peptide
LDN

SEQ ID NO: 225 - protein, 11 aa, Artificial Sequence
  Description of Artificial Sequence: Synthetic peptide
ATWDDSHPGWT

SEQ ID NO: 226 - protein, 8 aa, Artificial Sequence
  Description of Artificial Sequence: Synthetic peptide
GFTFSNYG

SEQ ID NO: 227 - protein, 8 aa, Artificial Sequence
  Description of Artificial Sequence: Synthetic peptide
ILYDGSNK

SEQ ID NO: 228 - protein, 14 aa, Artificial Sequence
  Description of Artificial Sequence: Synthetic peptide
ARGGSSWYPDSFDI

SEQ ID NO: 229 - protein, 6 aa, Artificial Sequence
  Description of Artificial Sequence: Synthetic peptide
QGISSW

SEQ ID NO: 230 - protein, 3 aa, Artificial Sequence
  Description of Artificial Sequence: Synthetic peptide
AAS

SEQ ID NO: 231 - protein, 9 aa, Artificial Sequence
  Description of Artificial Sequence: Synthetic peptide
QQYNSYPLT

SEQ ID NO: 232 - protein, 8 aa, Artificial Sequence
  Description of Artificial Sequence: Synthetic peptide
GFTFNSFA

SEQ ID NO: 233 - skipped

SEQ ID NO: 234 - protein, 8 aa, Artificial Sequence
  Description of Artificial Sequence: Synthetic peptide
ISGSGGGT

SEQ ID NO: 235 - protein, 15 aa, Artificial Sequence
  Description of Artificial Sequence: Synthetic peptide
AKDKILWFGEPVFDY

SEQ ID NO: 236 - protein, 6 aa, Artificial Sequence
  Description of Artificial Sequence: Synthetic peptide
QSVSSY

SEQ ID NO: 237 - protein, 3 aa, Artificial Sequence
  Description of Artificial Sequence: Synthetic peptide
DAS

SEQ ID NO: 238 - protein, 8 aa, Artificial Sequence
  Description of Artificial Sequence: Synthetic peptide
QRSNWPPT

SEQ ID NO: 239 - protein, 8 aa, Artificial Sequence
  Description of Artificial Sequence: Synthetic peptide
GFSLTNYG

SEQ ID NO: 240 - protein, 7 aa, Artificial Sequence
  Description of Artificial Sequence: Synthetic peptide
IWARGFT

SEQ ID NO: 241 - protein, 13 aa, Artificial Sequence
  Description of Artificial Sequence: Synthetic peptide
ARANDGVYYAMDY

SEQ ID NO: 242 - protein, 6 aa, Artificial Sequence
  Description of Artificial Sequence: Synthetic peptide
QSSVNY

SEQ ID NO: 243 - protein, 3 aa, Artificial Sequence
  Description of Artificial Sequence: Synthetic peptide
DTS

SEQ ID NO: 244 - protein, 9 aa, Artificial Sequence
  Description of Artificial Sequence: Synthetic peptide
QQWTTNPLT

SEQ ID NO: 245 - protein, 8 aa, Artificial Sequence
  Description of Artificial Sequence: Synthetic peptide
GYTFTSYW

SEQ ID NO: 246 - protein, 8 aa, Artificial Sequence
  Description of Artificial Sequence: Synthetic peptide
IDPSESNT

SEQ ID NO: 247 - protein, 14 aa, Artificial Sequence
  Description of Artificial Sequence: Synthetic peptide
ARGGYDGWDYAIDY

SEQ ID NO: 248 - protein, 11 aa, Artificial Sequence
  Description of Artificial Sequence: Synthetic peptide
QSLAKSYGNTY

SEQ ID NO: 249 - protein, 3 aa, Artificial Sequence
  Description of Artificial Sequence: Synthetic peptide
GIS

SEQ ID NO: 250 - protein, 9 aa, Artificial Sequence
  Description of Artificial Sequence: Synthetic peptide
LQGTHQPYT

SEQ ID NO: 251 - protein, 8 aa, Artificial Sequence
  Description of Artificial Sequence: Synthetic peptide
GFFITNNY

SEQ ID NO: 252 - protein, 7 aa, Artificial Sequence
  Description of Artificial Sequence: Synthetic peptide
ISYSGST

SEQ ID NO: 253 - protein, 11 aa, Artificial Sequence
  Description of Artificial Sequence: Synthetic peptide
ARTGSSGYFDF

SEQ ID NO: 254 - protein, 7 aa, Artificial Sequence
  Description of Artificial Sequence: Synthetic peptide
ESVDDLL

SEQ ID NO: 255 - protein, 3 aa, Artificial Sequence
  Description of Artificial Sequence: Synthetic peptide
YAS

SEQ ID NO: 256 - protein, 9 aa, Artificial Sequence
  Description of Artificial Sequence: Synthetic peptide
QQGNSLPNT

SEQ ID NO: 257 - protein, 8 aa, Artificial Sequence
  Description of Artificial Sequence: Synthetic peptide
GYIFTNYW

SEQ ID NO: 258 - protein, 8 aa, Artificial Sequence
  Description of Artificial Sequence: Synthetic peptide
IYPGDSDI

SEQ ID NO: 259 - protein, 10 aa, Artificial Sequence
  Description of Artificial Sequence: Synthetic peptide
ARHDIEGFDY

SEQ ID NO: 260 - protein, 7 aa, Artificial Sequence
  Description of Artificial Sequence: Synthetic peptide
QSVSSSF

SEQ ID NO: 261 - protein, 3 aa, Artificial Sequence
  Description of Artificial Sequence: Synthetic peptide
GAS

SEQ ID NO: 262 - protein, 9 aa, Artificial Sequence
  Description of Artificial Sequence: Synthetic peptide
QQYDSSAIT

SEQ ID NO: 263 - protein, 9 aa, Artificial Sequence
  Description of Artificial Sequence: Synthetic peptide
GFTFTYTMS

SEQ ID NO: 264 - protein, 7 aa, Artificial Sequence
  Description of Artificial Sequence: Synthetic peptide
PGDSFGY

SEQ ID NO: 265 - protein, 14 aa, Artificial Sequence
  Description of Artificial Sequence: Synthetic peptide
TRDIYYNYGAWFAY

SEQ ID NO: 266 - protein, 10 aa, Artificial Sequence
  Description of Artificial Sequence: Synthetic peptide
QSVDYDGDSY

SEQ ID NO: 267 - protein, 3 aa, Artificial Sequence
  Description of Artificial Sequence: Synthetic peptide
AAS

SEQ ID NO: 268 - protein, 9 aa, Artificial Sequence
  Description of Artificial Sequence: Synthetic peptide
QQANEDPRT

SEQ ID NO: 269 - protein, 8 aa, Artificial Sequence
  Description of Artificial Sequence: Synthetic peptide
GYTFTDYY

SEQ ID NO: 270 - protein, 8 aa, Artificial Sequence
  Description of Artificial Sequence: Synthetic peptide
IYPGSGNT

SEQ ID NO: 271 - protein, 10 aa, Artificial Sequence
  Description of Artificial Sequence: Synthetic peptide
ANYGNYWFAY

SEQ ID NO: 272 - protein, 10 aa, Artificial Sequence
  Description of Artificial Sequence: Synthetic peptide
QSVDFDGDSY

SEQ ID NO: 273 - protein, 3 aa, Artificial Sequence
  Description of Artificial Sequence: Synthetic peptide
AAS

SEQ ID NO: 274 - protein, 9 aa, Artificial Sequence
  Description of Artificial Sequence: Synthetic peptide
QQSNEDPWT

SEQ ID NO: 275 - protein, 8 aa, Artificial Sequence
  Description of Artificial Sequence: Synthetic peptide
GGSFSAYY

SEQ ID NO: 276 - protein, 7 aa, Artificial Sequence
  Description of Artificial Sequence: Synthetic peptide
INHGGGT

SEQ ID NO: 277 - protein, 6 aa, Artificial Sequence
  Description of Artificial Sequence: Synthetic peptide
ASLTAY

SEQ ID NO: 278 - protein, 6 aa, Artificial Sequence
  Description of Artificial Sequence: Synthetic peptide
QGISSW

SEQ ID NO: 279 - protein, 3 aa, Artificial Sequence
  Description of Artificial Sequence: Synthetic peptide
AAS

SEQ ID NO: 280 - protein, 9 aa, Artificial Sequence
  Description of Artificial Sequence: Synthetic peptide
QQYDSYPIT

SEQ ID NO: 281 - protein, 8 aa, Artificial Sequence
  Description of Artificial Sequence: Synthetic peptide
GYTFTSYN

SEQ ID NO: 282 - protein, 8 aa, Artificial Sequence
  Description of Artificial Sequence: Synthetic peptide
IYSGNGDT

SEQ ID NO: 283 - protein, 10 aa, Artificial Sequence
  Description of Artificial Sequence: Synthetic peptide
ARERDTRFGN

SEQ ID NO: 284 - protein, 10 aa, Artificial Sequence
  Description of Artificial Sequence: Synthetic peptide
ESVDIYGNSF

SEQ ID NO: 285 - protein, 3 aa, Artificial Sequence
  Description of Artificial Sequence: Synthetic peptide
LAS

SEQ ID NO: 286 - protein, 9 aa, Artificial Sequence
  Description of Artificial Sequence: Synthetic peptide
QQNNEDPYT

SEQ ID NO: 287 - protein, 8 aa, Artificial Sequence
  Description of Artificial Sequence: Synthetic peptide
GITFSNSG

SEQ ID NO: 288 - protein, 8 aa, Artificial Sequence
  Description of Artificial Sequence: Synthetic peptide
IWYDGSKR

SEQ ID NO: 289 - protein, 6 aa, Artificial Sequence
  Description of Artificial Sequence: Synthetic peptide
ATNDDY

SEQ ID NO: 290 - protein, 7 aa, Artificial Sequence
  Description of Artificial Sequence: Synthetic peptide
QSVSSYL

SEQ ID NO: 291 - protein, 3 aa, Artificial Sequence
  Description of Artificial Sequence: Synthetic peptide
DAS

SEQ ID NO: 292 - protein, 9 aa, Artificial Sequence
  Description of Artificial Sequence: Synthetic peptide
QQSSNWPRT

SEQ ID NO: 293 - protein, 8 aa, Artificial Sequence
  Description of Artificial Sequence: Synthetic peptide
GYTFTNYY

SEQ ID NO: 294 - protein, 8 aa, Artificial Sequence
  Description of Artificial Sequence: Synthetic peptide
INPSNGGT

SEQ ID NO: 295 - protein, 13 aa, Artificial Sequence
  Description of Artificial Sequence: Synthetic peptide
ARRDYRFDMGFDY

SEQ ID NO: 296 - protein, 10 aa, Artificial Sequence
  Description of Artificial Sequence: Synthetic peptide
KGVSTSGYSY

SEQ ID NO: 297 - protein, 3 aa, Artificial Sequence
  Description of Artificial Sequence: Synthetic peptide
LAS

SEQ ID NO: 298 - protein, 9 aa, Artificial Sequence
  Description of Artificial Sequence: Synthetic peptide
QHSRDLPLT

SEQ ID NO: 299 - protein, 107 aa, Artificial Sequence
  Description of Artificial Sequence: Synthetic polypeptide
EIVMTQSPATLSVSPGERATLSCKASQNVGTNVAWYQQKPGQAPRLLIYSASYRYSGIPARFSGSGSGTEFTLTISSLQSEDFAVYYCQQYNTDPLTFGGGTKVEIK

SEQ ID NO: 300 - protein, 119 aa, Artificial Sequence
  Description of Artificial Sequence: Synthetic polypeptide
QVTLRESGPALVKPTQTLTLTCTFSGFSLSTSGMGVGWIRQPPGKALEWLAHIWWDDDKYYNPSLKSRLTISKDTSKNQVVLTMTNMDPVDTATYYCARTRRYFPFAYWGQGTLVTVSS

SEQ ID NO: 301 - protein, 119 aa, Artificial Sequence
  Description of Artificial Sequence: Synthetic polypeptide
QVTLRESGPALVKPTQTLTLTCTFSGFSLSTSGMGVGWIRQPPGKALEWLAHIWWDDDKYYQPSLKSRLTISKDTSKNQVVLTMTNMDPVDTATYYCARTRRYFPFAYWGQGTLVTVSS

SEQ ID NO: 302 - protein, 120 aa, Artificial Sequence
  Description of Artificial Sequence: Synthetic polypeptide
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 303 - protein, 107 aa, Artificial Sequence
  Description of Artificial Sequence: Synthetic polypeptide
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 304 - protein, 121 aa, Artificial Sequence [sequence omitted]

SEQ ID NO: 305 - protein, 109 aa, Artificial Sequence [sequence omitted]

SEQ ID NO: 306 - protein, 114 aa, Artificial Sequence
  Description of Artificial Sequence: Synthetic polypeptide
EVQLVESGGGLVQPGGSLRLSCAASGFTFSDYWMSWVRQAPGKGLEWVADIKNDGSYTNYAPSLTNRFTISRDNAKNSLYLQMNSLRAEDTAVYYCARELTGTWGQGTMVTVSS

SEQ ID NO: 307 - protein, 113 aa, Artificial Sequence
  Description of Artificial Sequence: Synthetic polypeptide
DIVMTQSPDSLAVSLGERATINCKSSQSLLSSGNQKNYLAWYQQKPGQPPKLLIYYASTRQSGVPDRFSGSGSGTDFTLTISSLQAEDVAVYYCLQYDRYPFTFGQGTKLEIK

SEQ ID NO: 308 - protein, 116 aa, Artificial Sequence
  Description of Artificial Sequence: Synthetic polypeptide
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 309 - protein, 108 aa, Artificial Sequence
  Description of Artificial Sequence: Synthetic polypeptide
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 310 - protein, 118 aa, Artificial Sequence
  Description of Artificial Sequence: Synthetic polypeptide
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 311 - protein, 111 aa, Artificial Sequence
  Description of Artificial Sequence: Synthetic polypeptide
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 312 - protein, 122 aa, Artificial Sequence
  Description of Artificial Sequence: Synthetic polypeptide
QLQLQESGPGLVKPSETLSLTCTVSGGSITSSSYYWGWIRQPPGKGLEWIGSIYYRGSTNYNPSLKSRVTISVDSSKNQFYLKVSSVTAVDTAVYYCARQNGAARPSWFDPWGQGTLVTVSS

SEQ ID NO: 313 - protein, 108 aa, Artificial Sequence
  Description of Artificial Sequence: Synthetic polypeptide
EIVLTQSPDTISLSPGERATLSCRASQSISSIYLAWYQQKPGQAPRLLIYGASSRVTGIPDRFSVSGSGTDFTLTISRLEPEDFAVYYCQHYGISPFTFGPGTKVDIR

SEQ ID NO: 314 - protein, 117 aa, Artificial Sequence
  Description of Artificial Sequence: Synthetic polypeptide
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSS

SEQ ID NO: 315 - protein, 110 aa, Artificial Sequence
  Description of Artificial Sequence: Synthetic polypeptide
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVL

SEQ ID NO: 316 - protein, 124 aa, Artificial Sequence
  Description of Artificial Sequence: Synthetic polypeptide
QVQLQESGPGLVRPSQTLSLTCTASGYTFTDYVIHWVKQPPGRGLEWIGYINPYDDDTTYNQKFKGRVTMLVDTSSNTAYLRLSSVTAEDTAVYYCARRGNSYDGYFDYSMDYWGSGTPVTVSS

SEQ ID NO: 317 - protein, 108 aa, Artificial Sequence
  Description of Artificial Sequence: Synthetic polypeptide
DIQMTQSPSSLSASVGDRVTITCRASQNVGTAVAWLQQTPGKAPKLLIYSASNRYTGVPSRFSGSGSGTDYTFTISSLQPEDIATYYCQQYTNYPMYTFGQGTKVQIK

SEQ ID NO: 318 - protein, 121 aa, Artificial Sequence
  Description of Artificial Sequence: Synthetic polypeptide
QVQLQESGPGLVKPSQTLSLTCAISGDSVSSNSVTWNWIRQSPSRGLEWLGRTYYRSKWYNDYAVSVKGRITINPDTSKNQFYLQLKSVTPEDAAVYYCARDSSILYGDYWGQGTLVTVSS

SEQ ID NO: 319 - protein, 120 aa, Artificial Sequence
  Description of Artificial Sequence: Synthetic polypeptide
QVQLQQSGPGLVKPSQTLSLTCAISGDSVSSNSVTWNWIRQSPSRGLEWLGRTYYRSKWYNDYAVSVKGRITINPDTSKNQFYLQLKSVTPEDAAVYYCARDSSILYGDYWGQGTLVTVS

SEQ ID NO: 320 - protein, 115 aa, Artificial Sequence
  Description of Artificial Sequence: Synthetic polypeptide
QAVLTQPSSLSASPGASASLTCTLPSGINVGTYRIFWFQQKPGSPPQYLLSYKSDSDNHQGSGVPSRFSGSKDASANAGILLISGLQSEDEADYYCMIWHSSAWVFGGGTKLTVL

SEQ ID NO: 321 - protein, 122 aa, Artificial Sequence
  Description of Artificial Sequence: Synthetic polypeptide
QVQLVQSGAEVKKPGASVKVSCKTSGYTFTDYYIHWVRQAPGQGLEWMGWINPNRGGTNYAQKFQGRVTMTRDTSIATAYMELSRLRSDDTAVYYCATASLKIAAVGTFDCWGQGTLVTVSS

SEQ ID NO: 322 - protein, 107 aa, Artificial Sequence
  Description of Artificial Sequence: Synthetic polypeptide
SYELTQPPSVSVSPGQTARITCSGDALSKQYAFWFQQKPGQAPILVIYQDTKRPSGIPGRFSGSSSGTTVTLTISGAQADDEADYYCQSADSSGTYVFGTGTKVTVL

SEQ ID NO: 323 - protein, 116 aa, Artificial Sequence
  Description of Artificial Sequence: Synthetic polypeptide
EVQLVETGGGVVQPGRSLRLSCAASGFSFSTHGMYWVRQPPGKGLEWVAVISYDGSDKKYADSVKGRFTISRDNSKNTVYLEMSSVRAEDTALYYCFCRRDAFDLWGQGTMVTVSS

SEQ ID NO: 324 - protein, 108 aa, Artificial Sequence
  Description of Artificial Sequence: Synthetic polypeptide
SYVLTQPPSVSVSPGQTARITCSGDALPKKYAYWYQQKSGQAPVLVIYEDTKRPSGIPERFSGSSSGTMATLTISGAQVEDEADYYCYSTDSSGNYWVFGGGTEVTVL

SEQ ID NO: 325 - protein, 124 aa, Artificial Sequence
  Description of Artificial Sequence: Synthetic polypeptide
EVQLVESGGGLVEPGGSLRLSCAASGFTFSDAWMNWVRQAPGKGLEWVGRIKTKSDGGTTDYAAPVRGRFSISRDDSKNTLFLEMNSLKTEDTAIYYCFITVIVVSSESPLDHWGQGTLVTVSS

SEQ ID NO: 326 - protein, 108 aa, Artificial Sequence
  Description of Artificial Sequence: Synthetic polypeptide
SYELTQPPSVSVSPGQTARITCSGDELPKQYAYWYQQKPGQAPVLVIYKDRQRPSGIPERFSGSSSGTTVTLTISGVQAEDEADYYCQSAYSINTYVIFGGGTKLTVL

SEQ ID NO: 327 - protein, 125 aa, Artificial Sequence
  Description of Artificial Sequence: Synthetic polypeptide
EVQLVESGGGLVKPGGSLRLSCAASGFTFSDYYMSWIRQAPGKGLEWISYISSGSSYTNYADSVKGRFTISRDNAKKSVYLEVNGLTVEDTAVYYCARVRYGDREMATIGGFDFWGQGTLVTVSS

SEQ ID NO: 328 - protein, 108 aa, Artificial Sequence
  Description of Artificial Sequence: Synthetic polypeptide
SYELTQPPSVSVSPGQTARITCSGDALPKQYAYWYQQSPGQAPVLVIYKDSERPSGIPERFSGSSSGTTVTLTISGVQAEDEADYYCQSADSGGTSRIFGGGTKLTVL

SEQ ID NO: 329 - protein, 124 aa, Artificial Sequence
  Description of Artificial Sequence: Synthetic polypeptide
QVQLQESGPGLVRSTETLSLTCLVSGDSINSHYWSWLRQSPGRGLEWIGYIYYTGPTNYNPSLKSRVSISLGTSKDQFSLKLSSVTAADTARYYCARNKVFWRGSDFYYYMDVWGKGTTVTVSS

SEQ ID NO: 330 - protein, 110 aa, Artificial Sequence
  Description of Artificial Sequence: Synthetic polypeptide
EIVLTQSPGTLSLSLGERATLSCRASQSLANNYLAWYQQKPGQAPRLLMYDASTRATGIPDRFSGSGSGTDFTLTISRLEPEDFAVYYCQQFVTSHHMYIFGQGTKVEIK

SEQ ID NO: 331 - protein, 129 aa, Artificial Sequence
  Description of Artificial Sequence: Synthetic polypeptide
HVQLQESGPGLVKPSETLSLTCTVSGGSISSNNYYWGWIRQTPGKGLEWIGSIYYSGSTNYNPSLKSRVTISVDTSKNQFSLKLSSVTAADTAVYYCARGARWQARPATRIDGVAFDIWGQGTMVTVSS

SEQ ID NO: 332 - protein, 129 aa, Artificial Sequence
  Description of Artificial Sequence: Synthetic polypeptide
QVQLQESGPGLVKPSETLSLTCTVSGGSISSNNYYWGWIRQTPGKGLEWIGSIYYSGSTNYNPSLKSRVTISVDTSKNQFSLKLSSVTAADTAVYYCARGARWQARPATRIDGVAFDIWGQGTMVTVSS

SEQ ID NO: 333 - protein, 121 aa, Artificial Sequence
  Description of Artificial Sequence: Synthetic polypeptide
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDWSRSGYYLPDYWGQGTLVTVSS

SEQ ID NO: 334 - protein, 107 aa, Artificial Sequence
  Description of Artificial Sequence: Synthetic polypeptide
ETTLTQSPGTLSLSPGERATLSCRASQTVTRNYLAWYQQKPGQAPRLLMYGASNRAAGVPDRFSGSGSGTDFTLTISRLEPEDFAVYYCQQFGSPYTFGQGTKVEIK

SEQ ID NO: 335 - protein, 112 aa, Artificial Sequence
  Description of Artificial Sequence: Synthetic polypeptide
DVVMTQSPLSLPVTLGQPASISCRSSQSLLHSNGYNYLDWYLQRPGQSPHLLIFLGSNRASGVPDRFSGSGSGTDFTLKISRVEAEDVGIYYCMQALQTPPTFGQGTKVEIK

SEQ ID NO: 336 - protein, 124 aa, Artificial Sequence
  Description of Artificial Sequence: Synthetic polypeptide
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 337 - protein, 107 aa, Artificial Sequence
  Description of Artificial Sequence: Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 338 - protein, 117 aa, Artificial Sequence
  Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWLGNFDYWGQGTLVTVSS

SEQ ID NO: 339 - protein, 108 aa, Artificial Sequence
  Description of Artificial Sequence: Synthetic polypeptide
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 340 - protein, 117 aa, Artificial Sequence
  Description of Artificial Sequence: Synthetic polypeptide
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 341 - protein, 108 aa, Artificial Sequence
  Description of Artificial Sequence: Synthetic polypeptide
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 342 - protein, 116 aa, Artificial Sequence
  Description of Artificial Sequence: Synthetic polypeptide
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 343 - protein, 108 aa, Artificial Sequence; identical to SEQ ID NO: 339
  Description of Artificial Sequence: Synthetic polypeptide

SEQ ID NO: 344 - protein, 124 aa, Artificial Sequence
  Description of Artificial Sequence: Synthetic polypeptide
QVQLVQSGAEVKKPGASVKVSCKASGYTFTSYGINWVRQAPGQGLEWMGWISVYSGNTNYAQKVQGRVTMTADTSTSTAYMDLRSLRSDDTAVYYCAREGSSSSGDYYYGMDVWGQGTTVTVSS

SEQ ID NO: 345 - protein, 112 aa, Artificial Sequence
  Description of Artificial Sequence: Synthetic polypeptide
DIVMTQTPLSLSVTPGQPASISCKSSQSLLHTDGTTYLYWYLQKPGQPPQLLIYEVSNRFSGVPDRFSGSGSGTDFTLKISRVEAEDVGIYYCMQNIQLPWTFGQGTKVEIK

SEQ ID NO: 346 - protein, 124 aa, Artificial Sequence
  Description of Artificial Sequence: Synthetic polypeptide
QVQLVQSGAEVKKPGASVKVSCEASGYTFTSYGIDWVRQAPGQGLEWMGWISVYSGNTNYAQKLQGRVTMSTDTSTSTAYMELRSLRSDDTAVYYCAREGSSSSGDYYYGMDVWGQGTTVTVSS

SEQ ID NO: 347 - protein, 112 aa, Artificial Sequence
  Description of Artificial Sequence: Synthetic polypeptide
DIVMTQTPLSLSVTPGQPASISCKSNQSLLYSDGKTYLFWYLQKPGQPPQLLIYEVSNRFSGVPDRFSGSGSGTDFTLKISRVEAEDVGVYYCMQSIQLPWTFGQGTKVEIK

SEQ ID NO: 348 - protein, 120 aa, Artificial Sequence
  Description of Artificial Sequence: Synthetic polypeptide
EGQLVQSGGGLVHPGGSLRLSCAGSGFTFSSYGMHWVRQAPGKGLEWVSGIGTGGGTYSTDSVKGRFTISRDNAKNSLYLQMNSLRAEDMAVYYCARGDYYGSGSFFDCWGQGTLVTVSS

SEQ ID NO: 349 - protein, 107 aa, Artificial Sequence
  Description of Artificial Sequence: Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIK

SEQ ID NO: 350 - protein, 123 aa, Artificial Sequence [sequence omitted]

SEQ ID NO: 351 - protein, 106 aa, Artificial Sequence [sequence omitted]

SEQ ID NO: 352 - protein, 117 aa, Artificial Sequence
  Description of Artificial Sequence: Synthetic polypeptide
EVQLQQSGAELVKPGASVKISCKASGYTFTDYNMDWVKQSHGKSLEWIGDINPNYESTSYNQKFKGKATLTVDKSSSTAYMEVRSLTSEDTAVFYCARDKGWYFDVWGAGTTVTVSS

SEQ ID NO: 353 - protein, 106 aa, Artificial Sequence
  Description of Artificial Sequence: Synthetic polypeptide
ENVLTQSPAIMSASLGEKVTMSCRASSSVKNMYWYQQKSDASPKLWIYYTSNLAPGVPARFSGSGSGNSYSLTISSMEGEDAATYYCQQFTSSPYTFGGGTKLELK

SEQ ID NO: 354 - protein, 119 aa, Artificial Sequence
  Description of Artificial Sequence: Synthetic polypeptide
QVTLKESGPGILQPSQTLSLTCSFSGFSLSTSGMGVGWIRQPSGKGLEWLAHIWWDDDKFYNPSLKSQLTISKDTSRNQVFLKLTSVVTADTATYYCARLTGTRYFDYWGQGTTLTVSS

SEQ ID NO: 355 - protein, 106 aa, Artificial Sequence
  Description of Artificial Sequence: Synthetic polypeptide
DVQMTQSPSSLSASLGGKVTITCKASQDINKFIAWYQHKPGKGPRLLIHYTSTLQPGIPSKFSGSGSGRDYSFSISNLEPEDIATYYCLQYGNLWTFGGGTKLEIT

SEQ ID NO: 356 - protein, 119 aa, Artificial Sequence
  Description of Artificial Sequence: Synthetic polypeptide
QIQLVQSGPELKKPGETVKISCKASGYTFTDYSMHWVKQAPGKGLKWMGWINTETGEPTYADDFKGRFAFSSETSTSTAYLQINNLKNDDTTTYFCATYYGSSYVPDYWGQGTSLTVSS

SEQ ID NO: 357 - protein, 107 aa, Artificial Sequence
  Description of Artificial Sequence: Synthetic polypeptide
DIVMTQSHKFMSTSVGDRVSITCKASQNVGTAVAWYQHKPGQSPKLLIYWTSSRHTGVPDRFTGSGSGTEFTLTISNVQSEDLADYFCHQYSDYPYTFGGGTKLEIK

SEQ ID NO: 358 - protein, 119 aa, Artificial Sequence
  Description of Artificial Sequence: Synthetic polypeptide
EVQLQQSGPEVGRPGSSVKISCKASGYTFTDYIMHWVKQSPGQGLEWIGWVDPEYGSTDYAEKFKKKATLTADTSSNTAYIQLSSLTSEDTATYFCARDDGSYSPFDYWGQGVMVTVSS

SEQ ID NO: 359 - protein, 106 aa, Artificial Sequence
  Description of Artificial Sequence: Synthetic polypeptide
DIQMTQSPPSLSASLGDKVTITCQASQNINKYIAWYQQKPGKAPRLLIRYTSTLESGTPSRFSGSGSGRDYSFSISNVESEDIASYYCLQYVNLLTFGAGTKLEIK

SEQ ID NO: 360 - protein, 107 aa, Artificial Sequence
  Description of Artificial Sequence: Synthetic polypeptide
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 361 - protein, 120 aa, Artificial Sequence
  Description of Artificial Sequence: Synthetic polypeptide
EVQLVESGGGLVQPGGSLRLSCAASGFTFSDYAMSWVRQAPGKGLEWVAVISENGSDTYYADSVKGRFTISRDDSKNTLYLQMNSLRAEDTAVYYCARDRGGAVSYFDVWGQGTLVTVSS

SEQ ID NO: 362 - protein, 109 aa, Artificial Sequence
  Description of Artificial Sequence: Synthetic polypeptide
DIQMTQSPSSLSASVGDRVTITCRASQDVSSYLAWYQQKPGKAPKLLIYAASSLESGVPSRFSGSGSGTDFTLTISSLQPEDFATYYCQQYNSLPYTFGQGTKVEIKRT

SEQ ID NO: 363 - protein, 140 aa, Artificial Sequence
  Description of Artificial Sequence: Synthetic polypeptide
MAVLALLFCLVTFPSCILSQVQLKESGPGLVAPSQSLSITCTVSGFSLTGYGINWVRQPPGKGLEWLGMIWSDGSTDYNSVLTSRLRISKDNSNSQVFLKMNSLQVDDTARYYCARDRNYYDYDGAMDYWGQGTSVTVSS

SEQ ID NO: 364 - protein, 121 aa, Artificial Sequence
  Description of Artificial Sequence: Synthetic polypeptide
QVQLKESGPGLVAPSQSLSITCTVSGFSLTGYGINWVRQPPGKGLEWLGMIWSDGSTDYNSVLTSRLRISKDNSNSQVFLKMNSLQVDDTARYYCARDRNYYDYDGAMDYWGQGTSVTVSS

SEQ ID NO: 365 - protein, 127 aa, Artificial Sequence
  Description of Artificial Sequence: Synthetic polypeptide
MKFPSQLLLFLLFRITGIICDIQVTQSSSYLSVSLGDRVTITCKASDHIKNWLAWYQQKPGIAPRLLVSGATSLEAGVPSRFSGSGSGKNFTLSITSLQTEDVATYYCQQYWSTPWTFGGGTTLEIR

SEQ ID NO: 366 - protein, 107 aa, Artificial Sequence
  Description of Artificial Sequence: Synthetic polypeptide
DIQVTQSSSYLSVSLGDRVTITCKASDHIKNWLAWYQQKPGIAPRLLVSGATSLEAGVPSRFSGSGSGKNFTLSITSLQTEDVATYYCQQYWSTPWTFGGGTTLEIR

SEQ ID NO: 367 - protein, 126 aa, Artificial Sequence
  Description of Artificial Sequence: Synthetic polypeptide
EVQLVQPGAELRNSGASVKVSCKASGYRFTSYYIDWVRQAPGQGLEWMGRIDPEDGGTKYAQKFQGRVTFTADTSTSTAYVELSSLRSEDTAVYYCARNEWETVVVGDLMYEYEYWGQGTQVTVSS

SEQ ID NO: 368 - protein, 107 aa, Artificial Sequence
  Description of Artificial Sequence: Synthetic polypeptide
DIQMTQSPTSLSASLGDRVTITCQASQSISSYLAWYQQKPGQAPKLLIYGASRLQTGVPSRFSGSGSGTSFTLTISGLEAEDAGTYYCQQYDSLPVTFGQGTKVELK

SEQ ID NO: 369 - protein, 107 aa, Artificial Sequence
  Description of Artificial Sequence: Synthetic polypeptide
DIQMTQSPSSLSASLGDRVTITCQASQSIVSYLAWYQQKPGQAPKLLIYGASRLQTGVPSRFSGSGSGTSFTLTISGLEAEDAGTYYCQQYASAPVTFGQGTGVELK

SEQ ID NO: 370 - protein, 107 aa, Artificial Sequence
  Description of Artificial Sequence: Synthetic polypeptide
DIQMTQSPSSLSASLGDRVTITCQASQSISSYLAWYQQKPGQAPKLLIYGTSRLKTGVPSRFSGSGSGTSFTLTISGLEAEDAGTYYCQQYYSAPVTFGQGTKVELK

SEQ ID NO: 371 - protein, 107 aa, Artificial Sequence
  Description of Artificial Sequence: Synthetic polypeptide
DIQMTQSPSSLSPSLGDRVTITCQASQTISSFLAWYHQKPGQPPKLLIYRASIPQTGVPSRFSGSGSGTSFTLTIGGLEAEDAGTYYCQQYVSAPPTFGQGTKVELK

SEQ ID NO: 372 - protein, 107 aa, Artificial Sequence
  Description of Artificial Sequence: Synthetic polypeptide
DIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELK

SEQ ID NO: 373 - protein, 107 aa, Artificial Sequence
  Description of Artificial Sequence: Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 374 - protein, 121 aa, Artificial Sequence
  Description of Artificial Sequence: Synthetic polypeptide
QVQLVESGGGVVQPGRSLRLSCAASGFTFSNYGMHWVRQAPGKGLEWVAVILYDGSNKYYPDSVKGRFTISRDNSKNTLYLQMNSLRAEDTAVYYCARGGSSWYPDSFDIWGQGTMVTVSS

SEQ ID NO: 375 - protein, 122 aa, Artificial Sequence [sequence omitted]

SEQ ID NO: 376 - protein, 107 aa, Artificial Sequence [sequence omitted]

SEQ ID NO: 377 - protein, 117 aa, Artificial Sequence
  Description of Artificial Sequence: Synthetic polypeptide
QVQLVESGGGVVQPGRSLRLSCAASGFTFSSYDMSWVRQAPGKGLEWVAKVSSGGGSTYYLDTVQGRFTISRDNSKNTLYLQMNSLRAEDTAVYYCARHLHGSFASWGQGTTVTVSS

SEQ ID NO: 378 - protein, 107 aa, Artificial Sequence
  Description of Artificial Sequence: Synthetic polypeptide
EIVLTQSPATLSLSPGERATLSCQASQSISNFLHWYQQRPGQAPRLLIRYRSQSISGIPARFSGSGSGTDFTLTISSLEPEDFAVYYCQQSGSWPLTFGGGTKVEIK

SEQ ID NO: 379 - protein, 119 aa, Artificial Sequence
  Description of Artificial Sequence: Synthetic polypeptide
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSS

SEQ ID NO: 380 - protein, 108 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIK

SEQ ID NO: 381 - protein, 116 aa, Artificial Sequence
  Description of Artificial Sequence: Synthetic polypeptide
EVQLVESGGGLVQPGGSLRLSCAVSGFVFSRYWMSWVRQAPGKGLEWIGEINPDSSTINYTSSLKDRFTISRDNAKNSLYLQMNSLRAEDTAVYYCASLITTEDYWGQGTTVTVSS

SEQ ID NO: 382 - protein, 108 aa, Artificial Sequence
  Description of Artificial Sequence: Synthetic polypeptide
EIVLTQSPSSLSLSPGERVTITCKASQDINSYLSWYQQKPGKAPKLLIYYANRLVDGVPARFSGSGSGQDYTLTISSLEPEDFAVYYCLQYDEFPYTFGGGTKLEIKR

SEQ ID NO: 383 - protein, 107 aa, Artificial Sequence [sequence omitted]

SEQ ID NO: 384 - protein, 118 aa, Artificial Sequence [sequence omitted]

SEQ ID NO: 385 - protein, 124 aa, Artificial Sequence
  Description of Artificial Sequence: Synthetic polypeptide
QVQLQESGPGLVKPSQTLSLTCTVSGGSISSGVYYWTWIRQHPGNGLEWIGYIYYSGSTSYNPSLKSRVTISVDTSKKQFSLNLTSVTAADTAVYYCAREGPLRGDYYYGLDVWGQGTTVTVSS

SEQ ID NO: 386 - protein, 108 aa, Artificial Sequence
  Description of Artificial Sequence: Synthetic polypeptide
EIVLTQSPGTLSLSPGERATLSCRAGQTISSRYLAWYQQKPGQAPRPLIYGASSRATGIPDRFSGSGSGTDFTLTISRLEPEDFAVYYCQQYGSSPRTFGQGTKVEIK

SEQ ID NO: 387 - protein, 128 aa, Artificial Sequence
  Description of Artificial Sequence: Synthetic polypeptide
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAMYYCARYRGPAAGRGDFYYFGMDVWGQGTTVTVSS

SEQ ID NO: 388 - protein, 113 aa, Artificial Sequence
  Description of Artificial Sequence: Synthetic polypeptide
DIVMTQTPLSLSVTPGQPASIFCKSSQSLLNSDGKTYLCWYLQKPGQPPQLLIYEVSNRFSGVPDRFSGSGSGTDFTLKISRVEAEDVGVYYCMQGIQLPWAFFGQGTKVEIK

SEQ ID NO: 389 - protein, 125 aa, Artificial Sequence
  Description of Artificial Sequence: Synthetic polypeptide
QVQLVESGGGVVQPGRSLRLSCAASGFTFSSYGMHWVRQAPGKGLEWVAVIWYGGSNKYYADSVKGRFTISRDNSKNTLYLQMNSLRAEDTAVYYCARDLAARRGDYYYYGMDVWGQGTTVTVSS

SEQ ID NO: 390 - protein, 107 aa, Artificial Sequence
  Description of Artificial Sequence: Synthetic polypeptide
SSELTQDPVVSVALGQTVRITCQGDSLRSYYLSWYQQKPGQAPVLVIYGKNNRPSGIPDRFSGSNSGNTASLTITGAQAEDEADYYCNSRDSSGNHLFGGGTKLTVL

SEQ ID NO: 391 - protein, 125 aa, Artificial Sequence
  Description of Artificial Sequence: Synthetic polypeptide
QVQLQESGPGLVKPSQTLSLTCTVSGGSISSGGYYWSWIRQHPGKGLEWIGYIYYSGRTYNNPSLKSRVTISVDTSKNQFSLKLSSVTAADTAVYYCARVATGRADYHFYAMDVWGQGTTVTVSS

SEQ ID NO: 392 - protein, 106 aa, Artificial Sequence
  Description of Artificial Sequence: Synthetic polypeptide
SYELTQPSSVSVSPGQTARITCSGDVLAKKSARWFHQKPGQAPVLVIYKDSERPSGIPERFSGSSSGTTVTLTISGAQVEDEAAYYCYSAADNNLVFGGGTKLTVL

SEQ ID NO: 393 - protein, 116 aa, Artificial Sequence
  Description of Artificial Sequence: Synthetic polypeptide
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 394 - protein, 106 aa, Artificial Sequence
  Description of Artificial Sequence: Synthetic polypeptide
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

SEQ ID NO: 395 - protein, 121 aa, Artificial Sequence
  Description of Artificial Sequence: Synthetic polypeptide
QLQLQESGPGLLKPSETLSLTCTVSGGSISSPGYYGGWIRQPPGKGLEWIGSIYKSGSTYHNPSLKSRVTISVDTSKNQFSLKLSSVTAADTAVYYCTRPVVRYFGWFDPWGQGTLVTVSS

SEQ ID NO: 396 - protein, 106 aa, Artificial Sequence
  Description of Artificial Sequence: Synthetic polypeptide
AIQLTQSPSSLSASVGDRVTITCRASQGISSALAWYQQKPGKAPKLLIYDASNLESGVPSRFSGSGSGTDFTLTISSLQPEDFATYYCQQFNSYPTFGQGTKVEIK

SEQ ID NO: 397 - protein, 118 aa, Artificial Sequence
  Description of Artificial Sequence: Synthetic polypeptide
QVQLVESGGGVVQPGRSLRLSCAASGFTFSNYGMYWVRQAPGKGLEWVAVIWYDGSNKYYADSVKGRFTISRDNSKNTLYLQMNSLRAEDTAVYYCARDLWGWYFDYWGQGTLVTVSS

SEQ ID NO: 398 - protein, 107 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRRNWPLTFGGGTKVEIK

SEQ ID NO: 399 - protein, 116 aa, Artificial Sequence
  Description of Artificial Sequence: Synthetic polypeptide
EVQLVQSGAEVKKPGESLRISCKGSGDSFTTYWIGWVRQMPGKGLEWMGIIYPGDSDTIYSPSFQGQVTISADKSISTAYLQWSSLKASDTAMYYCTRGDRGVDYWGQGTLVTVSS

SEQ ID NO: 400 - protein, 107 aa, Artificial Sequence
  Description of Artificial Sequence: Synthetic polypeptide
DIQMTQSPSSLSASVGDRVTITCRASQGISRWLAWYQQKPEKAPKSLIYAASSLQSGVPSRFSGSGSGTDFTLTISGLQPEDFATYYCQQYNSYPRTFGQGTKVEIK

SEQ ID NO: 401 - protein, 119 aa, Artificial Sequence
  Description of Artificial Sequence: Synthetic polypeptide
QVTLKESGPALVKPTQTLTLTCTFSGFSLSTSGMGVGWIRQPPGKALEWLAHIWWDDDKFYNPSLKSRLTISKDTSKNQVVLTMTNMDPVDTATYYCARITGTRYFDYWGQGTTVTVSS

SEQ ID NO: 402 - protein, 106 aa, Artificial Sequence
  Description of Artificial Sequence: Synthetic polypeptide
DIQMTQSPSSLSASVGDRVTITCKASQDINKFIAWYQQKPGKAPKLLIHYTSTLQPGVPSRFSGSGSGTDYTFTISSLQPEDIATYYCLQYGNLWTFGGGTKVEIK

SEQ ID NO: 403 - protein, 125 aa, Artificial Sequence
  Description of Artificial Sequence: Synthetic polypeptide
EVQLQQSGPELEKPGASVKISCKASGYSFTDYNMNWVKQSNGKSLEWIGNIDPYYGGTSYNQKFKGKATLTVDKSSSTAYMQLNSLTSEDSAVYFCARWDYRYDDGRAYYVMDFWGQGTSVTVSS

SEQ ID NO: 404 - protein, 112 aa, Artificial Sequence
  Description of Artificial Sequence: Synthetic polypeptide
ELQMTQSPSSLAASAGEKVTMSCKSSQSVLYSSNQKNYLAWYQQKPGQSPKLLIYWASTRESGVPDRFTGSGSGTHFTLTVSSVQAEDLAVYYCHQYLYSWTFGGGTNLEIK

SEQ ID NO: 405 - protein, 125 aa, Artificial Sequence
  Description of Artificial Sequence: Synthetic polypeptide
QVQLVQSGAEVKKPGASVKVSCKASGYTFTDYNMNWVRQAPGQGLEWMGNIDPYYGGTSYNQKFKGRVTMTRDTSISTAYMELSRLRSDDTAVYYCARWDYRYDDGRAYYVMDFWGQGTTVTVSS

SEQ ID NO: 406 - protein, 112 aa, Artificial Sequence
  Description of Artificial Sequence: Synthetic polypeptide
DIVMTQSPDSLAVSLGERATINCKSSQSVLYSSNQKNYLAWYQQKPGQPPKLLIYWASTRESGVPDRFSGSGSGTDFTLTISSLQAEDVAVYYCHQYLYSWTFGQGTKLEIK

SEQ ID NO: 407 - protein, 118 aa, Artificial Sequence
  Description of Artificial Sequence: Synthetic polypeptide
EVQLVESGAEVKKPGASVKVSCKASGYTFTNYIIHWVKQEPGQGLEWIGYFNPYNHGTKYNEKFKGRATLTANKSISTAYMELSSLRSEDTAVYYCARSGPYAWFDTWGQGTTVTVSS

SEQ ID NO: 408 - protein, 107 aa, Artificial Sequence
  Description of Artificial Sequence: Synthetic polypeptide
DILLTQSPATLSLSPGERATFSCRASQNIGTSIQWYQQKTNGAPRLLIRSSSESISGIPSRFSGSGSGTDFTLTISSLEPEDFAVYYCQQSNTWPFTFGQGTKLEIK

SEQ ID NO: 409 - protein, 118 aa, Artificial Sequence; identical to SEQ ID NO: 407
  Description of Artificial Sequence: Synthetic polypeptide

SEQ ID NO: 410 - protein, 107 aa, Artificial Sequence
  Description of Artificial Sequence: Synthetic polypeptide
DILLTQSPATLSLSPGERATLSCRASQNIGTSIQWYQQKPGQAPRLLIRSSSESISGIPSRFSGSGSGTDFTLTISSLEPEDFAVYYCQQSNTWPFTFGQGTKLEIK

SEQ ID NO: 411 - protein, 118 aa, Artificial Sequence
  Description of Artificial Sequence: Synthetic polypeptide
QCQVQLVESGGGVVQPGRSLRVSCEASGFTFSNYGMHWVRQAPGKGLEWVAVIWYDGSKKFYADSVKGRFTISRDNSQNTLSLQMSSLRAEDTAVYYCARGGGDFDFWGQGTLVTVSS

SEQ ID NO: 412 - protein, 109 aa, Artificial Sequence
  Description of Artificial Sequence: Synthetic polypeptide
KIVMTQSPATLSVSPGERATLSCRASQSVSGNYLAWYQQRPGQAPRLLIYGASTRATGIPARFSGSGSGTEFTLTISSLQSEDFAVYYCQQYGKWPPLTFGGGTKVEIK

SEQ ID NO: 413 - protein, 120 aa, Artificial Sequence
  Description of Artificial Sequence: Synthetic polypeptide
QVQLKESGPALVKPTQTLTLTCTFSGFSLSTSGVGVGWIRQPPGKALEWLALIDWDDDKYYSTSLKTRLTISKDTSKNQVVLTMTNMDPVDTATYYCARSPRYRGAFDYWGQGTLVTVSS

SEQ ID NO: 414 - protein, 109 aa, Artificial Sequence
  Description of Artificial Sequence: Synthetic polypeptide
DIVLTQPPSVSGAPGQRVTISCSGSESNIGNNYVQWYQQLPGTAPKLLIYDNNQRPSGVPDRFSGSKSGTSASLAITGLQSEDEADYYCQSYDLIRHVFGGGTKLTVLG

SEQ ID NO: 415 - protein, 120 aa, Artificial Sequence; identical to SEQ ID NO: 413
  Description of Artificial Sequence: Synthetic polypeptide

SEQ ID NO: 416 - protein, 109 aa, Artificial Sequence
  Description of Artificial Sequence: Synthetic polypeptide
DIVLTQPPSVSGAPGQRVTISCSGSESNIGNNYVQWYQQLPGTAPKLLIYDNNQRPSGVPDRFSGSKSGTSASLAITGLQSEDEADYYCQSYDMNVHVFGGGTKLTVLG

SEQ ID NO: 417 - protein, 119 aa, Artificial Sequence
  Description of Artificial Sequence: Synthetic polypeptide
QVQLQESGPGLVKPSETLSLTCTVSGFSLTNYGIHWIRQPPGKGLEWLGVIWARGFTNYNSALMSRLTISKDNSKNQVSLKLSSVTAADTAVYYCARANDGVYYAMDYWGQGTLVTVSS

SEQ ID NO: 418 - protein, 106 aa, Artificial Sequence
  Description of Artificial Sequence: Synthetic polypeptide
DFVMTQSPAFLSVTPGEKVTITCSAQSSVNYIHWYQQKPDQAPKKLIYDTSKLASGVPSRFSGSGSGTDYTFTISSLEAEDAATYYCQQWTTNPLTFGQGTKVEIK

SEQ ID NO: 419 - protein, 121 aa, Artificial Sequence [sequence omitted]

SEQ ID NO: 420 - protein, 112 aa, Artificial Sequence [sequence omitted]

SEQ ID NO: 421 - protein, 117 aa, Artificial Sequence [sequence omitted]

SEQ ID NO: 422 - protein, 107 aa, Artificial Sequence [sequence omitted]

SEQ ID NO: 423 - protein, 117 aa, Artificial Sequence [sequence omitted]

SEQ ID NO: 424 - protein, 108 aa, Artificial Sequence [sequence omitted]

SEQ ID NO: 425 - protein, 122 aa, Artificial Sequence
  Description of Artificial Sequence: Synthetic polypeptide
DVQLVESGGGLVKPGGSLRLSCAASGFTFSTYTMSWVRQAPGKGLEWVATISPGDSFGYYYPDSVQGRFTISRDNAKNSLYLQMNSLRAEDTAVYYCTRDIYYNYGAWFAYWGQGTLVTVSS

SEQ ID NO: 426 - protein, 111 aa, Artificial Sequence
  Description of Artificial Sequence: Synthetic polypeptide
DIQLTQSPSSLSASVGDRVTITCKASQSVDYDGDSYMNWYQQKPGKAPKLLIYAASTLESGVPSRFSGSGSGTDFTLTISSLQPEDFATYYCQQANEDPRTFGQGTKVEIK

SEQ ID NO: 427 - protein, 117 aa, Artificial Sequence
  Description of Artificial Sequence: Synthetic polypeptide
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 428 - protein, 111 aa, Artificial Sequence
  Description of Artificial Sequence: Synthetic polypeptide
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 429 - protein, 112 aa, Artificial Sequence
  Description of Artificial Sequence: Synthetic polypeptide
QVQLQQWGAGLLKPSETLSLTCAVYGGSFSAYYWSWIRQPPGKGLEWIGDINHGGGTNYNPSLKSRVTISVDTSKNQFSLKLNSVTAADTAVYYCASLTAYWGQGSLVTVSS

SEQ ID NO: 430 - protein, 107 aa, Artificial Sequence
  Description of Artificial Sequence: Synthetic polypeptide
DIQMTQSPTSLSASVGDRVTITCRASQGISSWLTWYQQKPEKAPKSLIYAASSLQSGVPSRFSGSGSGTDFTLTISSLQPEDFATYYCQQYDSYPITFGQGTRLEIK

SEQ ID NO: 431 - protein, 117 aa, Artificial Sequence
  Description of Artificial Sequence: Synthetic polypeptide
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSS

SEQ ID NO: 432 - protein, 111 aa, Artificial Sequence
  Description of Artificial Sequence: Synthetic polypeptide
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIK

SEQ ID NO: 433 - protein, 113 aa, Artificial Sequence [sequence omitted]

SEQ ID NO: 434 - protein, 107 aa, Artificial Sequence [sequence omitted]

SEQ ID NO: 435 - protein, 120 aa, Artificial Sequence [sequence omitted]

SEQ ID NO: 436 - protein, 111 aa, Artificial Sequence [sequence omitted]

SEQ ID NO: 437 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 438 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 439 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 440 - protein, 5 aa, Artificial Sequence
  Description of Artificial Sequence: Synthetic peptide
SYWIE

SEQ ID NO: 441 - protein, 17 aa, Artificial Sequence
  Description of Artificial Sequence: Synthetic peptide
EILPGSDTTNYNEKFKD

SEQ ID NO: 442 - protein, 9 aa, Artificial Sequence
  Description of Artificial Sequence: Synthetic peptide
GNYRAWFGY

SEQ ID NO: 443 - protein, 11 aa, Artificial Sequence
  Description of Artificial Sequence: Synthetic peptide
RASQDISNYLN

SEQ ID NO: 444 - protein, 7 aa, Artificial Sequence
  Description of Artificial Sequence: Synthetic peptide
YTSRLHS

SEQ ID NO: 445 - protein, 9 aa, Artificial Sequence
  Description of Artificial Sequence: Synthetic peptide
QQGEALPWT

SEQ ID NO: 446 - protein, 5 aa, Artificial Sequence
  Description of Artificial Sequence: Synthetic peptide
DYYIH

SEQ ID NO: 447 - protein, 17 aa, Artificial Sequence
  Description of Artificial Sequence: Synthetic peptide
LVYPYIGGTNYNQKFKG

SEQ ID NO: 448 - protein, 10 aa, Artificial Sequence
  Description of Artificial Sequence: Synthetic peptide
GDNKYDAMDY

SEQ ID NO: 449 - protein, 15 aa, Artificial Sequence
  Description of Artificial Sequence: Synthetic peptide
RASQSVSTSSYSYMH

SEQ ID NO: 450 - protein, 7 aa, Artificial Sequence
  Description of Artificial Sequence: Synthetic peptide
YASSLES

SEQ ID NO: 451 - protein, 8 aa, Artificial Sequence
  Description of Artificial Sequence: Synthetic peptide
EQSWEIRT

SEQ ID NO: 452 - protein, 5 aa, Artificial Sequence
  Description of Artificial Sequence: Synthetic peptide
NYWMH

SEQ ID NO: 453 - protein, 17 aa, Artificial Sequence
  Description of Artificial Sequence: Synthetic peptide
MIHPNSGSTKHNEKFRG

SEQ ID NO: 454 - protein, 12 aa, Artificial Sequence
  Description of Artificial Sequence: Synthetic peptide
SDFGNYRWYFDV

SEQ ID NO: 455 - protein, 11 aa, Artificial Sequence
  Description of Artificial Sequence: Synthetic peptide
RASQSSSNNLH

SEQ ID NO: 456 - protein, 7 aa, Artificial Sequence
  Description of Artificial Sequence: Synthetic peptide
YVSQSIS

SEQ ID NO: 457 - protein, 9 aa, Artificial Sequence
  Description of Artificial Sequence: Synthetic peptide
QQSNSWPFT

SEQ ID NO: 458 - protein, 5 aa, Artificial Sequence
  Description of Artificial Sequence: Synthetic peptide
DYYIH

SEQ ID NO: 459 - protein, 17 aa, Artificial Sequence
  Description of Artificial Sequence: Synthetic peptide
LVYPYIGGSSYNQQFKG

SEQ ID NO: 460 - protein, 10 aa, Artificial Sequence
  Description of Artificial Sequence: Synthetic peptide
GDNNYDAMDY

SEQ ID NO: 461 - protein, 15 aa, Artificial Sequence
  Description of Artificial Sequence: Synthetic peptide
RASQSVSTSTYNYMH

SEQ ID NO: 462 - protein, 7 aa, Artificial Sequence
  Description of Artificial Sequence: Synthetic peptide
YASNLES

SEQ ID NO: 463 - protein, 8 aa, Artificial Sequence
  Description of Artificial Sequence: Synthetic peptide
HHTWEIRT

SEQ ID NO: 464 - protein, 5 aa, Artificial Sequence
  Description of Artificial Sequence: Synthetic peptide
SYGVH

SEQ ID NO: 465 - protein, 16 aa, Artificial Sequence
  Description of Artificial Sequence: Synthetic peptide
VIWAGGSTNYNSALMS

SEQ ID NO: 466 - protein, 12 aa, Artificial Sequence
  Description of Artificial Sequence: Synthetic peptide
HMITEDYYGMDY

SEQ ID NO: 467 - protein, 17 aa, Artificial Sequence
  Description of Artificial Sequence: Synthetic peptide
KSSQSLLNSRTRKNYLA

SEQ ID NO: 468 - protein, 7 aa, Artificial Sequence
  Description of Artificial Sequence: Synthetic peptide
WASTRES

SEQ ID NO: 469 - protein, 8 aa, Artificial Sequence
  Description of Artificial Sequence: Synthetic peptide
KQSYNLPT

SEQ ID NO: 470 - protein, 5 aa, Artificial Sequence
  Description of Artificial Sequence: Synthetic peptide
NYWLG

SEQ ID NO: 471 - protein, 17 aa, Artificial Sequence
  Description of Artificial Sequence: Synthetic peptide
DIYPGGGNTYYNEKLKG

SEQ ID NO: 472 - protein, 11 aa, Artificial Sequence
  Description of Artificial Sequence: Synthetic peptide
WGDKKGNYFAY

SEQ ID NO: 473 - protein, 12 aa, Artificial Sequence
  Description of Artificial Sequence: Synthetic peptide
TASSSVYSSYLH

SEQ ID NO: 474 - protein, 7 aa, Artificial Sequence
  Description of Artificial Sequence: Synthetic peptide
STSNLAS

SEQ ID NO: 475 - protein, 8 aa, Artificial Sequence
  Description of Artificial Sequence: Synthetic peptide
HQYHRSPT

SEQ ID NO: 476 - protein, 5 aa, Artificial Sequence
  Description of Artificial Sequence: Synthetic peptide
NFGMN

SEQ ID NO: 477 - protein, 17 aa, Artificial Sequence
  Description of Artificial Sequence: Synthetic peptide
WINLYTGEPTFADDFKG

SEQ ID NO: 478 - protein, 13 aa, Artificial Sequence
  Description of Artificial Sequence: Synthetic peptide
KGETYYRYDGFAY

SEQ ID NO: 479 - protein, 16 aa, Artificial Sequence
  Description of Artificial Sequence: Synthetic peptide
RSSKSLLHSNGNTHLY

SEQ ID NO: 480 - protein, 7 aa, Artificial Sequence
  Description of Artificial Sequence: Synthetic peptide
RMSNLAS

SEQ ID NO: 481 - protein, 9 aa, Artificial Sequence
  Description of Artificial Sequence: Synthetic peptide
MQLLEYPYT

SEQ ID NO: 482 - protein, 120 aa, Artificial Sequence
  Description of Artificial Sequence: Synthetic polypeptide
EVQLVQSGAEVKKPGASVKVSCKASGYKFSSYWIEWVKQAPGQGLEWIGEILPGSDTTNYNEKFKDRATFTSDTSINTAYMELSRLRSDDTAVYYCARDRGNYRAWFGYWGQGTLVTVSS

SEQ ID NO: 483 - protein, 107 aa, Artificial Sequence
  Description of Artificial Sequence: Synthetic polypeptide
DIQMTQSPSSLSASVGDRVTITCRASQDISNYLNWYQQKPGGAVKFLIYYTSRLHSGVPSRFSGSGSGTDYTLTISSLQPEDFATYFCQQGEALPWTFGGGTKVEIK

SEQ ID NO: 484 - protein, 119 aa, Artificial Sequence
  Description of Artificial Sequence: Synthetic polypeptide
EVQLVQSGAEVKKPGSSVKVSCKASGFTFTDYYIHWVKQAPGQGLEWIGLVYPYIGGTNYNQKFKGKATLTVDTSTTTAYMEMSSLRSEDTAVYYCARGDNKYDAMDYWGQGTTVTVSS

SEQ ID NO: 485 - protein, 110 aa, Artificial Sequence
  Description of Artificial Sequence: Synthetic polypeptide
DIVLTQSPDSLAVSLGERATINCRASQSVSTSSYSYMHWYQQKPGQPPKLLIKYASSLESGVPDRFSGSGSGTDFTLTISSLQAEDVAVYYCEQSWEIRTFGGGTKVEIK

SEQ ID NO: 486 - protein, 121 aa, Artificial Sequence
  Description of Artificial Sequence: Synthetic polypeptide
EVQLVQSGAEVKKPGSSVKVSCKASGYTFTNYWMHWVKQAPGQGLEWIGMIHPNSGSTKHNEKFRGKATLTVDESTTTAYMELSSLRSEDTAVYYCARSDFGNYRWYFDVWGQGTTVTVSS

SEQ ID NO: 487 - protein, 107 aa, Artificial Sequence
  Description of Artificial Sequence: Synthetic polypeptide
EIVLTQSPATLSLSPGERATLSCRASQSSSNNLHWYQQKPGQAPRVLIKYVSQSISGIPARFSGSGSGTDFTLTISSLEPEDFAVYFCQQSNSWPFTFGQGTKLEIK

SEQ ID NO: 488 - protein, 119 aa, Artificial Sequence
  Description of Artificial Sequence: Synthetic polypeptide
EVQLVQSGAEVKKPGSSVKVSCKASGFTFTDYYIHWVKQAPGQGLEWIGLVYPYIGGSSYNQQFKGKATLTVDTSTSTAYMELSSLRSEDTAVYYCARGDNNYDAMDYWGQGTTVTVSS

SEQ ID NO: 489 - protein, 110 aa, Artificial Sequence
  Description of Artificial Sequence: Synthetic polypeptide
DIVLTQSPDSLAVSLGERATISCRASQSVSTSTYNYMHWYQQKPGQPPKLLVKYASNLESGVPDRFSGSGSGTDFTLTISSLQAEDVAVYYCHHTWEIRTFGGGTKVEIK

SEQ ID NO: 490 - protein, 120 aa, Artificial Sequence
  Description of Artificial Sequence: Synthetic polypeptide
EVQLVESGGGLVQPGGSLRLSCAVSGFSLTSYGVHWVRQATGKGLEWLGVIWAGGSTNYNSALMSRLTISKENAKSSVYLQMNSLRAGDTAMYYCATHMITEDYYGMDYWGQGTTVTVSS

SEQ ID NO: 491 - protein, 112 aa, Artificial Sequence
  Description of Artificial Sequence: Synthetic polypeptide
DIVMTQSPDSLAVSLGERATINCKSSQSLLNSRTRKNYLAWYQQKPGQSPKLLIYWASTRESGVPDRFSGSGSGTDFTLTISSLQAEDVAVYYCKQSYNLPTFGGGTKVEIK

SEQ ID NO: 492 - protein, 5 aa, Unknown
  Description of Unknown: Sortase recognition motif
  MOD_RES (3)..(3): Any amino acid
LPXTG

SEQ ID NO: 493 - protein, 4 aa, Artificial Sequence
  Description of Artificial Sequence: Synthetic peptide
GGFG

SEQ ID NO: 494 - protein, 4 aa, Artificial Sequence
  Description of Artificial Sequence: Synthetic peptide
GFLG

SEQ ID NO: 495 - protein, 4 aa, Artificial Sequence
  Description of Artificial Sequence: Synthetic peptide
ALAL

(19) United States
(12) Patent Application Publication
(10) Pub. No.: US 2020/0199247 A1
(43) Pub. Date: Jun. 25, 2020

(54) ANTIBODY CONJUGATES OF IMMUNE-MODULATORY COMPOUNDS AND USES THEREOF

(71) Applicant: SILVERBACK THERAPEUTICS, INC., Seattle, WA (US)

(72) Inventors: Peter Armstrong THOMPSON, Seattle, WA (US); Badreddin EDRIS, Seattle, WA (US); Craig Alan COBURN, Seattle, WA (US); Peter Robert BAUM, Seattle, WA (US); Valerie ODEGARD, Seattle, WA (US)

(21) Appl. No.: 16/620,429

(22) PCT Filed: Jun. 7, 2018
(86) PCT No.: PCT/US2018/036554

§ 371 Date: Dec. 6, 2019

Related U.S. Application Data
(60) Provisional application No. 62/516,638, filed on Jun. 7, 2017.

(51) Int. Cl. C07K 16/32; C07K 16/28; C07K 16/30; A61K 47/68; A61P 35/00

(52) U.S. Cl. CPC C07K 16/32; C07K 16/2863; C07K 16/30; A61K 47/6803; A61K 2039/505; A61K 47/6845; A61K 47/6851; A61P 35/00; A61K 47/6849

(57) ABSTRACT

Antibody conjugates of immune-modulatory compounds and pharmaceutical compositions for use in the treatment of disease, such as fibrotic diseases, autoimmune, or autoinflammatory diseases, are disclosed herein. The disclosed conjugates are useful, among other things, in treating fibrotic diseases, autoimmune diseases, or autoinflammatory diseases, such as by modulating TGFβR1, TGFβR2, TNKS, TNIK, or mTOR.

### PRIORITY

This application claims the benefit of priority under 35 U.S.C. § 119(e) to U.S. Provisional Application No. 62/516,638, filed Jun. 7, 2017, the disclosure of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 27, 2018, is named 50358_720_601_SL.txt and is 252,334 bytes in size.

### BACKGROUND OF THE INVENTION

Autoimmune and autoinflammatory diseases can result from an abnormal response of the immune system to a normal part of the body. In an autoimmune disease, the adaptive immune system can attack the body's own tissues. For example, one hallmark of autoimmune disease can be the production of auto-antibodies to antigens in normal tissues of the patient. Persistent inflammation can be another symptom of autoimmune disease and can play a role in the pathogenesis of common autoimmune diseases such as rheumatoid arthritis, inflammatory bowel diseases, systemic lupus erythematosus, and multiple sclerosis. Treatment for autoimmune diseases generally focuses on reducing immune system activity, but many patients fail to respond to current therapies or their disease becomes refractory to the treatment. Thus, new more durable treatments are needed.

Fibrosis can be the formation of excess fibrous connective tissue or scar tissue in an organ or tissue in a reparative or reactive process. Fibrosis can occur in many tissues within the body, typically as a result of inflammation or damage, which can include the lungs, kidney, liver, heart, and brain. Scar tissue can block arteries, immobilize joints, and damage internal organs, which can negatively impact the body's ability to maintain vital functions. Every year, millions of people are hospitalized due to the damaging effects of fibrosis. However, current therapeutics for treating fibrotic diseases are lacking or have drawbacks. Thus, there remains a considerable need for alternative or improved treatments for fibrotic diseases.

### SUMMARY OF THE INVENTION

In various aspects, a composition of a conjugate is provided that comprises: an immune-modulatory compound; an antibody construct comprising a first antigen binding domain and an Fc domain, wherein: the first antigen binding domain specifically binds to a first antigen, wherein the first antigen has at least 80% sequence identity with an antigen selected from a group consisting of Cadherin 11, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, and CD25, and a fragment thereof; a linker attaching the antibody construct to the immune-modulatory compound, wherein the linker is covalently bound to the antibody construct and the linker is covalently bound to the immune-modulatory compound, and optionally wherein a molar ratio of immune-modulatory compound to antibody construct is less than 8. In some aspects, the first antigen is selected from LRRC15, Cadherin 11, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, MMP14, GPX8, and F2RL2. In some aspects, the first antigen is selected from FAP, LRRC15, Cadherin 11 (CDH11), and TNFR2. In some aspects, the immune-modulatory compound has activity on stellate cells, myofibroblasts, synovial fibroblasts, epithelial cells, podocytes or immune cells. In some aspects, the immune-modulatory compound has activity on stellate cells, myofibroblasts, or immune cells.

In various aspects, a conjugate comprises: an immune-modulatory compound; a second compound; a spacer comprising 1 to 100 linear, non-hydrogen atoms covalently attached to the immune-modulatory compound and to the second compound; an antibody construct comprising a first antigen binding domain and an Fc domain, wherein: the first antigen binding domain specifically binds to a first antigen, wherein the first antigen has at least 80% sequence identity with an antigen selected from a group consisting of Cadherin 11, PDPN, LRRC15, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, LRRC15, MMP14, GPX8, and F2RL2, and a fragment thereof; and a linker attaching the antibody construct to the immune-modulatory compound, the second compound, or the spacer, wherein the linker is covalently bound to the antibody construct and the linker is covalently bound to the immune-modulatory compound, the second compound, or the spacer. In some aspects, the second compound binds to to an E3 ubiquitin ligase. In some aspects, the second compound is a second immune-modulatory compound. In some aspects, the second immune-modulatory compound and the immune-modulatory compound are the same. In some further aspects, the first antigen is selected from Cadherin 11, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, and LRRC15. In some aspects, the first antigen is selected from FAP, LRRC15, Cadherin 11, and TNFR2. In some aspects, the immune-modulatory compound has activity on stellate cells, myofibroblasts, synovial fibroblasts, epithelial cells, podocytes or immune cells. In some aspects, the immune-modulatory compound has activity on stellate cells, myofibroblasts, or immune cells.

In various aspects, a conjugate comprises: an immune-modulatory compound; an antibody construct comprising a first antigen binding domain and an Fc domain, wherein: the first antigen binding domain specifically binds to a first antigen, wherein the first antigen comprises a protein complex, a protein conformer, a post-transcriptional modification, or a post-translational modification; a linker attaching the antibody construct to the immune-modulatory compound, wherein the linker is covalently bound to the antibody construct and the linker is covalently bound to the immune-modulatory compound, and wherein a molar ratio of immune-modulatory compound to antibody construct is less than 8. In some aspects, the first antigen is the post-translational modification or protein conformer such as a CD45RB splice variant or of a CD45RO splice variant. In some aspects, the first antigen is the protein complex of an integrin pair. In some aspects, the integrin pair comprises αvβ6.

In some aspects, the immune-modulatory compound comprises a first moiety that binds to a protein target and a second moiety that binds to an E3 ubiquitin ligase. In some aspects, the first moiety is covalently attached to the second moiety via a spacer comprising from 5 to 20 linear, non-hydrogen atoms. In some aspects, a Kd for binding of the first antigen binding domain to the first antigen in a presence of the immune-modulatory compound (when the immune-modulatory compound is attached to the antibody construct) is less than about 100 nM and no greater than about 100 times a Kd for binding of the first antigen binding domain to the first antigen in the absence of the immune-modulatory compound (i.e., the immune-modulatory compound is not attached to the antibody construct). In some aspects, a Kd for binding of the Fc domain to an Fcγ receptor in the presence of the immune-modulatory compound (i.e., the immune-modulatory compounds is attached to the antibody construct) is equivalent to or no greater than 2 times, 5 times, or 10 times a Kd for binding of the Fc domain to the Fcγ receptor in the absence of the immune-modulatory compound (i.e., the immune-modulatory compound is not attached to the antibody construct). In some aspects, a Kd for binding of the Fc domain to an Fcγ receptor is greater than 100 times a Kd for binding of an IgG1 Fc domain to the Fcγ receptor in the absence of the immune-modulatory compound, and wherein a Kd for binding of the Fc domain to an FcRn receptor in the presence of the immune-modulatory compound is at least equivalent to or at least no greater than about 2 times, 5 times, or 10 times a Kd for binding of the Fc domain to the FcRn receptor in the absence of the immune-modulatory compound. In some aspects, a Kd for binding of the Fc domain to an FcRn receptor in the presence of the immune-modulatory compound is at least equivalent to or at least no greater than about 2 times, 5 times, or 10 times a Kd for binding of the Fc domain to the FcRn receptor in the absence of the immune-modulatory compound. In some embodiments, the Fc domain is an Fc null.

In some aspects, the antibody construct further comprises a second binding domain. In some aspects, the immune-modulatory compound of the conjugate lowers activity of the protein target in a cell, the cell expressing the first antigen, the second antigen, or both, on the cell surface. In some aspects, the conjugate lowers activity of the protein target by increasing target protein degradation in a cell, the cell expressing the first antigen, the second antigen, or both, on the cell surface. In some aspects, the conjugate increases activity of the protein target in a cell, the cell expressing the first antigen, the second antigen, or both, on the cell surface. In some aspects, the conjugate alters activity of the protein target in a cell, the cell expressing the first antigen, the second antigen, or both, on the cell surface. In some aspects, the conjugate alters activity of the protein target in a cell, the cell expressing expressing the first antigen, the second antigen, or both, on the cell surface compared to a cell not expressing the first antigen, the second antigen, or both, on the cell surface. In some aspects, the conjugate increases activity of the protein target in a cell, the cell expressing the first antigen, the second antigen, or both, on the cell surface, and wherein the first moiety is an agonist for A2aR, PP2A, PPARg, Vitamin D Receptor (VDR), or KCA3.1. In some aspects, the conjugate lowers activity of the protein target in a cell, the cell expressing the first antigen, the second antigen, or both, on the cell surface, and wherein the first moiety is a kinase inhibitor, ion channel antagonist, or a PARP1 inhibitor. In some aspects, the conjugate lowers activity of the protein target by increasing target protein degradation in a cell, expressing the first antigen, the second antigen, or both, on the cell surface, and wherein the first moiety is a kinase inhibitor, ion channel antagonist, or a PARP1 inhibitor. In some aspects, the conjugate lowers fibrogenic activity of stellate cells or myofibroblasts. In some aspects, the conjugate lowers activation of an activated immune cell or decreases production of one or more pro-inflammatory mediators. In some aspects, the conjugate increases an immunosuppressive activity or tolerogenic activity of an immune cell. In some aspects, the second binding domain specifically binds to a second antigen. In some aspects, the second antigen is an antagonist of an immune cell immunomodulatory target or an agonist of an immune check point target on an immune cell or tissue. In some aspects, the second antigen comprises at least 80% sequence identity with TNFR2, CD40, CD86, PD-1, TIM3, BTLA, DEC205, DCIR, CD45RB, CD45RO, HLA DR, CD38, CD73, GARP, BDCA2, or CD30. In some aspects, the second antigen comprises at least 80% sequence identity with TNFR2, CD40, CD86, PD-1, PD-L1, TIM3, BTLA, DEC205, DCIR, CD45RB, CD45RO, HLA DR, CD38, CD73, GARP, BDCA2, or CD30. In some aspects, the second binding domain is attached to the antibody construct at a C-terminal end of the Fc domain. In some aspects, the second binding domain is attached to a C-terminal end of a light chain of the antibody construct. In some aspects, after administration of the conjugate to a subject, inflammation is decreased in the subject. In some aspects, after administration of the conjugate to a subject, fibrosis is decreased is the subject. In some aspects, after administration of the conjugate to a subject, immune suppression is increased in the subject. In some aspects, after administration of the conjugate to a subject, immune tolerance is increased in the subject. In some aspects, the first antigen binding domain is a CD40 antagonist. In some aspects, the second binding domain is attached to the Fc domain or the light chain of the first antigen binding domain: a) as an Fc domain-second binding domain fusion protein; b) as a light chain-second binding domain fusion protein; or c) by a conjugation via a first linker.

In some aspects, the Fc domain is attached to the first antigen binding domain: a) as an Fc domain-first antigen binding domain fusion protein; or b) by conjugation via a second linker.

In some aspects, the Fc domain is attached to both the second binding domain and to the first antigen binding domain as a second binding domain-Fc domain-first antigen binding domain fusion protein. In some aspects, the first antigen binding domain is attached to both the Fc domain and the second binding domain as a second binding domain-first antigen binding domain-Fc domain fusion protein. In some aspects, a) the first antigen binding domain and the Fc domain comprise an antibody and the second binding domain comprises a single chain variable fragment (scFv); or b) the second binding domain and the Fc domain comprise an antibody and the first antigen binding domain comprises a single chain variable fragment (scFv). In some aspects, the Fc domain is an Fc domain variant comprising at least one amino acid residue change as compared to a wild type sequence of the Fc domain. In some aspects, the Fc domain variant binds to an Fc receptor with altered affinity as compared to a wild type Fc domain. In some aspects, the Fc domain variant binds to an Fc receptor with decreased affinity as compared to a wild type Fc domain. In some aspects, an affinity of the Fc domain variant for an FcRn receptor is at least equivalent affinity or is not 10-fold lower an affinity of a wild type Fc domain for the FcRn receptor In some aspects, the Fc domain comprises at least one amino acid residue change selected from a group consisting of: a) N297A as in Kabat numbering and relative to SEQ ID NO: 437; b) N296G N297A as in Kabat numbering and relative to SEQ ID NO: 437; c) K322A/L234A/L235A N296A as in Kabat numbering and relative to SEQ ID NO: 437; d) L234F/L235E/P331S N296A as in Kabat numbering and relative to SEQ ID NO: 437. In some aspects, the Fc domain comprises an IgG4 Fc domain comprising S228P/L235E/P329G as in Kabat numbering.

In some aspects, a Kd for binding of the first antigen binding domain to the first antigen in the presence of the immune-modulatory compound is no greater than about two times, five times, ten times, or fifty times a Kd for binding of the first antigen binding domain to the first antigen in an absence of the immune-modulatory compound. In some aspects, a Kd for binding of the Fc domain to the Fc receptor in the presence of the immune-modulatory compound is no greater than about two times, five times, ten times, or fifty times a Kd for binding of the Fc domain to the Fc receptor in an absence of the immune-modulatory compound. In some aspects, a Kd for binding of the second binding domain to the second antigen in the presence of the immune-modulatory compound is no greater than about two times, five times, ten times, or fifty times a Kd for binding of the second binding domain to the second antigen in an absence of the immune-modulatory compound. In some aspects, the immune-modulatory compound is a PI3K inhibitor, Calcineurin inhibitor, mTOR inhibitor, BTK inhibitor, JAK inhibitor, CRAC inhibitor, PARP1 antagonist, PPARg agonist, Kv1.3 antagonist, KCa3.1 antagonist, PP2A agonist, IRAK4 inhibitor, MYD88 inhibitor, BCL-2 antagonist, A2aR agonist, TLR7 antagonist, c-KIT kinase inhibitor, KCA3.1 agonist, TGFβR inhibitor (e.g., TGFβR1 and/or TGFβR2 inhibitor), ACC antagonist, ASK1 antagonist, GLI1 antagonist, tankyrase (TNKS) antagonist, or TNIK antagonist. In some aspects, the immune-modulatory compound is Tacrolimus, rapamycin, everolimus, AZD8055, Filgotinib, Tofacitnib, Selonsertib, AMG1, AMG2, Rosiglitzone, Lobeglitzaone, or a non-PO4 accepting Fingolimod analogue. In some aspects, the first antigen binding domain comprises a single chain variable fragment from an antibody specific for the first antigen. In some aspects, the first antigen binding domain of the antibody construct comprises a set of six CDRs having at least 80% sequence identity to a set of CDRs set forth in TABLE 1, wherein the assignment of CDR residues are defined according to the IMGT (the international ImMunoGeneTics information system). In some aspects, the first antigen binding domain comprises a set of CDRs having at least 80% sequence identity to: HCDR1 comprising an amino acid sequence of SEQ ID NO: 1, HCDR2 comprising an amino acid sequence of SEQ ID NO: 2, HCDR3 comprising an amino acid sequence of SEQ ID NO: 3, LCDR1 comprising an amino acid sequence of SEQ ID NO: 4, LCDR2 comprising an amino acid sequence of SEQ ID NO: 5, and LCDR3 comprising an amino acid sequence of SEQ ID NO: 6; HCDR1 comprising an amino acid sequence of SEQ ID NO: 7, HCDR2 comprising an amino acid sequence of SEQ ID NO: 8, HCDR3 comprising an amino acid sequence of SEQ ID NO: 9, LCDR1 comprising an amino acid sequence of SEQ ID NO: 10, LCDR2 comprising an amino acid sequence of SEQ ID NO: 11, and LCDR3 comprising an amino acid sequence of SEQ ID NO: 12; HCDR1 comprising an amino acid sequence of SEQ ID NO: 13, HCDR2 comprising an amino acid sequence of SEQ ID NO: 14, HCDR3 comprising an amino acid sequence of SEQ ID NO: 15, LCDR1 comprising an amino acid sequence of SEQ ID NO: 16, LCDR2 comprising an amino acid sequence of SEQ ID NO: 17, and LCDR3 comprising an amino acid sequence of SEQ ID NO: 18; HCDR1 comprising an amino acid sequence of SEQ ID NO: 19, HCDR2 comprising an amino acid sequence of SEQ ID NO: 20, HCDR3 comprising an amino acid sequence of SEQ ID NO: 21, LCDR1 comprising an amino acid sequence of SEQ ID NO: 22, LCDR2 comprising an amino acid sequence of SEQ ID NO: 23, and LCDR3 comprising an amino acid sequence of SEQ ID NO: 24; HCDR1 comprising an amino acid sequence of SEQ ID NO: 25, HCDR2 comprising an amino acid sequence of SEQ ID NO: 26, HCDR3 comprising an amino acid sequence of SEQ ID NO: 27, LCDR1 comprising an amino acid sequence of SEQ ID NO: 28, LCDR2 comprising an amino acid sequence of SEQ ID NO: 29, and LCDR3 comprising an amino acid sequence of SEQ ID NO: 30; HCDR1 comprising an amino acid sequence of SEQ ID NO: 31, HCDR2 comprising an amino acid sequence of SEQ ID NO: 32, HCDR3 comprising an amino acid sequence of SEQ ID NO: 33, LCDR1 comprising an amino acid sequence of SEQ ID NO: 34, LCDR2 comprising an amino acid sequence of SEQ ID NO: 35, and LCDR3 comprising an amino acid sequence of SEQ ID NO: 36; HCDR1 comprising an amino acid sequence of SEQ ID NO: 37, HCDR2 comprising an amino acid sequence of SEQ ID NO: 38, HCDR3 comprising an amino acid sequence of SEQ ID NO: 39, LCDR1 comprising an amino acid sequence of SEQ ID NO: 40, LCDR2 comprising an amino acid sequence of SEQ ID NO: 41, and LCDR3 comprising an amino acid sequence of SEQ ID NO: 42; HCDR1 comprising an amino acid sequence of SEQ ID NO: 43, HCDR2 comprising an amino acid sequence of SEQ ID NO: 44, HCDR3 comprising an amino acid sequence of SEQ ID NO: 45, LCDR1 comprising an amino acid sequence of SEQ ID NO: 46, LCDR2 comprising an amino acid sequence of SEQ ID NO: 47, and LCDR3 comprising an amino acid sequence of SEQ ID NO: 48; HCDR1 comprising an amino acid sequence of SEQ ID NO: 49, HCDR2 comprising an amino acid sequence of SEQ ID NO: 50, HCDR3 comprising an amino acid sequence of SEQ ID NO: 51, LCDR1 comprising an amino acid sequence of SEQ ID NO: 52, LCDR2 comprising an amino acid sequence of SEQ ID NO: 53, and LCDR3 comprising an amino acid sequence of SEQ ID NO: 54; HCDR1 comprising an amino acid sequence of SEQ ID NO: 55, HCDR2 comprising an amino acid sequence of SEQ ID NO: 56, HCDR3 comprising an amino acid sequence of SEQ ID NO: 57, LCDR1 comprising an amino acid sequence of SEQ ID NO: 58, LCDR2 comprising an amino acid sequence of SEQ ID NO: 59, and LCDR3 comprising an amino acid sequence of SEQ ID NO: 60; HCDR1 comprising an amino acid sequence of SEQ ID NO: 61, HCDR2 comprising an amino acid sequence of SEQ ID NO: 62, HCDR3 comprising an amino acid sequence of SEQ ID NO: 63, LCDR1 comprising an amino acid sequence of SEQ ID NO: 64, LCDR2 comprising an amino acid sequence of SEQ ID NO: 65, and LCDR3 comprising an amino acid sequence of SEQ ID NO: 66; HCDR1 comprising an amino acid sequence of SEQ ID NO: 67, HCDR2 comprising an amino acid sequence of SEQ ID NO: 68, HCDR3 comprising an amino acid sequence of SEQ ID NO: 69, LCDR1 comprising an amino acid sequence of SEQ ID NO: 70, LCDR2 comprising an amino acid sequence of SEQ ID NO: 71, and LCDR3 comprising an amino acid sequence of SEQ ID NO: 72; HCDR1 comprising an amino acid sequence of SEQ ID NO: 73, HCDR2 comprising an amino acid sequence of SEQ ID NO: 74, HCDR3 comprising an amino acid sequence of SEQ ID NO: 75, LCDR1 comprising an amino acid sequence of SEQ ID NO: 76, LCDR2 comprising an amino acid sequence of SEQ ID NO: 77, and LCDR3 comprising an amino acid sequence of SEQ ID NO: 78; HCDR1 comprising an amino acid sequence of SEQ ID NO: 79, HCDR2 comprising an amino acid sequence of SEQ ID NO: 80, HCDR3 comprising an amino acid sequence of SEQ ID NO: 81, LCDR1 comprising an amino acid sequence of SEQ ID NO: 82, LCDR2 comprising an amino acid sequence of SEQ ID NO: 83, and LCDR3 comprising an amino acid sequence of SEQ ID NO: 84; HCDR1 comprising an amino acid sequence of SEQ ID NO: 85, HCDR2 comprising an amino acid sequence of SEQ ID NO: 86, HCDR3 comprising an amino acid sequence of SEQ ID NO: 87, LCDR1 comprising an amino acid sequence of SEQ ID NO: 88, LCDR2 comprising an amino acid sequence of SEQ ID NO: 89, and LCDR3 comprising an amino acid sequence of SEQ ID NO: 90; HCDR1 comprising an amino acid sequence of SEQ ID NO: 91, HCDR2 comprising an amino acid sequence of SEQ ID NO: 92, HCDR3 comprising an amino acid sequence of SEQ ID NO: 93, LCDR1 comprising an amino acid sequence of SEQ ID NO: 94, LCDR2 comprising an amino acid sequence of SEQ ID NO: 95, and LCDR3 comprising an amino acid sequence of SEQ ID NO: 96; HCDR1 comprising an amino acid sequence of SEQ ID NO: 97, HCDR2 comprising an amino acid sequence of SEQ ID NO: 98, HCDR3 comprising an amino acid sequence of SEQ ID NO: 99, LCDR1 comprising an amino acid sequence of SEQ ID NO: 100, LCDR2 comprising an amino acid sequence of SEQ ID NO: 101, and LCDR3 comprising an amino acid sequence of SEQ ID NO: 102; HCDR1 comprising an amino acid sequence of SEQ ID NO: 103, HCDR2 comprising an amino acid sequence of SEQ ID NO: 104, HCDR3 comprising an amino acid sequence of SEQ ID NO: 105, LCDR1 comprising an amino acid sequence of SEQ ID NO: 106, LCDR2 comprising an amino acid sequence of SEQ ID NO: 107, and LCDR3 comprising an amino acid sequence of SEQ ID NO: 108; HCDR1 comprising an amino acid sequence of SEQ ID NO: 109, HCDR2 comprising an amino acid sequence of SEQ ID NO: 110, HCDR3 comprising an amino acid sequence of SEQ ID NO: 111, LCDR1 comprising an amino acid sequence of SEQ ID NO: 112, LCDR2 comprising an amino acid sequence of SEQ ID NO: 113, and LCDR3 comprising an amino acid sequence of SEQ ID NO: 114; HCDR1 comprising an amino acid sequence of SEQ ID NO: 109, HCDR2 comprising an amino acid sequence of SEQ ID NO: 110, HCDR3 comprising an amino acid sequence of SEQ ID NO: 111, LCDR1 comprising an amino acid sequence of SEQ ID NO: 115, LCDR2 comprising an amino acid sequence of SEQ ID NO: 116, and LCDR3 comprising an amino acid sequence of SEQ ID NO: 117; HCDR1 comprising an amino acid sequence of SEQ ID NO: 118, HCDR2 comprising an amino acid sequence of SEQ ID NO: 119, HCDR3 comprising an amino acid sequence of SEQ ID NO: 120, LCDR1 comprising an amino acid sequence of SEQ ID NO: 121, LCDR2 comprising an amino acid sequence of SEQ ID NO: 122, and LCDR3 comprising an amino acid sequence of SEQ ID NO: 123; HCDR1 comprising an amino acid sequence of SEQ ID NO: 124, HCDR2 comprising an amino acid sequence of SEQ ID NO: 125, HCDR3 comprising an amino acid sequence of SEQ ID NO: 126, LCDR1 comprising an amino acid sequence of SEQ ID NO: 127, LCDR2 comprising an amino acid sequence of SEQ ID NO: 128, and LCDR3 comprising an amino acid sequence of SEQ ID NO: 129; HCDR1 comprising an amino acid sequence of SEQ ID NO: 130, HCDR2 comprising an amino acid sequence of SEQ ID NO: 131, HCDR3 comprising an amino acid sequence of SEQ ID NO: 132, LCDR1 comprising an amino acid sequence of SEQ ID NO: 133, LCDR2 comprising an amino acid sequence of SEQ ID NO: 134, and LCDR3 comprising an amino acid sequence of SEQ ID NO: 135; HCDR1 comprising an amino acid sequence of SEQ ID NO: 136, HCDR2 comprising an amino acid sequence of SEQ ID NO: 137, HCDR3 comprising an amino acid sequence of SEQ ID NO: 138, LCDR1 comprising an amino acid sequence of SEQ ID NO: 139, LCDR2 comprising an amino acid sequence of SEQ ID NO: 140, and LCDR3 comprising an amino acid sequence of SEQ ID NO: 141; CDR1 comprising an amino acid sequence of SEQ ID NO: 142, HCDR2 comprising an amino acid sequence of SEQ ID NO: 143, HCDR3 comprising an amino acid sequence of SEQ ID NO: 144, LCDR1 comprising an amino acid sequence of SEQ ID NO: 145, LCDR2 comprising an amino acid sequence of SEQ ID NO: 146, and LCDR3 comprising an amino acid sequence of SEQ ID NO: 147; HCDR1 comprising an amino acid sequence of SEQ ID NO: 148, HCDR2 comprising an amino acid sequence of SEQ ID NO: 149, HCDR3 comprising an amino acid sequence of SEQ ID NO: 150, LCDR1 comprising an amino acid sequence of SEQ ID NO: 151, LCDR2 comprising an amino acid sequence of SEQ ID NO: 152, and LCDR3 comprising an amino acid sequence of SEQ ID NO: 153; HCDR1 comprising an amino acid sequence of SEQ ID NO: 154, HCDR2 comprising an amino acid sequence of SEQ ID NO: 155, HCDR3 comprising an amino acid sequence of SEQ ID NO: 156, LCDR1 comprising an amino acid sequence of SEQ ID NO: 157, LCDR2 comprising an amino acid sequence of SEQ ID NO: 158, and LCDR3 comprising an amino acid sequence of SEQ ID NO: 159; HCDR1 comprising an amino acid sequence of SEQ ID NO: 160, HCDR2 comprising an amino acid sequence of SEQ ID NO: 161, HCDR3 comprising an amino acid sequence of SEQ ID NO: 162, LCDR1 comprising an amino acid sequence of SEQ ID NO: 163, LCDR2 comprising an amino acid sequence of SEQ ID NO: 164, and LCDR3 comprising an amino acid sequence of SEQ ID NO: 165; HCDR1 comprising an amino acid sequence of SEQ ID NO: 166, HCDR2 comprising an amino acid sequence of SEQ ID NO: 167, HCDR3 comprising an amino acid sequence of SEQ ID NO: 168, LCDR1 comprising an amino acid sequence of SEQ ID NO: 169, LCDR2 comprising an amino acid sequence of SEQ ID NO: 170, and LCDR3 comprising an amino acid sequence of SEQ ID NO: 171; HCDR1 comprising an amino acid sequence of SEQ ID NO: 172, HCDR2 comprising an amino acid sequence of SEQ ID NO: 173, HCDR3 comprising an amino acid sequence of SEQ ID NO: 174, LCDR1 comprising an amino acid sequence of SEQ ID NO: 175, LCDR2 comprising an amino acid sequence of SEQ ID NO: 176, and LCDR3 comprising an amino acid sequence of SEQ ID NO: 177; HCDR1 comprising an amino acid sequence of SEQ ID NO: 178, HCDR2 comprising an amino acid sequence of SEQ ID NO: 179, HCDR3 comprising an amino acid sequence of SEQ ID NO: 180, LCDR1 comprising an amino acid sequence of SEQ ID NO: 181, LCDR2 comprising an amino acid sequence of SEQ ID NO: 182, and LCDR3 comprising an amino acid sequence of SEQ ID NO: 183; HCDR1 comprising an amino acid sequence of SEQ ID NO: 184, HCDR2 comprising an amino acid sequence of SEQ ID NO: 185, HCDR3 comprising an amino acid sequence of SEQ ID NO: 186, LCDR1 comprising an amino acid sequence of SEQ ID NO: 187, LCDR2 comprising an amino acid sequence of SEQ ID NO: 188, and LCDR3 comprising an amino acid sequence of SEQ ID NO: 189; HCDR1 comprising an amino acid sequence of SEQ ID NO: 190, HCDR2 comprising an amino acid sequence of SEQ ID NO: 191, HCDR3 comprising an amino acid sequence of SEQ ID NO: 192, LCDR1 comprising an amino acid sequence of SEQ ID NO: 193, LCDR2 comprising an amino acid sequence of SEQ ID NO: 194, and LCDR3 comprising an amino acid sequence of SEQ ID NO: 195; HCDR1 comprising an amino acid sequence of SEQ ID NO: 196, HCDR2 comprising an amino acid sequence of SEQ ID NO: 197, HCDR3 comprising an amino acid sequence of SEQ ID NO: 198, LCDR1 comprising an amino acid sequence of SEQ ID NO: 199, LCDR2 comprising an amino acid sequence of SEQ ID NO: 200, and LCDR3 comprising an amino acid sequence of SEQ ID NO: 201; HCDR1 comprising an amino acid sequence of SEQ ID NO: 202, HCDR2 comprising an amino acid sequence of SEQ ID NO: 203, HCDR3 comprising an amino acid sequence of SEQ ID NO: 204, LCDR1 comprising an amino acid sequence of SEQ ID NO: 205, LCDR2 comprising an amino acid sequence of SEQ ID NO: 206, and LCDR3 comprising an amino acid sequence of SEQ ID NO: 207; HCDR1 comprising an amino acid sequence of SEQ ID NO: 208, HCDR2 comprising an amino acid sequence of SEQ ID NO: 209, HCDR3 comprising an amino acid sequence of SEQ ID NO: 210, LCDR1 comprising an amino acid sequence of SEQ ID NO: 211, LCDR2 comprising an amino acid sequence of SEQ ID NO: 212, and LCDR3 comprising an amino acid sequence of SEQ ID NO: 213; HCDR1 comprising an amino acid sequence of SEQ ID NO: 214, HCDR2 comprising an amino acid sequence of SEQ ID NO: 215, HCDR3 comprising an amino acid sequence of SEQ ID NO: 216, LCDR1 comprising an amino acid sequence of SEQ ID NO: 217, LCDR2 comprising an amino acid sequence of SEQ ID NO: 218, and LCDR3 comprising an amino acid sequence of SEQ ID NO: 219; HCDR1 comprising an amino acid sequence of SEQ ID NO: 220, HCDR2 comprising an amino acid sequence of SEQ ID NO: 221, HCDR3 comprising an amino acid sequence of SEQ ID NO: 222, LCDR1 comprising an amino acid sequence of SEQ ID NO: 223, LCDR2 comprising an amino acid sequence of SEQ ID NO: 224, and LCDR3 comprising an amino acid sequence of SEQ ID NO: 225; HCDR1 comprising an amino acid sequence of SEQ ID NO: 226, HCDR2 comprising an amino acid sequence of SEQ ID NO: 227, HCDR3 comprising an amino acid sequence of SEQ ID NO: 228, LCDR1 comprising an amino acid sequence of SEQ ID NO: 229, LCDR2 comprising an amino acid sequence of SEQ ID NO: 230, and LCDR3 comprising an amino acid sequence of SEQ ID NO: 231; HCDR1 comprising an amino acid sequence of SEQ ID NO: 232, HCDR2 comprising an amino acid sequence of SEQ ID NO: 234, HCDR3 comprising an amino acid sequence of SEQ ID NO: 235, LCDR1 comprising an amino acid sequence of SEQ ID NO: 236, LCDR2 comprising an amino acid sequence of SEQ ID NO: 237, and LCDR3 comprising an amino acid sequence of SEQ ID NO: 238; HCDR1 comprising an amino acid sequence of SEQ ID NO: 239, HCDR2 comprising an amino acid sequence of SEQ ID NO: 240, HCDR3 comprising an amino acid sequence of SEQ ID NO: 241, LCDR1 comprising an amino acid sequence of SEQ ID NO: 242, LCDR2 comprising an amino acid sequence of SEQ ID NO: 243, and LCDR3 comprising an amino acid sequence of SEQ ID NO: 244; HCDR1 comprising an amino acid sequence of SEQ ID NO: 245, HCDR2 comprising an amino acid sequence of SEQ ID NO: 246, HCDR3 comprising an amino acid sequence of SEQ ID NO: 247, LCDR1 comprising an amino acid sequence of SEQ ID NO: 248, LCDR2 comprising an amino acid sequence of SEQ ID NO: 249, and LCDR3 comprising an amino acid sequence of SEQ ID NO: 250; HCDR1 comprising an amino acid sequence of SEQ ID NO: 251, HCDR2 comprising an amino acid sequence of SEQ ID NO: 252, HCDR3 comprising an amino acid sequence of SEQ ID NO: 253, LCDR1 comprising an amino acid sequence of SEQ ID NO: 254, LCDR2 comprising an amino acid sequence of SEQ ID NO: 255, and LCDR3 comprising an amino acid sequence of SEQ ID NO: 256; HCDR1 comprising an amino acid sequence of SEQ ID NO: 257, HCDR2 comprising an amino acid sequence of SEQ ID NO: 258, HCDR3 comprising an amino acid sequence of SEQ ID NO: 259, LCDR1 comprising an amino acid sequence of SEQ ID NO: 260, LCDR2 comprising an amino acid sequence of SEQ ID NO: 261, and LCDR3 comprising an amino acid sequence of SEQ ID NO: 262; HCDR1 comprising an amino acid sequence of SEQ ID NO: 263, HCDR2 comprising an amino acid sequence of SEQ ID NO: 264, HCDR3 comprising an amino acid sequence of SEQ ID NO: 265, LCDR1 comprising an amino acid sequence of SEQ ID NO: 266, LCDR2 comprising an amino acid sequence of SEQ ID NO: 267, and LCDR3 comprising an amino acid sequence of SEQ ID NO: 268; HCDR1 comprising an amino acid sequence of SEQ ID NO: 269, HCDR2 comprising an amino acid sequence of SEQ ID NO: 270, HCDR3 comprising an amino acid sequence of SEQ ID NO: 271, LCDR1 comprising an amino acid sequence of SEQ ID NO: 272, LCDR2 comprising an amino acid sequence of SEQ ID NO: 273, and LCDR3 comprising an amino acid sequence of SEQ ID NO: 274; HCDR1 comprising an amino acid sequence of SEQ ID NO: 275, HCDR2 comprising an amino acid sequence of SEQ ID NO: 276, HCDR3 comprising an amino acid sequence of SEQ ID NO: 277, LCDR1 comprising an amino acid sequence of SEQ ID NO: 278, LCDR2 comprising an amino acid sequence of SEQ ID NO: 279, and LCDR3 comprising an amino acid sequence of SEQ ID NO: 280; HCDR1 comprising an amino acid sequence of SEQ ID NO: 281, HCDR2 comprising an amino acid sequence of SEQ ID NO: 282, HCDR3 comprising an amino acid sequence of SEQ ID NO: 283, LCDR1 comprising an amino acid sequence of SEQ ID NO: 284, LCDR2 comprising an amino acid sequence of SEQ ID NO: 285, and LCDR3 comprising an amino acid sequence of SEQ ID NO: 286; HCDR1 comprising an amino acid sequence of SEQ ID NO: 287, HCDR2 comprising an amino acid sequence of SEQ ID NO: 288, HCDR3 comprising an amino acid sequence of SEQ ID NO: 289, LCDR1 comprising an amino acid sequence of SEQ ID NO: 290, LCDR2 comprising an amino acid sequence of SEQ ID NO: 291, and LCDR3 comprising an amino acid sequence of SEQ ID NO: 292; or HCDR1 comprising an amino acid sequence of SEQ ID NO: 293, HCDR2 comprising an amino acid sequence of SEQ ID NO: 294, HCDR3 comprising an amino acid sequence of SEQ ID NO: 295, LCDR1 comprising an amino acid sequence of SEQ ID NO: 296, LCDR2 comprising an amino acid sequence of SEQ ID NO: 297, and LCDR3 comprising an amino acid sequence of SEQ ID NO: 298; HCDR1 comprising an amino acid sequence of SEQ ID NO: 440, HCDR2 comprising an amino acid sequence of SEQ ID NO: 441, HCDR3 comprising an amino acid sequence of SEQ ID NO: 442, LCDR1 comprising an amino acid sequence of SEQ ID NO: 443, LCDR2 comprising an amino acid sequence of SEQ ID NO: 444, and LCDR3 comprising an amino acid sequence of SEQ ID NO: 445; HCDR1 comprising an amino acid sequence of SEQ ID NO: 446, HCDR2 comprising an amino acid sequence of SEQ ID NO: 447, HCDR3 comprising an amino acid sequence of SEQ ID NO: 448, LCDR1 comprising an amino acid sequence of SEQ ID NO: 449, LCDR2 comprising an amino acid sequence of SEQ ID NO: 450, and LCDR3 comprising an amino acid sequence of SEQ ID NO: 451; HCDR1 comprising an amino acid sequence of SEQ ID NO: 452, HCDR2 comprising an amino acid sequence of SEQ ID NO: 453, HCDR3 comprising an amino acid sequence of SEQ ID NO: 454, LCDR1 comprising an amino acid sequence of SEQ ID NO: 455, LCDR2 comprising an amino acid sequence of SEQ ID NO: 456, and LCDR3 comprising an amino acid sequence of SEQ ID NO: 457; HCDR1 comprising an amino acid sequence of SEQ ID NO: 458, HCDR2 comprising an amino acid sequence of SEQ ID NO: 459, HCDR3 comprising an amino acid sequence of SEQ ID NO: 460, LCDR1 comprising an amino acid sequence of SEQ ID NO: 461, LCDR2 comprising an amino acid sequence of SEQ ID NO: 462, and LCDR3 comprising an amino acid sequence of SEQ ID NO: 463; HCDR1 comprising an amino acid sequence of SEQ ID NO: 464, HCDR2 comprising an amino acid sequence of SEQ ID NO: 465, HCDR3 comprising an amino acid sequence of SEQ ID NO: 466, LCDR1 comprising an amino acid sequence of SEQ ID NO: 467, LCDR2 comprising an amino acid sequence of SEQ ID NO: 468, and LCDR3 comprising an amino acid sequence of SEQ ID NO: 469; HCDR1 comprising an amino acid sequence of SEQ ID NO: 470, HCDR2 comprising an amino acid sequence of SEQ ID NO: 471, HCDR3 comprising an amino acid sequence of SEQ ID NO: 472, LCDR1 comprising an amino acid sequence of SEQ ID NO: 473, LCDR2 comprising an amino acid sequence of SEQ ID NO: 474, and LCDR3 comprising an amino acid sequence of SEQ ID NO: 475; or HCDR1 comprising an amino acid sequence of SEQ ID NO: 476, HCDR2 comprising an amino acid sequence of SEQ ID NO: 477, HCDR3 comprising an amino acid sequence of SEQ ID NO: 478, LCDR1 comprising an amino acid sequence of SEQ ID NO: 479, LCDR2 comprising an amino acid sequence of SEQ ID NO: 480, and LCDR3 comprising an amino acid sequence of SEQ ID NO: 481.

In some aspects, the second binding domain comprises a single chain variable fragment from an antibody specific for the second antigen. In some aspects, the second binding domain comprises a set of six CDRs having at least 80% sequence identity a set of CDRs set forth in Table 1 as SEQ ID NO: 85-SEQ ID NO: 298. In some aspects, the second binding domain comprises at least 80% sequence identity to: HCDR1 comprising an amino acid sequence of SEQ ID NO: 85, HCDR2 comprising an amino acid sequence of SEQ ID NO: 86, HCDR3 comprising an amino acid sequence of SEQ ID NO: 87, LCDR1 comprising an amino acid sequence of SEQ ID NO: 88, LCDR2 comprising an amino acid sequence of SEQ ID NO: 89, and LCDR3 comprising an amino acid sequence of SEQ ID NO: 90; HCDR1 comprising an amino acid sequence of SEQ ID NO: 91, HCDR2 comprising an amino acid sequence of SEQ ID NO: 92, HCDR3 comprising an amino acid sequence of SEQ ID NO: 93, LCDR1 comprising an amino acid sequence of SEQ ID NO: 94, LCDR2 comprising an amino acid sequence of SEQ ID NO: 95, and LCDR3 comprising an amino acid sequence of SEQ ID NO: 96; HCDR1 comprising an amino acid sequence of SEQ ID NO: 97, HCDR2 comprising an amino acid sequence of SEQ ID NO: 98, HCDR3 comprising an amino acid sequence of SEQ ID NO: 99, LCDR1 comprising an amino acid sequence of SEQ ID NO: 100, LCDR2 comprising an amino acid sequence of SEQ ID NO: 101, and LCDR3 comprising an amino acid sequence of SEQ ID NO: 102; HCDR1 comprising an amino acid sequence of SEQ ID NO: 103, HCDR2 comprising an amino acid sequence of SEQ ID NO: 104, HCDR3 comprising an amino acid sequence of SEQ ID NO: 105, LCDR1 comprising an amino acid sequence of SEQ ID NO: 106, LCDR2 comprising an amino acid sequence of SEQ ID NO: 107, and LCDR3 comprising an amino acid sequence of SEQ ID NO: 108; HCDR1 comprising an amino acid sequence of SEQ ID NO: 109, HCDR2 comprising an amino acid sequence of SEQ ID NO: 110, HCDR3 comprising an amino acid sequence of SEQ ID NO: 111, LCDR1 comprising an amino acid sequence of SEQ ID NO: 112, LCDR2 comprising an amino acid sequence of SEQ ID NO: 113, and LCDR3 comprising an amino acid sequence of SEQ ID NO: 114; HCDR1 comprising an amino acid sequence of SEQ ID NO: 109, HCDR2 comprising an amino acid sequence of SEQ ID NO: 110, HCDR3 comprising an amino acid sequence of SEQ ID NO: 111, LCDR1 comprising an amino acid sequence of SEQ ID NO: 115, LCDR2 comprising an amino acid sequence of SEQ ID NO: 116, and LCDR3 comprising an amino acid sequence of SEQ ID NO: 117; HCDR1 comprising an amino acid sequence of SEQ ID NO: 118, HCDR2 comprising an amino acid sequence of SEQ ID NO: 119, HCDR3 comprising an amino acid sequence of SEQ ID NO: 120, LCDR1 comprising an amino acid sequence of SEQ ID NO: 121, LCDR2 comprising an amino acid sequence of SEQ ID NO: 122, and LCDR3 comprising an amino acid sequence of SEQ ID NO: 123; HCDR1 comprising an amino acid sequence of SEQ ID NO: 124, HCDR2 comprising an amino acid sequence of SEQ ID NO: 125, HCDR3 comprising an amino acid sequence of SEQ ID NO: 126, LCDR1 comprising an amino acid sequence of SEQ ID NO: 127, LCDR2 comprising an amino acid sequence of SEQ ID NO: 128, and LCDR3 comprising an amino acid sequence of SEQ ID NO: 129; HCDR1 comprising an amino acid sequence of SEQ ID NO: 130, HCDR2 comprising an amino acid sequence of SEQ ID NO: 131, HCDR3 comprising an amino acid sequence of SEQ ID NO: 132, LCDR1 comprising an amino acid sequence of SEQ ID NO: 133, LCDR2 comprising an amino acid sequence of SEQ ID NO: 134, and LCDR3 comprising an amino acid sequence of SEQ ID NO: 135; HCDR1 comprising an amino acid sequence of SEQ ID NO: 136, HCDR2 comprising an amino acid sequence of SEQ ID NO: 137, HCDR3 comprising an amino acid sequence of SEQ ID NO: 138, LCDR1 comprising an amino acid sequence of SEQ ID NO: 139, LCDR2 comprising an amino acid sequence of SEQ ID NO: 140, and LCDR3 comprising an amino acid sequence of SEQ ID NO: 141; HCDR1 comprising an amino acid sequence of SEQ ID NO: 142, HCDR2 comprising an amino acid sequence of SEQ ID NO: 143, HCDR3 comprising an amino acid sequence of SEQ ID NO: 144, LCDR1 comprising an amino acid sequence of SEQ ID NO: 145, LCDR2 comprising an amino acid sequence of SEQ ID NO: 146, and LCDR3 comprising an amino acid sequence of SEQ ID NO: 147; HCDR1 comprising an amino acid sequence of SEQ ID NO: 148, HCDR2 comprising an amino acid sequence of SEQ ID NO: 149, HCDR3 comprising an amino acid sequence of SEQ ID NO: 150, LCDR1 comprising an amino acid sequence of SEQ ID NO: 151, LCDR2 comprising an amino acid sequence of SEQ ID NO: 152, and LCDR3 comprising an amino acid sequence of SEQ ID NO: 153; HCDR1 comprising an amino acid sequence of SEQ ID NO: 154, HCDR2 comprising an amino acid sequence of SEQ ID NO: 155, HCDR3 comprising an amino acid sequence of SEQ ID NO: 156, LCDR1 comprising an amino acid sequence of SEQ ID NO: 157, LCDR2 comprising an amino acid sequence of SEQ ID NO: 158, and LCDR3 comprising an amino acid sequence of SEQ ID NO: 159; HCDR1 comprising an amino acid sequence of SEQ ID NO: 160, HCDR2 comprising an amino acid sequence of SEQ ID NO: 161, HCDR3 comprising an amino acid sequence of SEQ ID NO: 162, LCDR1 comprising an amino acid sequence of SEQ ID NO: 163, LCDR2 comprising an amino acid sequence of SEQ ID NO: 164, and LCDR3 comprising an amino acid sequence of SEQ ID NO: 165; HCDR1 comprising an amino acid sequence of SEQ ID NO: 166, HCDR2 comprising an amino acid sequence of SEQ ID NO: 167, HCDR3 comprising an amino acid sequence of SEQ ID NO: 168, LCDR1 comprising an amino acid sequence of SEQ ID NO: 169, LCDR2 comprising an amino acid sequence of SEQ ID NO: 170, and LCDR3 comprising an amino acid sequence of SEQ ID NO: 171; HCDR1 comprising an amino acid sequence of SEQ ID NO: 172, HCDR2 comprising an amino acid sequence of SEQ ID NO: 173, HCDR3 comprising an amino acid sequence of SEQ ID NO: 174, LCDR1 comprising an amino acid sequence of SEQ ID NO: 175, LCDR2 comprising an amino acid sequence of SEQ ID NO: 176, and LCDR3 comprising an amino acid sequence of SEQ ID NO: 177; HCDR1 comprising an amino acid sequence of SEQ ID NO: 178, HCDR2 comprising an amino acid sequence of SEQ ID NO: 179, HCDR3 comprising an amino acid sequence of SEQ ID NO: 180, LCDR1 comprising an amino acid sequence of SEQ ID NO: 181, LCDR2 comprising an amino acid sequence of SEQ ID NO: 182, and LCDR3 comprising an amino acid sequence of SEQ ID NO: 183; HCDR1 comprising an amino acid sequence of SEQ ID NO: 184, HCDR2 comprising an amino acid sequence of SEQ ID NO: 185, HCDR3 comprising an amino acid sequence of SEQ ID NO: 186, LCDR1 comprising an amino acid sequence of SEQ ID NO: 187, LCDR2 comprising an amino acid sequence of SEQ ID NO: 188, and LCDR3 comprising an amino acid sequence of SEQ ID NO: 189; HCDR1 comprising an amino acid sequence of SEQ ID NO: 190, HCDR2 comprising an amino acid sequence of SEQ ID NO: 191, HCDR3 comprising an amino acid sequence of SEQ ID NO: 192, LCDR1 comprising an amino acid sequence of SEQ ID NO: 193, LCDR2 comprising an amino acid sequence of SEQ ID NO: 194, and LCDR3 comprising an amino acid sequence of SEQ ID NO: 195; HCDR1 comprising an amino acid sequence of SEQ ID NO: 196, HCDR2 comprising an amino acid sequence of SEQ ID NO: 197, HCDR3 comprising an amino acid sequence of SEQ ID NO: 198, LCDR1 comprising an amino acid sequence of SEQ ID NO: 199, LCDR2 comprising an amino acid sequence of SEQ ID NO: 200, and LCDR3 comprising an amino acid sequence of SEQ ID NO: 201; HCDR1 comprising an amino acid sequence of SEQ ID NO: 202, HCDR2 comprising an amino acid sequence of SEQ ID NO: 203, HCDR3 comprising an amino acid sequence of SEQ ID NO: 204, LCDR1 comprising an amino acid sequence of SEQ ID NO: 205, LCDR2 comprising an amino acid sequence of SEQ ID NO: 206, and LCDR3 comprising an amino acid sequence of SEQ ID NO: 207; HCDR1 comprising an amino acid sequence of SEQ ID NO: 208, HCDR2 comprising an amino acid sequence of SEQ ID NO: 209, HCDR3 comprising an amino acid sequence of SEQ ID NO: 210, LCDR1 comprising an amino acid sequence of SEQ ID NO: 211, LCDR2 comprising an amino acid sequence of SEQ ID NO: 212, and LCDR3 comprising an amino acid sequence of SEQ ID NO: 213; HCDR1 comprising an amino acid sequence of SEQ ID NO: 214, HCDR2 comprising an amino acid sequence of SEQ ID NO: 215, HCDR3 comprising an amino acid sequence of SEQ ID NO: 216, LCDR1 comprising an amino acid sequence of SEQ ID NO: 217, LCDR2 comprising an amino acid sequence of SEQ ID NO: 218, and LCDR3 comprising an amino acid sequence of SEQ ID NO: 219; HCDR1 comprising an amino acid sequence of SEQ ID NO: 220, HCDR2 comprising an amino acid sequence of SEQ ID NO: 221, HCDR3 comprising an amino acid sequence of SEQ ID NO: 222, LCDR1 comprising an amino acid sequence of SEQ ID NO: 223, LCDR2 comprising an amino acid sequence of SEQ ID NO: 224, and LCDR3 comprising an amino acid sequence of SEQ ID NO: 225; HCDR1 comprising an amino acid sequence of SEQ ID NO: 226, HCDR2 comprising an amino acid sequence of SEQ ID NO: 227, HCDR3 comprising an amino acid sequence of SEQ ID NO: 228, LCDR1 comprising an amino acid sequence of SEQ ID NO: 229, LCDR2 comprising an amino acid sequence of SEQ ID NO: 230, and LCDR3 comprising an amino acid sequence of SEQ ID NO: 231; HCDR1 comprising an amino acid sequence of SEQ ID NO: 232, HCDR2 comprising an amino acid sequence of SEQ ID NO: 234, HCDR3 comprising an amino acid sequence of SEQ ID NO: 235, LCDR1 comprising an amino acid sequence of SEQ ID NO: 236, LCDR2 comprising an amino acid sequence of SEQ ID NO: 237, and LCDR3 comprising an amino acid sequence of SEQ ID NO: 238; HCDR1 comprising an amino acid sequence of SEQ ID NO: 239, HCDR2 comprising an amino acid sequence of SEQ ID NO: 240, HCDR3 comprising an amino acid sequence of SEQ ID NO: 241, LCDR1 comprising an amino acid sequence of SEQ ID NO: 242, LCDR2 comprising an amino acid sequence of SEQ ID NO: 243, and LCDR3 comprising an amino acid sequence of SEQ ID NO: 244; HCDR1 comprising an amino acid sequence of SEQ ID NO: 245, HCDR2 comprising an amino acid sequence of SEQ ID NO: 246, HCDR3 comprising an amino acid sequence of SEQ ID NO: 247, LCDR1 comprising an amino acid sequence of SEQ ID NO: 248, LCDR2 comprising an amino acid sequence of SEQ ID NO: 249, and LCDR3 comprising an amino acid sequence of SEQ ID NO: 250; HCDR1 comprising an amino acid sequence of SEQ ID NO: 251, HCDR2 comprising an amino acid sequence of SEQ ID NO: 252, HCDR3 comprising an amino acid sequence of SEQ ID NO: 253, LCDR1 comprising an amino acid sequence of SEQ ID NO: 254, LCDR2 comprising an amino acid sequence of SEQ ID NO: 255, and LCDR3 comprising an amino acid sequence of SEQ ID NO: 256; HCDR1 comprising an amino acid sequence of SEQ ID NO: 257, HCDR2 comprising an amino acid sequence of SEQ ID NO: 258, HCDR3 comprising an amino acid sequence of SEQ ID NO: 259, LCDR1 comprising an amino acid sequence of SEQ ID NO: 260, LCDR2 comprising an amino acid sequence of SEQ ID NO: 261, and LCDR3 comprising an amino acid sequence of SEQ ID NO: 262; HCDR1 comprising an amino acid sequence of SEQ ID NO: 263, HCDR2 comprising an amino acid sequence of SEQ ID NO: 264, HCDR3 comprising an amino acid sequence of SEQ ID NO: 265, LCDR1 comprising an amino acid sequence of SEQ ID NO: 266, LCDR2 comprising an amino acid sequence of SEQ ID NO: 267, and LCDR3 comprising an amino acid sequence of SEQ ID NO: 268; HCDR1 comprising an amino acid sequence of SEQ ID NO: 269, HCDR2 comprising an amino acid sequence of SEQ ID NO: 270, HCDR3 comprising an amino acid sequence of SEQ ID NO: 271, LCDR1 comprising an amino acid sequence of SEQ ID NO: 272, LCDR2 comprising an amino acid sequence of SEQ ID NO: 273, and LCDR3 comprising an amino acid sequence of SEQ ID NO: 274; HCDR1 comprising an amino acid sequence of SEQ ID NO: 275, HCDR2 comprising an amino acid sequence of SEQ ID NO: 276, HCDR3 comprising an amino acid sequence of SEQ ID NO: 277, LCDR1 comprising an amino acid sequence of SEQ ID NO: 278, LCDR2 comprising an amino acid sequence of SEQ ID NO: 279, and LCDR3 comprising an amino acid sequence of SEQ ID NO: 280; HCDR1 comprising an amino acid sequence of SEQ ID NO: 281, HCDR2 comprising an amino acid sequence of SEQ ID NO: 282, HCDR3 comprising an amino acid sequence of SEQ ID NO: 283, LCDR1 comprising an amino acid sequence of SEQ ID NO: 284, LCDR2 comprising an amino acid sequence of SEQ ID NO: 285, and LCDR3 comprising an amino acid sequence of SEQ ID NO: 286; HCDR1 comprising an amino acid sequence of SEQ ID NO: 287, HCDR2 comprising an amino acid sequence of SEQ ID NO: 288, HCDR3 comprising an amino acid sequence of SEQ ID NO: 289, LCDR1 comprising an amino acid sequence of SEQ ID NO: 290, LCDR2 comprising an amino acid sequence of SEQ ID NO: 291, and LCDR3 comprising an amino acid sequence of SEQ ID NO: 292; or HCDR1 comprising an amino acid sequence of SEQ ID NO: 293, HCDR2 comprising an amino acid sequence of SEQ ID NO: 294, HCDR3 comprising an amino acid sequence of SEQ ID NO: 295, LCDR1 comprising an amino acid sequence of SEQ ID NO: 296, LCDR2 comprising an amino acid sequence of SEQ ID NO: 297, and LCDR3 comprising an amino acid sequence of SEQ ID NO: 298.

In some aspects, the first antigen binding domain comprises a variable region heavy and light chain having at least 80% sequence identity to a pair of variable region heavy and light chains set forth in TABLE 2. In some aspects, the first antigen binding domain comprises a pair of variable region heavy and light chains having at least 80% sequence identity to a pair of variable region heavy and light chains set forth in TABLE 2. In some aspects, the first antigen binding domain comprises a pair of variable region heavy and light chains having at least 80% sequence identity to the non-CDR regions of a pair of variable region heavy and light chains set forth in TABLE 2.

In some aspects, the first antigen binding domain comprises: a VH sequence having at least 80% sequence identity to an amino acid sequence of SEQ ID NO: 300, and a VL sequence having at least 80% sequence identity to an amino acid sequence of SEQ ID NO: 299; a VH sequence having at least 80% sequence identity to an amino acid sequence of SEQ ID NO: 301, and a VL sequence having at least 80% sequence identity to an amino acid sequence of SEQ ID NO: 299; a VH sequence having at least 80% sequence identity to an amino acid sequence of SEQ ID NO: 302, and a VL sequence having at least 80% sequence identity to an amino acid sequence of SEQ ID NO: 303; a VH sequence having at least 80% sequence identity to an amino acid sequence of SEQ ID NO: 304, and a VL sequence having at least 80% sequence identity to an amino acid sequence of SEQ ID NO: 305; a VH sequence having at least 80% sequence identity to an amino acid sequence of SEQ ID NO: 306, and a VL sequence having at least 80% sequence identity to an amino acid sequence of SEQ ID NO: 307; a VH sequence having at least 80% sequence identity to an amino acid sequence of SEQ ID NO: 308, and a VL sequence having at least 80% sequence identity to an amino acid sequence of SEQ ID NO: 309; a VH sequence having at least 80% sequence identity to an amino acid sequence of SEQ ID NO: 310, and a VL sequence having at least 80% sequence identity to an amino acid sequence of SEQ ID NO: 311; a VH sequence having at least 80% sequence identity to an amino acid sequence of SEQ ID NO: 312, and a VL sequence having at least 80% sequence identity to an amino acid sequence of SEQ ID NO: 313; a VH sequence having at least 80% sequence identity to an amino acid sequence of SEQ ID NO: 314, and a VL sequence having at least 80% sequence identity to an amino acid sequence of SEQ ID NO: 315; a VH sequence having at least 80% sequence identity to an amino acid sequence of SEQ ID NO: 316, and a VL sequence having at least 80% sequence identity to an amino acid sequence of SEQ ID NO: 317; a VH sequence having at least 80% sequence identity to an amino acid sequence of SEQ ID NO: 318, and a VL sequence having at least 80% sequence identity to an amino acid sequence of SEQ ID NO: 320; a VH sequence having at least 80% sequence identity to an amino acid sequence of SEQ ID NO: 319, and a VL sequence having at least 80% sequence identity to an amino acid sequence of SEQ ID NO: 320; a VH sequence having at least 80% sequence identity to an amino acid sequence of SEQ ID NO: 321, and a VL sequence having at least 80% sequence identity to an amino acid sequence of SEQ ID NO: 322; a VH sequence having at least 80% sequence identity to an amino acid sequence of SEQ ID NO: 323, and a VL sequence having at least 80% sequence identity to an amino acid sequence of SEQ ID NO: 324; a VH sequence having at least 80% sequence identity to an amino acid sequence of SEQ ID NO: 325, and a VL sequence having at least 80% sequence identity to an amino acid sequence of SEQ ID NO: 326; a VH sequence having at least 80% sequence identity to an amino acid sequence of SEQ ID NO: 327, and a VL sequence having at least 80% sequence identity to an amino acid sequence of SEQ ID NO: 328; a VH sequence having at least 80% sequence identity to an amino acid sequence of SEQ ID NO: 329, and a VL sequence having at least 80% sequence identity to an amino acid sequence of SEQ ID NO: 330; a VH sequence having at least 80% sequence identity to an amino acid sequence of SEQ ID NO: 331, and a VL sequence having at least 80% sequence identity to an amino acid sequence of SEQ ID NO: 334; a VH sequence having at least 80% sequence identity to an amino acid sequence of SEQ ID NO: 331, and a VL sequence having at least 80% sequence identity to an amino acid sequence of SEQ ID NO: 335; a VH sequence having at least 80% sequence identity to an amino acid sequence of SEQ ID NO: 332, and a VL sequence having at least 80% sequence identity to an amino acid sequence of SEQ ID NO: 334; a VH sequence having at least 80% sequence identity to an amino acid sequence of SEQ ID NO: 332, and a VL sequence having at least 80% sequence identity to an amino acid sequence of SEQ ID NO: 335; a VH sequence having at least 80% sequence identity to an amino acid sequence of SEQ ID NO: 333, and a VL sequence having at least 80% sequence identity to an amino acid sequence of SEQ ID NO: 334; a VH sequence having at least 80% sequence identity to an amino acid sequence of SEQ ID NO: 333, and a VL sequence having at least 80% sequence identity to an amino acid sequence of SEQ ID NO: 335; a VH sequence having at least 80% sequence identity to an amino acid sequence of SEQ ID NO: 336, and a VL sequence having at least 80% sequence identity to an amino acid sequence of SEQ ID NO: 337; a VH sequence having at least 80% sequence identity to an amino acid sequence of SEQ ID NO: 338, and a VL sequence having at least 80% sequence identity to an amino acid sequence of SEQ ID NO: 339; a VH sequence having at least 80% sequence identity to an amino acid sequence of SEQ ID NO: 340, and a VL sequence having at least 80% sequence identity to an amino acid sequence of SEQ ID NO: 341; a VH sequence having at least 80% sequence identity to an amino acid sequence of SEQ ID NO: 342, and a VL sequence having at least 80% sequence identity to an amino acid sequence of SEQ ID NO: 343; a VH sequence having at least 80% sequence identity to an amino acid sequence of SEQ ID NO: 344, and a VL sequence having at least 80% sequence identity to an amino acid sequence of SEQ ID NO: 345; a VH sequence having at least 80% sequence identity to an amino acid sequence of SEQ ID NO: 346, and a VL sequence having at least 80% sequence identity to an amino acid sequence of SEQ ID NO: 347; a VH sequence having at least 80% sequence identity to an amino acid sequence of SEQ ID NO: 348, and a VL sequence having at least 80% sequence identity to an amino acid sequence of SEQ ID NO: 349; a VH sequence having at least 80% sequence identity to an amino acid sequence of SEQ ID NO: 350, and a VL sequence having at least 80% sequence identity to an amino acid sequence of SEQ ID NO: 351; a VH sequence having at least 80% sequence identity to an amino acid sequence of SEQ ID NO: 352, and a VL sequence having at least 80% sequence identity to an amino acid sequence of SEQ ID NO: 353; a VH sequence having at least 80% sequence identity to an amino acid sequence of SEQ ID NO: 354, and a VL sequence having at least 80% sequence identity to an amino acid sequence of SEQ ID NO: 355; a VH sequence having at least 80% sequence identity to an amino acid sequence of SEQ ID NO: 356, and a VL sequence having at least 80% sequence identity to an amino acid sequence of SEQ ID NO: 357; a VH sequence having at least 80% sequence identity to an amino acid sequence of SEQ ID NO: 358, and a VL sequence having at least 80% sequence identity to an amino acid sequence of SEQ ID NO: 359; a VH sequence having at least 80% sequence identity to an amino acid sequence of SEQ ID NO: 358, and a VL sequence having at least 80% sequence identity to an amino acid sequence of SEQ ID NO: 360; a VH sequence having at least 80% sequence identity to an amino acid sequence of SEQ ID NO: 361, and a VL sequence having at least 80% sequence identity to an amino acid sequence of SEQ ID NO: 362; a VH sequence having at least 80% sequence identity to an amino acid sequence of SEQ ID NO: 363, and a VL sequence having at least 80% sequence identity to an amino acid sequence of SEQ ID NO: 364; a VH sequence having at least 80% sequence identity to an amino acid sequence of SEQ ID NO: 365, and a VL sequence having at least 80% sequence identity to an amino acid sequence of SEQ ID NO: 366; a VH sequence having at least 80% sequence identity to an amino acid sequence of SEQ ID NO: 367, and a VL sequence having at least 80% sequence identity to an amino acid sequence of SEQ ID NO: 368; a VH sequence having at least 80% sequence identity to an amino acid sequence of SEQ ID NO: 367, and a VL sequence having at least 80% sequence identity to an amino acid sequence of SEQ ID NO: 369; a VH sequence having at least 80% sequence identity to an amino acid sequence of SEQ ID NO: 367, and a VL sequence having at least 80% sequence identity to an amino acid sequence of SEQ ID NO: 370; a VH sequence having at least 80% sequence identity to an amino acid sequence of SEQ ID NO: 367, and a VL sequence having at least 80% sequence identity to an amino acid sequence of SEQ ID NO: 371; a VH sequence having at least 80% sequence identity to an amino acid sequence of SEQ ID NO: 367, and a VL sequence having at least 80% sequence identity to an amino acid sequence of SEQ ID NO: 372; a VH sequence having at least 80% sequence identity to an amino acid sequence of SEQ ID NO: 374, and a VL sequence having at least 80% sequence identity to an amino acid sequence of SEQ ID NO: 373; a VH sequence having at least 80% sequence identity to an amino acid sequence of SEQ ID NO: 375, and a VL sequence having at least 80% sequence identity to an amino acid sequence of SEQ ID NO: 376; a VH sequence having at least 80% sequence identity to an amino acid sequence of SEQ ID NO: 377, and a VL sequence having at least 80% sequence identity to an amino acid sequence of SEQ ID NO: 378; a VH sequence having at least 80% sequence identity to an amino acid sequence of SEQ ID NO: 379, and a VL sequence having at least 80% sequence identity to an amino acid sequence of SEQ ID NO: 380; a VH sequence having at least 80% sequence identity to an amino acid sequence of SEQ ID NO: 381, and a VL sequence having at least 80% sequence identity to an amino acid sequence of SEQ ID NO: 382; a VH sequence having at least 80% sequence identity to an amino acid sequence of SEQ ID NO: 384, and a VL sequence having at least 80% sequence identity to an amino acid sequence of SEQ ID NO: 383; a VH sequence having at least 80% sequence identity to an amino acid sequence of SEQ ID NO: 385, and a VL sequence having at least 80% sequence identity to an amino acid sequence of SEQ ID NO: 386; a VH sequence having at least 80% sequence identity to an amino acid sequence of SEQ ID NO: 387, and a VL sequence having at least 80% sequence identity to an amino acid sequence of SEQ ID NO: 388; a VH sequence having at least 80% sequence identity to an amino acid sequence of SEQ ID NO: 389, and a VL sequence having at least 80% sequence identity to an amino acid sequence of SEQ ID NO: 390; a VH sequence having at least 80% sequence identity to an amino acid sequence of SEQ ID NO: 391, and a VL sequence having at least 80% sequence identity to an amino acid sequence of SEQ ID NO: 392; a VH sequence having at least 80% sequence identity to an amino acid sequence of SEQ ID NO: 393, and a VL sequence having at least 80% sequence identity to an amino acid sequence of SEQ ID NO: 394; a VH sequence having at least 80% sequence identity to an amino acid sequence of SEQ ID NO: 395, and a VL sequence having at least 80% sequence identity to an amino acid sequence of SEQ ID NO: 396; a VH sequence having at least 80% sequence identity to an amino acid sequence of SEQ ID NO: 397, and a VL sequence having at least 80% sequence identity to an amino acid sequence of SEQ ID NO: 398; a VH sequence having at least 80% sequence identity to an amino acid sequence of SEQ ID NO: 399, and a VL sequence having at least 80% sequence identity to an amino acid sequence of SEQ ID NO: 400; a VH sequence having at least 80% sequence identity to an amino acid sequence of SEQ ID NO: 401, and a VL sequence having at least 80% sequence identity to an amino acid sequence of SEQ ID NO: 402; a VH sequence having at least 80% sequence identity to an amino acid sequence of SEQ ID NO: 403, and a VL sequence having at least 80% sequence identity to an amino acid sequence of SEQ ID NO: 404; a VH sequence having at least 80% sequence identity to an amino acid sequence of SEQ ID NO: 405, and a VL sequence having at least 80% sequence identity to an amino acid sequence of SEQ ID NO: 406; a VH sequence having at least 80% sequence identity to an amino acid sequence of SEQ ID NO: 407, and a VL sequence having at least 80% sequence identity to an amino acid sequence of SEQ ID NO: 408; a VH sequence having at least 80% sequence identity to an amino acid sequence of SEQ ID NO: 409, and a VL sequence having at least 80% sequence identity to an amino acid sequence of SEQ ID NO: 410; a VH sequence having at least 80% sequence identity to an amino acid sequence of SEQ ID NO: 411, and a VL sequence having at least 80% sequence identity to an amino acid sequence of SEQ ID NO: 412; a VH sequence having at least 80% sequence identity to an amino acid sequence of SEQ ID NO: 413, and a VL sequence having at least 80% sequence identity to an amino acid sequence of SEQ ID NO: 414; a VH sequence having at least 80% sequence identity to an amino acid sequence of SEQ ID NO: 415, and a VL sequence having at least 80% sequence identity to an amino acid sequence of SEQ ID NO: 416; a VH sequence having at least 80% sequence identity to an amino acid sequence of SEQ ID NO: 417, and a VL sequence having at least 80% sequence identity to an amino acid sequence of SEQ ID NO: 418; a VH sequence having at least 80% sequence identity to an amino acid sequence of SEQ ID NO: 419, and a VL sequence having at least 80% sequence identity to an amino acid sequence of SEQ ID NO: 420; a VH sequence having at least 80% sequence identity to an amino acid sequence of SEQ ID NO: 421, and a VL sequence having at least 80% sequence identity to an amino acid sequence of SEQ ID NO: 422; a VH sequence having at least 80% sequence identity to an amino acid sequence of SEQ ID NO: 423, and a VL sequence having at least 80% sequence identity to an amino acid sequence of SEQ ID NO: 424; a VH sequence having at least 80% sequence identity to an amino acid sequence of SEQ ID NO: 425, and a VL sequence having at least 80% sequence identity to an amino acid sequence of SEQ ID NO: 426; a VH sequence having at least 80% sequence identity to an amino acid sequence of SEQ ID NO: 427, and a VL sequence having at least 80% sequence identity to an amino acid sequence of SEQ ID NO: 428; a VH sequence having at least 80% sequence identity to an amino acid sequence of SEQ ID NO: 429, and a VL sequence having at least 80% sequence identity to an amino acid sequence of SEQ ID NO: 430; a VH sequence having at least 80% sequence identity to an amino acid sequence of SEQ ID NO: 431, and a VL sequence having at least 80% sequence identity to an amino acid sequence of SEQ ID NO: 432; a VH sequence having at least 80% sequence identity to an amino acid sequence of SEQ ID NO: 433, and a VL sequence having at least 80% sequence identity to an amino acid sequence of SEQ ID NO: 434; a VH sequence having at least 80% sequence identity to an amino acid sequence of SEQ ID NO: 435, and a VL sequence having at least 80% sequence identity to an amino acid sequence of SEQ ID NO: 436; a VH sequence having at least 80% sequence identity to an amino acid sequence of SEQ ID NO: 482, and a VL sequence having at least 80%, sequence identity to an amino acid sequence of SEQ ID NO: 483; a VH sequence having at least 80% sequence identity to an amino acid sequence of SEQ ID NO: 484, and a VL sequence having at least 80% sequence identity to an amino acid sequence of SEQ ID NO: 485; a VH sequence having at least 80% sequence identity to an amino acid sequence of SEQ ID NO: 486, and a VL sequence having at least 80% sequence identity to an amino acid sequence of SEQ ID NO: 487; a VH sequence having at least 80% sequence identity to an amino acid sequence of SEQ ID NO: 488, and a VL sequence having at least 80% sequence identity to an amino acid sequence of SEQ ID NO: 489; or a VH sequence having at least 80% sequence identity to an amino acid sequence of SEQ ID NO: 490, and a VL sequence having at least 80% sequence identity to an amino acid sequence of SEQ ID NO: 491.

In some aspects, the second binding domain comprises a variable region heavy and light chain having at least 80% sequence identity to a pair of variable region heavy and light chains set forth in Table 2 as SEQ ID NO: 352-SEQ ID NO: 436. In some aspects, the second binding domain comprises a variable region heavy and light chain having at least 80% sequence identity to the CDR sequences of a pair of variable region heavy and light chains set forth in Table 2 as SEQ ID NO: 352-SEQ ID NO: 436. In some aspects, the second binding domain comprises a variable region heavy and light chain having sequences selected from the pairs of variable region heavy and light chains set forth in Table 2 as SEQ ID NO: 352-SEQ ID NO: 436.

In some aspects, the second binding domain comprises: a VH sequence having at least 80% sequence identity to an amino acid sequence of SEQ ID NO: 352, and a VL sequence having at least 80% sequence identity to an amino acid sequence of SEQ ID NO: 353; a VH sequence having at least 80% sequence identity to an amino acid sequence of SEQ ID NO: 354, and a VL sequence having at least 80% sequence identity to an amino acid sequence of SEQ ID NO: 355; a VH sequence having at least 80% sequence identity to an amino acid sequence of SEQ ID NO: 356, and a VL sequence having at least 80% sequence identity to an amino acid sequence of SEQ ID NO: 357; a VH sequence having at least 80% sequence identity to an amino acid sequence of SEQ ID NO: 358, and a VL sequence having at least 80% sequence identity to an amino acid sequence of SEQ ID NO: 359; a VH sequence having at least 80% sequence identity to an amino acid sequence of SEQ ID NO: 358, and a VL sequence having at least 80% sequence identity to an amino acid sequence of SEQ ID NO: 360; a VH sequence having at least 80% sequence identity to an amino acid sequence of SEQ ID NO: 361, and a VL sequence having at least 80% sequence identity to an amino acid sequence of SEQ ID NO: 362; a VH sequence having at least 80% sequence identity to an amino acid sequence of SEQ ID NO: 363, and a VL sequence having at least 80% sequence identity to an amino acid sequence of SEQ ID NO: 364; a VH sequence having at least 80% sequence identity to an amino acid sequence of SEQ ID NO: 365, and a VL sequence having at least 80% sequence identity to an amino acid sequence of SEQ ID NO: 366; a VH sequence having at least 80% sequence identity to an amino acid sequence of SEQ ID NO: 367, and a VL sequence having at least 80% sequence identity to an amino acid sequence of SEQ ID NO: 368; a VH sequence having at least 80% sequence identity to an amino acid sequence of SEQ ID NO: 367, and a VL sequence having at least 80% sequence identity to an amino acid sequence of SEQ ID NO: 369; a VH sequence having at least 80% sequence identity to an amino acid sequence of SEQ ID NO: 367, and a VL sequence having at least 80% sequence identity to an amino acid sequence of SEQ ID NO: 370; a VH sequence having at least 80% sequence identity to an amino acid sequence of SEQ ID NO: 367, and a VL sequence having at least 80% sequence identity to an amino acid sequence of SEQ ID NO: 371; a VH sequence having at least 80% sequence identity to an amino acid sequence of SEQ ID NO: 367, and a VL sequence having at least 80% sequence identity to an amino acid sequence of SEQ ID NO: 372; a VH sequence having at least 80% sequence identity to an amino acid sequence of SEQ ID NO: 374, and a VL sequence having at least 80% sequence identity to an amino acid sequence of SEQ ID NO: 373; a VH sequence having at least 80% sequence identity to an amino acid sequence of SEQ ID NO: 375, and a VL sequence having at least 80% sequence identity to an amino acid sequence of SEQ ID NO: 376; a VH sequence having at least 80% sequence identity to an amino acid sequence of SEQ ID NO: 377, and a VL sequence having at least 80% sequence identity to an amino acid sequence of SEQ ID NO: 378; a VH sequence having at least 80% sequence identity to an amino acid sequence of SEQ ID NO: 379, and a VL sequence having at least 80% sequence identity to an amino acid sequence of SEQ ID NO: 380; a VH sequence having at least 80% sequence identity to an amino acid sequence of SEQ ID NO: 381, and a VL sequence having at least 80% sequence identity to an amino acid sequence of SEQ ID NO: 382; a VH sequence having at least 80% sequence identity to an amino acid sequence of SEQ ID NO: 384, and a VL sequence having at least 80% sequence identity to an amino acid sequence of SEQ ID NO: 383; a VH sequence having at least 80% sequence identity to an amino acid sequence of SEQ ID NO: 385, and a VL sequence having at least 80% sequence identity to an amino acid sequence of SEQ ID NO: 386; a VH sequence having at least 80% sequence identity to an amino acid sequence of SEQ ID NO: 387, and a VL sequence having at least 80% sequence identity to an amino acid sequence of SEQ ID NO: 388; a VH sequence having at least 80% sequence identity to an amino acid sequence of SEQ ID NO: 389, and a VL sequence having at least 80% sequence identity to an amino acid sequence of SEQ ID NO: 390; a VH sequence having at least 80% sequence identity to an amino acid sequence of SEQ ID NO: 391, and a VL sequence having at least 80% sequence identity to an amino acid sequence of SEQ ID NO: 392; a VH sequence having at least 80% sequence identity to an amino acid sequence of SEQ ID NO: 393, and a VL sequence having at least 80% sequence identity to an amino acid sequence of SEQ ID NO: 394; a VH sequence having at least 80% sequence identity to an amino acid sequence of SEQ ID NO: 395, and a VL sequence having at least 80% sequence identity to an amino acid sequence of SEQ ID NO: 396; a VH sequence having at least 80% sequence identity to an amino acid sequence of SEQ ID NO: 397, and a VL sequence having at least 80% sequence identity to an amino acid sequence of SEQ ID NO: 398; a VH sequence having at least 80% sequence identity to an amino acid sequence of SEQ ID NO: 399, and a VL sequence having at least 80% sequence identity to an amino acid sequence of SEQ ID NO: 400; a VH sequence having at least 80% sequence identity to an amino acid sequence of SEQ ID NO: 401, and a VL sequence having at least 80% sequence identity to an amino acid sequence of SEQ ID NO: 402; a VH sequence having at least 80% sequence identity to an amino acid sequence of SEQ ID NO: 403, and a VL sequence having at least 80% sequence identity to an amino acid sequence of SEQ ID NO: 404; a VH sequence having at least 80% sequence identity to an amino acid sequence of SEQ ID NO: 405, and a VL sequence having at least 80% sequence identity to an amino acid sequence of SEQ ID NO: 406; a VH sequence having at least 80% sequence identity to an amino acid sequence of SEQ ID NO: 407, and a VL sequence having at least 80% sequence identity to an amino acid sequence of SEQ ID NO: 408; a VH sequence having at least 80% sequence identity to an amino acid sequence of SEQ ID NO: 409, and a VL sequence having at least 80% sequence identity to an amino acid sequence of SEQ ID NO: 410; a VH sequence having at least 80% sequence identity to an amino acid sequence of SEQ ID NO: 411, and a VL sequence having at least 80% sequence identity to an amino acid sequence of SEQ ID NO: 412; a VH sequence having at least 80% sequence identity to an amino acid sequence of SEQ ID NO: 413, and a VL sequence having at least 80% sequence identity to an amino acid sequence of SEQ ID NO: 414; a VH sequence having at least 80% sequence identity to an amino acid sequence of SEQ ID NO: 415, and a VL sequence having at least 80% sequence identity to an amino acid sequence of SEQ ID NO: 416; a VH sequence having at least 80% sequence identity to an amino acid sequence of SEQ ID NO: 417, and a VL sequence having at least 80% sequence identity to an amino acid sequence of SEQ ID NO: 418; a VH sequence having at least 80% sequence identity to an amino acid sequence of SEQ ID NO: 419, and a VL sequence having at least 80% sequence identity to an amino acid sequence of SEQ ID NO: 420; a VH sequence having at least 80% sequence identity to an amino acid sequence of SEQ ID NO: 421, and a VL sequence having at least 80% sequence identity to an amino acid sequence of SEQ ID NO: 422; a VH sequence having at least 80% sequence identity to an amino acid sequence of SEQ ID NO: 423, and a VL sequence having at least 80% sequence identity to an amino acid sequence of SEQ ID NO: 424; a VH sequence having at least 80% sequence identity to an amino acid sequence of SEQ ID NO: 425, and a VL sequence having at least 80% sequence identity to an amino acid sequence of SEQ ID NO: 426; a VH sequence having at least 80% sequence identity to an amino acid sequence of SEQ ID NO: 427, and a VL sequence having at least 80% sequence identity to an amino acid sequence of SEQ ID NO: 428; a VH sequence having at least 80% sequence identity to an amino acid sequence of SEQ ID NO: 429, and a VL sequence having at least 80% sequence identity to an amino acid sequence of SEQ ID NO: 430; a VH sequence having at least 80% sequence identity to an amino acid sequence of SEQ ID NO: 431, and a VL sequence having at least 80% sequence identity to an amino acid sequence of SEQ ID NO: 432; a VH sequence having at least 80% sequence identity to an amino acid sequence of SEQ ID NO: 433, and a VL sequence having at least 80% sequence identity to an amino acid sequence of SEQ ID NO: 434; or a VH sequence having at least 80% sequence identity to an amino acid sequence of SEQ ID NO: 435, and a VL sequence having at least 80% sequence identity to an amino acid sequence of SEQ ID NO: 436.

In some aspects, the second binding domain-Fc domain-first antigen binding domain fusion protein comprises the first antigen binding domain of the second binding domain-Fc domain-first antigen binding domain fusion protein comprises at least 80% sequence identity to any one of SEQ ID NO: 1-SEQ ID NO: 436 or SEQ ID NO: 440-SEQ ID NO: 481; the second binding domain of second binding domain-Fc domain-first antigen binding domain fusion protein comprises at least 80% sequence identity to any one of SEQ ID NO: 85-SEQ ID NO: 299, SEQ ID NO: 352-SEQ ID NO: 436; and the Fc domain of the second binding domain-Fc domain-first antigen binding domain fusion protein comprises at least 80% sequence identity to any one of SEQ ID NO: 437-SEQ ID NO: 439, or any fragment thereof, or an Fc domain as described herein, or a fragment thereof.

In some aspects, the second binding domain-first antigen binding domain-Fc domain fusion protein of claim as described herein comprises: the first antigen binding domain of the second binding domain-first antigen binding domain-Fc domain fusion protein comprises a set of six CDRs having at least 80% sequence identity to a set of CDRs set forth in Table 1 as SEQ ID NO: 1-SEQ ID NO: 436 or SEQ ID NO: 440-SEQ ID NO: 481; the second binding domain of the second binding domain-first antigen binding domain-Fc domain fusion protein comprises a set of CDRS having at least 80% sequence identity to a set of CDRs set forth in Table 1 as SEQ ID NO: 85-SEQ ID NO: 299, SEQ ID NO: 352-SEQ ID NO: 436, or any fragment thereof; and the Fc domain of the second binding domain-first antigen binding domain-Fc domain fusion protein comprises at least 80% sequence identity to any one of SEQ ID NO: 437-SEQ ID NO: 439 or any fragment thereof, or the Fc domain as described herein, or a fragment thereof.

In some aspects, the first antigen is an antigen expressed by stellate cells, myofibroblasts, synovial fibroblasts, epithelial cells, podocytes or immune cells. In some aspects, the first antigen is an antigen expressed by stellate cells, myofibroblasts, synovial fibroblasts, epithelial cells or podocytes. In some aspects, the first antigen is an antigen expressed by stellate cells or myofibroblasts. In some aspects, the first antigen is an antigen expressed by stellate cells, a myofibroblasts or podocytes. In some aspects, the second antigen is an antigen expressed by stellate cells or a myofibroblasts.

In various aspects, an isolated nucleic acid is provided that encodes the amino acid sequence of any antibody construct or a portion as described herein.

In some aspects, a vector is provided that includes a nucleic acid encoding an antibody construct as described herein.

In some aspects, a host cell is provided that comprises a vector that includes a nucleic acid encoding an antibody construct as described herein.

In some aspects, a host cell is provided that is a mammalian cell.

In various aspects, a method of producing a conjugate is provided, comprising culturing a host cell so that an antibody construct is produced and then attaching at least one immune-modulatory compounds and a linker to the antibody construct to form a conjugate.

In various aspects, a pharmaceutical composition is provided that comprises any conjugate as described herein and a pharmaceutically acceptable carrier.

In various aspects, a method of treatment is provided for a subject in need thereof, comprising administering a therapeutically effective dose of a conjugate described herein or a pharmaceutical composition as described herein. In some aspects, the subject has a fibrotic disease, an autoimmune disease or inflammatory disease. In some aspects, the pharmaceutical composition or conjugate is administered intravenously, cutaneously, subcutaneously, or injected at a site of affliction. In some aspects, the pharmaceutical composition or conjugate is administered intraveneously. In some aspects, the pharmaceutical composition or conjugate is administered subcutaneously.

In various aspects, a kit comprises a pharmaceutically acceptable dosage unit of a pharmaceutically effective amount of any conjugate described herein or any pharmaceutical composition described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings of which:

FIG. 1A, FIG. 1B, and FIG. 1C illustrate several formats of a conjugate comprising an antibody construct, a linker (L), an immune-modulatory compound (C1), a spacer (S), and a second compound (C2).

FIG. 2 shows inhibition of the TGFβ/SMAD signaling pathway by an LRRC15 conjugate (LRRC15 antibody attached to a TGFβR inhibitor via a cleavable linker), as compared to the control antibody alone and an anti-digoxin conjugate control.

FIG. 3A, FIG. 3B, and FIG. 3C show the results of an assay for degradation of TFGβR2 by a TGFβR2-VHL PROTAC anti-HER2 antibody conjugate.

FIG. 4A and FIG. 4B show the results of an assay for antigen targeted degradation of TGFβR2 by an antibody conjugate with a PROTAC having VHL or Cereblon E3 binding moieties.

FIG. 5A and FIG. 5B show the results of an assay for cellular levels of TGFβR2 and TGFβR1 in the presence of a TGFβR2/TGFβR1-VHL PROTAC with or without the addition of a proteasome inhibitor.

### DETAILED DESCRIPTION OF THE INVENTION

While preferred embodiments of the present invention have been shown and described herein, it will be evident to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Every year, millions of people are hospitalized due to the damaging effects of fibrosis, autoimmune diseases, and autoinflammatory diseases. Fibrosis is the formation of excess fibrous connective tissue or scar tissue in an organ or tissue in a reparative or reactive process. Fibrosis can occur in many tissues within the body, typically as a result of inflammation or damage, which include the lungs, liver, kidney, heart, and brain. Scar tissue blocks arteries, immobilizes joints and damages internal organs, wreaking havoc on the body's ability to maintain vital functions. Autoimmune and autoinflammatory diseases can result from an abnormal response of the immune system to a normal part of the body, or a lack of an immune response to, for example, an infection. In an autoimmune disease, the immune system can produce auto-antibodies that attack the body's own tissues, instead of fighting infections or foreign invaders. Acute or chronic immune-mediated rejection of a transplanted organ or tissue is another area of unmet need. Transplant rejection is a process in which a transplant recipient's immune system can recognize the transplanted organ or tissue as foreign and can attack the transplanted organ or tissue, leading to failure of the transplanted organ or tissue. Although there are marketed treatments for fibrosis, autoimmune disease, autoinflammatory diseases, and transplantation, these treatments, these treatments have limited effectiveness. Thus, there remains a considerable need for alternative or improved treatments for fibrotic diseases, autoimmune diseases, autoinflammatory diseases, and transplantation rejection.

The present disclosure provides antibody construct immune-modulatory compound conjugates (also referred to as “conjugates” or “antibody conjugates”) and pharmaceutical compositions for use in the treatment or prevention of autoimmune disease, autoinflammatory disease, and/or fibrotic disease. In certain embodiments, the antibody construct immune-modulatory compound conjugates and pharmaceutical compositions are used in the treatment or prevention of fibrotic diseases. In certain embodiments, the antibody construct immune-modulatory compound conjugates and pharmaceutical compositions are used in the treatment or prevention of autoimmune diseases. In certain embodiments, the antibody construct immune-modulatory compound conjugates and pharmaceutical compositions are used in the treatment or prevention of autoinflammatory diseases.

Challenges to developing targeted drug therapies include achieving high selectivity for the primary pharmacological target and maintaining prolonged target inhibition or modulation of disease while minimizing toxicity. In overcoming these two challenges, it is possible to develop pharmaceutical products with maximal therapeutic efficacy and minimal systemic toxicity. One approach to addressing these two challenges is developing a conjugate that can deliver a drug to a localized area or targeted tissue without interfering with the activity of the conjugated drug. In some embodiments, the targeting aspect of the conjugate can further inhibit or modulate fibrotic disease, autoimmune disease, autoinflammatory disease, or transplant rejection.

As there is a current need for therapeutics that can inhibit or modulate fibrotic disease, autoimmune disease, autoinflammatory disease, or transplant rejection, the present disclosure provides conjugates, pharmaceutical compositions, and methods that address this need and related needs.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a,” “an,” and “the” includes plural references unless the context clearly dictates otherwise.

As used herein, the term “antibody” refers to an immunoglobulin molecule that specifically binds to, or is immunologically reactive toward, a specific antigen. Antibody can include, for example, polyclonal, monoclonal, genetically engineered, and antigen binding fragments thereof. An antibody can be, for example, murine, chimeric, humanized, heteroconjugate, bispecific, diabody, triabody, or tetrabody. The antigen binding fragment can include, for example, a Fab′, F(ab′)₂, Fab, Fv, rIgG, and scFv.

As used herein, an “antigen binding domain” refers to a region of a molecule that specifically binds to an antigen. An antigen binding domain can be an antigen-binding portion of an antibody or an antibody fragment. An antigen binding domain can be one or more fragments of an antibody that can retain the ability to specifically bind to an antigen. An antigen binding domain can be an antigen binding fragment. In some embodiments, an antigen binding domain can recognize a single antigen. An antigen binding domain can recognize, for example, two or three antigens.

As used herein, a “target binding domain” refers to a construct that contains an antigen binding domain from an antibody or from a non-antibody that can bind to the antigen.

The term “targeting moiety” refers to a structure that has a selective affinity for a target molecule relative to other non-target molecules. The targeting moiety binds to a target molecule. A targeting moiety may include, for example, an antibody, a peptide, a ligand, a receptor, or a binding portion thereof. The target molecule may be an antigen, such as a biological receptor or other structure of a cell.

A “linker-payload” or “LP” refers to an immune-modulatory compound(s) attached to a linker.

As used herein, an “Fc domain” can be an Fc domain from an antibody or from a non-antibody that can bind to an Fc receptor.

As used herein, an “Fc null” refers to a domain that exhibits weak to no binding to any of the Fcgamma receptors. In some embodiments, an Fc null domain or region exhibits a reduction in binding affinity (e.g., increase in Kd) to Fc gamma receptors of at least 1000-fold.

As used herein, “recognize” with regard to antibody interactions refers to specific association or binding between an antigen binding domain of an antibody or portion thereof and an antigen.

As used herein, “sequence identity”, “identity” and “identical” refer to the identity between a DNA, RNA, nucleotide, amino acid, or protein sequence to another DNA, RNA, nucleotide, amino acid, or protein sequence, respectively, according to context. Sequence identity can be expressed in terms of a percentage of sequence identity of a first sequence to a second sequence. Percent (%) sequence identity with respect to a reference DNA sequence is the percentage of DNA nucleotides in a candidate sequence that are identical with the DNA nucleotides in the reference DNA sequence after aligning the sequences and introducing gaps, as necessary. Percent (%) sequence identity with respect to a reference amino acid sequence is the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity.

As used herein, the abbreviations for the natural L-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R).

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond respectively.

The term “carbocycle” as used herein refers to a saturated, unsaturated or aromatic ring in which each atom of the ring is carbon. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, and 6-6 fused ring systems. Exemplary carbocycles include cyclopentyl, cyclohexyl, cyclohexenyl, adamantyl, phenyl, indanyl, and naphthyl.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, and 6-6 fused ring systems.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also include polycyclic ring systems having two or more cyclic rings in which two or more carbons are common to two adjoining rings wherein at least one of the rings is heteroaromatic, e.g., the other cyclic rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—

NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—
NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—
NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants, unless specified otherwise.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

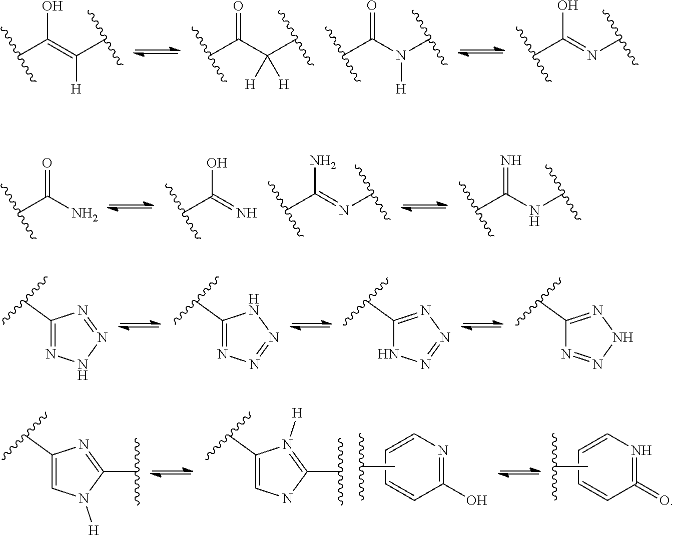

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds useful in the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, “attached” refers to a bond, i.e., a covalent bond, between two or more groups. Alternatively, attached may refer to the association of two or more groups via a linker, e.g., a linker binding an antigen binding domain to an Fc domain to form an antibody construct. A fusion may refer to a nucleic acid sequence of two separate domains being expressed in frame. For example, a binding domain can be attached as a fusion or by conjugation via a linker to form an antibody construct. For example, a portion of an antibody construct can be fused with a second binding domain to create an antibody construct comprising the second binding domain as a fusion protein. The fusion protein can be the result of the nucleic acid sequence encoding the second binding domain being expressed in frame with the nucleic acid sequence encoding the rest of the antibody construct. The fusion protein can be the result of an in-frame genetic nucleotide sequence encoding the antibody construct with the binding domain or a contiguous protein sequence linking the portion of the antibody construct with the binding domain. As another example, a second binding domain can be attached to an antibody construct via a linker, wherein the linker is attached (e.g., conjugated) to the binding domain and the linker is attached (e.g., conjugated) to the rest of the antibody construct. The binding domain can be linked to the linker by a chemical conjugation and the remainder of the antibody construct can be linked to the linker by a chemical conjugation. The binding domain can be a second binding domain and/or a third binding domain as described herein. Furthermore, a binding domain can be a first antigen binding domain attached to an Fc domain to produce the antibody construct as described herein, which may produce the first antigen binding domain as a fusion with the Fc domain wherein the first antigen binding domain can be linked to a linker and the linker can be linked to the Fc domain. As used herein, an “immune-modulatory compound” may refer to a small molecule, or an entity that binds to a target. An immune-modulatory compound may be an entity that can bind to a target and may activate the target's function, or an entity that binds to a target and can inhibit the target's function. A target may be a protein target. The inhibition of a protein target's function may be a result of an increase ubiquitin-mediated degradation. The ubiquitin-mediated degradation may be ubiquitin-mediated degradation of the protein target. The immune-modulatory compound may decrease inflammation, decrease an immune response, decrease fibrosis, or any combination thereof.

### Antibody Construct of Antibody Construct Immune-Modulatory Compound Conjugates

Disclosed herein are antibody constructs that may be used together with immune-modulatory compounds in conjugates. In certain embodiments, immune-modulatory compounds of the disclosure are attached (e.g., conjugated) either directly or through a linker group to an immune-modulatory compound forming antibody conjugates. In certain embodiments, antibody conjugates are represented by the following formula:

AL-D)ₙ,

wherein A is an antibody construct, L is a linker, D is one or more immune-modulatory compounds, e.g., 1, 2, 3, or 4 compounds, and n is from 1 to 20. In certain embodiments, n is from 1 to 10, such as from 1 to 9, such as from 1 to 8, such as from 2 to 8, such as from 1 to 6, such as from 3-5 or such as about 2. In certain embodiments, n is 4. In some embodiments, n is an average.

Also disclosed herein are antibody constructs that may be used together with immune-modulatory compounds as disclosed herein. In certain embodiments, immune-modulatory compounds are attached either directly or through a linker group to an antibody construct of the disclosure forming antibody conjugates. In certain embodiments, antibody conjugates may be represented by the following formula:

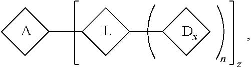

wherein [symbol] is an antibody construct, L is a linker, D is an immune-modulatory compound, x may be from 1 to 20 (wherein each such x denotes a different immune-modulatory compound), n may be from 1-20 and z may be from 1 to 20.

In some embodiments, x may be 1, n may be 1 and z may be from 1 to 10, such as from 1 to 9, such as from 1 to 8, such as from 2 to 8, such as from 1 to 6, such as from 3-5 or such as about 2. In certain embodiments, z may be 4.

In some embodiments, D may be an immune-modulatory compound (IMC), x may be from 1-20, n may be from 1 to 20 and z may be from 1 to 20.

In certain embodiments, antibody conjugates may be represented by the following formula:

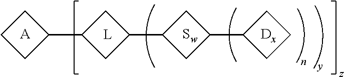

wherein [symbol] is an antibody construct, L is a linker, S is a spacer, D is an immune-modulatory compound, x may be from 1 to 20 (wherein each x denotes a distinct immune-modulatory compound), n may be from 1 to 20, w may be from 1 to 20, y may be from 1 to 20, and z may be from 1 to 20.

In some embodiments x may be 1, n is 2, y may be 1 and z may be from 1 to 10, such as from 1 to 9, such as from 1 to 8, such as from 2 to 8, such as from 1 to 6, such as from 3 to 5 or such as 2. In certain embodiments, z may be 4.

In some embodiments, D may be an immune-modulatory compound (IMC), x may be from 1-20, n may be from 1-20, w may be from 1 to 20, y may be from 1 to 20, and z may be from 1 to 20.

In some embodiments, D may be a proteolysis targeting chimera (PROTAC) which may comprise an immune-modulatory compound (IMC) that may be covalently attached to an E3 ubiquitin ligase binding moiety (ULM) through a spacer (S) and where linker (L) may be covalently attached to spacer (S), n may be from 1 to 20 and z may be from 1 to 20 as represented by the formula:

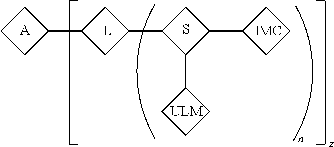

In some embodiments, D may be a proteolysis targeting chimera (PROTAC) which may comprise an immune-modulatory compound (IMC) that may be covalently attached to an E3 ubiquitin ligase binding moiety (ULM) through a spacer (S) and where linker (L) may be covalently attached to the immune-modulatory compound (IMC), n may be from 1 to 20 and z may be from 1 to 20 as represented by the formula:

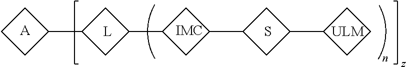

In some embodiments, D may be a proteolysis targeting chimera (PROTAC) which may comprise an immune-modulatory compound (IMC) that may be covalently attached to an E3 ubiquitin ligase binding moiety (ULM) through a spacer (S) and where linker L may be covalently attached to the ubiquitin E3 ligase moiety (ULM), n may be from 1 to 20 and z may be from 1 to 20 as represented by the formula:

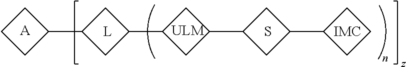

In some embodiments, immune-modulatory compounds are conjugated either directly or through a linker group to an antibody construct forming antibody conjugates (“conjugates”), and may take the form of any conjugate as disclosed in U.S. Pat. Nos. 9,254,339, 9,144,615, 8,821,850, 8,808,679, 8,685,383, 8,524,214, or US Published Application No. US 2011/0243892 (U.S. application Ser. No. 13/163,080), in which each of these references are herein incorporated by reference in their entirety. As used herein, “potency” generally may be measured bioactivity and may be quantified as an EC50 or IC50. Potency may refer to the amount of a conjugate or compound needed to give an effect. For example, a potency of an immune-modulatory compound which requires a lower amount of the immune-modulatory compound to achieve an effect compared with a different immune-modulatory compound can be considered to have greater potency. Furthermore, the different immune-modulatory compound requires a greater amount of the different immune-modulatory compound to generate a response, and can therefore be considered lower potency. Potencies of bioactive compositions may be measured over a concentration range and can be reported as those molar concentrations required to elicit or inhibit a percentage of the measured bioresponse. For example, a concentration required to stimulate 50% of observed maximal activity in the assay may be reported as an effective concentration 50 (EC50), to stimulate 90% activity as an EC90, or to stimulate 10% activity as an EC10. For example, a concentration of an antagonist required to give 50% maximal inhibition of a biological activity may be reported as an inhibitory concentration 50 (IC50), to inhibit 90% as an IC90, or to inhibit 10% as an IC10. This may allow for a comparison of the potencies of bioactive compounds on a molar basis by comparison of their EC or IC values for a given bioassay. For example, an immune-modulatory compound with an EC50 or IC50 that is greater than 300 times or more the EC50 to IC50 of a control requires 300-fold higher, or more than 300-fold higher, concentration compared to the control to achieve a 50% bioresponse and has a potency weaker than the control by at least 300-fold. Therefore, an immune-modulatory compound that has an EC50 or IC50 not greater than about 300 times the EC50 or IC50 of a control compound may require no more than a 300-fold higher concentration than the control compound to achieve a 50% maximal bioresponse, no greater than 100 times the EC50 or IC50 requires no more than 100-fold higher concentration and no greater than 10 times the EC50 or IC50 requires no more than 10 times the concentration of the control. The potency of the immune-modulatory compound may be within 300-fold or better, 100-fold or better, or 10-fold or better the potency of the control.

As used herein, “control compound” refers to the immune-modulatory compound before linker attachment and antibody conjugation or, in the case of conjugates including an E3 ubiquitin ligase binding moiety, control compound refers to the a) immune-modulatory compound attached to b) the second linker that is attached to c) the E3 ubiquitin ligase moiety. In some embodiments, the potency or protein binding of an immune-modulatory compound in the conjugate may be retained or increased. In some embodiments, the K_{d} for the protein target of the immune-modulatory compound as a conjugate is no greater than 100-fold, 25-fold, 10-fold, or 2-fold the control compound. In some embodiments, the EC50 or IC50 of the immune-modulatory compound as a conjugate is no greater than 300-fold, 50-fold, 10-fold, or 2-fold of the control compound. In some embodiments, the EC50 or IC50 is equal to or lower than the control compound indicating increased potency of immune-modulation by the conjugate.

An antibody construct of the disclosure may contain, for example, two, three, four, five, six, seven, eight, nine, ten, or more antigen binding domains. An antibody construct may contain two antigen binding domains in which each antigen binding domain can recognize the same antigen. An antibody construct may contain two antigen binding domains in which each antigen binding domain can recognize different antigens. An antigen binding domain may be in a scaffold, in which a scaffold is a supporting framework for the antigen binding domain. An antigen binding domain may be in a non-antibody scaffold. An antigen binding domain may be in an antibody scaffold. An antibody construct may comprise an antigen binding domain in a scaffold. The antibody construct may comprise a Fc fusion protein product. In some embodiments, the antibody construct is a Fc fusion protein product. An antigen binding domain may specifically bind to an antigen associated with fibrotic disease, autoimmune disease, or autoinflammatory disease. An antigen binding domain may specifically bind to an antigen that is at least 80%, at least 90%, at least 95%, at least 99%, or 100% identical to an antigen associated with fibrotic disease, autoimmune disease, or autoinflammatory disease. An antigen binding domain may specifically bind to an antigen on an antigen presenting cell. An antigen binding domain may specifically bind to an antigen that is at least 80%, at least 90%, at least 95%, at least 99%, or 100% identical to an antigen on an antigen presenting cell. An antigen binding domain may specifically bind to an antigen on a T cell. An antigen binding domain may specifically bind to an antigen that is at least 80%, at least 90%, at least 95%, at least 99%, or 100% identical to an antigen on a T cell.

A conjugate described herein can contain, for example, an immune-modulatory compound, an antibody construct, and a linker attaching the antibody construct to the immune-modulatory compound. The antibody construct of the conjugate can contain, for example, a first antigen binding domain and an Fc domain, where the first antigen binding domain binds to a first antigen. The first antigen can have about 50%, about 60%, about 70%, about 80%, or about 90% or about 100% sequence identity to, for example, Cadherin 11, PDPN, Integrin α4β7, Integrin α2b1, MADCAM, Nephrin, Podocin, IFNAR1, BDCA, CD30, c-KIT, FAP, CD73, CD38, PDGFRB, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, or CD25. In some aspects, the first antigen is selected from Cadherin 11, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, LRRC15, and Cadherin11. In some embodiment, the first antigen is selected from Cadherin 11, PDPN, LRRC15, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, MMP14, GPX8, and F2RL2. In some embodiments, the first antigen is selected from Cadherin 11, FAP, TNFR2, or LRRC15. In some aspects, the first antigen is selected from LRRC15, FAP, Cadherin 11, and TNFR2.

In certain embodiments, the first antigen binding domain specifically binds to an antigen that is at least 80% identical to an antigen on an immune cell, such as a T cell, a B cell and an APC, a stellate cell, an endothelial cell, an epithelial cell, a tumor cell, a fibroblast cell, a fibrocyte cell, a podocyte, a myofibroblast, a synovial fibroblast, or other cell associated with the pathogenesis of fibrosis. In certain embodiments, the first antigen binding domain specifically binds to an antigen that is at least 80% identical to an antigen on a T cell, an APC, and/or a B cell. In certain embodiments, the first antigen binding domain may specifically bind to an antigen that is at least 80% identical to an antigen selected from the group consisting of PD-1, GARP, CD25, PD-L1, or TNFR2. In certain embodiments, the first antigen binding domain specifically binds to an antigen that is at least 80% identical to an antigen on a stellate cell, an endothelial cell, a fibroblast cell, a fibrocyte cell, a myofibroblast, a synovial fibroblast, a podocyte or a cell associated with the pathogenesis of fibrosis. In certain embodiments, the first antigen binding domain may specifically bind to an antigen that is at least 80% identical to an antigen selected from the group consisting of PDGFRβ, integrin αvβ1, integrin αvβ3, integrin αvβ6, integrin αvβ8, Endosialin, FAP, ADAM12, LRRC15, MMP14, PDPN, CDH11 and F2RL2. In certain embodiments, the first antigen binding domain may specifically bind to an antigen that is at least 80% identical to an antigen selected from the group consisting of FAP, ADAM12, LRRC15, MMP14, PDPN, CDH11, and F2RL2.

In certain embodiments, the first antigen binding domain specifically binds to an antigen on a T cell, a B cell, a stellate cell, an endothelial cell, a tumor cell, an APC, a fibroblast cell, a fibrocyte cell, a myofibroblast, a synovial fibroblast, a podocyte or other cell associated with the pathogenesis of fibrosis. In certain embodiments, the first antigen binding domain specifically binds to an antigen on a T cell, an APC, and/or a B cell. In certain embodiments, the first antigen binding domain may specifically bind to an antigen selected from the group consisting of PD-1, GARP, CD25, PD-L1, or TNFR2. In certain embodiments, the first antigen binding domain specifically binds to an antigen on a stellate cell, an endothelial cell, a fibroblast cell, a fibrocyte cell, a myofibroblast, a synovial fibroblast, a podocyte or a cell associated with the pathogenesis of fibrosis. In certain embodiments, the first antigen binding domain may specifically bind to an antigen selected from the group consisting of PDGFRβ, integrin αvβ1, integrin αvβ3, integrin αvβ6, integrin αvβ8, Endosialin, FAP, ADAM12, LRRC15, MMP14, PDPN, CDH11 and F2RL2. In certain embodiments, the first antigen binding domain specifically binds to an antigen selected from the group consisting of FAP, ADAM12, LRRC15, MMP14, PDPN, CDH11, and F2RL2.

A binding domain of an antibody construct can bind to not only a particular amino acid sequence on an antigen, but also exhibit specificity for particular protein complexes, protein conformations, protein conformers, post-transcriptional modifications, or post-translational modifications. For example, the antigen of a binding domain of the conjugate can comprise a splice junction, a protein complex, a protein conformer or a post-translational modification. A non-limiting example of a splice variant antigen that can be specifically recognized by a binding domain is a binding domain for the EGFRviii slice variant. Some non-limiting examples of binding domains for specific antigens generated by a post-translational modification or protein conformer can be a binding domain for a splice variant of CD45RB or CD45RO. A non-limiting example of a binding domain that can bind to a protein complex can be a binding domain that can bind to specific integrin pair, such as αvβ6. For example, the binding domain can bind tightly to αvβ6, but weakly or not at all to the individual subunits or one subunit paired with a different subunit. Some additional non-limiting examples of these types of binding domains can include an anti-CD45RB antibody, an anti-CD45RO antibody, an anti-αvβ6 antibody, and an anti-αvβ8 antibody.

An antigen may be PDCD1. The PDCD1 gene encodes programmed cell death protein 1, also known as PD-1 and CD279 (cluster of differentiation 279), which is a cell surface receptor that plays a cell surface receptor that plays an important role in down-regulating the immune system and promoting self-tolerance by suppressing T cell inflammatory activity. PD-1 is a cell surface receptor that belongs to the immunoglobulin superfamily and is expressed on T cells and pro-B cells. PD-1 is an immune checkpoint and guards against autoimmunity through a dual mechanism of promoting apoptosis (programmed cell death) in antigen specific T-cells in lymph nodes while simultaneously reducing apoptosis in regulatory T cells (anti-inflammatory, suppressive T cells).

An antigen may be TNFRSF4. The TNFRSF4 gene encodes OX40, also known as TNFRSF4 (tumor necrosis factor receptor superfamily, member 4), a member of the TNFR-superfamily of receptors which is not constitutively expressed on resting naïve T cells, unlike CD28. OX40 is a secondary co-stimulatory immune checkpoint molecule, expressed after 24 to 72 hours following activation; its ligand, OX40L, is also not expressed on resting antigen presenting cells, but is expressed following their activation. Expression of OX40 is dependent on full activation of the T cell; without CD28, expression of OX40 is delayed and of fourfold lower levels.

An antigen may be CD27. CD27 is a member of the tumor necrosis factor receptor superfamily. The protein encoded by this gene is a member of the TNF-receptor superfamily. This receptor is required for generation and long-term maintenance of T cell immunity. It binds to ligand CD70, and plays a key role in regulating B-cell activation and immunoglobulin synthesis. This receptor transduces signals that lead to the activation of NF-κB and MAPK8/JNK. Adaptor proteins TRAF2 and TRAF5 have been shown to mediate the signaling process of this receptor. CD27-binding protein (SIVA), a proapoptotic protein, can bind to this receptor and is thought to play an important role in the apoptosis induced by this receptor.

An antigen may be IL2RA. The IL2RA gene encodes CD25, also known as IL2RA (interleukin-2 receptor alpha chain), which is a type I transmembrane protein present on activated T cells, activated B cells, some thymocytes, myeloid precursors, and oligodendrocytes. IL2RA is expressed in most B-cell neoplasms, some acute nonlymphocytic leukemias, neuroblastomas, mastocytosis and tumor infiltrating lymphocytes. It functions as the receptor for HTLV-1 and is consequently expressed on neoplastic cells in adult T cell lymphoma/leukemia. Its soluble form, called sIL-2R may be elevated in these diseases and is occasionally used to track disease progression.

An antigen may be TNFRSF18. The TNFRSF18 gene encodes GITR (glucocorticoid-induced TNFR-related protein), also known as TNFRSF18 (tumor necrosis factor receptor superfamily member 18) and AITR (activation-inducible TNFR family receptor), which is a protein that is a member of the tumor necrosis factor receptor (TNF-R) superfamily. GITR (glucocorticoid-induced tumor necrosis factor receptor) is a surface receptor molecule that has been shown to be involved in inhibiting the suppressive activity of T-regulatory cells and extending the survival of T-effector cells.

An antigen may be LAG-3. The LAG-3 (lymphocyte-activation gene 3) gene encodes a cell surface molecule with diverse biologic effects on T cell function. LAG-3 is an immune checkpoint receptor. The LAG3 protein, which belongs to immunoglobulin (Ig) superfamily, comprises a 503-amino acid type I transmembrane protein with four extracellular Ig-like domains, designated D1 to D4. LAG-3 is expressed on activated T cells, natural killer cells, B cells and plasmacytoid dendritic cells.

An antigen may be GARP. GARP (glycoprotein A repetitions predominant) is a transmembrane protein containing leucine rich repeats, which is present on the surface of stimulated Treg clones but not on Th clones.

An antigen may be 4-1BB. 4-1BB is a type 2 transmembrane glycoprotein belonging to the TNF superfamily, expressed on activated T Lymphocytes. 4-1BB can be expressed by activated T cells. 4-1BB expression can be found on dendritic cells, B cells, follicular dendritic cells, natural killer cells, granulocytes and cells of blood vessel walls at sites of inflammation.

An antigen may be ICOS. The ICOS (Inducible T-cell COStimulator) gene encodes a CD28-superfamily costimulatory molecule that is expressed on activated T cells. The protein encoded by this gene belongs to the CD28 and CTLA-4 cell-surface receptor family. ICOS forms homodimers and plays an important role in cell-cell signaling, immune responses and regulation of cell proliferation.

An antigen may be CD70. CD70 is expressed on highly activated lymphocytes, such as in T- and B-cell lymphomas. CD70 is a cytokine that belongs to the tumor necrosis factor (TNF) ligand family. This cytokine is a ligand for TNFRSF27/CD27. It is a surface antigen on activated, but not on resting, T and B lymphocytes. CD70 induces proliferation of co-stimulated T cells, enhances the generation of cytolytic T cells, and contributes to T cell activation. This cytokine is also reported to play a role in regulating B-cell activation, cytotoxic function of natural killer cells, and immunoglobulin synthesis.

An antigen may be PDGFRβ. The PDGFRβ (beta-type platelet-derived growth factor receptor) gene encodes a typical receptor tyrosine kinase, which is a transmembrane protein consisting of an extracellular ligand binding domain, a transmembrane domain and an intracellular tyrosine kinase domain. The molecular mass of the mature, glycosylated PDGFRβ protein is approximately 180 kDA.

An antigen may be CD73. CD73 (cluster of differentiation 73), known as ecto-5′-nucleotidase (ecto-5′-NT, EC 3.1.3.5) is a glycosyl-phosphatidylinositol (GPI)-linked 70-kDa cell surface enzyme found in most tissues. CD73 commonly serves to convert AMP to adenosine. Ecto-5-prime-nucleotidase (5-prime-ribonucleotide phosphohydrolase; EC 3.1.3.5) catalyzes the conversion at neutral pH of purine 5-prime mononucleotides to nucleosides, the preferred substrate being AMP. The enzyme consists of a dimer of 2 identical 70-kD subunits bound by a glycosyl phosphatidyl inositol linkage to the external face of the plasma membrane. The enzyme is used as a marker of lymphocyte differentiation.

An antigen may be CD38. CD38 (cluster of differentiation 38), also known as cyclic ADP ribose hydrolase, is a glycoprotein found on the surface of many immune cells (white blood cells), including CD4⁺, CD8⁺, B lymphocytes, and natural killer cells. CD38 also functions in cell adhesion, signal transduction and calcium signaling. The loss of CD38 function is associated with impaired immune responses, metabolic disturbances, and behavioral modifications including social amnesia possibly related to autism. The CD38 protein is a marker of cell activation. It has been connected to HIV infection, leukemias, myelomas, solid tumors, type II diabetes mellitus and bone metabolism, as well as some genetically determined conditions. CD38 produces an enzyme which regulates the release of oxytocin within the central nervous system.

An antigen may be Integrin αvβ3. Integrin αvβ3 is a type of integrin that is a receptor for vitronectin. Integrin αvβ3 consists of two components, integrin alpha V and integrin beta 3 (CD61), and is expressed by platelets. Integrin αvβ3 is a receptor for phagocytosis on macrophages or dendritic cells.

An antigen may be Integrin αvβ8. Integrin αvβ8, a VN receptor, is identified as a potential negative regulator of cell growth. The cytoplasmic domain of β8 is divergent in sequence, lacking all amino acid sequence identity with the highly homologous cytoplasmic domains of the other αv-associating integrin jβ subunits (β1, β3, β5, and β6). The β8 cytoplasmic domain is divergent in function. αvβ8 has a restricted distribution and is most highly expressed in nonproliferating cell types.

An antigen may be CD248. The CD248 gene encodes endosialin. Endosialin is a member of the “Group XIV”, a novel family of C-type lectin transmembrane receptors which play a role not only in cell-cell adhesion processes but also in host defense. Endosialin has been associated with angiogenesis in the embryo, uterus and in tumor development and growth.

An antigen may be FAP. FAP (fibroblast activation protein alpha) is a 170 kDa melanoma membrane-bound gelatinase protein that in humans is encoded by the FAP gene. The protein encoded by this gene is a homodimeric integral membrane gelatinase belonging to the serine protease family. It is selectively expressed in reactive stromal fibroblasts of epithelial cancers, granulation tissue of healing wounds, and malignant cells of bone and soft tissue sarcomas. This protein is thought to be involved in the control of fibroblast growth or epithelial-mesenchymal interactions during development, tissue repair, and epithelial carcinogenesis.

An antigen may be LRRC15. LRRC15, also known as Leucine Rich Repeat Containing 15 or LIB, is a single pass type 1 membrane protein.

An antigen may be ADAM12. ADAM12 is a disintegrin and metalloprotease. It is reported to be involved in skeletal muscle regeneration, specifically at the onset of cell fusion. It is interacts with alpha-actinin-2 and with syndecans and with RACK1; this interaction is required for PKC-dependent translocation of ADAM12 to the cell membrane.

An antigen may be MMP14. MMP14 is an endopeptidase that degrades various components of the extracellular matrix such as collagen. It activates progelatinase A. MMP14 may be involved in actin cytoskeleton reorganization by cleaving PTK7. MMP14 acts as a positive regulator of cell growth and migration via activation of MMP15, and is involved in the formation of the fibrovascular tissues in association with pro-MMP2.

An antigen may be F2RL2. F2RL2 is a receptor for activated thrombin coupled to G proteins that stimulate phosphoinositide hydrolysis.

An antigen may be Integrin αv. Integrin αv subunit associates with one of five integrin β subunits, β1, β3, β5, β6, or β8, to form five distinct αVβ heterodimers. The integrin αVβ heterodimers on the cell surface interact with cell adhesive proteins, such as collagen, fibrinogen, fibronectin, and vitronectin. These interactions play an important role in cell adhesion or migration, especially in tumor metastasis.

An antigen may be Integrin αvβ6. Integrin αvβ6 is an epithelial-specific integrin that is a receptor for the extracellular matrix (ECM) proteins fibronectin, vitronectin, tenascin and the latency associated peptide (LAP) of TGF-β. Integrin αvβ6 is not expressed in healthy adult epithelia but is upregulated during wound healing and in cancer. Integrin αvβ6 has been shown to modulate invasion, inhibit apoptosis, regulate the expression of matrix metalloproteases (MMPs) and activate TGF-β1.

An antigen may be Cadherin 11. Cadherin 11 is a type II classical cadherin from the cadherin superfamily, which are integral membrane proteins that mediate calcium-dependent cell-cell adhesion. Mature cadherin proteins are composed of a large N-terminal extracellular domain, a single membrane-spanning domain, and a small, highly conserved C-terminal cytoplasmic domain. Type II (atypical) cadherins are defined based on their lack of a HAV cell adhesion recognition sequence specific to type I cadherins. Cadherin 11 is expressed in osteoblastic cell lines, and is upregulated during osteoblast differentiation.

An antigen may be PDPN. PDPN (podoplanin) is a type-I integral membrane glycoprotein with diverse distribution in human tissues. The physiological function of PDPN can be related to its mucin-type character. Alternatively spliced transcript variants encoding different isoforms have been identified.

An antigen may be MADCAM. MADCAM (mucosal vascular addressin cell adhesion molecule is an endothelial cell adhesion molecule that interacts with the leukocyte beta7 integrin LPAM-1 (alpha4beta7), L-selectin and VLA-4 (alpha4beta1) on myeloid cells to direct leukocytes into mucosal and inflamed tissues. MADCAM is a member of the immunoglobulin family.

An antigen may be Nephrin. Nephrin is a member of the immunoglobulin family of cell adhesion molecules, which function in the glomerular filtration barrier in the kidney. Nephrin is expressed in renal tissues, and the protein is a type-1 transmembrane protein found at the slit diaphragm of glomerular podocytes. The slit diaphragm is an ultrafilter that can exclude albumin and other plasma macromolecules in the formation of urine. Mutations in the gene encoding nephrin can result in Finnish-type congenital nephrosis 1, characterized by severe proteinuria and loss of the slit diaphragm and foot processes.

An antigen may be Podocin. Podocin (NPHS2) is a protein that can regulate of glomerular permeability. Mutations in the gene encoding for podocin can cause steroid-resistant nephrotic syndrome.

An antigen may be IFNAR1. IFNAR1 (Interferon Alpha And Beta Receptor Subunit 1) is a type I membrane protein that forms one of the two chains of a receptor for interferons alpha and beta. Binding and activation of IFNAR1 stimulates Janus protein kinases, which in turn phosphorylate several proteins, including STAT1 and STAT2. IFNAR1 can also function as an antiviral factor.

An antigen may be BDCA2. BDCA2 (Interferon Alpha And Beta Receptor Subunit 1) is a type II C-type lectin receptor selectively expressed on plasmacytoid dendritic cells (PDCs), where it is involved in antigen capture and in regulation of the production of interferon type I.

An antigen may be CD30. CD30 (TNF Receptor Superfamily Member 8) is expressed by activated, but not by resting, T and B cells. TRAF2 and TRAF5 can interact with CD30 and mediate the signal transduction that leads to the activation of NF-kappaB. CD30 is a positive regulator of apoptosis, and also has been shown to limit the proliferative potential of autoreactive CD8 effector T cells and protect the body against autoimmunity. Two alternatively spliced transcript variants of the gene encoding CD30 have been reporting leading to the translation of distinct isoforms of CD30.

An antigen may be c-KIT. c-KIT (KIT Proto-Oncogene Receptor Tyrosine Kinase/CD117) is a type 3 transmembrane receptor for MGF (mast cell growth factor, also known as stem cell factor). Mutations in the gene encoding for c-KIT are associated with gastrointestinal stromal tumors, mast cell disease, acute myelogenous leukemia, and piebaldism. Multiple transcript variants encoding different isoforms have been found for the gene encoding c-KIT.

An antigen may be CTGF. CTGF (Connective Tissue Growth Factor) is a mitogen that is secreted by vascular endothelial cells. CTGF plays a role in chondrocyte proliferation and differentiation, cell adhesion in many cell types, and is related to platelet-derived growth factor. Certain polymorphisms in the gene encoding CTGF have been linked with a higher incidence of systemic sclerosis.

An antigen may be CD40. Cluster of Differentiation 40 (CD40) is a member of the Tumor Necrosis Factor Receptor (TNF-R) family. CD40 can be a 50 kDa cell surface glycoprotein that can be constitutively expressed in normal cells, such as monocytes, macrophages, B lymphocytes, dendritic cells, endothelial cells, smooth muscle cells, fibroblasts and epithelium, and in tumor cells, including B-cell lymphomas and many types of solid tumors. Expression of CD40 can be increased in antigen presenting cells in response to IL-1βp, IFN-γ, GM-CSF, and LPS induced signaling events.

An antigen may be TIM-3. TIM-3 (T-cell immunoglobulin and mucin-domain containing—3) can function as a T-cell inhibitory receptor. Galectin-9 triggering of Tim-3 can induce cell death in Tim-3+ Th1 cells and ameliorate experimental autoimmune encephalomyelitis. Tim-3 can also be required for the induction of tolerance, as both Tim-3-deficient mice and mice treated with a Tim-3-Ig fusion protein exhibit defects in the induction of antigen-specific tolerance. Overall, TIM-3 is an immune checkpoint receptor that functions specifically to limit the duration and magnitude of Th1 and Tc1 T-cell responses.

An antigen may be TNFR2. TNFR2 (tumor necrosis factor receptor 2), also known as TNFRSF1B (tumor necrosis factor receptor super family 1B) and CD120b, is a single-pass type I membrane protein and the member of TNFR superfamily containing 4 cysteine-rich domains (CRD) repeats. In addition to the full length membrane-anchored form, soluble TNFR2 can be generated via two distinct mechanisms: (1) shedding via proteolytic processing of the full membrane anchored from, and (2) translation from an alternatively spliced message encoding the extracellular domains of TNFR2. TNFR2 is the receptor with high affinity for TNF-alpha and approximately 5-fold lower affinity for homotrimeric lymphotoxin-alpha. TNFR2 (Tumor Necrosis Factor Receptor Type II) and TNF-receptor 1 form a heterocomplex that mediates the recruitment of two anti-apoptotic proteins, c-IAP1 and c-IAP2, which possess E3 ubiquitin ligase activity. c-IAP1 can potentiate TNF-induced apoptosis by the ubiquitination and degradation of TNF-receptor-associated factor 2, which mediates anti-apoptotic signals. Knockout studies in mice suggest a role of TNFR2 in protecting neurons from apoptosis by stimulating antioxidative pathways.

An antigen may be DEC205. DEC205 is a type I cell surface protein expressed primarily by dendritic cells. DEC205 is found on interdigitating dendritic cells in T-cell areas of lymphoid tissues, bone marrow-derived DC, Langerhan's cells, and at low levels on macrophages and T cells. DEC205 can be upregulated during the maturation of dendritic cells. DEC-205 has also been shown to be expressed at moderate levels by B cells and can be upregulated during the pre-B cell to B cell transition. Structurally, the DEC205 family is characterized by a cysteine rich N-terminal domain followed by a fibronectin type II domain and multiple carbohydrate recognition domains (CRDs). DEC-205 has ten CRDs. The single transmembrane domain is followed by a short cytoplasmic tail.

An antigen may be DCIR. DCIR (Dendritic cell immunoreceptor/CLEC4A) is a member of the C-type lectin/C-type lectin-like domain (CTL/CTLD) superfamily. DCIR can have diverse functions such as cell adhesion, cell-cell signaling, glycoprotein turnover, and roles in inflammation and immune response. The encoded type 2 transmembrane protein can play a role in inflammatory and immune response. Multiple transcript variants encoding distinct isoforms have been identified for the gene encoding DCIR.

An antigen may be CD86. CD86 (Cluster of Differentiation 86) is a type I membrane protein that is a member of the immunoglobulin superfamily. CD86 is expressed by antigen-presenting cells, and is the ligand for two proteins at the cell surface of T cells, CD28 antigen and cytotoxic T-lymphocyte-associated protein 4. Binding of CD86 with CD28 antigen is a costimulatory signal for activation of the T-cell. Binding of CD86 protein with cytotoxic T-lymphocyte-associated protein 4 negatively regulates T-cell activation and diminishes the immune response.

An antigen may be CD45RB or CD45RB/RO. CD45RB is an isoform of CD45 with exon 5 splicing. CD45RB is a 220 kD glycoprotein expressed on peripheral B cells, naïve T cells, thymocytes, macrophages, and dendritic cells. CD45RB can play a role in TCR and BCR signaling. As T cells become activated and progress from naïve to memory cells, CD45RB expression is downregulated. Additionally, functionally distinct CD4+ T cell subsets, which secrete differing cytokine profiles, can be separated by CD45RB intensity. The primary ligands for CD45 are galectin-1, CD2, CD3, CD4, and Thy-1. In contrast to CD45RB, CD45RO is the antigenic isoform expressed on effector or memory T cells as they downregulate the CD45A and CD45B isoforms.

An antigen binding domain of an antibody may comprise one or more light chain (L) CDRs and one or more heavy chain (H) CDRs. For example, an antibody binding domain of an antibody may comprise one or more of the following: a light chain complementary determining region 1 (LCDR1), a light chain complementary determining region 2 (LCDR2), or a light chain complementary determining region 3 (LCDR3). For another example, an antibody binding domain may comprise one or more of the following: a heavy chain complementary determining region 1 (HCDR1), a heavy chain complementary determining region 2 (HCDR2), or a heavy chain complementary determining region 3 (HCDR3). As an additional example, an antibody binding domain of an antibody may comprise one or more of the following: LCDR1, LCDR2, LCDR3, HCDR1, HCDR2, and HCDR3. As another example, an antibody binding domain of an antibody may comprise all six of the following: LCDR1, LCDR2, LCDR3, HCDR1, HCDR2, and HCDR3.

The antigen binding domain of an antibody construct may be selected from any domain that specifically binds to the antigen including, but not limited to, a monoclonal antibody, a polyclonal antibody, a recombinant antibody, or a functional (antigen binding) fragment thereof, for example, a heavy chain variable domain (V_{H}) and a light chain variable domain (V_{L}), or may be a DARPin, an affimer, an avimer, a knottin, a monobody, an affinity clamp, an ectodomain, a receptor ectodomain, a receptor, a cytokine, a ligand, an immunocytokine, a T cell receptor, a bicyclic peptide, a fynomer, or a recombinant T cell receptor.

The antigen binding domain of an antibody construct may be at least 80% identical to a particular antigen binding domain that binds to an antigen, where the antigen binding domain is selected from, but not limited to, a monoclonal antibody, a polyclonal antibody, a recombinant antibody, or a functional fragment thereof, for example, a heavy chain variable domain (V_{H}) and a light chain variable domain (V_{L}), or may be a DARPin, an affimer, an avimer, a knottin, a monobody, an affinity clamp, an ectodomain, a receptor ectodomain, a receptor, a cytokine, a ligand, an immunocytokine, a T cell receptor, a bicyclic peptide, a fynomer, an anticalin, a VNAR, or a recombinant T cell receptor.

In certain embodiments, an antibody construct comprises an Fc region comprising an Fc domain, in which the Fc domain may be the part of the Fc region that interacts with Fc receptors. The Fc domain of an antibody construct may interact with Fc-receptors (FcRs) found on immune cells. The Fc domain may also mediate the interaction between effector molecules and cells, which can lead to activation of the immune system. The Fc region may be derived from IgG, IgA, or IgD antibody isotypes, and may comprise two identical protein fragments, which are derived from the second and third constant domains of the antibody's heavy chains. In an Fc region derived from an IgG antibody isotype, the Fc region may comprise a highly-conserved N-glycosylation site, which may be essential for FcR-mediated downstream effects. The Fc region may be derived from IgM or IgE antibody isotypes, in which the Fc region may comprise three heavy chain constant domains.

An Fc domain may interact with different types of FcRs. The different types of FcRs may include, for example, FcγRI, FcγRI, FcγRIIA, FcγRIIB, FcγRIIIA, FcγRIIIB, FcαRI, FcμR, FcεRI, FcεRII, and FcRn. FcRs may be located on the membrane of certain immune cells including, for example, B lymphocytes, natural killer cells, macrophages, neutrophils, follicular dendritic cells, eosinophils, basophils, platelets, and mast cells. Once the FcR is engaged by the Fc domain, the FcR may initiate functions including, for example, clearance of an antigen-antibody complex via receptor-mediated endocytosis, antibody-dependent cell-mediated cytotoxicity (ADCC), antibody dependent cell-mediated phagocytosis (ADCP), and ligand-triggered transmission of signals across the plasma membrane that can result in alterations in secretion, exocytosis, and cellular metabolism. FcRs may deliver signals when FcRs are aggregated by antibodies and multivalent antigens at the cell surface. The aggregation of FcRs with immunoreceptor tyrosine-based activation motifs (ITAMs) may sequentially activate SRC family tyrosine kinases and SYK family tyrosine kinases. ITAM comprises a twice-repeated YxxL sequence flanking seven variable residues. The SRC and SYK kinases may connect the transduced signals with common activation pathways.

In some embodiments, an Fc domain or region can exhibit reduced binding affinity to one or more Fc receptors. In some embodiments, an Fc domain or region can exhibit reduced binding affinity to one or more Fcgamma receptors. In some embodiments, an Fc domain or region can exhibit reduced binding affinity to FcRn receptors. In some embodiments, an Fc domain or region can exhibit reduced binding affinity to Fcgamma and FcRn receptors. In some embodiments, an Fc domain is an Fc null domain or region. As used herein, an “Fc null” refers to a domain that exhibits weak to no binding to any of the Fcgamma receptors. In some embodiments, an Fc null domain or region exhibits a reduction in binding affinity (e.g., increase in Kd) to Fc gamma receptors of at least 1000-fold.

The Fc domain may have one or more, two or more, three or more, or four or more amino acid substitutions that decrease binding of the Fc domain to an Fc receptor. In certain embodiments, an Fc domain exhibits decreased binding to FcγRI (CD64), FcγRIIA (CD32), FcγRIIIA (CD16a), FcγRIIIB (CD16b), or any combination thereof. In order to decrease binding affinity of an Fc domain or region to an Fc receptor, the Fc domain or region may comprise one or more substitutions that have the effect of reducing the affinity of the Fc domain or region to an Fc receptor. In certain embodiments, the one or more substitutions comprise any one or more of IgG1 heavy chain mutations corresponding to E233P, L234V, L234A, L235A, L235E, AG236, G237A, E318A, K320A, K322A, A327G, A330S, or P331S according to the EU index of Kabat numbering.

In some embodiments, the Fc domain or region can comprise a sequence of the IgG1 isoform that has been modified from the wild-type IgG1 sequence. A modification can comprise a substitution at more than one amino acid residue, such as at 5 different amino acid residues including L235V/F243L/R292P/Y300L/P396L (IgG1VLPLL) according to the EU index of Kabat numbering. A modification can comprise a substitution at more than one amino acid residue such as at 2 different amino acid residues including S239D/I332E (IgG1DE) according to the EU index of Kabat numbering. A modification can comprise a substitution at more than one amino acid residue such as at 3 different amino acid residues including S298A/E333A/K334A (IgG1AAA) according to the EU index of Kabat numbering.

In some embodiments, the Fc domain or region can comprise a sequence of an IgG isoform that has been modified from the wild-type IgG sequence. In some embodiments, the Fc domain or region can comprise a sequence of the IgG1 isoform that has been modified from the wild-type IgG1 sequence. In some embodiments, the modification comprises substitution of one or more amino acids that reduce binding affinity of an IgG Fc domain or region to all Fcγ receptors. A modification can be substitution of E233, L234 and L235, such as E233P/L234V/L235A or E233P/L234V/L235A/AG236, according to the EU index of Kabat. A modification can be substitution of L235, F243, R292, Y300 and P396, such as L235V/F243L/R292P/Y300L/P396L (IgG1VLPLL) according to the EU index of Kabat. A modification can be a substitution of P238, such as P238A, according to the EU index of Kabat. A modification can be a substitution of D265, such as D265A, according to the EU index of Kabat. A modification can be a substitution of N297, such as N297A, according to the EU index of Kabat. A modification can be a substitution of A327, such as A327Q, according to the EU index of Kabat. A modification can be a substitution of P329, such as P239A, according to the EU index of Kabat.

In some embodiments, an IgG Fc domain or region comprises at least one amino acid substitution that reduces its binding affinity to FcγR1, as compared to a wild-type or reference IgG Fc domain. A modification can comprise a substitution at F241, such as F241A, according to the EU index of Kabat. A modification can comprise a substitution at F243, such as F243A, according to the EU index of Kabat. A modification can comprise a substitution at V264, such as V264A, according to the EU index of Kabat. A modification can comprise a substitution at D265, such as D265A according to the EU index of Kabat.

In some embodiments, an IgG Fc domain or region comprises at least one amino acid substitution that increases its binding affinity to FcγR1, as compared to a wild-type or reference IgG Fc domain. A modification can comprise a substitution at A327 and P329, such as A327Q/P329A, according to the EU index of Kabat.

In some embodiments, the modification comprises substitution of one or more amino acids that reduce binding affinity of an IgG Fc domain or region to FcγRII and FcγRIIIA receptors. A modification can be a substitution of D270, such as D270A, according to the EU index of Kabat. A modification can be a substitution of Q295, such as Q295A, according to the EU index of Kabat. A modification can be a substitution of A327, such as A237S, according to the EU index of Kabat.

In some embodiments, the modification comprises substitution of one or more amino acids that increases binding affinity of an IgG Fc domain or region to FcγRII and FcγRIIIA receptors. A modification can be a substitution of T256, such as T256A, according to the EU index of Kabat. A modification can be a substitution of K290, such as K290A, according to the EU index of Kabat.

In some embodiments, the modification comprises substitution of one or more amino acids that increases binding affinity of an IgG Fc domain or region to FcγRII receptor. A modification can be a substitution of R255, such as R255A, according to the EU index of Kabat. A modification can be a substitution of E258, such as E258A, according to the EU index of Kabat. A modification can be a substitution of S267, such as S267A, according to the EU index of Kabat. A modification can be a substitution of E272, such as E272A, according to the EU index of Kabat. A modification can be a substitution of N276, such as N276A, according to the EU index of Kabat. A modification can be a substitution of D280, such as D280A, according to the EU index of Kabat. A modification can be a substitution of H285, such as H285A, according to the EU index of Kabat. A modification can be a substitution of N286, such as N286A, according to the EU index of Kabat. A modification can be a substitution of T307, such as T307A, according to the EU index of Kabat. A modification can be a substitution of L309, such as L309A, according to the EU index of Kabat. A modification can be a substitution of N315, such as N315A, according to the EU index of Kabat. A modification can be a substitution of K326, such as K326A, according to the EU index of Kabat. A modification can be a substitution of P331, such as P331A, according to the EU index of Kabat. A modification can be a substitution of S337, such as S337A, according to the EU index of Kabat. A modification can be a substitution of A378, such as A378A, according to the EU index of Kabat. A modification can be a substitution of E430, such as E430, according to the EU index of Kabat.

In some embodiments, the modification comprises substitution of one or more amino acids that increases binding affinity of an IgG Fc domain or region to FcγRII receptor and reduces the binding affinity to FcγRIIIA receptor. A modification can be a substitution of H268, such as H268A, according to the EU index of Kabat. A modification can be a substitution of R301, such as R301A, according to the EU index of Kabat. A modification can be a substitution of K322, such as K322A, according to the EU index of Kabat.

In some embodiments, the modification comprises substitution of one or more amino acids that decreases binding affinity of an IgG Fc domain or region to FcγRII receptor but does not affect the binding affinity to FcγRIIIA receptor. A modification can be a substitution of R292, such as R292A, according to the EU index of Kabat. A modification can be a substitution of K414, such as K414A, according to the EU index of Kabat.

In some embodiments, the modification comprises substitution of one or more amino acids that decreases binding affinity of an IgG Fc domain or region to FcγRII receptor and increases the binding affinity to FcγRIIIA receptor. A modification can be a substitution of S298, such as S298A, according to the EU index of Kabat. A modification can be substitution of S239, 1332 and A330, such as S239D/I332E/A330L. A modification can be substitution of S239 and 1332, such as S239D/I332E.

In some embodiments, the modification comprises substitution of one or more amino acids that decreases binding affinity of an IgG Fc domain or region to FcγRIIIA receptor and does not affect the binding affinity to FcγRII receptor. A modification can be a substitution of S239, such as S239A, according to the EU index of Kabat. A modification can be a substitution of E269, such as E269A, according to the EU index of Kabat. A modification can be a substitution of E293, such as E293A, according to the EU index of Kabat. A modification can be a substitution of Y296, such as Y296F, according to the EU index of Kabat. A modification can be a substitution of V303, such as V303A, according to the EU index of Kabat. A modification can be a substitution of A327, such as A327G, according to the EU index of Kabat. A modification can be a substitution of K338, such as K338A, according to the EU index of Kabat. A modification can be a substitution of D376, such as D376A, according to the EU index of Kabat.

In some embodiments, the modification comprises substitution of one or more amino acids that increases binding affinity of an IgG Fc domain or region to FcγRIIIA receptor and does not affect the binding affinity to FcγRII receptor. A modification can be a substitution of E333, such as E333A, according to the EU index of Kabat. A modification can be a substitution of K334, such as K334A, according to the EU index of Kabat. A modification can be a substitution of A339, such as A339T, according to the EU index of Kabat. A modification can be substitution of S239 and 1332, such as S239D/I332E.

In some embodiments, an IgG Fc domain or region comprises at least one amino acid substitution that reduces the binding affinity to FcRn, as compared to a wild-type or reference IgG Fc domain. A modification can comprise a substitution at H435, such as H435A according to the EU index of Kabat. A modification can comprise a substitution at 1253, such as I253A according to the EU index of Kabat. A modification can comprise a substitution at H310, such as H310A according to the EU index of Kabat. A modification can comprise substitutions at 1253, H310 and H435, such as I253A/H310A/H435A according to the EU index of Kabat.

A modification can comprise a substitution of one amino acid residue that increases the binding affinity of an IgG Fc domain for FcRn, relative to a wildtype or reference IgG Fc domain. A modification can comprise a substitution at V308, such as V308P according to the EU index of Kabat. A modification can comprise a substitution at M428, such as M428L according to the EU index of Kabat. A modification can comprise a substitution at N434, such as N434A according to the EU index of Kabat or N434H according to the EU index of Kabat. A modification can comprise substitutions at T250 and M428, such as T250Q and M428L according to the EU index of Kabat. A modification can comprise substitutions at M428 and N434, such as M428L and N434S, N434A or N434H according to the EU index of Kabat. A modification can comprise substitutions at M252, S254 and T256, such as M252Y/S254T/T256E according to the EU index of Kabat. A modification can be a substitution of one or more amino acids selected from P257L, P257N, P257I, V279E, V279Q, V279Y, A281S, E283F, V284E, L306Y, T307V, V308F, Q311V, D376V, and N434H. Other substitutions in an IgG Fc domain that affect its interaction with FcRn are disclosed in U.S. Pat. No. 9,803,023 (the disclosure of which is incorporated by reference herein).

An antibody of the disclosure may consist of two identical light protein chains and two identical heavy protein chains, all held together covalently by disulfide linkages. The N-terminal regions of the light and heavy chains together may form the antigen recognition site of an antibody. Structurally, various functions of an antibody may be confined to discrete protein domains (i.e., regions). The sites that can recognize and can bind antigen are the three complementarities determining regions (CDRs) that may lie within the variable heavy chain region and variable light chain region at the N-terminal end of the heavy chain and the light chain. The constant domains provide the general framework of the antibody and may not be involved directly in binding the antibody to an antigen, but may be involved in various effector functions, such as participation of the antibody in antibody-dependent cellular cytotoxicity, and may bind Fc receptors. The constant domains may form an Fc region. The constant domains may include an Fc domain. The domains of natural light and heavy chain variable regions may have the same general structures, and each variable domain may comprise four framework regions, whose sequences can be somewhat conserved, connected by the CDRs. The four framework regions may largely adopt a β-sheet conformation and the CDRs can form loops connecting, and in some aspects forming part of, the β-sheet structure. The CDRs in each chain may be held in close proximity by the framework regions and, with the CDRs from the other chain, may contribute to the formation of the antigen binding site.

An antibody construct may comprise a light chain of an amino acid sequence having at least one, two, three, four, five, six, seven, eight, nine, or ten modifications and in certain embodiments, not more than 40, 35, 30, 25, 20, 15, or 10 modifications of the amino acid sequence relative to the natural or original amino acid sequence. An antibody construct may comprise a heavy chain of an amino acid sequence having at least one, two, three, four, five, six, seven, eight, nine, or ten modifications and in certain embodiments, not more than 40, 35, 30, 25, 20, 15, or 10 modifications of the amino acid sequence relative to the natural or original amino acid sequence. An antibody of an antibody construct may include an antibody of any type, which may be assigned to different classes of immunoglobins, e.g., IgA, IgD, IgE, IgG, and IgM. Several different classes may be further divided into isotypes, e.g., IgG1, IgG2, IgG3, IgG4, IgA1, and IgA2. An antibody may further comprise a light chain and a heavy chain, often more than one chain. An antibody with an IgG4 Fc domain paired with a wild type IgG4-hinge region can undergo strand swap, in which one arm of the bivalent antibody dissociates and pairs with a strand of another IgG4 antibody with a different antigen specificity. Strand swap may be prevented by pairing the IgG4 Fc-domain with a S228P mutation of the IgG4 hinge.

Exemplary heavy chain sequences of reference antibodies can be used to identify residue variants and mutants. An exemplary heavy chain sequence for human IgG1 heavy chain is that of the human IgG1 antibody, and can comprise: ASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVF LFPPKPKDTLMISRTPEVTCVVVDVSHEDPEVKFNWYVDGVEVHNAKTKPREEQYNSTYRVV SVLTVLHQDWLNGKEYKCKVSNKALPAPIEKTISKAKGQPREPQVYTLPPSREEMTKNQVSL TCLVKGFYPSDIAVEWESNGQPENNYKTTPPVLDSDGSFFLYSKLTVDKSRWQQGNVFSCSV MHEALHNHYTQKSLSLSPGK (SEQ ID NO: 437). An exemplary heavy chain reference sequence for human IgG2 heavy chain can comprise: ASTKGPSVFPLAPCSRSTSESTAALGCLVKDYFPEPVTVSWNSGALTSGVHTFPAVLQSSGL YSLSSVVTVPSSNFGTQTYTCNVDHKPSNTKVDKTVERKCCVECPPCPAPPVAGPSVFLFPP KPKDTLMISRTPEVTCVVVDVSHEDPEVQFNWYVDGVEVHNAKTKPREEQFNSTFRVVSVLT VVHQDWLNGKEYKCKVSNKGLPAPIEKTISKTKGQPREPQVYTLPPSREEMTKNQVSLTCLV KGFYPSDIAVEWESNGQPENNYKTTPPMLDSDGSFFLYSKLTVDKSRWQQGNVFSCSVMHEA LHNHYTQKSLSLSPGK (SEQ ID NO: 438). An exemplary heavy chain reference sequence for human IgG4 heavy chain can comprise:

(SEQ ID NO: 439)
ASTKGPSVFPLAPCSRSTSESTAALGCLVKDYFPEPVTVSWNSGALTSGVH
TFPAVLQSSGLYSLSSVVTVPSSSLGTKTYTCNVDHKPSNTKVDKRVESKY
GPPCPSCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCS
VMHEALHNHYTQKSLSLSLGK.

The heavy-chain constant regions (Fc) that corresponds to the different classes of immunoglobulins may be α, δ, ε, γ, and μ, respectively. The light chains may be one of either kappa (κ) or lambda (λ), based on the amino acid sequences of the constant domains. The Fc region may contain an Fc domain. An Fc receptor may bind an Fc domain. An Fc domain can comprise amino acid residues 216 to 447 of a human IgG1, which are included in SEQ ID NO: 437. An Fc domain can comprise amino acid residues 216 to 442 of a human IgG2, which are included in SEQ ID NO: 438. An Fc domain can comprise amino acid residues 216 to 44 of an IgG4, which are included in SEQ ID NO: 439.

An antibody construct may comprise an antigen-binding antibody fragment. An antibody fragment may include: (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; and (iii) a Fv fragment consisting of the V_{L} and V_{H} domains of a single arm of an antibody. Although the two domains of the Fv fragment, V_{L} and V_{H}, may be coded for by separate genes, they may be linked by a synthetic linker to be made as a single protein chain in which the V_{L} and V_{H} regions pair to form monovalent molecules.

F(ab′)₂ and Fab′ moieties may be produced recombinantly or by treating immunoglobulin (e.g., monoclonal antibody) with a protease such as pepsin and papain, and may include an antibody fragment generated by digesting immunoglobulin near the disulfide bonds existing between the hinge regions in each of the two H chains. The Fab fragment may also contain the constant domain of the light chain and the first constant domain (C_{H1}) of the heavy chain. Fab′ fragments may differ from Fab fragments by the addition of a few residues at the carboxyl terminus of the heavy chain C_{H1} domain including one or more cysteine(s) from the antibody hinge region.

An Fv may be the minimum antibody fragment which contains a complete antigen-recognition and antigen-binding site. This region may consist of a dimer of one heavy chain and one light chain variable domain in tight, non-covalent association. In this configuration, the three CDRs of each variable domain may interact to define an antigen-binding site on the surface of the V_{H}-V_{L} dimer. A single variable domain (or half of an Fv comprising only three CDRs specific for an antigen) may recognize and bind antigen, although the binding can be at a lower affinity than the affinity of the entire binding site.

An antibody may include an Fc region comprising an Fc domain. The Fc domain of an antibody may interact with FcRs found on immune cells. The Fc domain may also mediate the interaction between effector molecules and cells, which may lead to activation of the immune system. In the IgG, IgA, and IgD antibody isotypes, the Fc region may comprise two identical protein fragments, which can be derived from the second and third constant domains of the antibody's heavy chains. In the IgM and IgE antibody isotypes, the Fc regions may comprise three heavy chain constant domains. In the IgG antibody isotype, the Fc regions may comprise a highly-conserved N-glycosylation site, which may be important for FcR-mediated downstream effects.

An antibody used herein may be “chimeric” or “humanized.” Chimeric or humanized forms of non-human (e.g., murine) antibodies can be chimeric immunoglobulins, immunoglobulin chains or fragments thereof (such as Fv, Fab, Fab′, F(ab′)₂ or other target-binding subdomains of antibodies), which may contain minimal sequences derived from non-human immunoglobulin. In general, the humanized antibody may comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody may also comprise at least a portion of an immunoglobulin constant region (Fc), typically that of a human immunoglobulin consensus sequence.

An antibody described herein may be a human antibody. As used herein, “human antibodies” can include antibodies having, for example, the amino acid sequence of a human immunoglobulin and may include antibodies isolated from human immunoglobulin libraries or from animals transgenic for one or more human immunoglobulins that do not express endogenous immunoglobulins. Human antibodies may be produced using transgenic mice which are incapable of expressing functional endogenous immunoglobulins, but which may express human immunoglobulin genes. Completely human antibodies that recognize a selected epitope may be generated using guided selection. In this approach, a selected non-human monoclonal antibody, e.g., a mouse antibody, may be used to guide the selection of a completely human antibody recognizing the same epitope.

An antibody described herein may be a bispecific antibody or a dual variable domain antibody (DVD). Bispecific and DVD antibodies may be monoclonal, often human or humanized, antibodies that can have binding specificities for at least two different antigens.

An antibody described herein may be a derivatized antibody. For example, derivatized antibodies may be modified by glycosylation, acetylation, pegylation, phosphorylation, amidation, derivatization by known protecting/blocking groups, proteolytic cleavage, linkage to a cellular ligand or other protein.

An antibody described herein may have a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced binding to the Fc receptor (FcR). FcR binding may be reduced by, for example, mutating the immunoglobulin constant region segment of the antibody at particular regions necessary for FcR interactions.

An antibody or Fc domain as described herein may be modified to acquire or improve at least one constant region-mediated biological effector function relative to an unmodified antibody or Fc domain, e.g., to enhance FcγR interactions. For example, an antibody with a constant region that binds to FcγRIIA, FcγRIIB, and/or FcγRIIIA with greater affinity than the corresponding wild type constant region may be produced according to the methods described herein. An Fc domain that binds to FcγRIIA, FcγRIIB, and/or FcγRIIIA with greater affinity than the corresponding wild type Fc domain may be produced according to the methods described herein.

An antibody construct may comprise an antibody with modifications of at least one amino acid residue. Modifications may be substitutions, additions, mutations, deletions, or the like. An antibody modification can be an insertion of an unnatural amino acid.

An antibody construct may comprise an antigen binding domain that specifically binds to an antigen on an immune cell, such as an immune cell (e.g., a T cell, a B cell or an APC), a stellate cell, an epithelial cell, a fibroblast cell, a fibrocyte cell, a myofibroblast, a synovial fibroblast, a podocyte, or other cell associated with the pathogenesis of fibrosis. An antibody construct may comprise an antigen binding domain comprising one or more CDRs that facilitate specific binding to an antigen. An antigen binding domain may comprise a set of CDRs, or pair of variable regions having at least 80% sequence identity to a set of CDRs or pair of variable regions set forth in TABLE 1 or TABLE 2, respectively.

An antibody construct may comprise an antigen binding domain that binds to an antigen, wherein the antigen binding domain comprises a set of CDRs having at least 80%, at least 90%, at least 95%, at least 97%, at least 98%, at least 99%, or at least 100% sequence identity to a set of CDRs set forth in in TABLE 1. An antibody construct may comprise an antigen binding domain that binds to an antigen, wherein the antigen binding domain comprises at least at least 80%, at least 90%, at least 95%, at least 97%, at least 98%, at least 99%, or at least 100% sequence identity to: a) HCDR1 comprising an amino acid sequence of SEQ ID NO: 1, HCDR2 comprising an amino acid sequence of SEQ ID NO: 2, HCDR3 comprising an amino acid sequence of SEQ ID NO: 3, LCDR1 comprising an amino acid sequence of SEQ ID NO: 4, LCDR2 comprising an amino acid sequence of SEQ ID NO: 5, and LCDR3 comprising an amino acid sequence of SEQ ID NO: 6; b) HCDR1 comprising an amino acid sequence of SEQ ID NO: 7, HCDR2 comprising an amino acid sequence of SEQ ID NO: 8, HCDR3 comprising an amino acid sequence of SEQ ID NO: 9, LCDR1 comprising an amino acid sequence of SEQ ID NO: 10, LCDR2 comprising an amino acid sequence of SEQ ID NO: 11, and LCDR3 comprising an amino acid sequence of SEQ ID NO: 12; c) HCDR1 comprising an amino acid sequence of SEQ ID NO: 13, HCDR2 comprising an amino acid sequence of SEQ ID NO: 14, HCDR3 comprising an amino acid sequence of SEQ ID NO: 15, LCDR1 comprising an amino acid sequence of SEQ ID NO: 16, LCDR2 comprising an amino acid sequence of SEQ ID NO: 17, and LCDR3 comprising an amino acid sequence of SEQ ID NO: 18; d) HCDR1 comprising an amino acid sequence of SEQ ID NO: 19, HCDR2 comprising an amino acid sequence of SEQ ID NO: 20, HCDR3 comprising an amino acid sequence of SEQ ID NO: 21, LCDR1 comprising an amino acid sequence of SEQ ID NO: 22, LCDR2 comprising an amino acid sequence of SEQ ID NO: 23, and LCDR3 comprising an amino acid sequence of SEQ ID NO: 24; e) HCDR1 comprising an amino acid sequence of SEQ ID NO: 25, HCDR2 comprising an amino acid sequence of SEQ ID NO: 26, HCDR3 comprising an amino acid sequence of SEQ ID NO: 27, LCDR1 comprising an amino acid sequence of SEQ ID NO: 28, LCDR2 comprising an amino acid sequence of SEQ ID NO: 29, and LCDR3 comprising an amino acid sequence of SEQ ID NO: 30; f) HCDR1 comprising an amino acid sequence of SEQ ID NO: 31, HCDR2 comprising an amino acid sequence of SEQ ID NO: 32, HCDR3 comprising an amino acid sequence of SEQ ID NO: 33, LCDR1 comprising an amino acid sequence of SEQ ID NO: 34, LCDR2 comprising an amino acid sequence of SEQ ID NO: 35, and LCDR3 comprising an amino acid sequence of SEQ ID NO: 36; g) HCDR1 comprising an amino acid sequence of SEQ ID NO: 37, HCDR2 comprising an amino acid sequence of SEQ ID NO: 38, HCDR3 comprising an amino acid sequence of SEQ ID NO: 39, LCDR1 comprising an amino acid sequence of SEQ ID NO: 40, LCDR2 comprising an amino acid sequence of SEQ ID NO: 41, and LCDR3 comprising an amino acid sequence of SEQ ID NO: 42; h) HCDR1 comprising an amino acid sequence of SEQ ID NO: 43, HCDR2 comprising an amino acid sequence of SEQ ID NO: 44, HCDR3 comprising an amino acid sequence of SEQ ID NO: 45, LCDR1 comprising an amino acid sequence of SEQ ID NO: 46, LCDR2 comprising an amino acid sequence of SEQ ID NO: 47, and LCDR3 comprising an amino acid sequence of SEQ ID NO: 48; i) HCDR1 comprising an amino acid sequence of SEQ ID NO: 49, HCDR2 comprising an amino acid sequence of SEQ ID NO: 50, HCDR3 comprising an amino acid sequence of SEQ ID NO: 51, LCDR1 comprising an amino acid sequence of SEQ ID NO: 52, LCDR2 comprising an amino acid sequence of SEQ ID NO: 53, and LCDR3 comprising an amino acid sequence of SEQ ID NO: 54; j) HCDR1 comprising an amino acid sequence of SEQ ID NO: 55, HCDR2 comprising an amino acid sequence of SEQ ID NO: 56, HCDR3 comprising an amino acid sequence of SEQ ID NO: 57, LCDR1 comprising an amino acid sequence of SEQ ID NO: 58, LCDR2 comprising an amino acid sequence of SEQ ID NO: 59, and LCDR3 comprising an amino acid sequence of SEQ ID NO: 60; k) HCDR1 comprising an amino acid sequence of SEQ ID NO: 61, HCDR2 comprising an amino acid sequence of SEQ ID NO: 62, HCDR3 comprising an amino acid sequence of SEQ ID NO: 63, LCDR1 comprising an amino acid sequence of SEQ ID NO: 64, LCDR2 comprising an amino acid sequence of SEQ ID NO: 65, and LCDR3 comprising an amino acid sequence of SEQ ID NO: 66; l) HCDR1 comprising an amino acid sequence of SEQ ID NO: 67, HCDR2 comprising an amino acid sequence of SEQ ID NO: 68, HCDR3 comprising an amino acid sequence of SEQ ID NO: 69, LCDR1 comprising an amino acid sequence of SEQ ID NO: 70, LCDR2 comprising an amino acid sequence of SEQ ID NO: 71, and LCDR3 comprising an amino acid sequence of SEQ ID NO: 72; m) HCDR1 comprising an amino acid sequence of SEQ ID NO: 73, HCDR2 comprising an amino acid sequence of SEQ ID NO: 74, HCDR3 comprising an amino acid sequence of SEQ ID NO: 75, LCDR1 comprising an amino acid sequence of SEQ ID NO: 76, LCDR2 comprising an amino acid sequence of SEQ ID NO: 77, and LCDR3 comprising an amino acid sequence of SEQ ID NO: 78; n) HCDR1 comprising an amino acid sequence of SEQ ID NO: 79, HCDR2 comprising an amino acid sequence of SEQ ID NO: 80, HCDR3 comprising an amino acid sequence of SEQ ID NO: 81, LCDR1 comprising an amino acid sequence of SEQ ID NO: 82, LCDR2 comprising an amino acid sequence of SEQ ID NO: 83, and LCDR3 comprising an amino acid sequence of SEQ ID NO: 84; o) HCDR1 comprising an amino acid sequence of SEQ ID NO: 85, HCDR2 comprising an amino acid sequence of SEQ ID NO: 86, HCDR3 comprising an amino acid sequence of SEQ ID NO: 87, LCDR1 comprising an amino acid sequence of SEQ ID NO: 88, LCDR2 comprising an amino acid sequence of SEQ ID NO: 89, and LCDR3 comprising an amino acid sequence of SEQ ID NO: 90; p) HCDR1 comprising an amino acid sequence of SEQ ID NO: 91, HCDR2 comprising an amino acid sequence of SEQ ID NO: 92, HCDR3 comprising an amino acid sequence of SEQ ID NO: 93, LCDR1 comprising an amino acid sequence of SEQ ID NO: 94, LCDR2 comprising an amino acid sequence of SEQ ID NO: 95, and LCDR3 comprising an amino acid sequence of SEQ ID NO: 96; q) HCDR1 comprising an amino acid sequence of SEQ ID NO: 97, HCDR2 comprising an amino acid sequence of SEQ ID NO: 98, HCDR3 comprising an amino acid sequence of SEQ ID NO: 99, LCDR1 comprising an amino acid sequence of SEQ ID NO: 100, LCDR2 comprising an amino acid sequence of SEQ ID NO: 101, and LCDR3 comprising an amino acid sequence of SEQ ID NO: 102; r) HCDR1 comprising an amino acid sequence of SEQ ID NO: 103, HCDR2 comprising an amino acid sequence of SEQ ID NO: 104, HCDR3 comprising an amino acid sequence of SEQ ID NO: 105, LCDR1 comprising an amino acid sequence of SEQ ID NO: 106, LCDR2 comprising an amino acid sequence of SEQ ID NO: 107, and LCDR3 comprising an amino acid sequence of SEQ ID NO: 108; s) HCDR1 comprising an amino acid sequence of SEQ ID NO: 109, HCDR2 comprising an amino acid sequence of SEQ ID NO: 110, HCDR3 comprising an amino acid sequence of SEQ ID NO: 111, LCDR1 comprising an amino acid sequence of SEQ ID NO: 112, LCDR2 comprising an amino acid sequence of SEQ ID NO: 113, and LCDR3 comprising an amino acid sequence of SEQ ID NO: 114; t) HCDR1 comprising an amino acid sequence of SEQ ID NO: 109, HCDR2 comprising an amino acid sequence of SEQ ID NO: 110, HCDR3 comprising an amino acid sequence of SEQ ID NO: 111, LCDR1 comprising an amino acid sequence of SEQ ID NO: 115, LCDR2 comprising an amino acid sequence of SEQ ID NO: 116, and LCDR3 comprising an amino acid sequence of SEQ ID NO: 117; u) HCDR1 comprising an amino acid sequence of SEQ ID NO: 118, HCDR2 comprising an amino acid sequence of SEQ ID NO: 119, HCDR3 comprising an amino acid sequence of SEQ ID NO: 120, LCDR1 comprising an amino acid sequence of SEQ ID NO: 121, LCDR2 comprising an amino acid sequence of SEQ ID NO: 122, and LCDR3 comprising an amino acid sequence of SEQ ID NO: 123; v) HCDR1 comprising an amino acid sequence of SEQ ID NO: 124, HCDR2 comprising an amino acid sequence of SEQ ID NO: 125, HCDR3 comprising an amino acid sequence of SEQ ID NO: 126, LCDR1 comprising an amino acid sequence of SEQ ID NO: 127, LCDR2 comprising an amino acid sequence of SEQ ID NO: 128, and LCDR3 comprising an amino acid sequence of SEQ ID NO: 129; w) HCDR1 comprising an amino acid sequence of SEQ ID NO: 130, HCDR2 comprising an amino acid sequence of SEQ ID NO: 131, HCDR3 comprising an amino acid sequence of SEQ ID NO: 132, LCDR1 comprising an amino acid sequence of SEQ ID NO: 133, LCDR2 comprising an amino acid sequence of SEQ ID NO: 134, and LCDR3 comprising an amino acid sequence of SEQ ID NO: 135; x) HCDR1 comprising an amino acid sequence of SEQ ID NO: 136, HCDR2 comprising an amino acid sequence of SEQ ID NO: 137, HCDR3 comprising an amino acid sequence of SEQ ID NO: 138, LCDR1 comprising an amino acid sequence of SEQ ID NO: 139, LCDR2 comprising an amino acid sequence of SEQ ID NO: 140, and LCDR3 comprising an amino acid sequence of SEQ ID NO: 141; y) HCDR1 comprising an amino acid sequence of SEQ ID NO: 142, HCDR2 comprising an amino acid sequence of SEQ ID NO: 143, HCDR3 comprising an amino acid sequence of SEQ ID NO: 144, LCDR1 comprising an amino acid sequence of SEQ ID NO: 145, LCDR2 comprising an amino acid sequence of SEQ ID NO: 146, and LCDR3 comprising an amino acid sequence of SEQ ID NO: 147; z) HCDR1 comprising an amino acid sequence of SEQ ID NO: 148, HCDR2 comprising an amino acid sequence of SEQ ID NO: 149, HCDR3 comprising an amino acid sequence of SEQ ID NO: 150, LCDR1 comprising an amino acid sequence of SEQ ID NO: 151, LCDR2 comprising an amino acid sequence of SEQ ID NO: 152, and LCDR3 comprising an amino acid sequence of SEQ ID NO: 153; aa) HCDR1 comprising an amino acid sequence of SEQ ID NO: 154, HCDR2 comprising an amino acid sequence of SEQ ID NO: 155, HCDR3 comprising an amino acid sequence of SEQ ID NO: 156, LCDR1 comprising an amino acid sequence of SEQ ID NO: 157, LCDR2 comprising an amino acid sequence of SEQ ID NO: 158, and LCDR3 comprising an amino acid sequence of SEQ ID NO: 159; bb) HCDR1 comprising an amino acid sequence of SEQ ID NO: 160, HCDR2 comprising an amino acid sequence of SEQ ID NO: 161, HCDR3 comprising an amino acid sequence of SEQ ID NO: 162, LCDR1 comprising an amino acid sequence of SEQ ID NO: 163, LCDR2 comprising an amino acid sequence of SEQ ID NO: 164, and LCDR3 comprising an amino acid sequence of SEQ ID NO: 165; cc) HCDR1 comprising an amino acid sequence of SEQ ID NO: 166, HCDR2 comprising an amino acid sequence of SEQ ID NO: 167, HCDR3 comprising an amino acid sequence of SEQ ID NO: 168, LCDR1 comprising an amino acid sequence of SEQ ID NO: 169, LCDR2 comprising an amino acid sequence of SEQ ID NO: 170, and LCDR3 comprising an amino acid sequence of SEQ ID NO: 171; dd) HCDR1 comprising an amino acid sequence of SEQ ID NO: 172, HCDR2 comprising an amino acid sequence of SEQ ID NO: 173, HCDR3 comprising an amino acid sequence of SEQ ID NO: 174, LCDR1 comprising an amino acid sequence of SEQ ID NO: 175, LCDR2 comprising an amino acid sequence of SEQ ID NO: 176, and LCDR3 comprising an amino acid sequence of SEQ ID NO: 177; ee) HCDR1 comprising an amino acid sequence of SEQ ID NO: 178, HCDR2 comprising an amino acid sequence of SEQ ID NO: 179, HCDR3 comprising an amino acid sequence of SEQ ID NO: 180, LCDR1 comprising an amino acid sequence of SEQ ID NO: 181, LCDR2 comprising an amino acid sequence of SEQ ID NO: 182, and LCDR3 comprising an amino acid sequence of SEQ ID NO: 183; ff) HCDR1 comprising an amino acid sequence of SEQ ID NO: 184, HCDR2 comprising an amino acid sequence of SEQ ID NO: 185, HCDR3 comprising an amino acid sequence of SEQ ID NO: 186, LCDR1 comprising an amino acid sequence of SEQ ID NO: 187, LCDR2 comprising an amino acid sequence of SEQ ID NO: 188, and LCDR3 comprising an amino acid sequence of SEQ ID NO: 189; gg) HCDR1 comprising an amino acid sequence of SEQ ID NO: 190, HCDR2 comprising an amino acid sequence of SEQ ID NO: 191, HCDR3 comprising an amino acid sequence of SEQ ID NO: 192, LCDR1 comprising an amino acid sequence of SEQ ID NO: 193, LCDR2 comprising an amino acid sequence of SEQ ID NO: 194, and LCDR3 comprising an amino acid sequence of SEQ ID NO: 195; hh) HCDR1 comprising an amino acid sequence of SEQ ID NO: 196, HCDR2 comprising an amino acid sequence of SEQ ID NO: 197, HCDR3 comprising an amino acid sequence of SEQ ID NO: 198, LCDR1 comprising an amino acid sequence of SEQ ID NO: 199, LCDR2 comprising an amino acid sequence of SEQ ID NO: 200, and LCDR3 comprising an amino acid sequence of SEQ ID NO: 201; ii) HCDR1 comprising an amino acid sequence of SEQ ID NO: 202, HCDR2 comprising an amino acid sequence of SEQ ID NO: 203, HCDR3 comprising an amino acid sequence of SEQ ID NO: 204, LCDR1 comprising an amino acid sequence of SEQ ID NO: 205, LCDR2 comprising an amino acid sequence of SEQ ID NO: 206, and LCDR3 comprising an amino acid sequence of SEQ ID NO: 207; jj) HCDR1 comprising an amino acid sequence of SEQ ID NO: 208, HCDR2 comprising an amino acid sequence of SEQ ID NO: 209, HCDR3 comprising an amino acid sequence of SEQ ID NO: 210, LCDR1 comprising an amino acid sequence of SEQ ID NO: 211, LCDR2 comprising an amino acid sequence of SEQ ID NO: 212, and LCDR3 comprising an amino acid sequence of SEQ ID NO: 213; kk) HCDR1 comprising an amino acid sequence of SEQ ID NO: 214, HCDR2 comprising an amino acid sequence of SEQ ID NO: 215, HCDR3 comprising an amino acid sequence of SEQ ID NO: 216, LCDR1 comprising an amino acid sequence of SEQ ID NO: 217, LCDR2 comprising an amino acid sequence of SEQ ID NO: 218, and LCDR3 comprising an amino acid sequence of SEQ ID NO: 219; ll) HCDR1 comprising an amino acid sequence of SEQ ID NO: 220, HCDR2 comprising an amino acid sequence of SEQ ID NO: 221, HCDR3 comprising an amino acid sequence of SEQ ID NO: 222, LCDR1 comprising an amino acid sequence of SEQ ID NO: 223, LCDR2 comprising an amino acid sequence of SEQ ID NO: 224, and LCDR3 comprising an amino acid sequence of SEQ ID NO: 225; mm) HCDR1 comprising an amino acid sequence of SEQ ID NO: 226, HCDR2 comprising an amino acid sequence of SEQ ID NO: 227, HCDR3 comprising an amino acid sequence of SEQ ID NO: 228, LCDR1 comprising an amino acid sequence of SEQ ID NO: 229, LCDR2 comprising an amino acid sequence of SEQ ID NO: 230, and LCDR3 comprising an amino acid sequence of SEQ ID NO: 231; nn) HCDR1 comprising an amino acid sequence of SEQ ID NO: 232, HCDR2 comprising an amino acid sequence of SEQ ID NO: 234, HCDR3 comprising an amino acid sequence of SEQ ID NO: 235, LCDR1 comprising an amino acid sequence of SEQ ID NO: 236, LCDR2 comprising an amino acid sequence of SEQ ID NO: 237, and LCDR3 comprising an amino acid sequence of SEQ ID NO: 238; oo) HCDR1 comprising an amino acid sequence of SEQ ID NO: 239, HCDR2 comprising an amino acid sequence of SEQ ID NO: 240, HCDR3 comprising an amino acid sequence of SEQ ID NO: 241, LCDR1 comprising an amino acid sequence of SEQ ID NO: 242, LCDR2 comprising an amino acid sequence of SEQ ID NO: 243, and LCDR3 comprising an amino acid sequence of SEQ ID NO: 244; pp) HCDR1 comprising an amino acid sequence of SEQ ID NO: 245, HCDR2 comprising an amino acid sequence of SEQ ID NO: 246, HCDR3 comprising an amino acid sequence of SEQ ID NO: 247, LCDR1 comprising an amino acid sequence of SEQ ID NO: 248, LCDR2 comprising an amino acid sequence of SEQ ID NO: 249, and LCDR3 comprising an amino acid sequence of SEQ ID NO: 250; qq) HCDR1 comprising an amino acid sequence of SEQ ID NO: 251, HCDR2 comprising an amino acid sequence of SEQ ID NO: 252, HCDR3 comprising an amino acid sequence of SEQ ID NO: 253, LCDR1 comprising an amino acid sequence of SEQ ID NO: 254, LCDR2 comprising an amino acid sequence of SEQ ID NO: 255, and LCDR3 comprising an amino acid sequence of SEQ ID NO: 256; rr) HCDR1 comprising an amino acid sequence of SEQ ID NO: 257, HCDR2 comprising an amino acid sequence of SEQ ID NO: 258, HCDR3 comprising an amino acid sequence of SEQ ID NO: 259, LCDR1 comprising an amino acid sequence of SEQ ID NO: 260, LCDR2 comprising an amino acid sequence of SEQ ID NO: 261, and LCDR3 comprising an amino acid sequence of SEQ ID NO: 262; ss) HCDR1 comprising an amino acid sequence of SEQ ID NO: 263, HCDR2 comprising an amino acid sequence of SEQ ID NO: 264, HCDR3 comprising an amino acid sequence of SEQ ID NO: 265, LCDR1 comprising an amino acid sequence of SEQ ID NO: 266, LCDR2 comprising an amino acid sequence of SEQ ID NO: 267, and LCDR3 comprising an amino acid sequence of SEQ ID NO: 268; tt) HCDR1 comprising an amino acid sequence of SEQ ID NO: 269, HCDR2 comprising an amino acid sequence of SEQ ID NO: 270, HCDR3 comprising an amino acid sequence of SEQ ID NO: 271, LCDR1 comprising an amino acid sequence of SEQ ID NO: 272, LCDR2 comprising an amino acid sequence of SEQ ID NO: 273, and LCDR3 comprising an amino acid sequence of SEQ ID NO: 274; uu) HCDR1 comprising an amino acid sequence of SEQ ID NO: 275, HCDR2 comprising an amino acid sequence of SEQ ID NO: 276, HCDR3 comprising an amino acid sequence of SEQ ID NO: 277, LCDR1 comprising an amino acid sequence of SEQ ID NO: 278, LCDR2 comprising an amino acid sequence of SEQ ID NO: 279, and LCDR3 comprising an amino acid sequence of SEQ ID NO: 280; vv) HCDR1 comprising an amino acid sequence of SEQ ID NO: 281, HCDR2 comprising an amino acid sequence of SEQ ID NO: 282, HCDR3 comprising an amino acid sequence of SEQ ID NO: 283, LCDR1 comprising an amino acid sequence of SEQ ID NO: 284, LCDR2 comprising an amino acid sequence of SEQ ID NO: 285, and LCDR3 comprising an amino acid sequence of SEQ ID NO: 286; ww) HCDR1 comprising an amino acid sequence of SEQ ID NO: 287, HCDR2 comprising an amino acid sequence of SEQ ID NO: 288, HCDR3 comprising an amino acid sequence of SEQ ID NO: 289, LCDR1 comprising an amino acid sequence of SEQ ID NO: 290, LCDR2 comprising an amino acid sequence of SEQ ID NO: 291, and LCDR3 comprising an amino acid sequence of SEQ ID NO: 292; xx) HCDR1 comprising an amino acid sequence of SEQ ID NO: 293, HCDR2 comprising an amino acid sequence of SEQ ID NO: 294, HCDR3 comprising an amino acid sequence of SEQ ID NO: 295, LCDR1 comprising an amino acid sequence of SEQ ID NO: 296, LCDR2 comprising an amino acid sequence of SEQ ID NO: 297, and LCDR3 comprising an amino acid sequence of SEQ ID NO: 298; yy) HCDR1 comprising an amino acid sequence of SEQ ID NO: 440, HCDR2 comprising an amino acid sequence of SEQ ID NO: 441, HCDR3 comprising an amino acid sequence of SEQ ID NO: 442, LCDR1 comprising an amino acid sequence of SEQ ID NO: 443, LCDR2 comprising an amino acid sequence of SEQ ID NO: 444, and LCDR3 comprising an amino acid sequence of SEQ ID NO: 445; zz) HCDR1 comprising an amino acid sequence of SEQ ID NO: 446, HCDR2 comprising an amino acid sequence of SEQ ID NO: 447, HCDR3 comprising an amino acid sequence of SEQ ID NO: 448, LCDR1 comprising an amino acid sequence of SEQ ID NO: 449, LCDR2 comprising an amino acid sequence of SEQ ID NO: 450, and LCDR3 comprising an amino acid sequence of SEQ ID NO: 451; aaa) HCDR1 comprising an amino acid sequence of SEQ ID NO: 452, HCDR2 comprising an amino acid sequence of SEQ ID NO: 453, HCDR3 comprising an amino acid sequence of SEQ ID NO: 454, LCDR1 comprising an amino acid sequence of SEQ ID NO: 455, LCDR2 comprising an amino acid sequence of SEQ ID NO: 456, and LCDR3 comprising an amino acid sequence of SEQ ID NO: 457; bbb) HCDR1 comprising an amino acid sequence of SEQ ID NO: 458, HCDR2 comprising an amino acid sequence of SEQ ID NO: 459, HCDR3 comprising an amino acid sequence of SEQ ID NO: 460, LCDR1 comprising an amino acid sequence of SEQ ID NO: 461, LCDR2 comprising an amino acid sequence of SEQ ID NO: 462, and LCDR3 comprising an amino acid sequence of SEQ ID NO: 463; ccc) HCDR1 comprising an amino acid sequence of SEQ ID NO: 464, HCDR2 comprising an amino acid sequence of SEQ ID NO: 465, HCDR3 comprising an amino acid sequence of SEQ ID NO: 466, LCDR1 comprising an amino acid sequence of SEQ ID NO: 467, LCDR2 comprising an amino acid sequence of SEQ ID NO: 468, and LCDR3 comprising an amino acid sequence of SEQ ID NO: 469; ddd) HCDR1 comprising an amino acid sequence of SEQ ID NO: 470, HCDR2 comprising an amino acid sequence of SEQ ID NO: 471, HCDR3 comprising an amino acid sequence of SEQ ID NO: 472, LCDR1 comprising an amino acid sequence of SEQ ID NO: 473, LCDR2 comprising an amino acid sequence of SEQ ID NO: 474, and LCDR3 comprising an amino acid sequence of SEQ ID NO: 475; or eee) HCDR1 comprising an amino acid sequence of SEQ ID NO: 476, HCDR2 comprising an amino acid sequence of SEQ ID NO: 477, HCDR3 comprising an amino acid sequence of SEQ ID NO: 478, LCDR1 comprising an amino acid sequence of SEQ ID NO: 479, LCDR2 comprising an amino acid sequence of SEQ ID NO: 480, and LCDR3 comprising an amino acid sequence of SEQ ID NO: 481.

An antibody construct may comprise an antigen binding domain comprising one or more variable domains. An antibody construct may comprise an antigen binding domain comprising a light chain variable domain (V_{L} domain). A binding domain may comprise a light chain variable region shaving at least 80%, at least 90%, at least 95%, at least 97%, at least 98%, at least 99%, or at least 100% sequence identity to any V_{L} sequence in Table 2. An antibody construct may comprise an antigen binding domain comprising a heavy chain variable domain (V_{H} domain). An antigen binding domain may comprise a heavy chain variable region having at least 80%, at least 90%, at least 95%, at least 97%, at least 98%, at least 99%, or at least 100% sequence identity to any V_{H} sequence in TABLE 2. An antigen binding domain can comprise a pair of heavy and light chain variable regions having at least 80% sequence identity a pair of variable regions set forth in TABLE 2. An antigen binding domain can comprise a pair of heavy and light chain variable regions having at least 80% sequence identity to the non-CDR regions of a pair of variable regions set forth in TABLE 2.

An antibody construct may comprise an antigen binding domain that specifically binds to an antigen, wherein the antigen binding domain comprises: a) a V_{H} sequence having at least 80%, at least 90%, at least 95%, at least 97%, at least 98%, at least 99%, or at least 100% sequence identity to an amino acid sequence of SEQ ID NO: 300, and a V_{L} sequence having at least 80%, at least 90%, at least 95%, at least 97%, at least 98%, at least 99%, or at least 100% sequence identity to an amino acid sequence of SEQ ID NO: 299; b) a V_{H} sequence having at least 80%, at least 90%, at least 95%, at least 97%, at least 98%, at least 99%, or at least 100% sequence identity to an amino acid sequence of SEQ ID NO: 301, and a V_{L} sequence having at least 80%, at least 90%, at least 95%, at least 97%, at least 98%, at least 99%, or at least 100% sequence identity to an amino acid sequence of SEQ ID NO: 299; c) a V_{H} sequence having at least 80%, at least 90%, at least 95%, at least 97%, at least 98%, at least 99%, or at least 100% sequence identity to an amino acid sequence of SEQ ID NO: 302, and a V_{L} sequence having at least 80%, at least 90%, at least 95%, at least 97%, at least 98%, at least 99%, or at least 100% sequence identity to an amino acid sequence of SEQ ID NO: 303; d) a V_{H} sequence having at least 80%, at least 90%, at least 95%, at least 97%, at least 98%, at least 99%, or at least 100% sequence identity to an amino acid sequence of SEQ ID NO: 304, and a V_{L} sequence having at least 80%, at least 90%, at least 95%, at least 97%, at least 98%, at least 99%, or at least 100% sequence identity to an amino acid sequence of SEQ ID NO: 305; e) a V_{H} sequence having at least 80%, at least 90%, at least 95%, at least 97%, at least 98%, at least 99%, or at least 100% sequence identity to an amino acid sequence of SEQ ID NO: 306, and a V_{L} sequence having at least 80%, at least 90%, at least 95%, at least 97%, at least 98%, at least 99%, or at least 100% sequence identity to an amino acid sequence of SEQ ID NO: 307; f) a V_{H} sequence having at least 80%, at least 90%, at least 95%, at least 97%, at least 98%, at least 99%, or at least 100% sequence identity to an amino acid sequence of SEQ ID NO: 308, and a V_{L} sequence having at least 80%, at least 90%, at least 95%, at least 97%, at least 98%, at least 99%, or at least 100% sequence identity to an amino acid sequence of SEQ ID NO: 309; g) a V_{H} sequence having at least 80%, at least 90%, at least 95%, at least 97%, at least 98%, at least 99%, or at least 100% sequence identity to an amino acid sequence of SEQ ID NO: 310, and a V_{L} sequence having at least 80%, at least 90%, at least 95%, at least 97%, at least 98%, at least 99%, or at least 100% sequence identity to an amino acid sequence of SEQ ID NO: 311; h) a V_{H} sequence having at least 80%, at least 90%, at least 95%, at least 97%, at least 98%, at least 99%, or at least 100% sequence identity to an amino acid sequence of SEQ ID NO: 312, and a V_{L} sequence having at least 80%, at least 90%, at least 95%, at least 97%, at least 98%, at least 99%, or at least 100% sequence identity to an amino acid sequence of SEQ ID NO: 313; i) a V_{H} sequence having at least 80%, at least 90%, at least 95%, at least 97%, at least 98%, at least 99%, or at least 100% sequence identity to an amino acid sequence of SEQ ID NO: 314, and a V_{L} sequence having at least 80%, at least 90%, at least 95%, at least 97%, at least 98%, at least 99%, or at least 100% sequence identity to an amino acid sequence of SEQ ID NO: 315; j) a V_{H} sequence having at least 80%, at least 90%, at least 95%, at least 97%, at least 98%, at least 99%, or at least 100% sequence identity to an amino acid sequence of SEQ ID NO: 316, and a V_{L} sequence having at least 80%, at least 90%, at least 95%, at least 97%, at least 98%, at least 99%, or at least 100% sequence identity to an amino acid sequence of SEQ ID NO: 317; k) a V_{H} sequence having at least 80%, at least 90%, at least 95%, at least 97%, at least 98%, at least 99%, or at least 100% sequence identity to an amino acid sequence of SEQ ID NO: 318, and a V_{L} sequence having at least 80%, at least 90%, at least 95%, at least 97%, at least 98%, at least 99%, or at least 100% sequence identity to an amino acid sequence of SEQ ID NO: 320; l) a V_{H} sequence having at least 80%, at least 90%, at least 95%, at least 97%, at least 98%, at least 99%, or at least 100% sequence identity to an amino acid sequence of SEQ ID NO: 319, and a V_{L} sequence having at least 80%, at least 90%, at least 95%, at least 97%, at least 98%, at least 99%, or at least 100% sequence identity to an amino acid sequence of SEQ ID NO: 320; m) a V_{H} sequence having at least 80%, at least 90%, at least 95%, at least 97%, at least 98%, at least 99%, or at least 100% sequence identity to an amino acid sequence of SEQ ID NO: 321, and a V_{L} sequence having at least 80%, at least 90%, at least 95%, at least 97%, at least 98%, at least 99%, or at least 100% sequence identity to an amino acid sequence of SEQ ID NO: 322; n) a V_{H} sequence having at least 80%, at least 90%, at least 95%, at least 97%, at least 98%, at least 99%, or at least 100% sequence identity to an amino acid sequence of SEQ ID NO: 323, and a V_{L} sequence having at least 80%, at least 90%, at least 95%, at least 97%, at least 98%, at least 99%, or at least 100% sequence identity to an amino acid sequence of SEQ ID NO: 324; o) a V_{H} sequence having at least 80%, at least 90%, at least 95%, at least 97%, at least 98%, at least 99%, or at least 100% sequence identity to an amino acid sequence of SEQ ID NO: 325, and a V_{L} sequence having at least 80%, at least 90%, at least 95%, at least 97%, at least 98%, at least 99%, or at least 100% sequence identity to an amino acid sequence of SEQ ID NO: 326; p) a V_{H} sequence having at least 80%, at least 90%, at least 95%, at least 97%, at least 98%, at least 99%, or at least 100% sequence identity to an amino acid sequence of SEQ ID NO: 327, and a V_{L} sequence having at least 80%, at least 90%, at least 95%, at least 97%, at least 98%, at least 99%, or at least 100% sequence identity to an amino acid sequence of SEQ ID NO: 328; q) a V_{H} sequence having at least 80%, at least 90%, at least 95%, at least 97%, at least 98%, at least 99%, or at least 100% sequence identity to an amino acid sequence of SEQ ID NO: 329, and a V_{L} sequence having at least 80%, at least 90%, at least 95%, at least 97%, at least 98%, at least 99%, or at least 100% sequence identity to an amino acid sequence of SEQ ID NO: 330; r) a V_{H} sequence having at least 80%, at least 90%, at least 95%, at least 97%, at least 98%, at least 99%, or at least 100% sequence identity to an amino acid sequence of SEQ ID NO: 331, and a V_{L} sequence having at least 80%, at least 90%, at least 95%, at least 97%, at least 98%, at least 99%, or at least 100% sequence identity to an amino acid sequence of SEQ ID NO: 334; s) a V_{H} sequence having at least 80%, at least 90%, at least 95%, at least 97%, at least 98%, at least 99%, or at least 100% sequence identity to an amino acid sequence of SEQ ID NO: 331, and a V_{L} sequence having at least 80%, at least 90%, at least 95%, at least 97%, at least 98%, at least 99%, or at least 100% sequence identity to an amino acid sequence of SEQ ID NO: 335; t) a V_{H} sequence having at least 80%, at least 90%, at least 95%, at least 97%, at least 98%, at least 99%, or at least 100% sequence identity to an amino acid sequence of SEQ ID NO: 332, and a V_{L} sequence having at least 80%, at least 90%, at least 95%, at least 97%, at least 98%, at least 99%, or at least 100% sequence identity to an amino acid sequence of SEQ ID NO: 334; u) a V_{H} sequence having at least 80%, at least 90%, at least 95%, at least 97%, at least 98%, at least 99%, or at least 100% sequence identity to an amino acid sequence of SEQ ID NO: 332, and a V_{L} sequence having at least 80%, at least 90%, at least 95%, at least 97%, at least 98%, at least 99%, or at least 100% sequence identity to an amino acid sequence of SEQ ID NO: 335; v) a V_{H} sequence having at least 80%, at least 90%, at least 95%, at least 97%, at least 98%, at least 99%, or at least 100% sequence identity to an amino acid sequence of SEQ ID NO: 333, and a V_{L} sequence having at least 80%, at least 90%, at least 95%, at least 97%, at least 98%, at least 99%, or at least 100% sequence identity to an amino acid sequence of SEQ ID NO: 334; w) a V_{H} sequence having at least 80%, at least 90%, at least 95%, at least 97%, at least 98%, at least 99%, or at least 100% sequence identity to an amino acid sequence of SEQ ID NO: 333, and a V_{L} sequence having at least 80%, at least 90%, at least 95%, at least 97%, at least 98%, at least 99%, or at least 100% sequence identity to an amino acid sequence of SEQ ID NO: 335; x) a V_{H} sequence having at least 80%, at least 90%, at least 95%, at least 97%, at least 98%, at least 99%, or at least 100% sequence identity to an amino acid sequence of SEQ ID NO: 336, and a V_{L} sequence having at least 80%, at least 90%, at least 95%, at least 97%, at least 98%, at least 99%, or at least 100% sequence identity to an amino acid sequence of SEQ ID NO: 337; y) a V_{H} sequence having at least 80%, at least 90%, at least 95%, at least 97%, at least 98%, at least 99%, or at least 100% sequence identity to an amino acid sequence of SEQ ID NO: 338, and a V_{L} sequence having at least 80%, at least 90%, at least 95%, at least 97%, at least 98%, at least 99%, or at least 100% sequence identity to an amino acid sequence of SEQ ID NO: 339; z) a V_{H} sequence having at least 80%, at least 90%, at least 95%, at least 97%, at least 98%, at least 99%, or at least 100% sequence identity to an amino acid sequence of SEQ ID NO: 340, and a V_{L} sequence having at least 80%, at least 90%, at least 95%, at least 97%, at least 98%, at least 99%, or at least 100% sequence identity to an amino acid sequence of SEQ ID NO: 341; aa) a V_{H} sequence having at least 80%, at least 90%, at least 95%, at least 97%, at least 98%, at least 99%, or at least 100% sequence identity to an amino acid sequence of SEQ ID NO: 342, and a V_{L} sequence having at least 80%, at least 90%, at least 95%, at least 97%, at least 98%, at least 99%, or at least 100% sequence identity to an amino acid sequence of SEQ ID NO: 343; bb) a V_{H} sequence having at least 80%, at least 90%, at least 95%, at least 97%, at least 98%, at least 99%, or at least 100% sequence identity to an amino acid sequence of SEQ ID NO: 344, and a V_{L} sequence having at least 80%, at least 90%, at least 95%, at least 97%, at least 98%, at least 99%, or at least 100% sequence identity to an amino acid sequence of SEQ ID NO: 345; cc) a V_{H} sequence having at least 80%, at least 90%, at least 95%, at least 97%, at least 98%, at least 99%, or at least 100% sequence identity to an amino acid sequence of SEQ ID NO: 346, and a V_{L} sequence having at least 80%, at least 90%, at least 95%, at least 97%, at least 98%, at least 99%, or at least 100% sequence identity to an amino acid sequence of SEQ ID NO: 347; dd) a V_{H} sequence having at least 80%, at least 90%, at least 95%, at least 97%, at least 98%, at least 99%, or at least 100% sequence identity to an amino acid sequence of SEQ ID NO: 348, and a V_{L} sequence having at least 80%, at least 90%, at least 95%, at least 97%, at least 98%, at least 99%, or at least 100% sequence identity to an amino acid sequence of SEQ ID NO: 349; ee) a V_{H} sequence having at least 80%, at least 90%, at least 95%, at least 97%, at least 98%, at least 99%, or at least 100% sequence identity to an amino acid sequence of SEQ ID NO: 350, and a V_{L} sequence having at least 80%, at least 90%, at least 95%, at least 97%, at least 98%, at least 99%, or at least 100% sequence identity to an amino acid sequence of SEQ ID NO: 351; ff) a V_{H} sequence having at least 80%, at least 90%, at least 95%, at least 97%, at least 98%, at least 99%, or at least 100% sequence identity to an amino acid sequence of SEQ ID NO: 352, and a V_{L} sequence having at least 80%, at least 90%, at least 95%, at least 97%, at least 98%, at least 99%, or at least 100% sequence identity to an amino acid sequence of SEQ ID NO: 353; gg) a V_{H} sequence having at least 80%, at least 90%, at least 95%, at least 97%, at least 98%, at least 99%, or at least 100% sequence identity to an amino acid sequence of SEQ ID NO: 354, and a V_{L} sequence having at least 80%, at least 90%, at least 95%, at least 97%, at least 98%, at least 99%, or at least 100% sequence identity to an amino acid sequence of SEQ ID NO: 355; hh) a V_{H} sequence having at least 80%, at least 90%, at least 95%, at least 97%, at least 98%, at least 99%, or at least 100% sequence identity to an amino acid sequence of SEQ ID NO: 356, and a V_{L} sequence having at least 80%, at least 90%, at least 95%, at least 97%, at least 98%, at least 99%, or at least 100% sequence identity to an amino acid sequence of SEQ ID NO: 357; ii) a V_{H} sequence having at least 80%, at least 90%, at least 95%, at least 97%, at least 98%, at least 99%, or at least 100% sequence identity to an amino acid sequence of SEQ ID NO: 358, and a V_{L} sequence having at least 80%, at least 90%, at least 95%, at least 97%, at least 98%, at least 99%, or at least 100% sequence identity to an amino acid sequence of SEQ ID NO: 359; jj) a V_{H} sequence having at least 80%, at least 90%, at least 95%, at least 97%, at least 98%, at least 99%, or at least 100% sequence identity to an amino acid sequence of SEQ ID NO: 358, and a V_{L} sequence having at least 80%, at least 90%, at least 95%, at least 97%, at least 98%, at least 99%, or at least 100% sequence identity to an amino acid sequence of SEQ ID NO: 360; kk) a V_{H} sequence having at least 80%, at least 90%, at least 95%, at least 97%, at least 98%, at least 99%, or at least 100% sequence identity to an amino acid sequence of SEQ ID NO: 361, and a V_{L} sequence having at least 80%, at least 90%, at least 95%, at least 97%, at least 98%, at least 99%, or at least 100% sequence identity to an amino acid sequence of SEQ ID NO: 362; ll) a V_{H} sequence having at least 80%, at least 90%, at least 95%, at least 97%, at least 98%, at least 99%, or at least 100% sequence identity to an amino acid sequence of SEQ ID NO: 363, and a V_{L} sequence having at least 80%, at least 90%, at least 95%, at least 97%, at least 98%, at least 99%, or at least 100% sequence identity to an amino acid sequence of SEQ ID NO: 364; mm) a V_{H} sequence having at least 80%, at least 90%, at least 95%, at least 97%, at least 98%, at least 99%, or at least 100% sequence identity to an amino acid sequence of SEQ ID NO: 365, and a V_{L} sequence having at least 80%, at least 90%, at least 95%, at least 97%, at least 98%, at least 99%, or at least 100% sequence identity to an amino acid sequence of SEQ ID NO: 366; nn) a V_{H} sequence having at least 80%, at least 90%, at least 95%, at least 97%, at least 98%, at least 99%, or at least 100% sequence identity to an amino acid sequence of SEQ ID NO: 367, and a V_{L} sequence having at least 80%, at least 90%, at least 95%, at least 97%, at least 98%, at least 99%, or at least 100% sequence identity to an amino acid sequence of SEQ ID NO: 368; oo) a V_{H} sequence having at least 80%, at least 90%, at least 95%, at least 97%, at least 98%, at least 99%, or at least 100% sequence identity to an amino acid sequence of SEQ ID NO: 367, and a V_{L} sequence having at least 80%, at least 90%, at least 95%, at least 97%, at least 98%, at least 99%, or at least 100% sequence identity to an amino acid sequence of SEQ ID NO: 369; pp) a V_{H} sequence having at least 80%, at least 90%, at least 95%, at least 97%, at least 98%, at least 99%, or at least 100% sequence identity to an amino acid sequence of SEQ ID NO: 367, and a V_{L} sequence having at least 80%, at least 90%, at least 95%, at least 97%, at least 98%, at least 99%, or at least 100% sequence identity to an amino acid sequence of SEQ ID NO: 370; qq) a V_{H} sequence having at least 80%, at least 90%, at least 95%, at least 97%, at least 98%, at least 99%, or at least 100% sequence identity to an amino acid sequence of SEQ ID NO: 367, and a V_{L} sequence having at least 80%, at least 90%, at least 95%, at least 97%, at least 98%, at least 99%, or at least 100% sequence identity to an amino acid sequence of SEQ ID NO: 371; rr) a V_{H} sequence having at least 80%, at least 90%, at least 95%, at least 97%, at least 98%, at least 99%, or at least 100% sequence identity to an amino acid sequence of SEQ ID NO: 367, and a V_{L} sequence having at least 80%, at least 90%, at least 95%, at least 97%, at least 98%, at least 99%, or at least 100% sequence identity to an amino acid sequence of SEQ ID NO: 372; ss) a V_{H} sequence having at least 80%, at least 90%, at least 95%, at least 97%, at least 98%, at least 99%, or at least 100% sequence identity to an amino acid sequence of SEQ ID NO: 374, and a V_{L} sequence having at least 80%, at least 90%, at least 95%, at least 97%, at least 98%, at least 99%, or at least 100% sequence identity to an amino acid sequence of SEQ ID NO: 373; tt) a V_{H} sequence having at least 80%, at least 90%, at least 95%, at least 97%, at least 98%, at least 99%, or at least 100% sequence identity to an amino acid sequence of SEQ ID NO: 375, and a V_{L} sequence having at least 80%, at least 90%, at least 95%, at least 97%, at least 98%, at least 99%, or at least 100% sequence identity to an amino acid sequence of SEQ ID NO: 376; uu) a V_{H} sequence having at least 80%, at least 90%, at least 95%, at least 97%, at least 98%, at least 99%, or at least 100% sequence identity to an amino acid sequence of SEQ ID NO: 377, and a V_{L} sequence having at least 80%, at least 90%, at least 95%, at least 97%, at least 98%, at least 99%, or at least 100% sequence identity to an amino acid sequence of SEQ ID NO: 378; vv) a V_{H} sequence having at least 80%, at least 90%, at least 95%, at least 97%, at least 98%, at least 99%, or at least 100% sequence identity to an amino acid sequence of SEQ ID NO: 379, and a V_{L} sequence having at least 80%, at least 90%, at least 95%, at least 97%, at least 98%, at least 99%, or at least 100% sequence identity to an amino acid sequence of SEQ ID NO: 380; ww) a V_{H} sequence having at least 80%, at least 90%, at least 95%, at least 97%, at least 98%, at least 99%, or at least 100% sequence identity to an amino acid sequence of SEQ ID NO: 381, and a V_{L} sequence having at least 80%, at least 90%, at least 95%, at least 97%, at least 98%, at least 99%, or at least 100% sequence identity to an amino acid sequence of SEQ ID NO: 382; xx) a V_{H} sequence having at least 80%, at least 90%, at least 95%, at least 97%, at least 98%, at least 99%, or at least 100% sequence identity to an amino acid sequence of SEQ ID NO: 384, and a V_{L} sequence having at least 80%, at least 90%, at least 95%, at least 97%, at least 98%, at least 99%, or at least 100% sequence identity to an amino acid sequence of SEQ ID NO: 383; yy) a V_{H} sequence having at least 80%, at least 90%, at least 95%, at least 97%, at least 98%, at least 99%, or at least 100% sequence identity to an amino acid sequence of SEQ ID NO: 385, and a V_{L} sequence having at least 80%, at least 90%, at least 95%, at least 97%, at least 98%, at least 99%, or at least 100% sequence identity to an amino acid sequence of SEQ ID NO: 386; zz) a V_{H} sequence having at least 80%, at least 90%, at least 95%, at least 97%, at least 98%, at least 99%, or at least 100% sequence identity to an amino acid sequence of SEQ ID NO: 387, and a V_{L} sequence having at least 80%, at least 90%, at least 95%, at least 97%, at least 98%, at least 99%, or at least 100% sequence identity to an amino acid sequence of SEQ ID NO: 388; aaa) a V_{H} sequence having at least 80%, at least 90%, at least 95%, at least 97%, at least 98%, at least 99%, or at least 100% sequence identity to an amino acid sequence of SEQ ID NO: 389, and a V_{L} sequence having at least 80%, at least 90%, at least 95%, at least 97%, at least 98%, at least 99%, or at least 100% sequence identity to an amino acid sequence of SEQ ID NO: 390; bbb) a V_{H} sequence having at least 80%, at least 90%, at least 95%, at least 97%, at least 98%, at least 99%, or at least 100% sequence identity to an amino acid sequence of SEQ ID NO: 391, and a V_{L} sequence having at least 80%, at least 90%, at least 95%, at least 97%, at least 98%, at least 99%, or at least 100% sequence identity to an amino acid sequence of SEQ ID NO: 392; ccc) a V_{H} sequence having at least 80%, at least 90%, at least 95%, at least 97%, at least 98%, at least 99%, or at least 100% sequence identity to an amino acid sequence of SEQ ID NO: 393, and a V_{L} sequence having at least 80%, at least 90%, at least 95%, at least 97%, at least 98%, at least 99%, or at least 100% sequence identity to an amino acid sequence of SEQ ID NO: 394; ddd) a V_{H} sequence having at least 80%, at least 90%, at least 95%, at least 97%, at least 98%, at least 99%, or at least 100% sequence identity to an amino acid sequence of SEQ ID NO: 395, and a V_{L} sequence having at least 80%, at least 90%, at least 95%, at least 97%, at least 98%, at least 99%, or at least 100% sequence identity to an amino acid sequence of SEQ ID NO: 396; eee) a V_{H} sequence having at least 80%, at least 90%, at least 95%, at least 97%, at least 98%, at least 99%, or at least 100% sequence identity to an amino acid sequence of SEQ ID NO: 397, and a V_{L} sequence having at least 80%, at least 90%, at least 95%, at least 97%, at least 98%, at least 99%, or at least 100% sequence identity to an amino acid sequence of SEQ ID NO: 398; fff) a V_{H} sequence having at least 80%, at least 90%, at least 95%, at least 97%, at least 98%, at least 99%, or at least 100% sequence identity to an amino acid sequence of SEQ ID NO: 399, and a V_{L} sequence having at least 80%, at least 90%, at least 95%, at least 97%, at least 98%, at least 99%, or at least 100% sequence identity to an amino acid sequence of SEQ ID NO: 400; ggg) a V_{H} sequence having at least 80%, at least 90%, at least 95%, at least 97%, at least 98%, at least 99%, or at least 100% sequence identity to an amino acid sequence of SEQ ID NO: 401, and a V_{L} sequence having at least 80%, at least 90%, at least 95%, at least 97%, at least 98%, at least 99%, or at least 100% sequence identity to an amino acid sequence of SEQ ID NO: 402; hhh) a V_{H} sequence having at least 80%, at least 90%, at least 95%, at least 97%, at least 98%, at least 99%, or at least 100% sequence identity to an amino acid sequence of SEQ ID NO: 403, and a V_{L} sequence having at least 80%, at least 90%, at least 95%, at least 97%, at least 98%, at least 99%, or at least 100% sequence identity to an amino acid sequence of SEQ ID NO: 404; iii) a V_{H} sequence having at least 80%, at least 90%, at least 95%, at least 97%, at least 98%, at least 99%, or at least 100% sequence identity to an amino acid sequence of SEQ ID NO: 405, and a V_{L} sequence having at least 80%, at least 90%, at least 95%, at least 97%, at least 98%, at least 99%, or at least 100% sequence identity to an amino acid sequence of SEQ ID NO: 406; jjj) a V_{H} sequence having at least 80%, at least 90%, at least 95%, at least 97%, at least 98%, at least 99%, or at least 100% sequence identity to an amino acid sequence of SEQ ID NO: 407, and a V_{L} sequence having at least 80%, at least 90%, at least 95%, at least 97%, at least 98%, at least 99%, or at least 100% sequence identity to an amino acid sequence of SEQ ID NO: 408; kkk) a V_{H} sequence having at least 80%, at least 90%, at least 95%, at least 97%, at least 98%, at least 99%, or at least 100% sequence identity to an amino acid sequence of SEQ ID NO: 409, and a V_{L} sequence having at least 80%, at least 90%, at least 95%, at least 97%, at least 98%, at least 99%, or at least 100% sequence identity to an amino acid sequence of SEQ ID NO: 410; lll) a V_{H} sequence having at least 80%, at least 90%, at least 95%, at least 97%, at least 98%, at least 99%, or at least 100% sequence identity to an amino acid sequence of SEQ ID NO: 411, and a V_{L} sequence having at least 80%, at least 90%, at least 95%, at least 97%, at least 98%, at least 99%, or at least 100% sequence identity to an amino acid sequence of SEQ ID NO: 412; mmm) a V_{H} sequence having at least 80%, at least 90%, at least 95%, at least 97%, at least 98%, at least 99%, or at least 100% sequence identity to an amino acid sequence of SEQ ID NO: 413, and a V_{L} sequence having at least 80%, at least 90%, at least 95%, at least 97%, at least 98%, at least 99%, or at least 100% sequence identity to an amino acid sequence of SEQ ID NO: 414; ooo) a V_{H} sequence having at least 80%, at least 90%, at least 95%, at least 97%, at least 98%, at least 99%, or at least 100% sequence identity to an amino acid sequence of SEQ ID NO: 415, and a V_{L} sequence having at least 80%, at least 90%, at least 95%, at least 97%, at least 98%, at least 99%, or at least 100% sequence identity to an amino acid sequence of SEQ ID NO: 416; ppp) a V_{H} sequence having at least 80%, at least 90%, at least 95%, at least 97%, at least 98%, at least 99%, or at least 100% sequence identity to an amino acid sequence of SEQ ID NO: 417, and a V_{L} sequence having at least 80%, at least 90%, at least 95%, at least 97%, at least 98%, at least 99%, or at least 100% sequence identity to an amino acid sequence of SEQ ID NO: 418; qqq) a V_{H} sequence having at least 80%, at least 90%, at least 95%, at least 97%, at least 98%, at least 99%, or at least 100% sequence identity to an amino acid sequence of SEQ ID NO: 419, and a V_{L} sequence having at least 80%, at least 90%, at least 95%, at least 97%, at least 98%, at least 99%, or at least 100% sequence identity to an amino acid sequence of SEQ ID NO: 420; rrr) a V_{H} sequence having at least 80%, at least 90%, at least 95%, at least 97%, at least 98%, at least 99%, or at least 100% sequence identity to an amino acid sequence of SEQ ID NO: 421, and a V_{L} sequence having at least 80%, at least 90%, at least 95%, at least 97%, at least 98%, at least 99%, or at least 100% sequence identity to an amino acid sequence of SEQ ID NO: 422; sss) a V_{H} sequence having at least 80%, at least 90%, at least 95%, at least 97%, at least 98%, at least 99%, or at least 100% sequence identity to an amino acid sequence of SEQ ID NO: 423, and a V_{L} sequence having at least 80%, at least 90%, at least 95%, at least 97%, at least 98%, at least 99%, or at least 100% sequence identity to an amino acid sequence of SEQ ID NO: 424; ttt) a V_{H} sequence having at least 80%, at least 90%, at least 95%, at least 97%, at least 98%, at least 99%, or at least 100% sequence identity to an amino acid sequence of SEQ ID NO: 425, and a V_{L} sequence having at least 80%, at least 90%, at least 95%, at least 97%, at least 98%, at least 99%, or at least 100% sequence identity to an amino acid sequence of SEQ ID NO: 426; uuu) a V_{H} sequence having at least 80%, at least 90%, at least 95%, at least 97%, at least 98%, at least 99%, or at least 100% sequence identity to an amino acid sequence of SEQ ID NO: 427, and a V_{L} sequence having at least 80%, at least 90%, at least 95%, at least 97%, at least 98%, at least 99%, or at least 100% sequence identity to an amino acid sequence of SEQ ID NO: 428; vvv) a V_{H} sequence having at least 80%, at least 90%, at least 95%, at least 97%, at least 98%, at least 99%, or at least 100% sequence identity to an amino acid sequence of SEQ ID NO: 429, and a V_{L} sequence having at least 80%, at least 90%, at least 95%, at least 97%, at least 98%, at least 99%, or at least 100% sequence identity to an amino acid sequence of SEQ ID NO: 430; www) a V_{H} sequence having at least 80%, at least 90%, at least 95%, at least 97%, at least 98%, at least 99%, or at least 100% sequence identity to an amino acid sequence of SEQ ID NO: 431, and a V_{L} sequence having at least 80%, at least 90%, at least 95%, at least 97%, at least 98%, at least 99%, or at least 100% sequence identity to an amino acid sequence of SEQ ID NO: 432; xxx) a V_{H} sequence having at least 80%, at least 90%, at least 95%, at least 97%, at least 98%, at least 99%, or at least 100% sequence identity to an amino acid sequence of SEQ ID NO: 433, and a V_{L} sequence having at least 80%, at least 90%, at least 95%, at least 97%, at least 98%, at least 99%, or at least 100% sequence identity to an amino acid sequence of SEQ ID NO: 434; yyy) a V_{H} sequence having at least 80%, at least 90%, at least 95%, at least 97%, at least 98%, at least 99%, or at least 100% sequence identity to an amino acid sequence of SEQ ID NO: 435, and a V_{L} sequence having at least 80%, at least 90%, at least 95%, at least 97%, at least 98%, at least 99%, or at least 100% sequence identity to an amino acid sequence of SEQ ID NO: 436; zzz) a V_{H} sequence having at least 80%, at least 90%, at least 95%, at least 97%, at least 98%, at least 99%, or at least 100% sequence identity to an amino acid sequence of SEQ ID NO: 482, and a V_{L} sequence having at least 80%, at least 90%, at least 95%, at least 97%, at least 98%, at least 99%, or at least 100% sequence identity to an amino acid sequence of SEQ ID NO: 483; aaaa) a V_{H} sequence having at least 80%, at least 90%, at least 95%, at least 97%, at least 98%, at least 99%, or at least 100% sequence identity to an amino acid sequence of SEQ ID NO: 484, and a V_{L} sequence having at least 80%, at least 90%, at least 95%, at least 97%, at least 98%, at least 99%, or at least 100% sequence identity to an amino acid sequence of SEQ ID NO: 485; bbbb) a V_{H} sequence having at least 80%, at least 90%, at least 95%, at least 97%, at least 98%, at least 99%, or at least 100% sequence identity to an amino acid sequence of SEQ ID NO: 486, and a V_{L} sequence having at least 80%, at least 90%, at least 95%, at least 97%, at least 98%, at least 99%, or at least 100% sequence identity to an amino acid sequence of SEQ ID NO: 487; cccc) a V_{H} sequence having at least 80%, at least 90%, at least 95%, at least 97%, at least 98%, at least 99%, or at least 100% sequence identity to an amino acid sequence of SEQ ID NO: 488, and a V_{L} sequence having at least 80%, at least 90%, at least 95%, at least 97%, at least 98%, at least 99%, or at least 100% sequence identity to an amino acid sequence of SEQ ID NO: 489; or dddd) a V_{H} sequence having at least 80%, at least 90%, at least 95%, at least 97%, at least 98%, at least 99%, or at least 100% sequence identity to an amino acid sequence of SEQ ID NO: 490, and a V_{L} sequence having at least 80%, at least 90%, at least 95%, at least 97%, at least 98%, at least 99%, or at least 100% sequence identity to an amino acid sequence of SEQ ID NO: 491.

The antibody construct as described herein may comprise a sequence from TABLE 1 and/or TABLE 2. The antibody construct may comprise a set of six CDRs selected from a select of CDRs set forth in TABLE 1. The antibody construct may comprise a pair of variable regions selected from the pairs of variable regions set forth in TABLE 2.

TABLE 1
Antibody CDRs

| ANTIBODY | REGION | SEQ ID NO: | SEQUENCE: |
|---|---|---|---|
| Antibody to GITR | HCDR1 | 1 | SYGMH |
| | HCDR2 | 2 | VIWYEGSNKYYADSVKG |
| | HCDR3 | 3 | GGSMVRGDYYYGMDV |
| | LCDR1 | 4 | RASQGISSALA |
| | LCDR2 | 5 | DASSLES |
| | LCDR3 | 6 | QQFNSYPYT |
| Antibody to LAG-3 | HCDR1 | 7 | DYYWN |
| | HCDR2 | 8 | EINHRGSTNSNPSLKS |
| | HCDR3 | 9 | GYSDYEYNWFDP |
| | LCDR1 | 10 | RASQSISSYLA |
| | LCDR2 | 11 | DASNRAT |
| | LCDR3 | 12 | QQRSNWPLT |
| Utomilumab | HCDR1 | 13 | GYSFSTYW |
| (CD137) | HCDR2 | 14 | IYPGDSYT |
| | HCDR3 | 15 | ARGYGIFDY |
| | LCDR1 | 16 | NIGDQY |
| | LCDR2 | 17 | QDK |
| | LCDR3 | 18 | ATYTGFGSLAV |
| 4G8 | HCDR1 | 19 | GFTFSSYA |
| | HCDR2 | 20 | ISGSGGST |
| | HCDR3 | 21 | AKGWLGNFDY |
| | LCDR1 | 22 | QSVSRSY |
| | LCDR2 | 23 | GAS |
| | LCDR3 | 24 | QQGQVIPPT |
| 4B9 | HCDR1 | 25 | GFTFSSYA |
| | HCDR2 | 26 | IIGSGAST |
| | HCDR3 | 27 | AKGWFGGFNY |
| | LCDR1 | 28 | QSVTSSY |
| | LCDR2 | 29 | VGS |
| | LCDR3 | 30 | QQGIMLPPT |
| 28H1 | HCDR1 | 31 | GFTFSSHA |
| | HCDR2 | 32 | IWASGEQ |
| | HCDR3 | 33 | AKGWLGNFDY |
| | LCDR1 | 34 | QSVSRSY |
| | LCDR2 | 35 | GAS |
| | LCDR3 | 36 | QQGQVIPPT |
| Ontuxizumab | HCDR1 | 37 | GYTFTDYV |
| (endosialin) | HCDR2 | 38 | INPYDDDT |
| | HCDR3 | 39 | ARRGNSYDGYFDYSMDY |
| | LCDR1 | 40 | QNVGTA |
| | LCDR2 | 41 | SAS |
| | LCDR3 | 42 | QQYTNYPMYT |
| Rinucumab | HCDR1 | 43 | GGSITSSSYY |
| (PDGFRβ) | HCDR2 | 44 | IYYRGST |
| | HCDR3 | 45 | ARQNGAARPSWFDP |
| | LCDR1 | 46 | QSISSIY |
| | LCDR2 | 47 | GAS |
| | LCDR3 | 48 | QHYGISPFT |
| Antibody 1 to | HCDR1 | 49 | GYTFTSYG |
| MADCAM | HCDR2 | 50 | ISVYSGNT |
| | HCDR3 | 51 | AREGSSSSGDYYYGMDV |
| | LCDR1 | 52 | QSLLHTDGTTY |
| | LCDR2 | 53 | EVS |
| | LCDR3 | 54 | MQNIQLPWT |
| Antibody 2 to | HCDR1 | 55 | GYTFTSYG |
| MADCAM | HCDR2 | 56 | ISVYSGNT |
| | HCDR3 | 57 | AREGSSSSGDYYYGMDV |
| | LCDR1 | 58 | QSLLYSDGKTY |
| | LCDR2 | 59 | EVS |
| | LCDR3 | 60 | MQSIQLPWT |
| Pamrevlumab | HCDR1 | 61 | GFTFSSYG |
| (CTGF) | HCDR2 | 62 | IGTGGGT |
| | HCDR3 | 63 | ARGDYYGSGSFFDC |
| | LCDR1 | 64 | QGISSW |
| | LCDR2 | 65 | AAS |
| | LCDR3 | 66 | QQYNSYPPT |
| Antibody 1 to PDPN | HCDR1 | 67 | GYTFTSYTIH |
| | HCDR2 | 68 | YINPGSGYTNYNEKFQD |
| | HCDR3 | 69 | WDRGY |
| | LCDR1 | 70 | RSSQTIVHSNGNTYLE |
| | LCDR2 | 71 | KVSNRFS |
| | LCDR3 | 72 | FQGSHVPYT |
| Antibody 2 to PDPN | HCDR1 | 73 | GFTFSNYG |
| | HCDR2 | 74 | ISAGGDKT |
| | HCDR3 | 75 | AKTSR |
| | LCDR1 | 76 | TGNIGSNY |
| | LCDR2 | 77 | RDD |
| | LCDR3 | 78 | HSYSSGIV |
| Natalizumab | HCDR1 | 79 | GFNIKDTY |
| (integrin α) | HCDR2 | 80 | IDPANGYT |
| | HCDR3 | 81 | AREGYYGNYGVYAMDY |
| | LCDR1 | 82 | QDINKY |
| | LCDR2 | 83 | YTS |
| | LCDR3 | 84 | LQYDNLWT |
| Zinbryta ™ | HCDR1 | 85 | GYTFTSYR |
| (Daclizumab) | HCDR2 | 86 | INPSTGYT |
| | HCDR3 | 87 | ARGGGVFDY |
| | LCDR1 | 88 | SSSISY |
| | LCDR2 | 89 | TTS |
| | LCDR3 | 90 | HQRSTYPLT |
| Antibody to TNFR2 | HCDR1 | 91 | GYTFTDYN |
| variant 1 | HCDR2 | 92 | INPNYEST |
| | HCDR3 | 93 | RDKGWYFDV |
| | LCDR1 | 94 | SSVKN |
| | LCDR2 | 95 | YTS |
| | LCDR3 | 96 | QQFTSSPYT |
| Antibody to TNFR2 | HCDR1 | 97 | GFSLSTSGMG |
| variant 2 | HCDR2 | 98 | IWWDDDK |
| | HCDR3 | 99 | ARLTGTRYFDY |
| | LCDR1 | 100 | QDINKF |
| | LCDR2 | 101 | YTS |
| | LCDR3 | 102 | LQYGNLWT |
| Antibody to TNFR2 | HCDR1 | 103 | GYTFTDYS |
| variant 3 | HCDR2 | 104 | INTETGEP |
| | HCDR3 | 105 | ATYYGSSYVPDY |
| | LCDR1 | 106 | QNVGTA |
| | LCDR2 | 107 | WTS |
| | LCDR3 | 108 | QYSDYPYT |
| Antibody to TNFR2 | HCDR1 | 109 | GYTFTDY |
| variant 4 | HCDR2 | 110 | WVDPEYGS |
| | HCDR3 | 111 | ARDDGSYSPFDY |
| | LCDR1 (major) | 112 | QNINKY |
| | LCDR2 (major) | 113 | YTS |
| | LCDR3 (major) | 114 | LQYVNLLT |
| | LCDR1 (minor) | 115 | ENVVTY |
| | LCDR2 (minor) | 116 | GAS |
| | LCDR3 (minor) | 117 | QGYSYPYT |
| Bleselumab | HCDR1 | 118 | GGSISSPGYY |
| (CD40) | HCDR2 | 119 | IYKSGST |
| | HCDR3 | 120 | TRPVVRYFGWFDP |
| | LCDR1 | 121 | QGISSA |
| | LCDR2 | 122 | DAS |
| | LCDR3 | 123 | QQFNSYPT |
| Antibody to DEC-205 | HCDR1 | 124 | GFTFSNYG |
| variant 1 | HCDR2 | 125 | IWYDGSNK |
| | HCDR3 | 126 | ARDLWGWYFDY |
| | LCDR1 | 127 | QSVSSY |
| | LCDR2 | 128 | DAS |
| | LCDR3 | 129 | QQRRNWPLT |
| Antibody to DEC-205 | HCDR1 | 130 | GDSFTTYW |
| variant 2 | HCDR2 | 131 | IYPGDSDT |
| | HCDR3 | 132 | TRGDRGVDY |
| | LCDR1 | 133 | QGISRW |
| | LCDR2 | 134 | AAS |
| | LCDR3 | 135 | QQYNSYPRT |
| Antibody 5 to TNFR2 | HCDR1 | 136 | GFSLSTSGMG |
| | HCDR2 | 137 | IWWDDDK |
| | HCDR3 | 138 | ARITGTRYFDY |
| | LCDR1 | 139 | QDINKF |
| | LCDR2 | 140 | YTS |
| | LCDR3 | 141 | LQYGNLWT |
| Fun1 | HCDR1 | 142 | GYSFTDYN |
| (CD86) | HCDR2 | 143 | IDPYYGGT |
| | HCDR3 | 144 | ARWDYRYDDGRAYYVMDF |
| | LCDR1 | 145 | QSVLYSSNQKNY |
| | LCDR2 | 146 | WAS |
| | LCDR3 | 147 | HQYLYSWT |
| hzFun1 | HCDR1 | 148 | GYSFTDYN |
| | HCDR2 | 149 | IDPYYGGT |
| | HCDR3 | 150 | ARWDYRYDDGRAYYVMDF |
| | LCDR1 | 151 | QSVLYSSNQKNY |
| | LCDR2 | 152 | WAS |
| | LCDR3 | 153 | HQYLYSWT |
| Antibody 1 to | HCDR1 | 154 | GYTFTNYII |
| CD45RB/RO | HCDR2 | 155 | FNPYNHGT |
| | HCDR3 | 156 | ARSGPYAWFDT |
| | LCDR1 | 157 | QNIGTS |
| | LCDR2 | 158 | SSS |
| | LCDR3 | 159 | QQSNTWPFT |
| Antibody 2 to | HCDR1 | 160 | GYTFTNYII |
| CD45RB/RO | HCDR2 | 161 | FNPYNHGT |
| | HCDR3 | 162 | ARSGPYAWFDT |
| | LCDR1 | 163 | QNIGTS |
| | LCDR2 | 164 | SSS |
| | LCDR3 | 165 | QQSNTWPFT |
| Antibody to CD45RB | HCDR1 | 166 | GFTFSNYG |
| | HCDR2 | 167 | IWYDGSKK |
| | HCDR3 | 168 | ARGGGDFDF |
| | LCDR1 | 169 | QSVSGNY |
| | LCDR2 | 170 | GAS |
| | LCDR3 | 171 | QQYGKWPPLT |
| Antibody 1 to MHC- | HCDR1 | 172 | GFSLSTSGVG |
| DR | HCDR2 | 173 | IDWDDDK |
| | HCDR3 | 174 | ARSPRYRGAFDY |
| | LCDR1 | 175 | ESNIGNNY |
| | LCDR2 | 176 | DNN |
| | LCDR3 | 177 | QSYDLIRHV |
| Antibody 2 to MHC- | HCDR1 | 178 | GFSLSTSGVG |
| DR | HCDR2 | 179 | IDWDDDK |
| | HCDR3 | 180 | ARSPRYRGAFDY |
| | LCDR1 | 181 | ESNIGNNY |
| | LCDR2 | 182 | DNN |
| | LCDR3 | 183 | QSYDMNVH |
| DE8 | HCDR1 | 184 | GFSLSTSGMG |
| | HCDR2 | 185 | IYWDDK |
| | HCDR3 | 186 | ARSSHYYGYGYGGYFDV |
| | LCDR1 | 187 | ESIHSYGNSF |
| | LCDR2 | 188 | LAS |
| | LCDR3 | 189 | QQNNEDPWT |
| Etaracizumab | HCDR1 | 190 | GFTFSSYD |
| (Integrin αvβ3) | HCDR2 | 191 | VSSGGGST |
| | HCDR3 | 192 | ARHLHGSFAS |
| | LCDR1 | 193 | QSISNFL |
| | LCDR2 | 194 | YRS |
| | LCDR3 | 195 | QQSGSWPLT |
| Antibody to | HCDR1 | 196 | GFVFSRYW |
| avb8 | HCDR2 | 197 | INPDSSTI |
| | HCDR3 | 198 | ASLITTEDY |
| | LCDR1 | 199 | QDINSY |
| | LCDR2 | 200 | YAN |
| | LCDR3 | 201 | LQYDEFPYT |
| Intetumumab | HCDR1 | 202 | GFTFSRYT |
| | HCDR2 | 203 | ISFDGSNK |
| | HCDR3 | 204 | AREARGSYAFDI |
| | LCDR1 | 205 | QSVSSY |
| | LCDR2 | 206 | DAS |
| | LCDR3 | 207 | QQRSNWPPFT |
| Antibody to Integrin | HCDR1 | 208 | GYTFSSFW |
| αv | HCDR2 | 209 | INPRSGYT |
| | HCDR3 | 210 | ASFLGRGAMDY |
| | LCDR1 | 211 | QDISNY |
| | LCDR2 | 212 | YTS |
| | LCDR3 | 213 | QQGNTFPYT |
| Antibody to Integrin | HCDR1 | 214 | GGSISSGVYY |
| αvβ6 variant 1 | HCDR2 | 215 | IYYSGST |
| | HCDR3 | 216 | AREGPLRGDYYYGLDV |
| | LCDR1 | 217 | QTISSRY |
| | LCDR2 | 218 | GAS |
| | LCDR3 | 219 | QQYGSSPRT |
| Oleclumab | HCDR1 | 220 | GFTFSSYA |
| (CD73) | HCDR2 | 221 | ISGSGGRT |
| | HCDR3 | 222 | ARLGYGRVDE |
| | LCDR1 | 223 | LSNIGRNP |
| | LCDR2 | 224 | LDN |
| | LCDR3 | 225 | ATWDDSHPGWT |
| Antibody to CD73 | HCDR1 | 226 | GFTFSNYG |
| | HCDR2 | 227 | ILYDGSNK |
| | HCDR3 | 228 | ARGGSSWYPDSFDI |
| | LCDR1 | 229 | QGISSW |
| | LCDR2 | 230 | AAS |
| | LCDR3 | 231 | QQYNSYPLT |
| Daratumumab | HCDR1 | 232 | GFTFNSFA |
| (CD38) | HCDR2 | 234 | ISGSGGGT |
| | HCDR3 | 235 | AKDKILWFGEPVFDY |
| | LCDR1 | 236 | QSVSSY |
| | LCDR2 | 237 | DAS |
| | LCDR3 | 238 | QRSNWPPT |
| Vatelizumab | HCDR1 | 239 | GFSLTNYG |
| (integrin α2) | HCDR2 | 240 | IWARGFT |
| | HCDR3 | 241 | ARANDGVYYAMDY |
| | LCDR1 | 242 | QSSVNY |
| | LCDR2 | 243 | DTS |
| | LCDR3 | 244 | QQWTTNPLT |
| Vedolizumab | HCDR1 | 245 | GYTFTSYW |
| (Integrin α4β7) | HCDR2 | 246 | IDPSESNT |
| | HCDR3 | 247 | ARGGYDGWDYAIDY |
| | LCDR1 | 248 | QSLAKSYGNTY |
| | LCDR2 | 249 | GIS |
| | LCDR3 | 250 | LQGTHQPYT |
| Etrolizumab | HCDR1 | 251 | GFFITNNY |
| | HCDR2 | 252 | ISYSGST |
| | HCDR3 | 253 | ARTGSSGYFDF |
| | LCDR1 | 254 | ESVDDLL |
| | LCDR2 | 255 | YAS |
| | LCDR3 | 256 | QQGNSLPNT |
| Anifrolumab | HCDR1 | 257 | GYIFTNYW |
| (IFNAR1) | HCDR2 | 258 | IYPGDSDI |
| | HCDR3 | 259 | ARHDIEGFDY |
| | LCDR1 | 260 | QSVSSSF |
| | LCDR2 | 261 | GAS |
| | LCDR3 | 262 | QQYDSSAIT |
| BIIB059 | HCDR1 | 263 | GFTFTYTMS |
| (BDCA2) | HCDR2 | 264 | PGDSFGY |
| | HCDR3 | 265 | TRDIYYNYGAWFAY |
| | LCDR1 | 266 | QSVDYDGDSY |
| | LCDR2 | 267 | AAS |
| | LCDR3 | 268 | QQANEDPRT |
| Brentuximab of | HCDR1 | 269 | GYTFTDYY |
| Brentuximab Vedotin | HCDR2 | 270 | IYPGSGNT |
| (CD30) | HCDR3 | 271 | ANYGNYWFAY |
| | LCDR1 | 272 | QSVDFDGDSY |
| | LCDR2 | 273 | AAS |
| | LCDR3 | 274 | QQSNEDPWT |
| Iratumumab | HCDR1 | 275 | GGSFSAYY |
| (CD30) | HCDR2 | 276 | INHGGGT |
| | HCDR3 | 277 | ASLTAY |
| | LCDR1 | 278 | QGISSW |
| | LCDR2 | 279 | AAS |
| | LCDR3 | 280 | QQYDSYPIT |
| Antibody to c-KIT | HCDR1 | 281 | GYTFTSYN |
| | HCDR2 | 282 | IYSGNGDT |
| | HCDR3 | 283 | ARERDTRFGN |
| | LCDR1 | 284 | ESVDIYGNSF |
| | LCDR2 | 285 | LAS |
| | LCDR3 | 286 | QQNNEDPYT |
| Opdivo ™ | HCDR1 | 287 | GITFSNSG |
| (nivolumab) | HCDR2 | 288 | IWYDGSKR |
| | HCDR3 | 289 | ATNDDY |
| | LCDR1 | 290 | QSVSSYL |
| | LCDR2 | 291 | DAS |
| | LCDR3 | 292 | QQSSNWPRT |
| Keytruda ™ | HCDR1 | 293 | GYTFTNYY |
| (pembrolizumab) | HCDR2 | 294 | INPSNGGT |
| | HCDR3 | 295 | ARRDYRFDMGFDY |
| | LCDR1 | 296 | KGVSTSGYSY |
| | LCDR2 | 297 | LAS |
| | LCDR3 | 298 | QHSRDLPLT |
| Antibody M25 to | HCDR1 | 440 | SYWIE |
| LRRC15 | HCDR2 | 441 | EILPGSDTTNYNEKFKD |
| | HCDR3 | 442 | GNYRAWFGY |
| | LCDR1 | 443 | RASQDISNYLN |
| | LCDR2 | 444 | YTSRLHS |
| | LCDR3 | 445 | QQGEALPWT |
| Antibody | HCDR1 | 446 | DYYIH |
| huAD208.4.1 to | HCDR2 | 447 | LVYPYIGGTNYNQKFKG |
| LRRC15 | HCDR3 | 448 | GDNKYDAMDY |
| | LCDR1 | 449 | RASQSVSTSSYSYMH |
| | LCDR2 | 450 | YASSLES |
| | LCDR3 | 451 | EQSWEIRT |
| Antibody | HCDR1 | 452 | NYWMH |
| huAD208.12.1 to | HCDR2 | 453 | MIHPNSGSTKHNEKFRG |
| LRRC15 | HCDR3 | 454 | SDFGNYRWYFDV |
| | LCDR1 | 455 | RASQSSSNNLH |
| | LCDR2 | 456 | YVSQSIS |
| | LCDR3 | 457 | QQSNSWPFT |
| Antibody | HCDR1 | 458 | DYYIH |
| huAD208.14.1 to | HCDR2 | 459 | LVYPYIGGSSYNQQFKG |
| LRRC15 | HCDR3 | 460 | GDNNYDAMDY |
| | LCDR1 | 461 | RASQSVSTSTYNYMH |
| | LCDR2 | 462 | YASNLES |
| | LCDR3 | 463 | HHTWEIRT |
| Antibody hu139.10 to | HCDR1 | 464 | SYGVH |
| LRRC15 | HCDR2 | 465 | VIWAGGSTNYNSALMS |
| | HCDR3 | 466 | HMITEDYYGMDY |
| | LCDR1 | 467 | KSSQSLLNSRTRKNYLA |
| | LCDR2 | 468 | WASTRES |
| | LCDR3 | 469 | KQSYNLPT |
| Antibody | HCDR1 | 470 | NYWLG |
| muAD210.40.9 to | HCDR2 | 471 | DIYPGGGNTYYNEKLKG |
| LRRC15 | HCDR3 | 472 | WGDKKGNYFAY |
| | LCDR1 | 473 | TASSSVYSSYLH |
| | LCDR2 | 474 | STSNLAS |
| | LCDR3 | 475 | HQYHRSPT |
| Antibody | HCDR1 | 476 | NFGMN |
| muAD209.9.1 to | HCDR2 | 477 | WINLYTGEPTFADDFKG |
| LRRC15 | HCDR3 | 478 | KGETYYRYDGFAY |
| | LCDR1 | 479 | RSSKSLLHSNGNTHLY |
| | LCDR2 | 480 | RMSNLAS |
| | LCDR3 | 481 | MQLLEYPYT |

TABLE 2
Antibody V_{H} sequence and V_{L} sequences

| ANTIBODY | REGION | SEQ ID NO | SEQUENCE |
|---|---|---|---|
| TRX518 | V_{L} | 299 | EIVMTQSPATLSVSPGERATLSCKASQNVGTNVAWY |
| (GITR) | | | QQKPGQAPRLLIYSASYRYSGIPARFSGSGSGTEFTLTI |
| | | | SSLQSEDFAVYYCQQYNTDPLTFGGGTKVEIK |
| | V_{H} | 300 | QVTLRESGPALVKPTQTLTLTCTFSGFSLSTSGMGVG |
| | | | WIRQPPGKALEWLAHIWWDDDKYYNPSLKSRLTISK |
| | | | DTSKNQVVLTMTNMDPVDTATYYCARTRRYFPFAY |
| | | | WGQGTLVTVSS |
| | V_{H} | 301 | QVTLRESGPALVKPTQTLTLTCTFSGFSLSTSGMGVG |
| | | | WIRQPPGKALEWLAHIWWDDDKYYQPSLKSRLTISK |
| | | | DTSKNQVVLTMTNMDPVDTATYYCARTRRYFPFAY |
| | | | WGQGTLVTVSS |
| Antibody to | V_{H} | 302 | QVQLQQWGAGLLKPSETLSLTCAVYGGSFSDYYWN |
| LAG-3 | | | WIRQPPGKGLEWIGEINHRGSTNSNPSLKSRVTLSLDT |
| | | | SKNQFSLKLRSVTAADTAVYYCAFGYSDYEYNWFDP |
| | | | WGQGTLVTVSS |
| | V_{L} | 303 | EIVLTQSPATLSLSPGERATLSCRASQSISSYLAWYQQ |
| | | | KPGQAPRLLIYDASNRATGIPARFSGSGSGTDFTLTISS |
| | | | LEPEDFAVYYCQQRSNWPLTFGQGTNLEIK |
| Antibody to 4- | V_{H} | 304 | QVQLQQWGAGLLKPSETLSLTCAVYGGSFSGYYWS |
| 1BB variant 1 | | | WIRQSPEKGLEWIGEINHGGYVTYNPSLESRVTISVDT |
| | | | SKNQFSLKLSSVTAADTAVYYCARDYGPGNYDWYF |
| | | | DLWGRGTLVTVSS |
| | V_{L} | 305 | EIVLTQSPATLSLSPGERATLSCRASQSVSSYLAWYQQ |
| | | | KPGQAPRLLIYDASNRATGIPARFSGSGSGTDFTLTISS |
| | | | LEPEDFAVYYCQQRSNWPPALTFGGGTKVEIK |
| Antibody to 4- | V_{H} | 306 | EVQLVESGGGLVQPGGSLRLSCAASGFTFSDYWMSW |
| 1BB variant 2 | | | VRQAPGKGLEWVADIKNDGSYTNYAPSLTNRFTISRD |
| | | | NAKNSLYLQMNSLRAEDTAVYYCARELTGTWGQGT |
| | | | MVTVSS |
| | V_{L} | 307 | DIVMTQSPDSLAVSLGERATINCKSSQSLLSSGNQKNY |
| | | | LAWYQQKPGQPPKLLIYYASTRQSGVPDRFSGSGSGT |
| | | | DFTLTISSLQAEDVAVYYCLQYDRYPFTFGQGTKLEIK |
| Utomilumab | V_{H} | 308 | EVQLVQSGAEVKKPGESLRISCKGSGYSFSTYWISWV |
| | | | RQMPGKGLEWMGKIYPGDSYTNYSPSFQGQVTISAD |
| | | | KSISTAYLQWSSLKASDTAMYYCARGYGIFDYWGQG |
| | | | TLVTVSS |
| | V_{L} | 309 | SYELTQPPSVSVSPGQTASITCSGDNIGDQYAHWYQQ |
| | | | KPGQSPVLVIYQDKNRPSGIPERFSGSNSGNTATLTISG |
| | | | TQAMDEADYYCATYTGFGSLAVFGGGTKLTVL |
| Vorsetuzumab | V_{H} | 310 | QVQLVQSGAEVKKPGASVKVSCKASGYTFTNYGMN |
| (CD70) | | | WVRQAPGQGLKWMGWINTYTGEPTYADAFKGRVT |
| | | | MTRDTSISTAYMELSRLRSDDTAVYYCARDYGDYGM |
| | | | DYWGQGTTVTVSS |
| | V_{L} | 311 | DIVMTQSPDSLAVSLGERATINCRASKSVSTSGYSFM |
| | | | HWYQQKPGQPPKLLIYLASNLESGVPDRFSGSGSGTD |
| | | | FTLTISSLQAEDVAVYYCQHSREVPWTFGQGTKVEIK |
| Rinucumab | V_{H} | 312 | QLQLQESGPGLVKPSETLSLTCTVSGGSITSSSYYWG |
| | | | WIRQPPGKGLEWIGSIYYRGSTNYNPSLKSRVTISVDS |
| | | | SKNQFYLKVSSVTAVDTAVYYCARQNGAARPSWFDP |
| | | | WGQGTLVTVSS |
| | V_{L} | 313 | EIVLTQSPDTISLSPGERATLSCRASQSISSIYLAWYQQ |
| | | | KPGQAPRLLIYGASSRVTGIPDRFSVSGSGTDFTLTISR |
| | | | LEPEDFAVYYCQHYGISPFTFGPGTKVDIR |
| Oleclumab | V_{H} | 314 | EVQLLESGGGLVQPGGSLRLSCAASGFTFSSYAYSWV |
| (CD73) | | | RQAPGKGLEWVSAISGSGGRTYYADSVKGRFTISRDN |
| | | | SKNTLYLQMNSLRAEDTAVYYCARLGYGRVDEWGR |
| | | | GTLVTVSS |
| | V_{L} | 315 | QSVLTQPPSASGTPGQRVTISCSGSLSNIGRNPVNWYQ |
| | | | QLPGTAPKLLIYLDNLRLSGVPDRFSGSKSGTSASLAIS |
| | | | GLQSEDEADYYCATWDDSHPGWTFGGGTKLTVL |
| Ontuxizumab | V_{H} | 316 | QVQLQESGPGLVRPSQTLSLTCTASGYTFTDYVIHWV |
| (endosialin) | | | KQPPGRGLEWIGYINPYDDDTTYNQKFKGRVTMLVD |
| | | | TSSNTAYLRLSSVTAEDTAVYYCARRGNSYDGYFDY |
| | | | SMDYWGSGTPVTVSS |
| | V_{L} | 317 | DIQMTQSPSSLSASVGDRVTITCRASQNVGTAVAWLQ |
| | | | QTPGKAPKLLIYSASNRYTGVPSRFSGSGSGTDYTFTI |
| | | | SSLQPEDIATYYCQQYTNYPMYTFGQGTKVQIK |
| Antibody to | V_{H} | 318 | QVQLQESGPGLVKPSQTLSLTCAISGDSVSSNSVTWN |
| FAP variant 1 | | | WIRQSPSRGLEWLGRTYYRSKWYNDYAVSVKGRITI |
| | | | NPDTSKNQFYLQLKSVTPEDAAVYYCARDSSILYGDY |
| | | | WGQGTLVTVSS |
| | V_{H} | 319 | QVQLQQSGPGLVKPSQTLSLTCAISGDSVSSNSVTWN |
| | | | WIRQSPSRGLEWLGRTYYRSKWYNDYAVSVKGRITI |
| | | | NPDTSKNQFYLQLKSVTPEDAAVYYCARDSSILYGDY |
| | | | WGQGTLVTVS |
| | V_{L} | 320 | QAVLTQPSSLSASPGASASLTCTLPSGINVGTYRIFWF |
| | | | QQKPGSPPQYLLSYKSDSDNHQGSGVPSRFSGSKDAS |
| | | | ANAGILLISGLQSEDEADYYCMIWHSSAWVFGGGTK |
| | | | LTVL |
| Antibody to | V_{H} | 321 | QVQLVQSGAEVKKPGASVKVSCKTSGYTFTDYYIHW |
| FAP variant 2 | | | VRQAPGQGLEWMGWINPNRGGTNYAQKFQGRVTMT |
| | | | RDTSIATAYMELSRLRSDDTAVYYCATASLKIAAVGT |
| | | | FDCWGQGTLVTVSS |
| | V_{L} | 322 | SYELTQPPSVSVSPGQTARITCSGDALSKQYAFWFQQ |
| | | | KPGQAPILVIYQDTKRPSGIPGRFSGSSSGTTVTLTISG |
| | | | AQADDEADYYCQSADSSGTYVFGTGTKVTVL |
| Antibody to | V_{H} | 323 | EVQLVETGGGVVQPGRSLRLSCAASGFSFSTHGMYW |
| FAP variant 3 | | | VRQPPGKGLEWVAVISYDGSDKKYADSVKGRFTISR |
| | | | DNSKNTVYLEMSSVRAEDTALYYCFCRRDAFDLWG |
| | | | QGTMVTVSS |
| | V_{L} | 324 | SYVLTQPPSVSVSPGQTARITCSGDALPKKYAYWYQQ |
| | | | KSGQAPVLVIYEDTKRPSGIPERFSGSSSGTMATLTISG |
| | | | AQVEDEADYYCYSTDSSGNYWVFGGGTEVTVL |
| Antibody to | V_{H} | 325 | EVQLVESGGGLVEPGGSLRLSCAASGFTFSDAWMNW |
| FAP variant 4 | | | VRQAPGKGLEWVGRIKTKSDGGTTDYAAPVRGRFSIS |
| | | | RDDSKNTLFLEMNSLKTEDTAIYYCFITVIVVSSESPL |
| | | | DHWGQGTLVTVSS |
| | V_{L} | 326 | SYELTQPPSVSVSPGQTARITCSGDELPKQYAYWYQQ |
| | | | KPGQAPVLVIYKDRQRPSGIPERFSGSSSGTTVTLTISG |
| | | | VQAEDEADYYCQSAYSINTYVIFGGGTKLTVL |
| Antibody to | V_{H} | 327 | EVQLVESGGGLVKPGGSLRLSCAASGFTFSDYYMSWI |
| FAP variant 5 | | | RQAPGKGLEWISYISSGSSYTNYADSVKGRFTISRDNA |
| | | | KKSVYLEVNGLTVEDTAVYYCARVRYGDREMATIG |
| | | | GFDFWGQGTLVTVSS |
| | V_{L} | 328 | SYELTQPPSVSVSPGQTARITCSGDALPKQYAYWYQQ |
| | | | SPGQAPVLVIYKDSERPSGIPERFSGSSSGTTVTLTISG |
| | | | VQAEDEADYYCQSADSGGTSRIFGGGTKLTVL |
| Antibody to | V_{H} | 329 | QVQLQESGPGLVRSTETLSLTCLVSGDSINSHYWSWL |
| FAP variant 6 | | | RQSPGRGLEWIGYIYYTGPTNYNPSLKSRVSISLGTSK |
| | | | DQFSLKLSSVTAADTARYYCARNKVFWRGSDFYYY |
| | | | MDVWGKGTTVTVSS |
| | V_{L} | 330 | EIVLTQSPGTLSLSLGERATLSCRASQSLANNYLAWY |
| | | | QQKPGQAPRLLMYDASTRATGIPDRFSGSGSGTDFTL |
| | | | TISRLEPEDFAVYYCQQFVTSHHMYIFGQGTKVEIK |
| Antibody to | V_{H} | 331 | HVQLQESGPGLVKPSETLSLTCTVSGGSISSNNYYWG |
| FAP variant 7 | | | WIRQTPGKGLEWIGSIYYSGSTNYNPSLKSRVTISVDT |
| | | | SKNQFSLKLSSVTAADTAVYYCARGARWQARPATRI |
| | | | DGVAFDIWGQGTMVTVSS |
| | V_{H} | 332 | QVQLQESGPGLVKPSETLSLTCTVSGGSISSNNYYWG |
| | | | WIRQTPGKGLEWIGSIYYSGSTNYNPSLKSRVTISVDT |
| | | | SKNQFSLKLSSVTAADTAVYYCARGARWQARPATRI |
| | | | DGVAFDIWGQGTMVTVSS |
| | V_{H} | 333 | EVQLVQSGAEVKKPGASVKVSCKASGYTFTSYGISW |
| | | | VRQAPGQGLEWMGWISAYNGNTNYAQKLQGRVTM |
| | | | TTDTSTSTAYMELRSLRSDDTAVYYCARDWSRSGYY |
| | | | LPDYWGQGTLVTVSS |
| | V_{L} | 334 | ETTLTQSPGTLSLSPGERATLSCRASQTVTRNYLAWY |
| | | | QQKPGQAPRLLMYGASNRAAGVPDRFSGSGSGTDFT |
| | | | LTISRLEPEDFAVYYCQQFGSPYTFGQGTKVEIK |
| | V_{L} | 335 | DVVMTQSPLSLPVTLGQPASISCRSSQSLLHSNGYNYL |
| | | | DWYLQRPGQSPHLLIFLGSNRASGVPDRFSGSGSGTD |
| | | | FTLKISRVEAEDVGIYYCMQALQTPPTFGQGTKVEIK |
| Antibody to | V_{H} | 336 | QVQLVESGGGVVQPGRSLRLSCAASGFTFSSYGMHW |
| GITR | | | VRQAPGKGLEWVAVIWYEGSNKYYADSVKGRFTISR |
| | | | DNSKNTLYLQMNSLRAEDTAVYYCARGGSMVRGDY |
| | | | YYGMDVWGQGTTVTVSS |
| | V_{L} | 337 | AIQLTQSPSSLSASVGDRVTITCRASQGISSALAWYQQ |
| | | | KPGKAPKLLIYDASSLESGVPSRFSGSGSGTDFTLTISS |
| | | | LQPEDFATYYCQQFNSYPYTFGQGTKLEIK |
| 4G8 | V_{H} | 338 | EVQLLESGGGLVQPGGSLRLSCAASGFTFSSYAMSWV |
| | | | RQAPGKGLEWVSAISGSGGSTYYADSVKGRFTISRDN |
| | | | SKNTLYLQMNSLRAEDTAVYYCAKGWLGNFDYWG |
| | | | QGTLVTVSS |
| | V_{L} | 339 | EIVLTQSPGTLSLSPGERATLSCRASQSVSRSYLAWYQ |
| | | | QKPGQAPRLLIIGASTRATGIPDRFSGSGSGTDFTLTIS |
| | | | RLEPEDFAVYYCQQGQVIPPTFGQGTKVEIK |
| 4B9 | V_{H} | 340 | EVQLLESGGGLVQPGGSLRLSCAASGFTFSSYAMSWV |
| | | | RQAPGKGLEWVSAIIGSGASTYYADSVKGRFTISRDN |
| | | | SKNTLYLQMNSLRAEDTAVYYCAKGWFGGFNYWGQ |
| | | | GTLVTVSS |
| | V_{L} | 341 | EIVLTQSPGTLSLSPGERATLSCRASQSVTSSYLAWYQ |
| | | | QKPGQAPRLLINVGSRRATGIPDRFSGSGSGTDFTLTIS |
| | | | RLEPEDFAVYYCQQGIMLPPTFGQGTKVEIK |
| 28H1 | V_{H} | 342 | EVQLLESGGGLVQPGGSLRLSCAASGFTFSSHAMSWV |
| | | | RQAPGKGLEWVSAIWASGEQYYADSVKGRFTISRDN |
| | | | SKNTLYLQMNSLRAEDTAVYYCAKGWLGNFDYWG |
| | | | QGTLVTVSS |
| | V_{L} | 343 | EIVLTQSPGTLSLSPGERATLSCRASQSVSRSYLAWYQ |
| | | | QKPGQAPRLLIIGASTRATGIPDRFSGSGSGTDFTLTIS |
| | | | RLEPEDFAVYYCQQGQVIPPTFGQGTKVEIK |
| Antibody 1 to | V_{H} | 344 | QVQLVQSGAEVKKPGASVKVSCKASGYTFTSYGINW |
| MADCAM | | | VRQAPGQGLEWMGWISVYSGNTNYAQKVQGRVTM |
| | | | TADTSTSTAYMDLRSLRSDDTAVYYCAREGSSSSGDY |
| | | | YYGMDVWGQGTTVTVSS |
| | V_{L} | 345 | DIVMTQTPLSLSVTPGQPASISCKSSQSLLHTDGTTYL |
| | | | YWYLQKPGQPPQLLIYEVSNRFSGVPDRFSGSGSGTD |
| | | | FTLKISRVEAEDVGIYYCMQNIQLPWTFGQGTKVEIK |
| Antibody 2 to | V_{H} | 346 | QVQLVQSGAEVKKPGASVKVSCEASGYTFTSYGIDW |
| MADCAM | | | VRQAPGQGLEWMGWISVYSGNTNYAQKLQGRVTMS |
| | | | TDTSTSTAYMELRSLRSDDTAVYYCAREGSSSSGDYY |
| | | | YGMDVWGQGTTVTVSS |
| | V_{L} | 347 | DIVMTQTPLSLSVTPGQPASISCKSNQSLLYSDGKTYL |
| | | | FWYLQKPGQPPQLLIYEVSNRFSGVPDRFSGSGSGTDF |
| | | | TLKISRVEAEDVGVYYCMQSIQLPWTFGQGTKVEIK |
| Pamrevlumab | V_{H} | 348 | EGQLVQSGGGLVHPGGSLRLSCAGSGFTFSSYGMHW |
| (CTGF) | | | VRQAPGKGLEWVSGIGTGGGTYSTDSVKGRFTISRDN |
| | | | AKNSLYLQMNSLRAEDMAVYYCARGDYYGSGSFFD |
| | | | CWGQGTLVTVSS |
| | V_{L} | 349 | DIQMTQSPSSLSASVGDRVTITCRASQGISSWLAWYQ |
| | | | QKPEKAPKSLIYAASSLQSGVPSRFSGSGSGTDFTLTIS |
| | | | SLQPEDFATYYCQQYNSYPPTFGQGTKLEIK |
| Natalizumab | V_{H} | 350 | QVQLVQSGAEVKKPGASVKVSCKASGFNIKDTYIHW |
| | | | VRQAPGQRLEWMGRIDPANGYTKYDPKFQGRVTITA |
| | | | DTSASTAYMELSSLRSEDEAVYYCAREGYYGNYGVY |
| | | | AMDYWGQGTLVTVSS |
| | V_{L} | 351 | DIQMTQSPSSLSASVGDRVTITCKTSQDINKYMAWYQ |
| | | | QTPGKAPRLLIHYTSALQPGIPSRFSGSGSGRDYTFTIS |
| | | | SLQPEDIATYYCLQYDNLWTFGQGTKVEIK |
| Antibody to | V_{H} | 352 | EVQLQQSGAELVKPGASVKISCKASGYTFTDYNMDW |
| TNFR2 variant 1 | | | VKQSHGKSLEWIGDINPNYESTSYNQKFKGKATLTVD |
| | | | KSSSTAYMEVRSLTSEDTAVFYCARDKGWYFDVWG |
| | | | AGTTVTVSS |
| | V_{L} | 353 | ENVLTQSPAIMSASLGEKVTMSCRASSSVKNMYWYQ |
| | | | QKSDASPKLWIYYTSNLAPGVPARFSGSGSGNSYSLTI |
| | | | SSMEGEDAATYYCQQFTSSPYTFGGGTKLELK |
| Antibody to | V_{H} | 354 | QVTLKESGPGILQPSQTLSLTCSFSGFSLSTSGMGVGW |
| TNFR2 variant 2 | | | IRQPSGKGLEWLAHIWWDDDKFYNPSLKSQLTISKDT |
| | | | SRNQVFLKLTSVVTADTATYYCARLTGTRYFDYWGQ |
| | | | GTTLTVSS |
| | V_{L} | 355 | DVQMTQSPSSLSASLGGKVTITCKASQDINKFIAWYQ |
| | | | HKPGKGPRLLIHYTSTLQPGIPSKFSGSGSGRDYSFSIS |
| | | | NLEPEDIATYYCLQYGNLWTFGGGTKLEIT |
| Antibody to | V_{H} | 356 | QIQLVQSGPELKKPGETVKISCKASGYTFTDYSMHWV |
| TNFR2 variant 3 | | | KQAPGKGLKWMGWINTETGEPTYADDFKGRFAFSSE |
| | | | TSTSTAYLQINNLKNDDTTTYFCATYYGSSYVPDYW |
| | | | GQGTSLTVSS |
| | V_{L} | 357 | DIVMTQSHKFMSTSVGDRVSITCKASQNVGTAVAWY |
| | | | QHKPGQSPKLLIYWTSSRHTGVPDRFTGSGSGTEFTLT |
| | | | ISNVQSEDLADYFCHQYSDYPYTFGGGTKLEIK |
| Antibody to | V_{H} | 358 | EVQLQQSGPEVGRPGSSVKISCKASGYTFTDYIMHWV |
| TNFR2 variant 4 | | | KQSPGQGLEWIGWVDPEYGSTDYAEKFKKKATLTAD |
| | | | TSSNTAYIQLSSLTSEDTATYFCARDDGSYSPFDYWG |
| | | | QGVMVTVSS |
| | V_{L} | 359 | DIQMTQSPPSLSASLGDKVTITCQASQNINKYIAWYQ |
| | (major) | | QKPGKAPRLLIRYTSTLESGTPSRFSGSGSGRDYSFSIS |
| | | | NVESEDIASYYCLQYVNLLTFGAGTKLEIK |
| | V_{L} | 360 | NIVMTQSPKSMSMSVGERVTLTCKASENVVTYVSWY |
| | (minor) | | QQKPEQSPKLLIYGASNRYTGVPDRFTGSGSATDFTLT |
| | | | ISSVQAEDLADYHCGQGYSYPYTFGGGTKLEIK |
| Antibody to | V_{H} | 361 | EVQLVESGGGLVQPGGSLRLSCAASGFTFSDYAMSW |
| TNFR2 variant 5 | | | VRQAPGKGLEWVAVISENGSDTYYADSVKGRFTISRD |
| | | | DSKNTLYLQMNSLRAEDTAVYYCARDRGGAVSYFD |
| | | | VWGQGTLVTVSS |
| | V_{L} | 362 | DIQMTQSPSSLSASVGDRVTITCRASQDVSSYLAWYQ |
| | | | QKPGKAPKLLIYAASSLESGVPSRFSGSGSGTDFTLTIS |
| | | | SLQPEDFATYYCQQYNSLPYTFGQGTKVEIKRT |
| Antibody to | V_{H} | 363 | MAVLALLFCLVTFPSCILSQVQLKESGPGLVAPSQSLS |
| GARP variant 1 | | | ITCTVSGFSLTGYGINWVRQPPGKGLEWLGMIWSDGS |
| | | | TDYNSVLTSRLRISKDNSNSQVFLKMNSLQVDDTARY |
| | | | YCARDRNYYDYDGAMDYWGQGTSVTVSS |
| | V_{L} | 364 | QVQLKESGPGLVAPSQSLSITCTVSGFSLTGYGINWVR |
| | | | QPPGKGLEWLGMIWSDGSTDYNSVLTSRLRISKDNSN |
| | | | SQVFLKMNSLQVDDTARYYCARDRNYYDYDGAMD |
| | | | YWGQGTSVTVSS |
| Antibody to | V_{H} | 365 | MKFPSQLLLFLLFRITGIICDIQVTQSSSYLSVSLGDRV |
| GARP variant 2 | | | TITCKASDHIKNWLAWYQQKPGIAPRLLVSGATSLEA |
| | | | GVPSRFSGSGSGKNFTLSITSLQTEDVATYYCQQYWS |
| | | | TPWTFGGGTTLEIR |
| | V_{L} | 366 | DIQVTQSSSYLSVSLGDRVTITCKASDHIKNWLAWYQ |
| | | | QKPGIAPRLLVSGATSLEAGVPSRFSGSGSGKNFTLSIT |
| | | | SLQTEDVATYYCQQYWSTPWTFGGGTTLEIR |
| Antibody to | V_{H} | 367 | EVQLVQPGAELRNSGASVKVSCKASGYRFTSYYIDW |
| GARP variant 3 | | | VRQAPGQGLEWMGRIDPEDGGTKYAQKFQGRVTFT |
| | | | ADTSTSTAYVELSSLRSEDTAVYYCARNEWETVVVG |
| | | | DLMYEYEYWGQGTQVTVSS |
| | V_{L} | 368 | DIQMTQSPTSLSASLGDRVTITCQASQSISSYLAWYQQ |
| | | | KPGQAPKLLIYGASRLQTGVPSRFSGSGSGTSFTLTISG |
| | | | LEAEDAGTYYCQQYDSLPVTFGQGTKVELK |
| | V_{L} | 369 | DIQMTQSPSSLSASLGDRVTITCQASQSIVSYLAWYQQ |
| | | | KPGQAPKLLIYGASRLQTGVPSRFSGSGSGTSFTLTISG |
| | | | LEAEDAGTYYCQQYASAPVTFGQGTGVELK |
| | V_{L} | 370 | DIQMTQSPSSLSASLGDRVTITCQASQSISSYLAWYQQ |
| | | | KPGQAPKLLIYGTSRLKTGVPSRFSGSGSGTSFTLTISG |
| | | | LEAEDAGTYYCQQYYSAPVTFGQGTKVELK |
| | V_{L} | 371 | DIQMTQSPSSLSPSLGDRVTITCQASQTISSFLAWYHQ |
| | | | KPGQPPKLLIYRASIPQTGVPSRFSGSGSGTSFTLTIGG |
| | | | LEAEDAGTYYCQQYVSAPPTFGQGTKVELK |
| | V_{L} | 372 | DIQMTQSPSSLSASLGDRVTITCQASQSISSYLAWYQQ |
| | | | KPGQAPNILIYGASRLKTGVPSRFSGSGSGTSFTLTISG |
| | | | LEAEDAGTYYCQQYASVPVTFGQGTKVELK |
| Antibody to | V_{L} | 373 | DIQMTQSPSSLSASVGDRVTITCRASQGISSWLAWYQ |
| CD73 | | | QKPEKAPKSLIYAASSLQSGVPSRFSGSGSGTDFTLTIS |
| | | | SLQPEDFATYYCQQYNSYPLTFGGGTKVEIK |
| | V_{H} | 374 | QVQLVESGGGVVQPGRSLRLSCAASGFTFSNYGMHW |
| | | | VRQAPGKGLEWVAVILYDGSNKYYPDSVKGRFTISR |
| | | | DNSKNTLYLQMNSLRAEDTAVYYCARGGSSWYPDSF |
| | | | DIWGQGTMVTVSS |
| Daratumumab | V_{H} | 375 | EVQLLESGGGLVQPGGSLRLSCAVSGFTFNSFAMSWV |
| (CD38) | | | RQAPGKGLEWVSAISGSGGGTYYADSVKGRFTISRDN |
| | | | SKNTLYLQMNSLRAEDTAVYFCAKDKILWFGEPVFD |
| | | | YWGQGTLVTVSS |
| | V_{L} | 376 | EIVLTQSPATLSLSPGERATLSCRASQSVSSYLAWYQQ |
| | | | KPGQAPRLLIYDASNRATGIPARFSGSGSGTDFTLTISS |
| | | | LEPEDFAVYYCQQRSNWPPTFGQGTKVEIK |
| Etaracizumab | V_{H} | 377 | QVQLVESGGGVVQPGRSLRLSCAASGFTFSSYDMSW |
| | | | VRQAPGKGLEWVAKVSSGGGSTYYLDTVQGRFTISR |
| | | | DNSKNTLYLQMNSLRAEDTAVYYCARHLHGSFASW |
| | | | GQGTTVTVSS |
| | V_{L} | 378 | EIVLTQSPATLSLSPGERATLSCQASQSISNFLHWYQQ |
| | | | RPGQAPRLLIRYRSQSISGIPARFSGSGSGTDFTLTISSL |
| | | | EPEDFAVYYCQQSGSWPLTFGGGTKVEIK |
| Intetumumab | V_{H} | 379 | QVQLVESGGGVVQPGRSRRLSCAASGFTFSRYTMHW |
| | | | VRQAPGKGLEWVAVISFDGSNKYYVDSVKGRFTISR |
| | | | DNSENTLYLQVNILRAEDTAVYYCAREARGSYAFDI |
| | | | WGQGTMVTVSS |
| | V_{L} | 380 | EIVLTQSPATLSLSPGERATLSCRASQSVSSYLAWYQQ |
| | | | KPGQAPRLLIYDASNRATGIPARFSGSGSGTDFTLTISS |
| | | | LEPEDFAVYYCQQRSNWPPFTFGPGTKVDIK |
| Antibody to | V_{H} | 381 | EVQLVESGGGLVQPGGSLRLSCAVSGFVFSRYWMSW |
| Integrin αvβ8 | | | VRQAPGKGLEWIGEINPDSSTINYTSSLKDRFTISRDN |
| | | | AKNSLYLQMNSLRAEDTAVYYCASLITTEDYWGQGT |
| | | | TVTVSS |
| | V_{L} | 382 | EIVLTQSPSSLSLSPGERVTITCKASQDINSYLSWYQQK |
| | | | PGKAPKLLIYYANRLVDGVPARFSGSGSGQDYTLTISS |
| | | | LEPEDFAVYYCLQYDEFPYTFGGGTKLEIKR |
| Antibody to | V_{L} | 383 | DIQMTQSPSSLSASVGDRVTITCRASQDISNYLAWYQ |
| Integrin αv | | | QKPGKAPKLLIYYTSKIHSGVPSRFSGSGSGTDYTFTIS |
| | | | SLQPEDIATYYCQQGNTFPYTFGQGTKVEIK |
| | V_{H} | 384 | QVQLQQSGGELAKPGASVKVSCKASGYTFSSFWMH |
| | | | WVRQAPGQGLEWIGYINPRSGYTEYNEIFRDKATMTT |
| | | | DTSTSTAYMELSSLRSEDTAVYYCASFLGRGAMDYW |
| | | | GQGTTVTVSS |
| Antibody to | V_{H} | 385 | QVQLQESGPGLVKPSQTLSLTCTVSGGSISSGVYYWT |
| Integrin αvβ6 | | | WIRQHPGNGLEWIGYIYYSGSTSYNPSLKSRVTISVDT |
| variant 1 | | | SKKQFSLNLTSVTAADTAVYYCAREGPLRGDYYYGL |
| | | | DVWGQGTTVTVSS |
| | V_{L} | 386 | EIVLTQSPGTLSLSPGERATLSCRAGQTISSRYLAWYQ |
| | | | QKPGQAPRPLIYGASSRATGIPDRFSGSGSGTDFTLTIS |
| | | | RLEPEDFAVYYCQQYGSSPRTFGQGTKVEIK |
| Antibody to | V_{H} | 387 | QVQLQESGPGLVKPSQTLSLTCTVSGGSISSGGYYWS |
| Integrin αvβ6 | | | WIRQHPGKGLEWIGYIYYSGSTYYNPSLKSRVTISVDT |
| variant 2 | | | SKNQFSLKLSSVTAADTAMYYCARYRGPAAGRGDFY |
| | | | YFGMDVWGQGTTVTVSS |
| | V_{L} | 388 | DIVMTQTPLSLSVTPGQPASIFCKSSQSLLNSDGKTYL |
| | | | CWYLQKPGQPPQLLIYEVSNRFSGVPDRFSGSGSGTD |
| | | | FTLKISRVEAEDVGVYYCMQGIQLPWAFFGQGTKVEI |
| | | | K |
| Antibody to | V_{H} | 389 | QVQLVESGGGVVQPGRSLRLSCAASGFTFSSYGMHW |
| Integrin αvβ6 | | | VRQAPGKGLEWVAVIWYGGSNKYYADSVKGRFTISR |
| variant 3 | | | DNSKNTLYLQMNSLRAEDTAVYYCARDLAARRGDY |
| | | | YYYGMDVWGQGTTVTVSS |
| | V_{L} | 390 | SSELTQDPVVSVALGQTVRITCQGDSLRSYYLSWYQQ |
| | | | KPGQAPVLVIYGKNNRPSGIPDRFSGSNSGNTASLTIT |
| | | | GAQAEDEADYYCNSRDSSGNHLFGGGTKLTVL |
| Antibody to | V_{H} | 391 | QVQLQESGPGLVKPSQTLSLTCTVSGGSISSGGYYWS |
| Integrin αvβ6 | | | WIRQHPGKGLEWIGYIYYSGRTYNNPSLKSRVTISVD |
| variant 4 | | | TSKNQFSLKLSSVTAADTAVYYCARVATGRADYHFY |
| | | | AMDVWGQGTTVTVSS |
| | V_{L} | 392 | SYELTQPSSVSVSPGQTARITCSGDVLAKKSARWFHQ |
| | | | KPGQAPVLVIYKDSERPSGIPERFSGSSSGTTVTLTISG |
| | | | AQVEDEAAYYCYSAADNNLVFGGGTKLTVL |
| Zinbryta ™ | V_{H} | 393 | QVQLVQSGAEVKKPGSSVKVSCKASGYTFTSYRMH |
| (Daclizumab) | | | WVRQAPGQGLEWIGYINPSTGYTEYNQKFKDKATIT |
| (CD25) | | | ADESTNTAYMELSSLRSEDTAVYYCARGGGVFDYW |
| | | | GQGTLVTVSS |
| | V_{L} | 394 | DIQMTQSPSTLSASVGDRVTITCSASSSISYMHWYQQ |
| | | | KPGKAPKLLIYTTSNLASGVPARFSGSGSGTEFTLTISS |
| | | | LQPDDFATYYCHQRSTYPLTFGQGTKVEVK |
| Bleselumab | V_{H} | 395 | QLQLQESGPGLLKPSETLSLTCTVSGGSISSPGYYGGW |
| (CD40) | | | IRQPPGKGLEWIGSIYKSGSTYHNPSLKSRVTISVDTSK |
| | | | NQFSLKLSSVTAADTAVYYCTRPVVRYFGWFDPWGQ |
| | | | GTLVTVSS |
| | V_{L} | 396 | AIQLTQSPSSLSASVGDRVTITCRASQGISSALAWYQQ |
| | | | KPGKAPKLLIYDASNLESGVPSRFSGSGSGTDFTLTISS |
| | | | LQPEDFATYYCQQFNSYPTFGQGTKVEIK |
| Antibody to | V_{H} | 397 | QVQLVESGGGVVQPGRSLRLSCAASGFTFSNYGMYW |
| DEC-205 variant | | | VRQAPGKGLEWVAVIWYDGSNKYYADSVKGRFTISR |
| 1 | | | DNSKNTLYLQMNSLRAEDTAVYYCARDLWGWYFDY |
| | | | WGQGTLVTVSS |
| | V_{L} | 398 | EIVLTQSPATLSLSPGERATLSCRASQSVSSYLAWYQQ |
| | | | KPGQAPRLLIYDASNRATGIPARFSGSGSGTDFTLTISS |
| | | | LEPEDFAVYYCQQRRNWPLTFGGGTKVEIK |
| Antibody to | V_{L} | 399 | EVQLVQSGAEVKKPGESLRISCKGSGDSFTTYWIGWV |
| DEC-205 variant | | | RQMPGKGLEWMGIIYPGDSDTIYSPSFQGQVTISADKS |
| 2 | | | ISTAYLQWSSLKASDTAMYYCTRGDRGVDYWGQGT |
| | | | LVTVSS |
| | V_{L} | 400 | DIQMTQSPSSLSASVGDRVTITCRASQGISRWLAWYQ |
| | | | QKPEKAPKSLIYAASSLQSGVPSRFSGSGSGTDFTLTIS |
| | | | GLQPEDFATYYCQQYNSYPRTFGQGTKVEIK |
| Antibody 5 to | V_{H} | 401 | QVTLKESGPALVKPTQTLTLTCTFSGFSLSTSGMGVG |
| TNFR2 | | | WIRQPPGKALEWLAHIWWDDDKFYNPSLKSRLTISK |
| | | | DTSKNQVVLTMTNMDPVDTATYYCARITGTRYFDY |
| | | | WGQGTTVTVSS |
| | V_{L} | 402 | DIQMTQSPSSLSASVGDRVTITCKASQDINKFIAWYQQ |
| | | | KPGKAPKLLIHYTSTLQPGVPSRFSGSGSGTDYTFTISS |
| | | | LQPEDIATYYCLQYGNLWTFGGGTKVEIK |
| Fun1 | V_{L} | 403 | EVQLQQSGPELEKPGASVKISCKASGYSFTDYNMNW |
| | | | VKQSNGKSLEWIGNIDPYYGGTSYNQKFKGKATLTV |
| | | | DKSSSTAYMQLNSLTSEDSAVYFCARWDYRYDDGRA |
| | | | YYVMDFWGQGTSVTVSS |
| | V_{L} | 404 | ELQMTQSPSSLAASAGEKVTMSCKSSQSVLYSSNQKN |
| | | | YLAWYQQKPGQSPKLLIYWASTRESGVPDRFTGSGS |
| | | | GTHFTLTVSSVQAEDLAVYYCHQYLYSWTFGGGTNL |
| | | | EIK |
| hzFun1 | V_{H} | 405 | QVQLVQSGAEVKKPGASVKVSCKASGYTFTDYNMN |
| | | | WVRQAPGQGLEWMGNIDPYYGGTSYNQKFKGRVTM |
| | | | TRDTSISTAYMELSRLRSDDTAVYYCARWDYRYDDG |
| | | | RAYYVMDFWGQGTTVTVSS |
| | V_{L} | 406 | DIVMTQSPDSLAVSLGERATINCKSSQSVLYSSNQKN |
| | | | YLAWYQQKPGQPPKLLIYWASTRESGVPDRFSGSGSG |
| | | | TDFTLTISSLQAEDVAVYYCHQYLYSWTFGQGTKLEI |
| | | | K |
| Antibody 1 to | V_{H} | 407 | EVQLVESGAEVKKPGASVKVSCKASGYTFTNYIIHWV |
| CD45RB/RO | | | KQEPGQGLEWIGYFNPYNHGTKYNEKFKGRATLTAN |
| | | | KSISTAYMELSSLRSEDTAVYYCARSGPYAWFDTWG |
| | | | QGTTVTVSS |
| | V_{L} | 408 | DILLTQSPATLSLSPGERATFSCRASQNIGTSIQWYQQ |
| | | | KTNGAPRLLIRSSSESISGIPSRFSGSGSGTDFTLTISSLE |
| | | | PEDFAVYYCQQSNTWPFTFGQGTKLEIK |
| Antibody 2 to | V_{H} | 409 | EVQLVESGAEVKKPGASVKVSCKASGYTFTNYIIHWV |
| CD45RB/RO | | | KQEPGQGLEWIGYFNPYNHGTKYNEKFKGRATLTAN |
| | | | KSISTAYMELSSLRSEDTAVYYCARSGPYAWFDTWG |
| | | | QGTTVTVSS |
| | V_{L} | 410 | DILLTQSPATLSLSPGERATLSCRASQNIGTSIQWYQQ |
| | | | KPGQAPRLLIRSSSESISGIPSRFSGSGSGTDFTLTISSLE |
| | | | PEDFAVYYCQQSNTWPFTFGQGTKLEIK |
| Antibody to | V_{H} | 411 | QCQVQLVESGGGVVQPGRSLRVSCEASGFTFSNYGM |
| CD45RB | | | HWVRQAPGKGLEWVAVIWYDGSKKFYADSVKGRFT |
| | | | ISRDNSQNTLSLQMSSLRAEDTAVYYCARGGGDFDF |
| | | | WGQGTLVTVSS |
| | V_{L} | 412 | KIVMTQSPATLSVSPGERATLSCRASQSVSGNYLAWY |
| | | | QQRPGQAPRLLIYGASTRATGIPARFSGSGSGTEFTLTI |
| | | | SSLQSEDFAVYYCQQYGKWPPLTFGGGTKVEIK |
| Antibody 1 to | V_{H} | 413 | QVQLKESGPALVKPTQTLTLTCTFSGFSLSTSGVGVG |
| MHC-DR | | | WIRQPPGKALEWLALIDWDDDKYYSTSLKTRLTISKD |
| | | | TSKNQVVLTMTNMDPVDTATYYCARSPRYRGAFDY |
| | | | WGQGTLVTVSS |
| | V_{L} | 414 | DIVLTQPPSVSGAPGQRVTISCSGSESNIGNNYVQWYQ |
| | | | QLPGTAPKLLIYDNNQRPSGVPDRFSGSKSGTSASLAI |
| | | | TGLQSEDEADYYCQSYDLIRHVFGGGTKLTVLG |
| Antibody 2 to | V_{H} | 415 | QVQLKESGPALVKPTQTLTLTCTFSGFSLSTSGVGVG |
| MHC-DR | | | WIRQPPGKALEWLALIDWDDDKYYSTSLKTRLTISKD |
| | | | TSKNQVVLTMTNMDPVDTATYYCARSPRYRGAFDY |
| | | | WGQGTLVTVSS |
| | V_{L} | 416 | DIVLTQPPSVSGAPGQRVTISCSGSESNIGNNYVQWYQ |
| | | | QLPGTAPKLLIYDNNQRPSGVPDRFSGSKSGTSASLAI |
| | | | TGLQSEDEADYYCQSYDMNVHVFGGGTKLTVLG |
| Vatelizumab | V_{H} | 417 | QVQLQESGPGLVKPSETLSLTCTVSGFSLTNYGIHWIR |
| | | | QPPGKGLEWLGVIWARGFTNYNSALMSRLTISKDNS |
| | | | KNQVSLKLSSVTAADTAVYYCARANDGVYYAMDY |
| | | | WGQGTLVTVSS |
| | V_{L} | 418 | DFVMTQSPAFLSVTPGEKVTITCSAQSSVNYIHWYQQ |
| | | | KPDQAPKKLIYDTSKLASGVPSRFSGSGSGTDYTFTIS |
| | | | SLEAEDAATYYCQQWTTNPLTFGQGTKVEIK |
| Vedolizumab | V_{H} | 419 | QVQLVQSGAEVKKPGASVKVSCKGSGYTFTSYWMH |
| | | | WVRQAPGQRLEWIGEIDPSESNTNYNQKFKGRVTLT |
| | | | VDISASTAYMELSSLRSEDTAVYYCARGGYDGWDYA |
| | | | IDYWGQGTLVTVSS |
| | V_{L} | 420 | DVVMTQSPLSLPVTPGEPASISCRSSQSLAKSYGNTYL |
| | | | SWYLQKPGQSPQLLIYGISNRFSGVPDRFSGSGSGTDF |
| | | | TLKISRVEAEDVGVYYCLQGTHQPYTFGQGTKVEIK |
| Etrolizumab | V_{H} | 421 | EVQLVESGGGLVQPGGSLRLSCAASGFFITNNYWGW |
| | | | VRQAPGKGLEWVGYISYSGSTSYNPSLKSRFTISRDTS |
| | | | KNTFYLQMNSLRAEDTAVYYCARTGSSGYFDFWGQ |
| | | | GTLVTVSS |
| | V_{L} | 422 | DIQMTQSPSSLSASVGDRVTITCRASESVDDLLHWYQ |
| | | | QKPGKAPKLLIKYASQSISGVPSRFSGSGSGTDFTLTIS |
| | | | SLQPEDFATYYCQQGNSLPNTFGQGTKVEIK |
| Anifrolumab | V_{H} | 423 | EVQLVQSGAEVKKPGESLKISCKGSGYIFTNYWIAWV |
| | | | RQMPGKGLESMGHYPGDSDIRYSPSFQGQVTISADKSI |
| | | | TTAYLQWSSLKASDTAMYYCARHDIEGFDYWGRGT |
| | | | LVTVSS |
| | V_{L} | 424 | EIVLTQSPGTLSLSPGERATLSCRASQSVSSSFFAWYQ |
| | | | QKPGQAPRLLIYGASSRATGIPDRLSGSGSGTDFTLTIT |
| | | | RLEPEDFAVYYCQQYDSSAITFGQGTRLEIK |
| BIIB059 | V_{H} | 425 | DVQLVESGGGLVKPGGSLRLSCAASGFTFSTYTMSW |
| | | | VRQAPGKGLEWVATISPGDSFGYYYPDSVQGRFTISR |
| | | | DNAKNSLYLQMNSLRAEDTAVYYCTRDIYYNYGAW |
| | | | FAYWGQGTLVTVSS |
| | V_{L} | 426 | DIQLTQSPSSLSASVGDRVTITCKASQSVDYDGDSYM |
| | | | NWYQQKPGKAPKLLIYAASTLESGVPSRFSGSGSGTD |
| | | | FTLTISSLQPEDFATYYCQQANEDPRTFGQGTKVEIK |
| Brentuximab of | V_{H} | 427 | QIQLQQSGPEVVKPGASVKISCKASGYTFTDYYITWV |
| Brentuximab | | | KQKPGQGLEWIGWIYPGSGNTKYNEKFKGKATLTVD |
| Vedotin | | | TSSSTAFMQLSSLTSEDTAVYFCANYGNYWFAYWGQ |
| | | | GTQVTVSA |
| | V_{L} | 428 | DIVLTQSPASLAVSLGQRATISCKASQSVDFDGDSYM |
| | | | NWYQQKPGQPPKVLIYAASNLESGIPARFSGSGSGTD |
| | | | FTLNIHPVEEEDAATYYCQQSNEDPWTFGGGTKLEIK |
| Iratumumab | V_{H} | 429 | QVQLQQWGAGLLKPSETLSLTCAVYGGSFSAYYWS |
| | | | WIRQPPGKGLEWIGDINHGGGTNYNPSLKSRVTISVD |
| | | | TSKNQFSLKLNSVTAADTAVYYCASLTAYWGQGSLV |
| | | | TVSS |
| | V_{L} | 430 | DIQMTQSPTSLSASVGDRVTITCRASQGISSWLTWYQ |
| | | | QKPEKAPKSLIYAASSLQSGVPSRFSGSGSGTDFTLTIS |
| | | | SLQPEDFATYYCQQYDSYPITFGQGTRLEIK |
| Antibody to c- | V_{H} | 431 | QVQLVQSGAEVKKPGASVKVSCKASGYTFTSYNMH |
| KIT | | | WVRQAPGQGLEWMGVIYSGNGDTSYNQKFKGRVTI |
| | | | TADKSTSTAYMELSSLRSEDTAVYYCARERDTRFGN |
| | | | WGQGTLVTVSS |
| | V_{L} | 432 | DIVMTQSPDSLAVSLGERATINCRASESVDIYGNSFMH |
| | | | WYQQKPGQPPKLLIYLASNLESGVPDRFSGSGSGTDF |
| | | | TLTISSLQAEDVAVYYCQQNNEDPYTFGGGTKVEIK |
| Opdivo ™ | V_{H} | 433 | QVQLVESGGGVVQPGRSLRLDCKASGITFSNSGMHW |
| (nivolumab) | | | VRQAPGKGLEWVAVIWYDGSKRYYADSVKGRFTISR |
| | | | DNSKNTLFLQMNSLRAEDTAVYYCATNDDYWGQGT |
| | | | LVTVSS |
| | V_{L} | 434 | EIVLTQSPATLSLSPGERATLSCRASQSVSSYLAWYQQ |
| | | | KPGQAPRLLIYDASNRATGIPARFSGSGSGTDFTLTISS |
| | | | LEPEDFAVYYCQQSSNWPRTFGQGTKVEIK |
| Keytruda ™ | V_{H} | 435 | QVQLVQSGVEVKKPGASVKVSCKASGYTFTNYYMY |
| (pembrolizumab) | | | WVRQAPGQGLEWMGGINPSNGGTNFNEKFKNRVTL |
| | | | TTDSSTTTAYMELKSLQFDD |
| | | | TAVYYCARRDYRFDMGFDYWGQGTTVTVSS |
| | V_{L} | 436 | EIVLTQSPATLSLSPGERATLSCRASKGVSTSGYSYLH |
| | | | WYQQKPGQAPRLLIYLASYLESGVPARFSGSGSGTDF |
| | | | TLTISSLEPEDFAVYYCQHSRDLPLTFGGGTKVEIK |
| Antibody | V_{H} | 482 | EVQLVQSGAEVKKPGASVKVSCKASGYKFSSYWIEW |
| huM25 to | | | VKQAPGQGLEWIGEILPGSDTTNYNEKFKDRATFTSD |
| LRRC15 | | | TSINTAYMELSRLRSDDTAVYYCARDRGNYRAWFGY |
| | | | WGQGTLVTVSS |
| | V_{L} | 483 | DIQMTQSPSSLSASVGDRVTITCRASQDISNYLNWYQ |
| | | | QKPGGAVKFLIYYTSRLHSGVPSRFSGSGSGTDYTLTI |
| | | | SSLQPEDFATYFCQQGEALPWTFGGGTKVEIK |
| Antibody | V_{H} | 484 | EVQLVQSGAEVKKPGSSVKVSCKASFTFTDYYIHW |
| huAD208.4.1 to | | | VKQAPGQGLEWIGLVYPYIGGTNYNQKFKGKATLTV |
| LRRC15 | | | DTSTTTAYMEMSSLRSEDTAVYYCARGDNKYDAMD |
| | | | YWGQGTTVTVSS |
| | V_{L} | 485 | DIVLTQSPDSLAVSLGERATINCRASQSVSTSSYSYMH |
| | | | WYQQKPGQPPKLLIKYASSLESGVPDRFSGSGSGTDF |
| | | | TLTISSLQAEDVAVYYCEQSWEIRTFGGGTKVEIK |
| Antibody | V_{H} | 486 | EVQLVQSGAEVKKPGSSVKVSCKASGYTFTNYWMH |
| huAD208.12.1 | | | WVKQAPGQGLEWIGMIHPNSGSTKHNEKFRGKATLT |
| to LRRC15 | | | VDESTTTAYMELSSLRSEDTAVYYCARSDFGNYRWY |
| | | | FDVWGQGTTVTVSS |
| | V_{L} | 487 | EIVLTQSPATLSLSPGERATLSCRASQSSSNNLHWYQQ |
| | | | KPGQAPRVLIKYVSQSISGIPARFSGSGSGTDFTLTISSL |
| | | | EPEDFAVYFCQQSNSWPFTFGQGTKLEIK |
| Antibody | V_{H} | 488 | EVQLVQSGAEVKKPGSSVKVSCKASGFTFTDYYIHW |
| huAD208.14.1 | | | VKQAPGQGLEWIGLVYPYIGGSSYNQQFKGKATLTV |
| to LRRC15 | | | DTSTSTAYMELSSLRSEDTAVYYCARGDNNYDAMDY |
| | | | WGQGTTVTVSS |
| | V_{L} | 489 | DIVLTQSPDSLAVSLGERATISCRASQSVSTSTYNYMH |
| | | | WYQQKPGQPPKLLVKYASNLESGVPDRFSGSGSGTD |
| | | | FTLTISSLQAEDVAVYYCHHTWEIRTFGGGTKVEIK |
| Antibody | V_{H} | 490 | EVQLVESGGGLVQPGGSLRLSCAVSGFSLTSYGVHW |
| hu139.10 to | | | VRQATGKGLEWLGVIWAGGSTNYNSALMSRLTISKE |
| LRRC15 | | | NAKSSVYLQMNSLRAGDTAMYYCATHMITEDYYGM |
| | | | DYWGQGTTVTVSS |
| | V_{L} | 491 | DIVMTQSPDSLAVSLGERATINCKSSQSLLNSRTRKNY |
| | | | LAWYQQKPGQSPKLLIYWASTRESGVPDRFSGSGSGT |
| | | | DFTLTISSLQAEDVAVYYCKQSYNLPTFGGGTKVEIK |

In some embodiments, a binding domain can modulate an immune response by binding its antigen. A binding domain can modulate the activity of a cell type or tissue by binding to its antigen on the cell type or in the tissue. Some non-limiting examples of binding domains that can modulate an immune response by binding its antigen are a DEC-205 binding domain or a DCIR binding domain that can modulate immune activity of dendritic cells, or a FAP binding domain that can modulate immune activity of myofibroblasts at fibrotic tissue sites. Other non-limiting examples of binding domains are an LRRC15 binding domain, a TNFR2 binding domain or a Cadherin11 binding domain.

In various embodiments, a binding domain can be a first antigen binding domain in an antibody construct as described herein. A first antigen binding domain can bind an antigen on a diseased tissue, which can thereby target an attached or linked immune-modulatory compound to disease sites in the body. In some embodiments, the first antigen binding domain can recognize an antigen expressed on a stellate cell or a myofibroblast at sites of fibrosis. In some embodiments, the first antigen binding domain can recognize an antigen expressed on cells at sites of tissue-specific inflammation and autoimmunity, such as synovial fibroblasts, gut epithelial cells, and podocytes. In some embodiments, the first antigen binding domain can recognize an antigen expressed on a cell of a transplanted organ. In some embodiments, the antigen of the first antigen binding domain can be found on stellate cells, myofibroblasts, synovial fibroblasts, epithelial cells, or podocytes, such as Cadherin 11, PDPN, Integrin α4β7, Integrin α2β, Nephrin, Podocin, FAP, CD73, CD38, PDGFRβ, Integrin αvβ1, Integrin αvβ3, GARP, Endosialin, CTGF, c-KIT, or Integrin αvβ6. In some embodiments, the antigen of the first antigen binding domain can be found on stellate cells, myofibroblasts, synovial fibroblasts, epithelial cells, or podocytes, such as Cadherin 11, LRRC15, PDPN, Integrin α4β7, Integrin α2β, Nephrin, Podocin, FAP, CD73, CD38, PDGFRβ, Integrin αvβ1, Integrin αvβ3, GARP, Endosialin, CTGF, c-KIT, or Integrin αvβ6. In some embodiments, the antigen of the first antigen binding domain can be found on stellate cells, myofibroblasts, synovial fibroblasts, epithelial cells, or podocytes, such as Cadherin 11, LRRC15, PDPN, Integrin α4β7, Integrin α2β, Nephrin, Podocin, FAP, CD73, CD38, PDGFRβ, Integrin αvβ1, Integrin αvβ3, GARP, Endosialin, CTGF, c-KIT, Integrin αvβ6, MMP14, GPX8, or F2RL2. In some embodiments, the antigen of the first antigen binding domain is Cadherin 11, LRRC15, or FAP. In some embodiments, the antigen of the first antigen binding domain is TNFR2. In other embodiments, the first antigen binding domain can bind an antigen on an immune cell, such as BDCA2, CD30, CD40, PD-1, TIM-3, TNFR2, DEC205, DCIR, CD86, CD45RB, CD45RO, MHC Class II, or CD25.

### Second Antigen Binding Domain

A conjugate described herein can contain an antibody construct, where the antibody construct contains a first antigen binding domain and a second antigen binding domain. The second antigen binding domain can bind to an antigen that is the same or different than the antigen bound by the first antigen binding domain. The second antigen binding domain may be an antigen-binding portion of an antibody or an antibody fragment. The second antigen binding domain may be one or more fragments of an antibody that can retain the ability to specifically bind to an antigen. The second antigen binding domain may be any antigen binding fragment. The second antigen binding domain may be in a scaffold, in which a scaffold is a supporting framework for the second antigen binding domain. The second antigen binding domain may comprise an antigen binding domain in a scaffold.

In some embodiments, the second antigen is selected from TNFR2, CD40, CD86, PD-1, TIM3, BTLA, DEC205, DCIR, CD45RB, CD45RO, HLA DR, CD38, CD73, GARP, BDCA2, or CD30. In some embodiments, the second antigen is selected from TNFR2, CD40, CD86, PD-1, TIM3, BTLA, DEC205, DCIR, CD45RB, CD45RO, HLA DR, CD38, CD73, GARP, BDCA2, PD-L1, or CD30.

The second antigen binding domain may have, for example, about 50%, about 60%, about 70%, about 80%, or about 90% sequence identity to TNFR2. The second antigen binding domain can be an antagonist of, for example, immune cell immune-modulatory targets, an agonist of an immune checkpoint target, which can be found, for example, on immune cells, or mediate internalization of a cell surface antigen on immune cell types, for example, on an antigen presenting cell, and immune tissues. The second antigen binding domain may be, for example, an antagonist of CD40, CD86, an agonist of PD-1, TIM-3, or BTLA, or a binding domain to DEC-205. The second antigen binding domain may have, for example, about 50%, about 60%, about 70%, about 80%, or about 90% sequence identity to an antagonist of CD40, CD86, or PD-L1, an agonist of PD-1, TIM-3, or BTLA, or a binding domain to DEC-205. The second antigen binding domain may be expressed from a single construct encoding the antibody construct and the first antigen binding domain.

A second antigen binding domain may comprise an antigen binding domain which can refer to a portion of an antibody comprising the antigen recognition portion, i.e., an antigenic determining variable region of an antibody sufficient to confer recognition and binding of the antigen recognition portion to a target, such as an antigen, i.e., the epitope. A second antigen binding domain may comprise an antigen binding domain of an antibody.

An Fv can be the minimum antibody fragment which contains a complete antigen-recognition and antigen-binding site. This region may consist of a dimer of one heavy chain and one light chain variable domain in tight, non-covalent association. In this configuration, the three CDRs of each variable domain may interact to define an antigen-binding site on the surface of the V_{H}-V_{L} dimer. A single variable domain (or half of an Fv comprising only three CDRs specific for an antigen) can recognize and bind antigen, although at a lower affinity than the entire binding site.

A second antigen binding domain may be at least 80% identical to a specific antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), a single chain variable fragment (scFv), a DARPin, an affimer, an avimer, a knottin, a monobody, an affinity clamp, an ectodomain, a receptor ectodomain, a receptor, a cytokine, a ligand, an immunocytokine, a T cell receptor, an anticalin, a VNAR, a bicyclic peptide, or a recombinant T cell receptor.

A second antigen binding domain may be attached to an antibody construct. For example, a portion of an antibody construct may be fused with a second antigen binding domain to create an antibody construct comprising the second antigen binding domain as a fusion protein. The fusion protein may be the result of the nucleic acid sequence encoding the second antigen binding domain being expressed in frame with the nucleic acid sequence encoding the remainder of the antibody construct. The fusion protein may be the result of an in-frame genetic nucleotide sequence encoding the antibody construct with the antigen binding domain or a contiguous protein sequence of the antibody construct with the antigen binding domain. As another example, a second antigen binding domain may be linked to a portion of an antibody construct. A second antigen binding domain may be linked to a portion of an antibody construct by a chemical conjugation. A second antigen binding domain may be attached to a terminus of an Fc region. A second antigen binding domain may be attached to a terminus of an Fc domain. A second antigen binding domain may be attached to a terminus of a portion of an antibody construct. A second antigen binding domain may be attached to a terminus of an antibody. A second antigen binding domain may be attached to a light chain of an antibody. A second antigen binding domain may be attached to a terminus of a light chain of an antibody. A second antigen binding domain may be attached to a heavy chain of an antibody. A second antigen binding domain may be attached to terminus of a heavy chain of an antibody. The terminus may be a C-terminus. An antibody construct may be attached to 1, 2, 3, and/or 4 second antigen binding domains. The second antigen binding domain may direct the antibody construct to, for example, a particular cell or cell type. A second antigen binding domain of an antibody construct may be selected in order to recognize an antigen, e.g., an antigen expressed on an immune cell, or an antigen associated with fibrotic disease, autoimmune disease, or autoinflammatory disease. An antigen can be a peptide or fragment thereof. An antigen may be expressed on an immune cell. An antigen may be expressed on an antigen-presenting cell. An antigen may be expressed on a dendritic cell, a macrophage, or a B cell. When multiple second antigen binding domains are attached to an antibody construct, the second antigen binding domains may bind to the same antigen. When multiple second antigen binding domains are part of an antibody construct, the second antigen binding domains may bind to a different antigen(s).

In some embodiments, an antibody construct as described herein can comprise a second binding domain to a second antigen specific to an immune cell. In some embodiments, the second binding domain can further increase an immune-modulatory activity of the conjugate as compared to a conjugate as described herein without a second binding domain. Some non-limiting examples of second binding domains can be a non-activating CD40 binding domain that can blocks CD40L binding to CD40, or a PD-1 binding domain that can increase a PD-1 signal without blocking PD-L1 or PD-L2 binding to PD-1.

In some embodiments, a second binding domain can bind to a TNFRSF member as a scFv at the C-terminus of the Fc domain of the antibody construct or C terminus of the light chain of the first antigen binding domain, which can confer a lack of agonism while retaining binding on the TNFRSF binding domain, allowing for targeting with appropriate immune-modulation.

A second binding domain can comprise a set of six CDRs having at least at least 80%, at least 90%, at least 95%, at least 97%, at least 98%, at least 99%, or at least 100% sequence identity to a set of CDRs set forth in Table 1 as SEQ ID NO: 85-SEQ ID NO: 298. A second binding domain can comprise a set of CDRs having at least at least 80%, at least 90%, at least 95%, at least 97%, at least 98%, at least 99%, or at least 100% sequence identity to: a) HCDR1 comprising an amino acid sequence of SEQ ID NO: 85, HCDR2 comprising an amino acid sequence of SEQ ID NO: 86, HCDR3 comprising an amino acid sequence of SEQ ID NO: 87, LCDR1 comprising an amino acid sequence of SEQ ID NO: 88, LCDR2 comprising an amino acid sequence of SEQ ID NO: 89, and LCDR3 comprising an amino acid sequence of SEQ ID NO: 90; b) HCDR1 comprising an amino acid sequence of SEQ ID NO: 91, HCDR2 comprising an amino acid sequence of SEQ ID NO: 92, HCDR3 comprising an amino acid sequence of SEQ ID NO: 93, LCDR1 comprising an amino acid sequence of SEQ ID NO: 94, LCDR2 comprising an amino acid sequence of SEQ ID NO: 95, and LCDR3 comprising an amino acid sequence of SEQ ID NO: 96; c) HCDR1 comprising an amino acid sequence of SEQ ID NO: 97, HCDR2 comprising an amino acid sequence of SEQ ID NO: 98, HCDR3 comprising an amino acid sequence of SEQ ID NO: 99, LCDR1 comprising an amino acid sequence of SEQ ID NO: 100, LCDR2 comprising an amino acid sequence of SEQ ID NO: 101, and LCDR3 comprising an amino acid sequence of SEQ ID NO: 102; d) HCDR1 comprising an amino acid sequence of SEQ ID NO: 103, HCDR2 comprising an amino acid sequence of SEQ ID NO: 104, HCDR3 comprising an amino acid sequence of SEQ ID NO: 105, LCDR1 comprising an amino acid sequence of SEQ ID NO: 106, LCDR2 comprising an amino acid sequence of SEQ ID NO: 107, and LCDR3 comprising an amino acid sequence of SEQ ID NO: 108; e) HCDR1 comprising an amino acid sequence of SEQ ID NO: 109, HCDR2 comprising an amino acid sequence of SEQ ID NO: 110, HCDR3 comprising an amino acid sequence of SEQ ID NO: 111, LCDR1 comprising an amino acid sequence of SEQ ID NO: 112, LCDR2 comprising an amino acid sequence of SEQ ID NO: 113, and LCDR3 comprising an amino acid sequence of SEQ ID NO: 114; f) HCDR1 comprising an amino acid sequence of SEQ ID NO: 109, HCDR2 comprising an amino acid sequence of SEQ ID NO: 110, HCDR3 comprising an amino acid sequence of SEQ ID NO: 111, LCDR1 comprising an amino acid sequence of SEQ ID NO: 115, LCDR2 comprising an amino acid sequence of SEQ ID NO: 116, and LCDR3 comprising an amino acid sequence of SEQ ID NO: 117; g) HCDR1 comprising an amino acid sequence of SEQ ID NO: 118, HCDR2 comprising an amino acid sequence of SEQ ID NO: 119, HCDR3 comprising an amino acid sequence of SEQ ID NO: 120, LCDR1 comprising an amino acid sequence of SEQ ID NO: 121, LCDR2 comprising an amino acid sequence of SEQ ID NO: 122, and LCDR3 comprising an amino acid sequence of SEQ ID NO: 123; h) HCDR1 comprising an amino acid sequence of SEQ ID NO: 124, HCDR2 comprising an amino acid sequence of SEQ ID NO: 125, HCDR3 comprising an amino acid sequence of SEQ ID NO: 126, LCDR1 comprising an amino acid sequence of SEQ ID NO: 127, LCDR2 comprising an amino acid sequence of SEQ ID NO: 128, and LCDR3 comprising an amino acid sequence of SEQ ID NO: 129; i) HCDR1 comprising an amino acid sequence of SEQ ID NO: 130, HCDR2 comprising an amino acid sequence of SEQ ID NO: 131, HCDR3 comprising an amino acid sequence of SEQ ID NO: 132, LCDR1 comprising an amino acid sequence of SEQ ID NO: 133, LCDR2 comprising an amino acid sequence of SEQ ID NO: 134, and LCDR3 comprising an amino acid sequence of SEQ ID NO: 135; j) HCDR1 comprising an amino acid sequence of SEQ ID NO: 136, HCDR2 comprising an amino acid sequence of SEQ ID NO: 137, HCDR3 comprising an amino acid sequence of SEQ ID NO: 138, LCDR1 comprising an amino acid sequence of SEQ ID NO: 139, LCDR2 comprising an amino acid sequence of SEQ ID NO: 140, and LCDR3 comprising an amino acid sequence of SEQ ID NO: 141; k) HCDR1 comprising an amino acid sequence of SEQ ID NO: 142, HCDR2 comprising an amino acid sequence of SEQ ID NO: 143, HCDR3 comprising an amino acid sequence of SEQ ID NO: 144, LCDR1 comprising an amino acid sequence of SEQ ID NO: 145, LCDR2 comprising an amino acid sequence of SEQ ID NO: 146, and LCDR3 comprising an amino acid sequence of SEQ ID NO: 147; l) HCDR1 comprising an amino acid sequence of SEQ ID NO: 148, HCDR2 comprising an amino acid sequence of SEQ ID NO: 149, HCDR3 comprising an amino acid sequence of SEQ ID NO: 150, LCDR1 comprising an amino acid sequence of SEQ ID NO: 151, LCDR2 comprising an amino acid sequence of SEQ ID NO: 152, and LCDR3 comprising an amino acid sequence of SEQ ID NO: 153; m) HCDR1 comprising an amino acid sequence of SEQ ID NO: 154, HCDR2 comprising an amino acid sequence of SEQ ID NO: 155, HCDR3 comprising an amino acid sequence of SEQ ID NO: 156, LCDR1 comprising an amino acid sequence of SEQ ID NO: 157, LCDR2 comprising an amino acid sequence of SEQ ID NO: 158, and LCDR3 comprising an amino acid sequence of SEQ ID NO: 159; n) HCDR1 comprising an amino acid sequence of SEQ ID NO: 160, HCDR2 comprising an amino acid sequence of SEQ ID NO: 161, HCDR3 comprising an amino acid sequence of SEQ ID NO: 162, LCDR1 comprising an amino acid sequence of SEQ ID NO: 163, LCDR2 comprising an amino acid sequence of SEQ ID NO: 164, and LCDR3 comprising an amino acid sequence of SEQ ID NO: 165; o) HCDR1 comprising an amino acid sequence of SEQ ID NO: 166, HCDR2 comprising an amino acid sequence of SEQ ID NO: 167, HCDR3 comprising an amino acid sequence of SEQ ID NO: 168, LCDR1 comprising an amino acid sequence of SEQ ID NO: 169, LCDR2 comprising an amino acid sequence of SEQ ID NO: 170, and LCDR3 comprising an amino acid sequence of SEQ ID NO: 171; p) HCDR1 comprising an amino acid sequence of SEQ ID NO: 172, HCDR2 comprising an amino acid sequence of SEQ ID NO: 173, HCDR3 comprising an amino acid sequence of SEQ ID NO: 174, LCDR1 comprising an amino acid sequence of SEQ ID NO: 175, LCDR2 comprising an amino acid sequence of SEQ ID NO: 176, and LCDR3 comprising an amino acid sequence of SEQ ID NO: 177; q) HCDR1 comprising an amino acid sequence of SEQ ID NO: 178, HCDR2 comprising an amino acid sequence of SEQ ID NO: 179, HCDR3 comprising an amino acid sequence of SEQ ID NO: 180, LCDR1 comprising an amino acid sequence of SEQ ID NO: 181, LCDR2 comprising an amino acid sequence of SEQ ID NO: 182, and LCDR3 comprising an amino acid sequence of SEQ ID NO: 183; r) HCDR1 comprising an amino acid sequence of SEQ ID NO: 184, HCDR2 comprising an amino acid sequence of SEQ ID NO: 185, HCDR3 comprising an amino acid sequence of SEQ ID NO: 186, LCDR1 comprising an amino acid sequence of SEQ ID NO: 187, LCDR2 comprising an amino acid sequence of SEQ ID NO: 188, and LCDR3 comprising an amino acid sequence of SEQ ID NO: 189; s) HCDR1 comprising an amino acid sequence of SEQ ID NO: 190, HCDR2 comprising an amino acid sequence of SEQ ID NO: 191, HCDR3 comprising an amino acid sequence of SEQ ID NO: 192, LCDR1 comprising an amino acid sequence of SEQ ID NO: 193, LCDR2 comprising an amino acid sequence of SEQ ID NO: 194, and LCDR3 comprising an amino acid sequence of SEQ ID NO: 195; t) HCDR1 comprising an amino acid sequence of SEQ ID NO: 196, HCDR2 comprising an amino acid sequence of SEQ ID NO: 197, HCDR3 comprising an amino acid sequence of SEQ ID NO: 198, LCDR1 comprising an amino acid sequence of SEQ ID NO: 199, LCDR2 comprising an amino acid sequence of SEQ ID NO: 200, and LCDR3 comprising an amino acid sequence of SEQ ID NO: 201; u) HCDR1 comprising an amino acid sequence of SEQ ID NO: 202, HCDR2 comprising an amino acid sequence of SEQ ID NO: 203, HCDR3 comprising an amino acid sequence of SEQ ID NO: 204, LCDR1 comprising an amino acid sequence of SEQ ID NO: 205, LCDR2 comprising an amino acid sequence of SEQ ID NO: 206, and LCDR3 comprising an amino acid sequence of SEQ ID NO: 207; v) HCDR1 comprising an amino acid sequence of SEQ ID NO: 208, HCDR2 comprising an amino acid sequence of SEQ ID NO: 209, HCDR3 comprising an amino acid sequence of SEQ ID NO: 210, LCDR1 comprising an amino acid sequence of SEQ ID NO: 211, LCDR2 comprising an amino acid sequence of SEQ ID NO: 212, and LCDR3 comprising an amino acid sequence of SEQ ID NO: 213; w) HCDR1 comprising an amino acid sequence of SEQ ID NO: 214, HCDR2 comprising an amino acid sequence of SEQ ID NO: 215, HCDR3 comprising an amino acid sequence of SEQ ID NO: 216, LCDR1 comprising an amino acid sequence of SEQ ID NO: 217, LCDR2 comprising an amino acid sequence of SEQ ID NO: 218, and LCDR3 comprising an amino acid sequence of SEQ ID NO: 219; x) HCDR1 comprising an amino acid sequence of SEQ ID NO: 220, HCDR2 comprising an amino acid sequence of SEQ ID NO: 221, HCDR3 comprising an amino acid sequence of SEQ ID NO: 222, LCDR1 comprising an amino acid sequence of SEQ ID NO: 223, LCDR2 comprising an amino acid sequence of SEQ ID NO: 224, and LCDR3 comprising an amino acid sequence of SEQ ID NO: 225; y) HCDR1 comprising an amino acid sequence of SEQ ID NO: 226, HCDR2 comprising an amino acid sequence of SEQ ID NO: 227, HCDR3 comprising an amino acid sequence of SEQ ID NO: 228, LCDR1 comprising an amino acid sequence of SEQ ID NO: 229, LCDR2 comprising an amino acid sequence of SEQ ID NO: 230, and LCDR3 comprising an amino acid sequence of SEQ ID NO: 231; z) HCDR1 comprising an amino acid sequence of SEQ ID NO: 232, HCDR2 comprising an amino acid sequence of SEQ ID NO: 234, HCDR3 comprising an amino acid sequence of SEQ ID NO: 235, LCDR1 comprising an amino acid sequence of SEQ ID NO: 236, LCDR2 comprising an amino acid sequence of SEQ ID NO: 237, and LCDR3 comprising an amino acid sequence of SEQ ID NO: 238; aa) HCDR1 comprising an amino acid sequence of SEQ ID NO: 239, HCDR2 comprising an amino acid sequence of SEQ ID NO: 240, HCDR3 comprising an amino acid sequence of SEQ ID NO: 241, LCDR1 comprising an amino acid sequence of SEQ ID NO: 242, LCDR2 comprising an amino acid sequence of SEQ ID NO: 243, and LCDR3 comprising an amino acid sequence of SEQ ID NO: 244; bb) HCDR1 comprising an amino acid sequence of SEQ ID NO: 245, HCDR2 comprising an amino acid sequence of SEQ ID NO: 246, HCDR3 comprising an amino acid sequence of SEQ ID NO: 247, LCDR1 comprising an amino acid sequence of SEQ ID NO: 248, LCDR2 comprising an amino acid sequence of SEQ ID NO: 249, and LCDR3 comprising an amino acid sequence of SEQ ID NO: 250; cc) HCDR1 comprising an amino acid sequence of SEQ ID NO: 251, HCDR2 comprising an amino acid sequence of SEQ ID NO: 252, HCDR3 comprising an amino acid sequence of SEQ ID NO: 253, LCDR1 comprising an amino acid sequence of SEQ ID NO: 254, LCDR2 comprising an amino acid sequence of SEQ ID NO: 255, and LCDR3 comprising an amino acid sequence of SEQ ID NO: 256; dd) HCDR1 comprising an amino acid sequence of SEQ ID NO: 257, HCDR2 comprising an amino acid sequence of SEQ ID NO: 258, HCDR3 comprising an amino acid sequence of SEQ ID NO: 259, LCDR1 comprising an amino acid sequence of SEQ ID NO: 260, LCDR2 comprising an amino acid sequence of SEQ ID NO: 261, and LCDR3 comprising an amino acid sequence of SEQ ID NO: 262; ee) HCDR1 comprising an amino acid sequence of SEQ ID NO: 263, HCDR2 comprising an amino acid sequence of SEQ ID NO: 264, HCDR3 comprising an amino acid sequence of SEQ ID NO: 265, LCDR1 comprising an amino acid sequence of SEQ ID NO: 266, LCDR2 comprising an amino acid sequence of SEQ ID NO: 267, and LCDR3 comprising an amino acid sequence of SEQ ID NO: 268; ff) HCDR1 comprising an amino acid sequence of SEQ ID NO: 269, HCDR2 comprising an amino acid sequence of SEQ ID NO: 270, HCDR3 comprising an amino acid sequence of SEQ ID NO: 271, LCDR1 comprising an amino acid sequence of SEQ ID NO: 272, LCDR2 comprising an amino acid sequence of SEQ ID NO: 273, and LCDR3 comprising an amino acid sequence of SEQ ID NO: 274; gg) HCDR1 comprising an amino acid sequence of SEQ ID NO: 275, HCDR2 comprising an amino acid sequence of SEQ ID NO: 276, HCDR3 comprising an amino acid sequence of SEQ ID NO: 277, LCDR1 comprising an amino acid sequence of SEQ ID NO: 278, LCDR2 comprising an amino acid sequence of SEQ ID NO: 279, and LCDR3 comprising an amino acid sequence of SEQ ID NO: 280; hh) HCDR1 comprising an amino acid sequence of SEQ ID NO: 281, HCDR2 comprising an amino acid sequence of SEQ ID NO: 282, HCDR3 comprising an amino acid sequence of SEQ ID NO: 283, LCDR1 comprising an amino acid sequence of SEQ ID NO: 284, LCDR2 comprising an amino acid sequence of SEQ ID NO: 285, and LCDR3 comprising an amino acid sequence of SEQ ID NO: 286; ii) HCDR1 comprising an amino acid sequence of SEQ ID NO: 287, HCDR2 comprising an amino acid sequence of SEQ ID NO: 288, HCDR3 comprising an amino acid sequence of SEQ ID NO: 289, LCDR1 comprising an amino acid sequence of SEQ ID NO: 290, LCDR2 comprising an amino acid sequence of SEQ ID NO: 291, and LCDR3 comprising an amino acid sequence of SEQ ID NO: 292; or jj) HCDR1 comprising an amino acid sequence of SEQ ID NO: 293, HCDR2 comprising an amino acid sequence of SEQ ID NO: 294, HCDR3 comprising an amino acid sequence of SEQ ID NO: 295, LCDR1 comprising an amino acid sequence of SEQ ID NO: 296, LCDR2 comprising an amino acid sequence of SEQ ID NO: 297, and LCDR3 comprising an amino acid sequence of SEQ ID NO: 298.

An antibody construct may comprise a second binding domain that specifically binds to an antigen, wherein the second binding domain comprises a pair of variable regions having at least 80%, at least 90%, at least 95%, at least 97%, at least 98%, at least 99%, or at least 100% sequence identity to a pair of variable regions set forth in Table 2 as SEQ ID NO: 352-SEQ ID NO: 436. An antibody construct may comprise a second binding domain that specifically binds to an antigen, wherein the second binding domain comprises: a) a V_{H} sequence having at least 80%, at least 90%, at least 95%, at least 97%, at least 98%, at least 99%, or at least 100% sequence identity to an amino acid sequence of SEQ ID NO: 352, and a V_{L} sequence having at least 80%, at least 90%, at least 95%, at least 97%, at least 98%, at least 99%, or at least 100% sequence identity to an amino acid sequence of SEQ ID NO: 353; b) a V_{H} sequence having at least 80%, at least 90%, at least 95%, at least 97%, at least 98%, at least 99%, or at least 100% sequence identity to an amino acid sequence of SEQ ID NO: 354, and a V_{L} sequence having at least 80%, at least 90%, at least 95%, at least 97%, at least 98%, at least 99%, or at least 100% sequence identity to an amino acid sequence of SEQ ID NO: 355; c) a V_{H} sequence having at least 80%, at least 90%, at least 95%, at least 97%, at least 98%, at least 99%, or at least 100% sequence identity to an amino acid sequence of SEQ ID NO: 356, and a V_{L} sequence having at least 80%, at least 90%, at least 95%, at least 97%, at least 98%, at least 99%, or at least 100% sequence identity to an amino acid sequence of SEQ ID NO: 357; d) a V_{H} sequence having at least 80%, at least 90%, at least 95%, at least 97%, at least 98%, at least 99%, or at least 100% sequence identity to an amino acid sequence of SEQ ID NO: 358, and a V_{L} sequence having at least 80%, at least 90%, at least 95%, at least 97%, at least 98%, at least 99%, or at least 100% sequence identity to an amino acid sequence of SEQ ID NO: 359; e) a V_{H} sequence having at least 80%, at least 90%, at least 95%, at least 97%, at least 98%, at least 99%, or at least 100% sequence identity to an amino acid sequence of SEQ ID NO: 358, and a V_{L} sequence having at least 80%, at least 90%, at least 95%, at least 97%, at least 98%, at least 99%, or at least 100% sequence identity to an amino acid sequence of SEQ ID NO: 360; f) a V_{H} sequence having at least 80%, at least 90%, at least 95%, at least 97%, at least 98%, at least 99%, or at least 100% sequence identity to an amino acid sequence of SEQ ID NO: 361, and a V_{L} sequence having at least 80%, at least 90%, at least 95%, at least 97%, at least 98%, at least 99%, or at least 100% sequence identity to an amino acid sequence of SEQ ID NO: 362; g) a V_{H} sequence having at least 80%, at least 90%, at least 95%, at least 97%, at least 98%, at least 99%, or at least 100% sequence identity to an amino acid sequence of SEQ ID NO: 363, and a V_{L} sequence having at least 80%, at least 90%, at least 95%, at least 97%, at least 98%, at least 99%, or at least 100% sequence identity to an amino acid sequence of SEQ ID NO: 364; h) a V_{H} sequence having at least 80%, at least 90%, at least 95%, at least 97%, at least 98%, at least 99%, or at least 100% sequence identity to an amino acid sequence of SEQ ID NO: 365, and a V_{L} sequence having at least 80%, at least 90%, at least 95%, at least 97%, at least 98%, at least 99%, or at least 100% sequence identity to an amino acid sequence of SEQ ID NO: 366; i) a V_{H} sequence having at least 80%, at least 90%, at least 95%, at least 97%, at least 98%, at least 99%, or at least 100% sequence identity to an amino acid sequence of SEQ ID NO: 367, and a V_{L} sequence having at least 80%, at least 90%, at least 95%, at least 97%, at least 98%, at least 99%, or at least 100% sequence identity to an amino acid sequence of SEQ ID NO: 368; j) a V_{H} sequence having at least 80%, at least 90%, at least 95%, at least 97%, at least 98%, at least 99%, or at least 100% sequence identity to an amino acid sequence of SEQ ID NO: 367, and a V_{L} sequence having at least 80%, at least 90%, at least 95%, at least 97%, at least 98%, at least 99%, or at least 100% sequence identity to an amino acid sequence of SEQ ID NO: 369; k) a V_{H} sequence having at least 80%, at least 90%, at least 95%, at least 97%, at least 98%, at least 99%, or at least 100% sequence identity to an amino acid sequence of SEQ ID NO: 367, and a V_{L} sequence having at least 80%, at least 90%, at least 95%, at least 97%, at least 98%, at least 99%, or at least 100% sequence identity to an amino acid sequence of SEQ ID NO: 370; l) a V_{H} sequence having at least 80%, at least 90%, at least 95%, at least 97%, at least 98%, at least 99%, or at least 100% sequence identity to an amino acid sequence of SEQ ID NO: 367, and a V_{L} sequence having at least 80%, at least 90%, at least 95%, at least 97%, at least 98%, at least 99%, or at least 100% sequence identity to an amino acid sequence of SEQ ID NO: 371; m) a V_{H} sequence having at least 80%, at least 90%, at least 95%, at least 97%, at least 98%, at least 99%, or at least 100% sequence identity to an amino acid sequence of SEQ ID NO: 367, and a V_{L} sequence having at least 80%, at least 90%, at least 95%, at least 97%, at least 98%, at least 99%, or at least 100% sequence identity to an amino acid sequence of SEQ ID NO: 372; n) a V_{H} sequence having at least 80%, at least 90%, at least 95%, at least 97%, at least 98%, at least 99%, or at least 100% sequence identity to an amino acid sequence of SEQ ID NO: 374, and a V_{L} sequence having at least 80%, at least 90%, at least 95%, at least 97%, at least 98%, at least 99%, or at least 100% sequence identity to an amino acid sequence of SEQ ID NO: 373; o) a V_{H} sequence having at least 80%, at least 90%, at least 95%, at least 97%, at least 98%, at least 99%, or at least 100% sequence identity to an amino acid sequence of SEQ ID NO: 375, and a V_{L} sequence having at least 80%, at least 90%, at least 95%, at least 97%, at least 98%, at least 99%, or at least 100% sequence identity to an amino acid sequence of SEQ ID NO: 376; p) a V_{H} sequence having at least 80%, at least 90%, at least 95%, at least 97%, at least 98%, at least 99%, or at least 100% sequence identity to an amino acid sequence of SEQ ID NO: 377, and a V_{L} sequence having at least 80%, at least 90%, at least 95%, at least 97%, at least 98%, at least 99%, or at least 100% sequence identity to an amino acid sequence of SEQ ID NO: 378; q) a V_{H} sequence having at least 80%, at least 90%, at least 95%, at least 97%, at least 98%, at least 99%, or at least 100% sequence identity to an amino acid sequence of SEQ ID NO: 379, and a V_{L} sequence having at least 80%, at least 90%, at least 95%, at least 97%, at least 98%, at least 99%, or at least 100% sequence identity to an amino acid sequence of SEQ ID NO: 380; r) a V_{H} sequence having at least 80%, at least 90%, at least 95%, at least 97%, at least 98%, at least 99%, or at least 100% sequence identity to an amino acid sequence of SEQ ID NO: 381, and a V_{L} sequence having at least 80%, at least 90%, at least 95%, at least 97%, at least 98%, at least 99%, or at least 100% sequence identity to an amino acid sequence of SEQ ID NO: 382; s) a V_{H} sequence having at least 80%, at least 90%, at least 95%, at least 97%, at least 98%, at least 99%, or at least 100% sequence identity to an amino acid sequence of SEQ ID NO: 384, and a V_{L} sequence having at least 80%, at least 90%, at least 95%, at least 97%, at least 98%, at least 99%, or at least 100% sequence identity to an amino acid sequence of SEQ ID NO: 383; t) a V_{H} sequence having at least 80%, at least 90%, at least 95%, at least 97%, at least 98%, at least 99%, or at least 100% sequence identity to an amino acid sequence of SEQ ID NO: 385, and a V_{L} sequence having at least 80%, at least 90%, at least 95%, at least 97%, at least 98%, at least 99%, or at least 100% sequence identity to an amino acid sequence of SEQ ID NO: 386; u) a V_{H} sequence having at least 80%, at least 90%, at least 95%, at least 97%, at least 98%, at least 99%, or at least 100% sequence identity to an amino acid sequence of SEQ ID NO: 387, and a V_{L} sequence having at least 80%, at least 90%, at least 95%, at least 97%, at least 98%, at least 99%, or at least 100% sequence identity to an amino acid sequence of SEQ ID NO: 388; v) a V_{H} sequence having at least 80%, at least 90%, at least 95%, at least 97%, at least 98%, at least 99%, or at least 100% sequence identity to an amino acid sequence of SEQ ID NO: 389, and a V_{L} sequence having at least 80%, at least 90%, at least 95%, at least 97%, at least 98%, at least 99%, or at least 100% sequence identity to an amino acid sequence of SEQ ID NO: 390; w) a V_{H} sequence having at least 80%, at least 90%, at least 95%, at least 97%, at least 98%, at least 99%, or at least 100% sequence identity to an amino acid sequence of SEQ ID NO: 391, and a V_{L} sequence having at least 80%, at least 90%, at least 95%, at least 97%, at least 98%, at least 99%, or at least 100% sequence identity to an amino acid sequence of SEQ ID NO: 392; x) a V_{H} sequence having at least 80%, at least 90%, at least 95%, at least 97%, at least 98%, at least 99%, or at least 100% sequence identity to an amino acid sequence of SEQ ID NO: 393, and a V_{L} sequence having at least 80%, at least 90%, at least 95%, at least 97%, at least 98%, at least 99%, or at least 100% sequence identity to an amino acid sequence of SEQ ID NO: 394; y) a V_{H} sequence having at least 80%, at least 90%, at least 95%, at least 97%, at least 98%, at least 99%, or at least 100% sequence identity to an amino acid sequence of SEQ ID NO: 395, and a V_{L} sequence having at least 80%, at least 90%, at least 95%, at least 97%, at least 98%, at least 99%, or at least 100% sequence identity to an amino acid sequence of SEQ ID NO: 396; z) a V_{H} sequence having at least 80%, at least 90%, at least 95%, at least 97%, at least 98%, at least 99%, or at least 100% sequence identity to an amino acid sequence of SEQ ID NO: 397, and a V_{L} sequence having at least 80%, at least 90%, at least 95%, at least 97%, at least 98%, at least 99%, or at least 100% sequence identity to an amino acid sequence of SEQ ID NO: 398; aa) a V_{H} sequence having at least 80%, at least 90%, at least 95%, at least 97%, at least 98%, at least 99%, or at least 100% sequence identity to an amino acid sequence of SEQ ID NO: 399, and a V_{L} sequence having at least 80%, at least 90%, at least 95%, at least 97%, at least 98%, at least 99%, or at least 100% sequence identity to an amino acid sequence of SEQ ID NO: 400; bb) a V_{H} sequence having at least 80%, at least 90%, at least 95%, at least 97%, at least 98%, at least 99%, or at least 100% sequence identity to an amino acid sequence of SEQ ID NO: 401, and a V_{L} sequence having at least 80%, at least 90%, at least 95%, at least 97%, at least 98%, at least 99%, or at least 100% sequence identity to an amino acid sequence of SEQ ID NO: 402; cc) a V_{H} sequence having at least 80%, at least 90%, at least 95%, at least 97%, at least 98%, at least 99%, or at least 100% sequence identity to an amino acid sequence of SEQ ID NO: 403, and a V_{L} sequence having at least 80%, at least 90%, at least 95%, at least 97%, at least 98%, at least 99%, or at least 100% sequence identity to an amino acid sequence of SEQ ID NO: 404; dd) a V_{H} sequence having at least 80%, at least 90%, at least 95%, at least 97%, at least 98%, at least 99%, or at least 100% sequence identity to an amino acid sequence of SEQ ID NO: 405, and a V_{L} sequence having at least 80%, at least 90%, at least 95%, at least 97%, at least 98%, at least 99%, or at least 100% sequence identity to an amino acid sequence of SEQ ID NO: 406; ee) a V_{H} sequence having at least 80%, at least 90%, at least 95%, at least 97%, at least 98%, at least 99%, or at least 100% sequence identity to an amino acid sequence of SEQ ID NO: 407, and a V_{L} sequence having at least 80%, at least 90%, at least 95%, at least 97%, at least 98%, at least 99%, or at least 100% sequence identity to an amino acid sequence of SEQ ID NO: 408; ff) a V_{H} sequence having at least 80%, at least 90%, at least 95%, at least 97%, at least 98%, at least 99%, or at least 100% sequence identity to an amino acid sequence of SEQ ID NO: 409, and a V_{L} sequence having at least 80%, at least 90%, at least 95%, at least 97%, at least 98%, at least 99%, or at least 100% sequence identity to an amino acid sequence of SEQ ID NO: 410; gg) a V_{H} sequence having at least 80%, at least 90%, at least 95%, at least 97%, at least 98%, at least 99%, or at least 100% sequence identity to an amino acid sequence of SEQ ID NO: 411, and a V_{L} sequence having at least 80%, at least 90%, at least 95%, at least 97%, at least 98%, at least 99%, or at least 100% sequence identity to an amino acid sequence of SEQ ID NO: 412; hh) a V_{H} sequence having at least 80%, at least 90%, at least 95%, at least 97%, at least 98%, at least 99%, or at least 100% sequence identity to an amino acid sequence of SEQ ID NO: 413, and a V_{L} sequence having at least 80%, at least 90%, at least 95%, at least 97%, at least 98%, at least 99%, or at least 100% sequence identity to an amino acid sequence of SEQ ID NO: 414; ii) a V_{H} sequence having at least 80%, at least 90%, at least 95%, at least 97%, at least 98%, at least 99%, or at least 100% sequence identity to an amino acid sequence of SEQ ID NO: 415, and a V_{L} sequence having at least 80%, at least 90%, at least 95%, at least 97%, at least 98%, at least 99%, or at least 100% sequence identity to an amino acid sequence of SEQ ID NO: 416; jj) a V_{H} sequence having at least 80%, at least 90%, at least 95%, at least 97%, at least 98%, at least 99%, or at least 100% sequence identity to an amino acid sequence of SEQ ID NO: 417, and a V_{L} sequence having at least 80%, at least 90%, at least 95%, at least 97%, at least 98%, at least 99%, or at least 100% sequence identity to an amino acid sequence of SEQ ID NO: 418; kk) a V_{H} sequence having at least 80%, at least 90%, at least 95%, at least 97%, at least 98%, at least 99%, or at least 100% sequence identity to an amino acid sequence of SEQ ID NO: 419, and a V_{L} sequence having at least 80%, at least 90%, at least 95%, at least 97%, at least 98%, at least 99%, or at least 100% sequence identity to an amino acid sequence of SEQ ID NO: 420; ll) a V_{H} sequence having at least 80%, at least 90%, at least 95%, at least 97%, at least 98%, at least 99%, or at least 100% sequence identity to an amino acid sequence of SEQ ID NO: 421, and a V_{L} sequence having at least 80%, at least 90%, at least 95%, at least 97%, at least 98%, at least 99%, or at least 100% sequence identity to an amino acid sequence of SEQ ID NO: 422; mm) a V_{H} sequence having at least 80%, at least 90%, at least 95%, at least 97%, at least 98%, at least 99%, or at least 100% sequence identity to an amino acid sequence of SEQ ID NO: 423, and a V_{L} sequence having at least 80%, at least 90%, at least 95%, at least 97%, at least 98%, at least 99%, or at least 100% sequence identity to an amino acid sequence of SEQ ID NO: 424; nn) a V_{H} sequence having at least 80%, at least 90%, at least 95%, at least 97%, at least 98%, at least 99%, or at least 100% sequence identity to an amino acid sequence of SEQ ID NO: 425, and a V_{L} sequence having at least 80%, at least 90%, at least 95%, at least 97%, at least 98%, at least 99%, or at least 100% sequence identity to an amino acid sequence of SEQ ID NO: 426; oo) a V_{H} sequence having at least 80%, at least 90%, at least 95%, at least 97%, at least 98%, at least 99%, or at least 100% sequence identity to an amino acid sequence of SEQ ID NO: 427, and a V_{L} sequence having at least 80%, at least 90%, at least 95%, at least 97%, at least 98%, at least 99%, or at least 100% sequence identity to an amino acid sequence of SEQ ID NO: 428; pp) a V_{H} sequence having at least 80%, at least 90%, at least 95%, at least 97%, at least 98%, at least 99%, or at least 100% sequence identity to an amino acid sequence of SEQ ID NO: 429, and a V_{L} sequence having at least 80%, at least 90%, at least 95%, at least 97%, at least 98%, at least 99%, or at least 100% sequence identity to an amino acid sequence of SEQ ID NO: 430; qq) a V_{H} sequence having at least 80%, at least 90%, at least 95%, at least 97%, at least 98%, at least 99%, or at least 100% sequence identity to an amino acid sequence of SEQ ID NO: 431, and a V_{L} sequence having at least 80%, at least 90%, at least 95%, at least 97%, at least 98%, at least 99%, or at least 100% sequence identity to an amino acid sequence of SEQ ID NO: 432; rr) a V_{H} sequence having at least 80%, at least 90%, at least 95%, at least 97%, at least 98%, at least 99%, or at least 100% sequence identity to an amino acid sequence of SEQ ID NO: 433, and a V_{L} sequence having at least 80%, at least 90%, at least 95%, at least 97%, at least 98%, at least 99%, or at least 100% sequence identity to an amino acid sequence of SEQ ID NO: 434; or ss) a V_{H} sequence having at least 80%, at least 90%, at least 95%, at least 97%, at least 98%, at least 99%, or at least 100% sequence identity to an amino acid sequence of SEQ ID NO: 435, and a V_{L} sequence having at least 80%, at least 90%, at least 95%, at least 97%, at least 98%, at least 99%, or at least 100% sequence identity to an amino acid sequence of SEQ ID NO: 436.

### Attachment of Linkers to Antibody Constructs

The antibody construct immune-modulatory compound conjugates may also be referred to as antibody conjugates. Antibody conjugates described herein may comprise a linker, e.g., a peptide linker. Linkers of the conjugates and methods described herein may not affect the binding of active portions of a conjugate (e.g., active portions include antigen binding domains, Fc domains, targeting binding domains, antibodies, immune modulators, inhibitors, or the like) to a target, which can be a cognate binding partner such as an antigen. A linker can form a linkage between different parts of a conjugate, e.g., between an antibody construct and a compound of the disclosure (an immune-modulatory compound). In certain embodiments, an antibody conjugate comprises multiple linkers. In certain embodiments, wherein an antibody conjugate comprises multiple linkers, the linkers may be the same linkers or different linkers.

A linker may be bound, i.e., covalently bound, to an antibody construct by a bond between the antibody construct and the linker. A linker may be bound covalently to an anti-fibrosis associated antigen antibody construct by a bond between the anti-fibrosis associated antigen antibody construct and the linker. A linker may be bound covalently to an anti-autoimmune associated antigen antibody construct by a bond between the anti-autoimmune associated antigen antibody construct and the linker. A linker may be bound covalently to an anti-autoinflammatory associated antigen antibody construct by a bond between the anti-autoinflammatory associated antigen antibody construct and the linker. A linker may be bound covalently to an anti-APC (antigen presenting cell) molecule antibody by a bond between the anti-APC molecule antibody and the linker. For example, a linker may be bound covalently to a terminus of an amino acid sequence of an antibody construct, or could be bound covalently to a side chain modification to the antibody construct, such as the side chain of a lysine, serine, threonine, cysteine, tyrosine, aspartic acid, a non-natural amino acid residue, glutamine or glutamic acid residue. A linker may be bound covalently to a terminus of an amino acid sequence of an Fc region of an antibody construct, or may be bound covalently to a side chain modification of an Fc region of an antibody construct, such as the side chain of a lysine, serine, threonine, cysteine, tyrosine, aspartic acid, a non-natural amino acid residue, glutamine or glutamic acid residue. A linker may be covalently bound to a terminus of an amino acid sequence of an Fc domain of an antibody construct, or may be bound covalently to a side chain modification of an Fc domain of an antibody construct, such as the side chain of a lysine, serine, threonine, cysteine, tyrosine, aspartic acid, a non-natural amino acid residue, glutamine or glutamic acid residue.

A linker may be bound covalently to an antibody construct at a hinge cysteine. A linker may be bound covalently to an antibody construct at a light chain constant domain lysine. A linker may be bound covalently to an antibody construct at an engineered cysteine in the light chain. A linker may be bound covalently to an antibody construct at an Fc region lysine. A linker may be bound covalently to an antibody construct at an Fc domain lysine. A linker may be bound covalently to an antibody construct at an Fc region cysteine. A linker may be bound covalently to an antibody construct at an Fc domain cysteine. A linker may be bound covalently to an antibody construct at an engineered light chain glutamine. A linker may be bound covalently to an antibody construct at an unnatural amino acid engineered into the light chain. A linker may be bound covalently to an antibody construct at an unnatural amino acid engineered into the heavy chain. A linker may be bound covalently to an antibody construct at a lysine in the heavy chain. A linker may be bound covalently to an antibody construct at an engineered cysteine in the heavy chain. Amino acids can be engineered into an amino acid sequence of an antibody construct as described herein, for example, attachment of a linker of a conjugate. Engineered amino acids may be added to a sequence of existing amino acids. Engineered amino acids may be substituted for one or more existing amino acids of a sequence of amino acids.

A linker may be conjugated to an antibody construct via a sulfhydryl group. A linker may be conjugated to an antibody construct via a primary amine. A linker may be a link created between an unnatural amino acid on an antibody construct reacting with oxime bond that was formed by modifying a ketone group with an alkoxyamine on an immune-modulatory compound.

In some embodiments, an engineered cysteine is introduced in an antibody construct so that a linker can be attached at such engineered cysteine. For example, an engineered cysteine can be introduced into an IgG (typically an IgG1) at T114 (heavy chain), A140 (heavy chain), L174 (heavy chain), L179 (heavy chain), T187 (heavy chain), T209 (heavy chain), S239 (heavy chain), V262 (heavy chain), G371 (heavy chain), Y373 (heavy chain), E382 (heavy chain), S400 (heavy chain), S424 (heavy chain), N434 (heavy chain), Q438 (heavy chain), 1106 (light chain), R108 (light chain), A118 (heavy chain), R142 (light chain), K149 (light chain), and/or V205 (light chain), according to the EU numbering of Kabat.

In some embodiments, when one or more linkers are bound covalently to an antibody construct at the sites described herein, an Fc domain of the antibody construct can bind to Fc receptors. In certain embodiments, an antibody construct bound to a linker or an antibody construct bound to a linker bound to a TGFβR1 inhibitor, retains the ability of the Fc domain of the antibody to bind to Fc receptors. In certain embodiments, an antibody construct bound to a linker or an antibody construct bound to a linker bound to a TGFβR2 inhibitor, retains the ability of the Fc domain of the antibody to bind to Fc receptors. In certain embodiments, an antibody construct bound to a linker or an antibody construct bound to a linker bound to a TNKS inhibitor, retains the ability of the Fc domain of the antibody to bind to Fc receptors. In certain embodiments, an antibody construct bound to a linker or an antibody construct bound to a linker bound to a TNIK, retains the ability of the Fc domain of the antibody to bind to Fc receptors. In certain embodiments, when a linker is connected to an antibody construct at the sites described herein, the antigen binding domain of an antibody construct bound to a linker or an antibody construct bound to a linker bound to an immune-modulatory compound can bind its antigen. In certain embodiments, when a linker is connected to an antibody construct at the sites described herein, a second antigen binding domain of an antibody construct bound to a linker or an antibody construct bound to a linker bound to an immune-modulatory compound can bind its antigen.

In certain embodiments, a linker or linker bound to an immune-modulatory compound disclosed herein may not be attached to an amino acid residue of an IgG1 Fc domain selected from: 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327, 328, 329, 330, 331, 332, 333, 335, 336, 396, or 428, wherein numbering of amino acid residues in the Fc domain is according to the EU index as in Kabat.

In certain embodiments, a linker or linker bound to an immune-modulatory compound disclosed herein may be attached to an amino acid residue of an IgG1 Fc domain selected from: 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327, 328, 329, 330, 331, 332, 333, 335, 336, 396, or 428, wherein numbering of amino acid residues in the Fc domain is according to the EU index as in Kabat.

In some embodiments, when the linker bound to an immune-modulatory compound is linked to the antibody construct, the K_{d} of the first antigen binding domain for the first antigen may be retained. The K_{d} for binding of the first antigen binding domain of an antibody construct immune-modulatory compound conjugate to the first antigen in the presence of an immune-modulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the first antigen binding domain to the first antigen of an antibody construct in the absence of the immune-modulatory compound. The K_{d} for binding of the first antigen binding domain of an antibody construct immune-modulatory compound conjugate to the first antigen in the presence of the immune-modulatory compound can be less than 10 nM. The K_{d} for binding of the first antigen binding domain of an antibody construct immune-modulatory compound conjugate to the first antigen in the presence of the immune-modulatory compound can be less than 100 nM, less than 50 nM, less than 20 nM, less than 5 nM, less than 1 nM, or less than 0.1 nM.

When the linker bound to an immune-modulatory compound is linked to the antibody construct, the K_{d} of the second antigen binding domain for the second antigen may be retained. The K_{d} for binding of the second antigen binding domain of an antibody construct immune-modulatory compound conjugate to the second antigen in the presence of an immune-modulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the second antigen binding domain to the second antigen of an antibody construct in the absence of the immune-modulatory compound. The K_{d} for binding of the second antigen binding domain of an antibody construct immune-modulatory compound conjugate to the second antigen in the presence of the immune-modulatory compound can be less than 10 nM. The K_{d} for binding of the second antigen binding domain of an antibody construct immune-modulatory compound conjugate to the second antigen in the presence of the immune-modulatory compound can be less than 100 nM, less than 50 nM, less than 20 nM, less than 5 nM, less than 1 nM, or less than 0.1 nM.

When the linker bound to an immune-modulatory compound is linked to the antibody construct, the K_{d} of the first antigen binding domain for the first antigen may be retained and the K_{d} of the second antigen binding domain for the second antigen may be retained. The K_{d} for binding of the first antigen binding domain of an antibody construct immune-modulatory compound conjugate to the first antigen in the presence of an immune-modulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the first antigen binding domain to the first antigen of an antibody construct in the absence of the immune-modulatory compound, and the K_{d} for binding of the second antigen binding domain of an antibody construct immune-modulatory compound conjugate to the second antigen in the presence of an immune-modulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the second antigen binding domain to the second antigen of an antibody construct in the absence of the immune-modulatory compound. The K_{d} for binding of the first antigen binding domain of an antibody construct immune-modulatory compound conjugate to the first antigen in the presence of the immune-modulatory compound can be less than 10 nM, and the K_{d} for binding of the second antigen binding domain of an antibody construct immune-modulatory compound conjugate to the second antigen in the presence of the immune-modulatory compound can be less than 10 nM. The K_{d} for binding of the first antigen binding domain of an antibody construct immune-modulatory compound conjugate to the first antigen in the presence of the immune-modulatory compound can be less than 100 nM, less than 50 nM, less than 20 nM, less than 5 nM, less than 1 nM, or less than 0.1 nM, and the K_{d} for binding of the second antigen binding domain of an antibody construct immune-modulatory compound conjugate to the second antigen in the presence of the immune-modulatory compound can be less than 100 nM, less than 50 nM, less than 20 nM, less than 5 nM, less than 1 nM, or less than 0.1 nM.

The K_{d} for binding of an Fc domain of an antibody construct immune-modulatory compound conjugate to a Fc receptor in the presence of the immune-modulatory compound can be about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times greater than the K_{d} for binding of the Fc domain to the Fc receptor in the absence of the immune-modulatory compound. The K_{d} for binding of an Fc domain of an antibody construct immune-modulatory compound conjugate to an Fc receptor in the presence of the immune-modulatory compound can be less than 10 nM. The K_{d} for binding of an Fc domain of an antibody construct immune-modulatory compound conjugate to an Fc receptor in the presence of the immune-modulatory compound can be less than 10 μM, less than 1 μM, less than 100 nM, less than 50 nM, less than 20 nM, less than 5 nM, less than 1 nM, or less than 0.1 nM.

The K_{d} for binding of an Fc domain of an antibody construct immune-modulatory compound conjugate to a Fcγ receptor in the presence of the immune-modulatory compound may be equivalent to or no less than 2 times, 5 times, or 10 times a Kd for binding of the Fc domain to the Fcγ receptor in the absence of the immune-modulatory compound. The K_{d} for binding of an Fc domain of an antibody construct immune-modulatory compound conjugate to a Fcγ receptor in the presence of the immune-modulatory compound no less than about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times a K_{d} for binding of the Fc domain to the Fcγ receptor in the absence of the immune-modulatory compound. The K_{d} for binding of an Fc domain of an antibody construct immune-modulatory compound conjugate to an Fcγ receptor in the presence of the immune-modulatory compound can be less than 10 nM. The K_{d} for binding of an Fc domain of an antibody construct immune-modulatory compound conjugate to an Fcγ receptor in the presence of the immune-modulatory compound can be less than 10 μM, less than 1 μM, less than 100 nM, less than 50 nM, less than 20 nM, less than 5 nM, less than 1 nM, or less than 0.1 nM.

The K_{d} for binding of an Fc domain of an antibody construct immune-modulatory compound conjugate to a FcRn receptor in the presence of the immune-modulatory compound may be at least equivalent to or at least no greater than about 2 times, 5 times, or 10 times a K_{d} for binding of the Fc domain to the FcRn receptor in the absence of the immune-modulatory compound. The K_{d} for binding of an Fc domain of an antibody construct immune-modulatory compound conjugate to a FcRn receptor in the presence of the immune-modulatory compound may be at least equivalent to or at least no greater than about 2 times, about 3 times, about 4 times, about 5 times, about 6 times, about 7 times, about 8 times, about 9 times, about 10 times, about 15 times, about 20 times, about 25 times, about 30 times, about 35 times, about 40 times, about 45 times, about 50 times, about 60 times, about 70 times, about 80 times, about 90 times, about 100 times, about 110 times, or about 120 times a K_{d} for binding of the Fc domain to the FcRn receptor in the absence of the immune-modulatory compound. The K_{d} for binding of an Fc domain of an antibody construct immune-modulatory compound conjugate to an FcRn receptor in the presence of the immune-modulatory compound can be less than 10 nM. The K_{d} for binding of an Fc domain of an antibody construct immune-modulatory compound conjugate to an FcRn receptor in the presence of the immune-modulatory compound can be less than 10 μM, less than 1 μM, less than 100 nM, less than 50 nM, less than 20 nM, less than 5 nM, less than 1 nM, or less than 0.1 nM.

In some embodiments, the Fc domain activity (e.g., binding to a specific profile of Fc receptors) can be retained after covalent attachment of an immune-modulatory compound to an antibody construct. In various embodiments, an Fc domain can retain binding to FcRn as determined by retained Kd for FcRn or retention of half-life by the conjugate in an animal. In some embodiments, the Fc domain of the antibody construct can retain binding to Fcγ receptors as determined by retained Kd for Fcγ receptors or the retained ability to generate Fcγ receptor-mediated activity in cells expressing a specific set of Fcγ receptors, but not an antigen of a binding domain of the conjugate.

In some embodiments, an Fc domain of an antibody construct of a conjugate can be selected to lack binding to Fcγ receptors, which can be shown to retain this lack of binding by binding assays, cell based assays, or a combination thereof.

### Lysine-Based Bioconjugation

An antibody construct can be conjugated to a linker via lysine-based bioconjugation. An antibody construct can be exchanged into an appropriate buffer, for example, phosphate, borate, PBS, histidine, Tris-Acetate at a concentration of about 2 mg/mL to about 10 mg/mL. An appropriate number of equivalents of a construct of an amino-pyrazinecarboxamide compound, and a linker, linker-payload, as described herein, can be added as a solution with stirring. Dependent on the physical properties of the linker-payload, a co-solvent can be introduced prior to the addition of the linker-payload to facilitate solubility. The reaction can be stirred at room temperature for 2 hours to about 12 hours depending on the observed reactivity. The progression of the reaction can be monitored by LC-MS. Once the reaction is deemed complete, the remaining linker-payloads can be removed by applicable methods and the antibody conjugate can be exchanged into the desired formulation buffer. Lysine-linked conjugates can be synthesized starting with ab antibody (mAb) and linker-payload, e.g., 10 equivalents, following Scheme A below (Conjugate=antibody conjugate). Monomer content and drug-antibody construct ratios (molar ratios) can be determined by methods described herein.

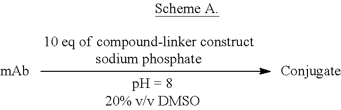

### Cysteine-Based Bioconjugation

An antibody construct can be conjugated to a linker via cysteine-based bioconjugation. An antibody construct can be exchanged into an appropriate buffer, for example, phosphate, borate, PBS, histidine, Tris-Acetate at a concentration of about 2 mg/mL to about 10 mg/mL with an appropriate number of equivalents of a reducing agent, for example, dithiothreitol or tris(2-carboxyethyl)phosphine. The resultant solution can be stirred for an appropriate amount of time and temperature to effect the desired reduction. A construct of an amino-pyrazinecarboxamide compound and a linker can be added as a solution with stirring. Dependent on the physical properties of the linker-payload, a co-solvent can be introduced prior to the addition of the linker-payload to facilitate solubility. The reaction can be stirred at room temperature for about 1 hour to about 12 hours depending on the observed reactivity. The progression of the reaction can be monitored by liquid chromatography-mass spectrometry (LC-MS). Once the reaction is deemed complete, the remaining free linker-payload can be removed by applicable methods and the antibody conjugate can be exchanged into the desired formulation buffer. Such cysteine-based conjugates can be synthesized starting with an antibody (mAb) and linker-payload, e.g., 7 equivalents, using the conditions described in Scheme B below (Conjugate=antibody conjugate). Monomer content and drug-antibody ratios can be determined by methods described herein.

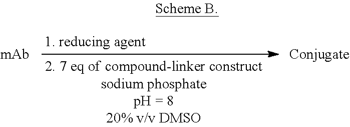

### Immune-Modulatory Compounds

An immune-modulatory compound can be a compound, such as a small molecule, large molecule, or other molecule that binds to a protein target and can activate the protein target's function, or an entity that binds to a protein target and can inhibit the protein target's function. In some embodiments, an immune-modulatory compound is not a nucleic acid. In some embodiments, an immune-modulatory compound binds to an intracellular protein target.

In some embodiments, an immune-modulatory compound can be designed to increase ubiquitin-mediated protein target destruction. Increased ubiquitin-mediated protein target destruction can use a small molecule(s) that binds to a protein subunit of an E3 ubiquitin ligase. The ubiquitin proteasome mediated protein degradation system of cells involves the covalent attachment of multiple ubiquitin molecules to lysine residues on a target protein, thereby marking the target protein for degradation by cellular proteasomes. The process of attaching ubiquitin molecules to a protein target typically involves 3 enzymes and steps: 1) an E1 enzyme that activates ubiquitin, and 2) an E2 enzyme that transfers activated ubiquitin to 3) a multi-subunit E3 enzyme ligase that catalyzes a ubiquitin attachment to the target protein.

Some examples of small molecules that can bind a subunit protein of a specific E3 ligase can be referred to as “PROTACs,” which can harness the ubiquitin proteasome system to degrade a chosen protein target for therapeutic use. PROTACs can comprise a small molecule that binds to a protein target and can be covalently attached by a linker to a small molecule that can bind an E3 ligase subunit. Harnessing the enzymatic machinery of the ubiquitin proteasome pathway can increase the potency of protein target inhibition. However, current PROTACs can face two challenges: 1) their size can make efficient delivery into cells more difficult, and 2) systemic delivery of potent protein target inhibition can exacerbate on-target or off-target toxicities often manifested by drugs. The conjugates as disclosed herein can be designed to overcome these difficulties while maintaining the benefit of harnessing the ubiquitin proteasome pathway.

An immune-modulatory compound can, for example, tolerize, suppress, repress, divert an immune response, or lower an inflammatory response against a patient tissue, patient cell, or patient antigen. In some embodiments, an immune-modulatory compound described herein can be, for example, a PI3K inhibitor, a Calcineurin inhibitor, an mTOR inhibitor, a BTK inhibitor, a JAK inhibitor, a CRAC inhibitor, a PARP1 antagonist, a PPARg agonist, a Kv1.3 antagonist, a KCa3.1 antagonist, a PP2A agonist, an IRAK4 inhibitor, an MYD88 inhibitor, a BCL-2 antagonist, an A2ar agonist, a TLR7 antagonist, a c-KIT kinase inhibitor, a KCA3.1 agonist, a TGFβR1 inhibitor, a TGFβR2 inhibitor, an ACC antagonist, an ASK1 antagonist, GLI1 inhibitor, a TNKS antagonist, or a TNIK antagonist.

In some embodiments, an immune-modulatory compound can be, for example, a PI3K inhibitor, a calcineurin inhibitor, an mTOR inhibitor, a BTK inhibitor, a JAK inhibitor, a CRAC (ORA11) inhibitor, a PARP1 antagonist, a PPARγ agonist, a Kv1.3 antagonist, a KCa3.1 antagonist, a PP2A agonist, an IRAK4 inhibitor, a MYD88 inhibitor, BCL-2, A2aR agonist, a vitamin D receptor (VDR) agonist, or GLI1 inhibitor.

In some embodiments, an immune-modulatory compound is a TGFβR1 inhibitor TGFβR2 inhibitor, TNKS antagonist, or TNIK antagonist.

In some embodiments, inhibitors of TGFβR1 kinase include those disclosed in US Published Application 2018/0127426, U.S. Pat. No. 8,080,568, WO 2012/002680, WO 2009/009059, WO 2007/076127, WO 2007076086, WO 2006026306, Bioorg. Med. Chem., 2014, 22, 2724-2732 and J. Med. Chem. 2014, 57, 4213-4238, the disclosures of which are incorporated by reference herein.

In some embodiments, inhibitors of the TGFβR2 kinase include those disclosed in WO 2015/136073, Bioorg. Med. Chem. Lett., 2013, 23, 3248-3252, Acta Cryst., 2016, D72, 658-674, WO 2016/020864, US Published Application 2014/0249135, US Published Application 20120225061 and compounds such as 3-amino-6-(4-(aminomethyl)phenyl)-N-(4-morpholinopyridin-3-yl)pyrazine-2-carboxamide, the disclosures of which are incorporated by reference herein.

In some embodiments, inhibitors of TNKS include those disclosed CN 107226808, EP 3313177, U.S. Pat. No. 9,505,749, US Published Application No. 2015/0045368, WO 2014/036022, WO 2017/076484, WO 2018/046933, WO 2018/003962, Eur. J. Med. Chem., 2017, 142, 506-522, the disclosures of which are incorporated by reference herein.

In some embodiments, inhibitors of TNIK include those disclosed US Published Application 2016/0264555, WO 2015/083833, US Published Application 2010/0216795, US Published Application 20100137386, Med. Chem. Commun., 2015, 6, 1564-1572, and Bioorg. Med. Chem. Lett., 2013, 23, 569-573, the disclosures of which are incorporated by reference herein.

In some embodiments, systemic lupus erythematosus can be treated using an antibody conjugate described herein. In some embodiments, the compound conjugated to the antibody conjugate can be a TLR7 antagonist.

In some embodiments, mastocytosis/uticaria pigmentosa can be treated using an antibody conjugate described herein. In some embodiments, the compound conjugated to the antibody construct to form a conjugate can be a c-KIT kinase inhibitor.

In some embodiments, a fibrotic disease described herein can be treated using an antibody conjugate described herein. In some embodiments, the compound conjugated to the antibody construct to form a conjugate can be a KCa3.1 agonist. In some embodiments, the compound conjugated to the antibody construct to form a conjugate can be an ACC inhibitor, such as GS-0976. In some embodiments, the compound conjugated to the antibody construct to form a conjugate can be an ASK1 inhibitor. In some embodiments, the compound conjugated to the antibody construct to form a conjugate can be a TGFβR1 inhibitor. In some embodiments, the compound conjugated to the antibody construct to form a conjugate can be a TGFβR2 inhibitor. In some embodiments, the compound conjugated to the antibody construct to form a conjugate can be a TNKS inhibitor. In some embodiments, the compound conjugated to the antibody construct to form a conjugate can be a TNIK inhibitor. In some embodiments, the compound conjugated to the antibody construct to form a conjugate can be a GLI1 inhibitor.

In some embodiments, multiple sclerosis can be treated using an antibody conjugate described herein. In some embodiments, the compound conjugated to the antibody construct to form a conjugate can be fingolimod.

Binding of an immune-modulatory compound to its target or target protein can increase the activity of a protein expressed in a myofibroblast, an immune cell, or both. Some non-limiting examples can include immune-modulatory compounds that are agonists of the adenine-receptor A2Ra such as CGS-21680 or sphingosine-1 analogues that increase activity of the phosphatase PP2A such as FTY720 and derived analogues.

Binding of an immune-modulatory compound to its target or protein target can inhibit the function of the protein target expressed in a myofibroblast, an immune cell, or both. Some non-limiting examples of immune-modulatory compounds can include: protein kinase inhibitors for mTOR kinases such as rapamycin, LIST, in immune cells; inhibitors of the TGFβR2 kinase such as 3-amino-6-(4-(aminomethyl)phenyl)-N-(4-morpholinopyridin-3-yl)pyrazine-2-carboxamide, in myofibroblasts, immune cells or both; inhibitors of one or both of PI3Kγ and PI3K6 such as Duvelisib, TG 100713, and PF 04691502 in immune cells; and inhibitors of TNIK such as KY-05009 and NCB-0846 [4-((2-((4-(aminomethyl)-1H-benzo[d]imidazol-6-yl)amino)quinazolin-8-yl)oxy)cyclohexan-1-ol] in myofibroblasts, immune cells or both.

An immune-modulatory compound can mediate target inhibition by covalent attachment to a target or target protein. A non-limiting example of an immune-modulatory compound that can inhibit a target protein can be a compound that can bind to an active site of TGFβR1 kinase. A non-limiting example of an immune-modulatory compound that can inhibit a target protein can be a compound that can bind to an active site of TGFβR2 kinase. A non-limiting example of an immune-modulatory compound that can inhibit a target protein can be a compound that can bind to an active site of TNKS. A non-limiting example of an immune-modulatory compound that can inhibit a target protein can be a compound that can bind to an active site of TNIK.

An immune-modulatory compound can bind to a protein target and can inhibit the function of the protein target by mediating degradation of the target protein expressed in a myofibroblast, an immune cell, or both. Non-limiting examples of immune-modulatory compounds can include inhibitors of TGFβR1, TGFβR2, TNKS and TNIK described above. Non-limiting examples of immune-modulatory compounds can further include inhibitors of TGFβR1, TGFβR2, TNKS and TNIK described above covalently attached or linked to an E3 ubiquitin ligase binding moiety, such as from a VHL binding moiety such as (S)-2-amino-N1-(4-(5-amino-6-((4-morpholinopyridin-3-yl)carbamoyl)pyrazin-2-yl)benzyl)-N5-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethyl)pentanediamide (Compound 1.1) or a cereblon binding moiety such as 3-amino-6-(4-(2-((2S)-2-amino-6-(2-((2-(2,6-dioxopiperidin-3-yl)-1,3-dioxoisoindolin-4-yl)oxy)acetamido)hexanamido)ethyl)phenyl)-N-(4-morpholinopyridin-3-yl)pyrazine-2-carboxamide (Compound 1.2). Other compounds that bind VHL may be hydroxyproline compounds such as those disclosed in WO2013/106643, and other compounds described in US 2016/0045607, WO 2014/187777, US 2014/0356322, and U.S. Pat. No. 9,249,153. Other compounds that bind to cereblon include thalidomide, lenalidomide, pomalidomide and analogs thereof. Other small molecule compounds that bind to cereblon are also known, e.g., the compounds disclosed as an in US 2016/0058872 and US2015/0291562.

In some embodiments, an E3 ubiquitin ligase binding moiety can be a second moiety. In some embodiments, an E3 ubiquitin ligase binding moiety can bind to an E3 ubiquitin ligase, such as Von Hippel-Lindaue E3 ubiquitin ligase (VHL), cereblon, mouse double minute 2 homolog (MDM2), AMFR, APC/Cdc20, APC/Cdh1, C6orf157, Cb1, CBLL1, CHFR, CHIP, DTL (Cdt2), E6-AP, HACE1, HECTD1, HECTD2, HECTD3, HECW1, HECW2, HERC2, HERC3, HERC4, HERC5, HUWE1, HYD, ITCH, LNX1, mahogunin, MARCH-I, MARCH-II, MARCH-III, MARCH-IV, MARCH-VI, MARCH-VII, MARCH-VIII, MARCH-X, MEKK1, MIB1, MIB2, MycBP2, NEDD4, NEDD4L, Parkin, PELI1, Pirh2, PJA1, PJA2, RFFL, RFWD2, Rictor, RNF5, RNF8, RNF19, RNF190, RNF20, RNF34, RNF40, RNF125, RNF128, RNF138, RNF168, SCF/β-TrCP, SCF/FBW7, SCF/Skp2, SHPRH, SIAH1, SIAH2, SMURF1, SMURF2, TOPORS, TRAF6, TRAF7, TRIM63, UBE3B, UBE3C, UBR1, UBR2, UHRF2, WWP1, WWP2, or ZNRF1.

A conjugate as described herein can alter the activity of a protein target of the immune-modulatory compound within a target cell.

In various embodiments, a conjugate can increase activity of a protein target of the immune-modulatory compound in a cell comprising a first antigen binding domain, a second binding domain, or a combination thereof.

In various embodiments, the conjugate can lower activity of the protein target of the immune-modulatory compound in a cell comprising a first antigen binding domain, a second binding domain, or a combination thereof. The conjugate can lower activity of the protein target of the immune-modulatory compound by increasing target protein degradation in a cell comprising a first antigen binding domain, a second binding domain, or a combination thereof.

The antigen targeted delivery of the conjugate to immune cells, myofibroblasts, or inflamed tissues can lower systemic toxicity of the immune-modulatory compound. Some non-limiting examples are immune-modulatory compounds that can inhibit TGFβR1, TGFβR2 or mTOR kinases. The antigen targeted delivery of the conjugate to an immune cell or myofibroblast can increase the potency of the immune-modulation. Some non-limiting examples can be comprised of conjugates that can promote target protein degradation using an immune-modulatory compound or first moiety linked with a second moiety or an E3 ubiquitin ligase binding moiety due to the relatively low cell permeability of larger non-attached immune-modulatory compound or first moiety.

In some aspects, the present disclosure provides a method for treating fibrosis, comprising administering an immune-modulatory compound or salt as described herein to a subject in need thereof. In some aspects, the present disclosure provides a method for treating fibrosis, comprising administering a conjugate comprising an immune-modulatory compound or salt as described herein to a subject in need thereof.

Included in the present disclosure are salts, particularly pharmaceutically acceptable salts, of the immune-modulatory compounds described herein. The immune-modulatory compounds of the present disclosure that possess a sufficiently acidic, a sufficiently basic, or both functional groups, may react with any of a number of inorganic bases, and inorganic and organic acids, to form a salt. Alternatively, immune-modulatory compounds that are inherently charged, such as those with a quaternary nitrogen, may form a salt with an appropriate counterion, e.g., a halide such as bromide, chloride, or fluoride, particularly bromide.

The immune-modulatory compounds described herein may in some cases exist as diastereomers, enantiomers, or other stereoisomeric forms. The immune-modulatory compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and conjugates and compositions described herein include the use of amorphous forms as well as crystalline forms (also known as polymorphs) of immune-modulatory compounds. The immune-modulatory compounds described herein may be in the form of pharmaceutically acceptable salts. As well, active metabolites of these immune-modulatory compounds having the same type of activity are included in the scope of the present disclosure. In addition, the immune-modulatory compounds described herein may exist in unsolvated as well as solvated forms with pharmaceutically acceptable solvents such as water, ethanol, and the like. The solvated forms of the immune-modulatory compounds presented herein are also considered to be disclosed herein.

In certain embodiments, immune-modulatory compounds or salts may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce an immune-modulatory compound are included within the scope of the claims. In some cases, some of the herein-described immune-modulatory compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In certain embodiments, the prodrug may be converted, e.g., enzymatically or chemically, to the parent compound under the conditions within a cell. In certain embodiments, the parent compound comprises an acidic moiety, e.g., resulting from the hydrolysis of the prodrug, which may be charged under the conditions within the cell. In particular embodiments, the prodrug is converted to the parent compound once it has passed through the cell membrane into a cell. In certain embodiments, the parent compound has diminished cell membrane permeability properties relative to the prodrug, such as decreased lipophilicity and increased hydrophilicity.

In particular embodiments, the parent compound with the acidic moiety is retained within a cell for a longer duration than the same compound without the acidic moiety.

The parent compound, with an acidic moiety, may be retained within the cell, i.e., drug residence, for 10% or longer, such as 15% or longer, such as 20% or longer, such as 25% or longer, such as 30% or longer, such as 35% or longer, such as 40% or longer such as 45% or longer, such as 50% or longer, such as 55% or longer, such as 60% or longer, such as 65% or longer, such as 70% or longer, such as 75% or longer, such as 80% or longer, such as 85% or longer, or even 90% or longer relative to the same compound without an acidic moiety.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J Pharmaceutics, 47, 103 (1988); Sinkula et al., J Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Linkers

The immune-modulatory compounds and salts described herein are bound covalently to a linker, e.g., a cleavable or non-cleavable linker. In certain embodiments, the linker is also bound covalently to an antibody and referred to as an antibody conjugate or conjugate. Linkers of the conjugates described herein may not affect the binding of active portions of a conjugate or antibody construct, e.g., the first antigen binding domains, Fc domains, second antigen binding domains, antibodies, immune-modulatory compounds, antagonists, agonists, or the like, to a target, which can be a cognate binding partner such as an antigen. A conjugate can comprise multiple linkers. These linkers can be the same linkers or different linkers. A linker described herein can be a multi-functional linker linking two small molecule binding moieties and linking the linked small molecules to an antibody.

A linker can be short, flexible, rigid, cleavable, non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable. The linker can include linkages that are designed to cleave and/or immolate or otherwise breakdown specifically or non-specifically inside cells. A cleavable linker can be sensitive to enzymes. A cleavable linker can be cleaved by enzymes such as proteases. A cleavable linker can contain a valine-citrulline peptide or a valine-alanine peptide. A valine-citrulline or valine-alanine containing linker can contain a pentafluorophenyl group. A valine-citrulline or valine-alanine containing linker can contain a succinimide group. A valine-citrulline or valine-alanine containing linker can contain a para aminobenzoic acid (PABA) group. A linker containing a valine-citrulline, valine-alanine (VA), or a glycine-glycine-phenylalanine-glycine (GGFG) (SEQ ID NO: 493) tetrapeptide can contain a PABA group and a pentafluorophenyl group. A peptide based linker can contain a PABA group and a succinimide group.

A non-cleavable linker can be protease insensitive. A non-cleavable linker can be maleimidocaproyl linker. A maleimidocaproyl linker can comprise N-maleimidomethylcyclohexane-1-carboxylate. A maleimidocaproyl linker can contain a succinimide group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can contain maleimides linked to polyethylene glycol molecules in which the polyethylene glycol can allow for more linker flexibility or can be used.

A linker can also contain an alkylene, alkenylene, alkynylene, polyether, polyester or polyamide group(s) and/or contain polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker. A Sortase A linker can be created by a Sortase A enzyme fusing an LPXTG (SEQ ID NO: 492) recognition motif to an N-terminal GGG motif to regenerate a native amide bond. The linker created can therefore link a moiety attached to the LPXTG (SEQ ID NO: 492) recognition motif with a moiety attached to the N-terminal GGG motif.

In the conjugates described herein, an immune-modulatory compound or salt is linked to the antibody construct by way of linkers. The linker linking the compound or salt to the antibody of a conjugate may be short, long, hydrophobic, hydrophilic, flexible or rigid, or may be composed of segments that each independently have one or more of the above-mentioned properties such that the linker may include segments having different properties. The linkers may be polyvalent such that they covalently link more than one compound or salt to a single site on the antibody, or monovalent such that covalently they link a single compound or salt to a single site on the antibody.

As will be appreciated by skilled artisans, the linkers may link an immune-modulatory compound to the antibody by a covalent linkage between the linker and the antibody construct and compound. As used herein, the expression “linker” is intended to include (i) unconjugated forms of the linker that include a functional group capable of covalently linking the linker to an immune-modulatory compound and a functional group capable of covalently linking the linker to an antibody; (ii) partially conjugated forms of the linker that include a functional group capable of covalently linking the linker to an antibody construct and that is covalently linked to an immune-modulatory compound or vice versa; and (iii) fully conjugated forms of the linker that is covalently linked to both an immune-modulatory compound and an antibody construct. One embodiment pertains to a conjugate formed by contacting an antibody that binds to a cell surface receptor or tumor associated antigen expressed on a tumor cell with a linker or linker-immune-modulatory compound described herein under conditions in which the linker or linker-immune-modulatory compound covalently links to the antibody. One embodiment pertains to a method of making a conjugate formed by contacting a linker or linker-immune-modulatory compound described herein under conditions in which the linker or linker-immune-modulatory compound covalently links to the antibody.

Exemplary polyvalent linkers that may be used to link many immune-modulatory compounds to an antibody construct (e.g., an antibody) are described. For example, Fleximer® linker technology has the potential to enable high-DAR ADCs with good physicochemical properties. As shown below, the Fleximer® linker technology is based on incorporating drug molecules into a solubilizing poly-acetal backbone via a sequence of ester bonds. The methodology renders highly-loaded conjugates (DAR up to 20) whilst maintaining good physicochemical properties.

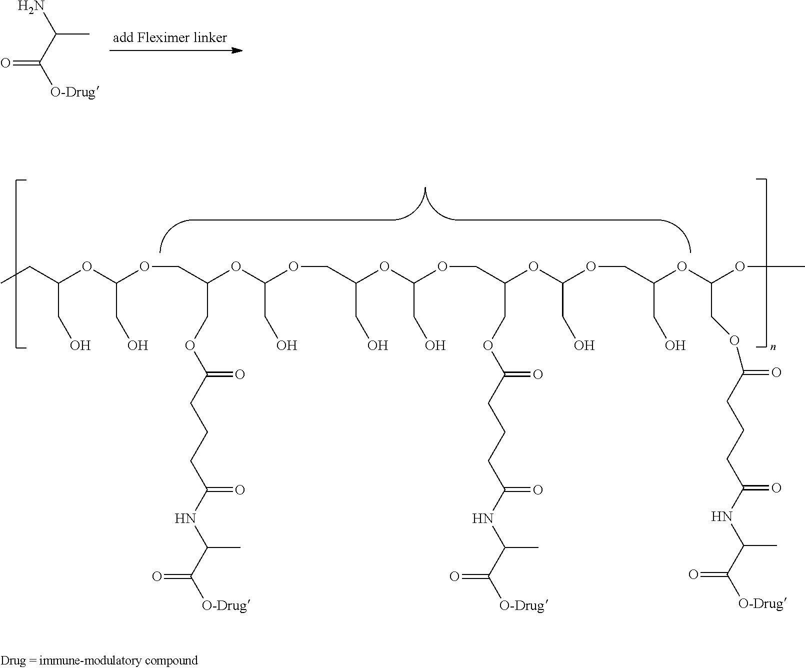

To utilize the Fleximer® linker technology depicted in the scheme above, an aliphatic alcohol can be present or introduced into the immune-modulatory compound. The alcohol moiety is then conjugated to an alanine moiety, which is then synthetically incorporated into the Fleximer® linker. Liposomal processing of the ADC in vitro releases the parent alcohol-containing drug.

By way of example and not limitation, some cleavable and noncleavable linkers that may be included in the conjugates described herein are described below.

Cleavable linkers can be cleavable in vitro and in vivo. Cleavable linkers can include chemically or enzymatically unstable or degradable linkages. Cleavable linkers can rely on processes inside the cell to liberate an immune-modulatory compound, such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

A linker can contain a chemically labile group such as hydrazone and/or disulfide groups. Linkers comprising chemically labile groups can exploit differential properties between the plasma and some cytoplasmic compartments. The intracellular conditions that can facilitate immune-modulatory compound release for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release the immune-modulatory compound once the antibody construct immune-modulatory compound conjugate is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Antibody construct immune-modulatory compound conjugates including exemplary hydrazone-containing linkers can include, for example, the following structures:

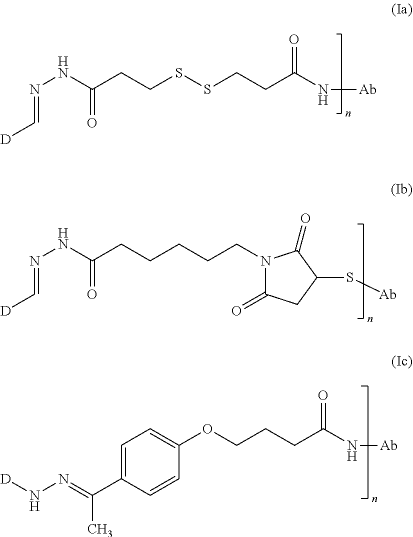

wherein D is an immune-modulatory compound, and Ab is an antibody construct, respectively, and n represents the number of—compounds bound to linkers (LP) bound to the antibody construct. In certain linkers, such as linker (Ia), the linker can comprise two cleavable groups—a disulfide and a hydrazone moiety. For such linkers, effective release of the unmodified free immune-modulatory compound can require acidic pH or disulfide reduction and acidic pH. Linkers such as (Ib) and (Ic) can be effective with a single hydrazone cleavage site.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release the immune-modulatory compound upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing the immune-modulatory compound in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Antibody conjugates including exemplary disulfide-containing linkers can include the following structures:

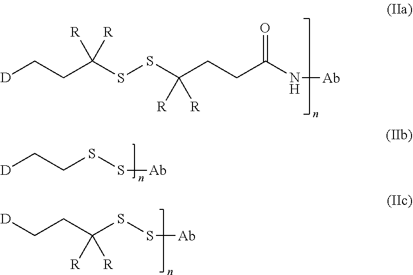

wherein is an immune-modulatory compound, and Ab is an antibody construct, respectively, n represents the number of compounds bound to linkers (LP) bound to the antibody construct and R is independently selected at each occurrence from hydrogen or alkyl, for example. Increasing steric hindrance adjacent to the disulfide bond can increase the stability of the linker

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of an immune-modulatory compound from an antibody construct can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

The cleavable peptide can be selected from tetrapeptides such as Gly-Phe-Leu-Gly (SEQ ID NO: 494), Ala-Leu-Ala-Leu (SEQ ID NO: 495), Gly-Gly-Phe-Gly (SEQ ID NO: 493), or dipeptides such as Val-Cit, Val-Ala, and Phe-Lys. Dipeptides can have lower hydrophobicity compared to longer peptides.

A variety of dipeptide-based cleavable linkers can be used in the antibody constructs immune-modulatory compound conjugates described herein.

Enzymatically cleavable linkers can include a self-immolative spacer to spatially separate the immune-modulatory compound from the site of enzymatic cleavage. The direct attachment of an immune-modulatory compound to a peptide linker can result in proteolytic release of an amino acid adduct of the immune-modulatory compound, thereby impairing its activity. The use of a self-immolative spacer can allow for the elimination of the fully active, chemically unmodified immune-modulatory compound upon amide bond hydrolysis.

One self-immolative spacer can be a bifunctional para-aminobenzyl alcohol group, which can link to the peptide through the amino group, forming an amide bond, while amine containing immune-modulatory compounds can be attached through carbamate functionalities to the benzylic hydroxyl group of the linker (to give a p-amidobenzylcarbamate, PABC). The resulting pro-immune-modulatory compound can be activated upon protease-mediated cleavage, leading to a 1,6-elimination reaction releasing the unmodified immune-modulatory compound, carbon dioxide, and remnants of the linker group. The following scheme depicts the fragmentation of p-amidobenzyl carbamate and release of the immune-modulatory compound:

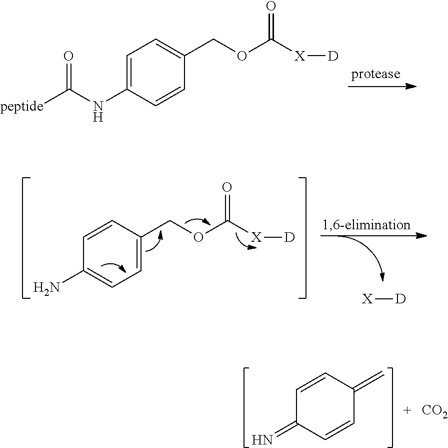

wherein X-D represents the unmodified immune-modulatory compound.

Heterocyclic variants of this self-immolative group have also been described.

The enzymatically cleavable linker can be a ß-glucuronic acid-based linker. Facile release of the immune-modulatory compound can be realized through cleavage of the ß-glucuronide glycosidic bond by the lysosomal enzyme ß-glucuronidase. This enzyme can be abundantly present within lysosomes and can be overexpressed in some tumor types, while the enzyme activity outside cells can be low. ß-Glucuronic acid-based linkers can be used to circumvent the tendency of an antibody construct immune-modulatory compound conjugate to undergo aggregation due to the hydrophilic nature of ß-glucuronides. In certain embodiments, ß-glucuronic acid-based linkers can link an antibody construct to a hydrophobic immune-modulatory compound. The following scheme depicts the release of an immune-modulatory compound (D) from an antibody construct (Ab) immune-modulatory compound conjugate containing a ß-glucuronic acid-based linker:

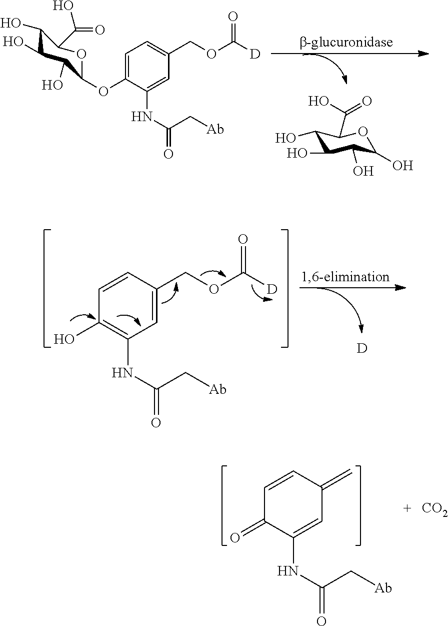

A variety of cleavable β-glucuronic acid-based linkers useful for linking drugs such as auristatins, camptothecin and doxorubicin analogues, CBI minor-groove binders, and psymberin to antibodies have been described. These β-glucuronic acid-based linkers may be used in the conjugates described herein. In certain embodiments, the enzymatically cleavable linker is a β-galactoside-based linker. β-Galactoside is present abundantly within lysosomes, while the enzyme activity outside cells is low.

Additionally, compounds containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on an immune-modulatory compound, wherein such ester groups can hydrolyze under physiological conditions to release the immune-modulatory compound. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can contain an enzymatically cleavable peptide moiety, for example, a linker comprising structural formula (IIIa), (IIIb), (IIIc), or (IIId):

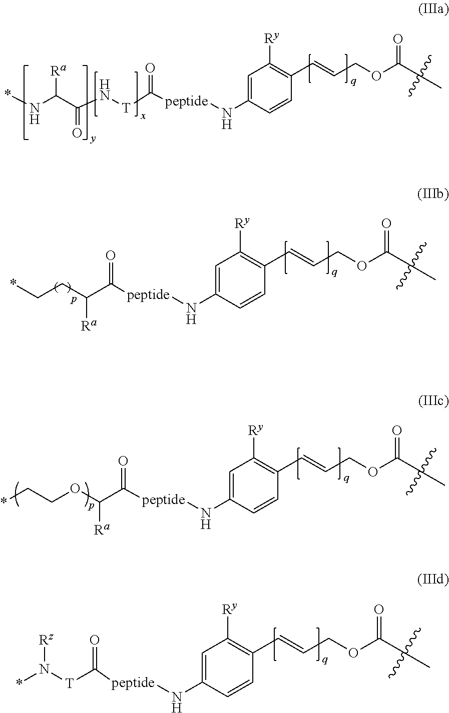

or a salt thereof, wherein: peptide represents a peptide (illustrated N→C, wherein peptide includes the amino and carboxy “termini”) cleavable by a lysosomal enzyme; T represents a polymer comprising one or more ethylene glycol units or an alkylene chain, or combinations thereof; R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂; R^{z} is C₁₋₄ alkyl-(O)ᵣ—(C₁₋₄ alkylene)ₛ-G²; G¹ is SO₃H, CO₂H, PEG 4-32, or sugar moiety; G² is SO₃H, CO₂H, or PEG 4-32 moiety; r is 0 or 1; s is 0 or 1; p is an integer ranging from 0 to 5; q is 0 or 1; x is 0 or 1; y is 0 or 1; [symbol] represents the point of attachment of the linker to an immune-modulatory compound or salt thereof; and * represents the point of attachment to the remainder of the linker.

In certain embodiments, the peptide can be selected from a tripeptide or a dipeptide. In particular embodiments, the dipeptide can be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (IIIa) that can be included in the conjugates described herein can include the linkers illustrated below (as illustrated, the linkers include a group suitable for covalently linking the linker to an antibody construct):

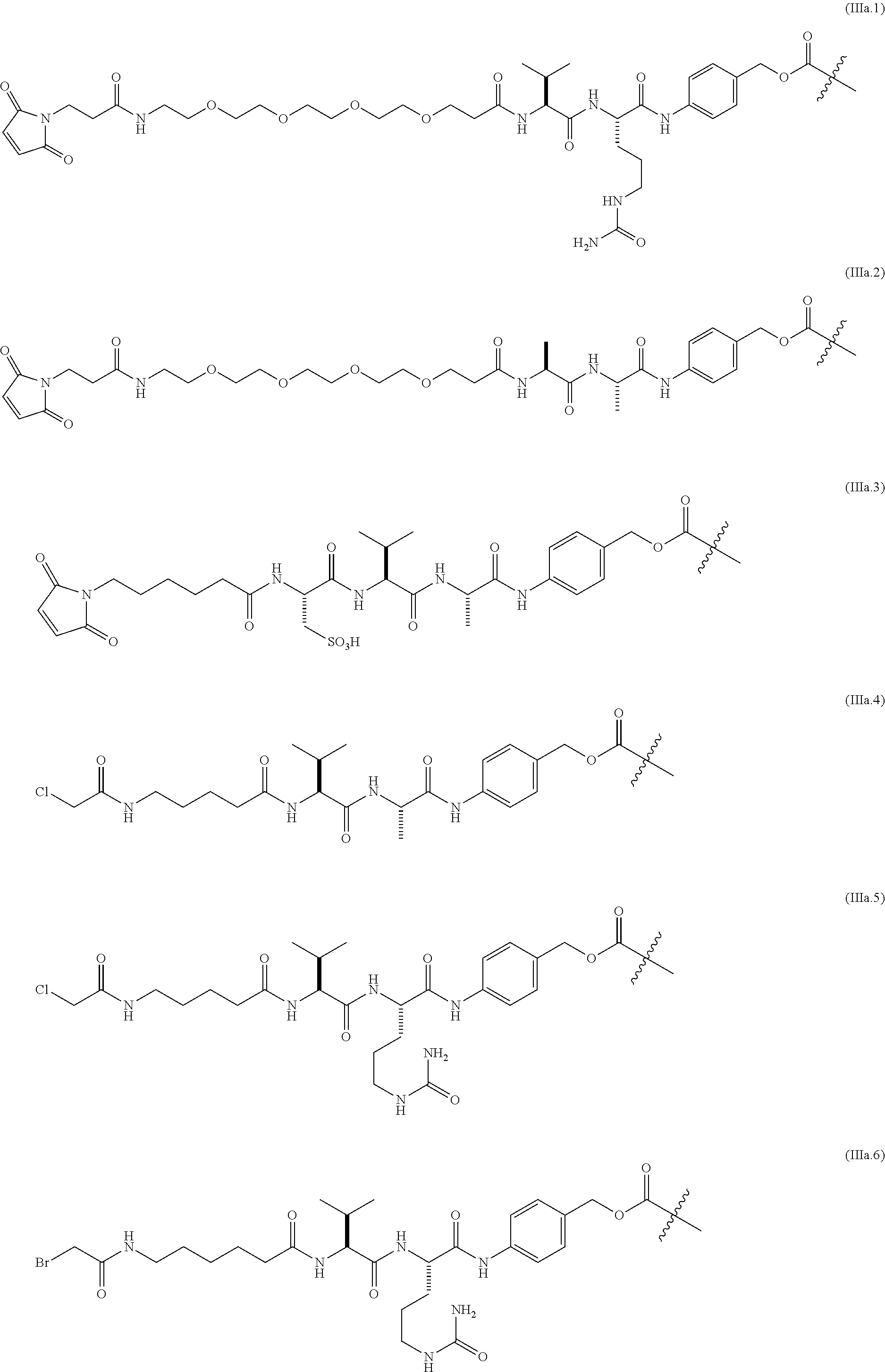

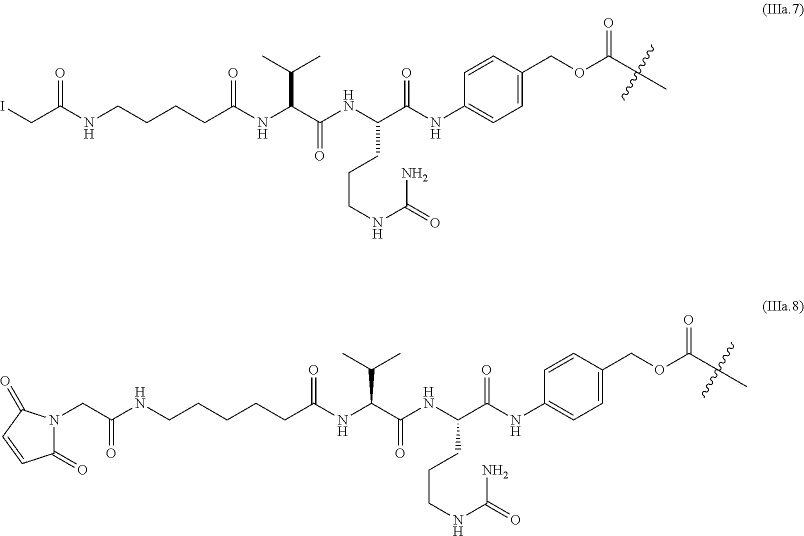

wherein [symbol] represents the point of attachment of the linker to an immune-modulatory compound or salt thereof.

Exemplary embodiments of linkers according to structural formula (IIIb), (IIIc), or (IIId) that can be included in the conjugates described herein can include the linkers illustrated below (as illustrated, the linkers can include a group suitable for covalently linking the linker to an antibody construct):

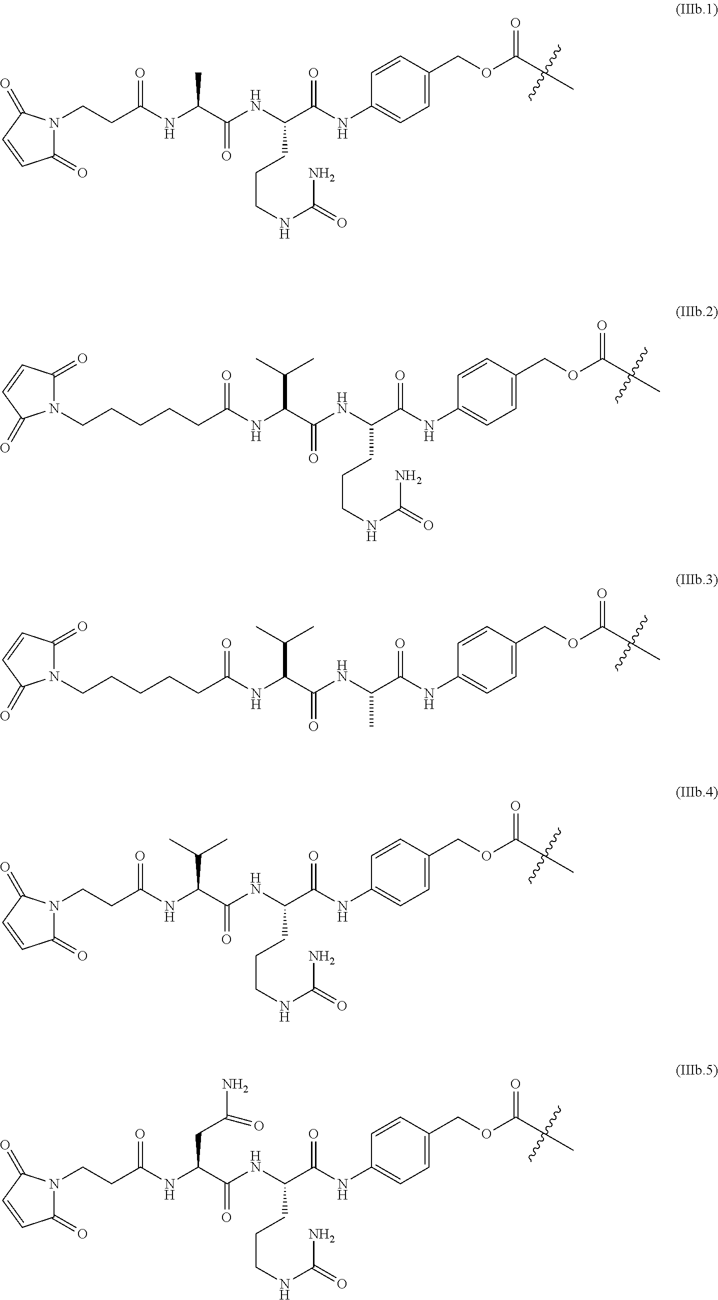

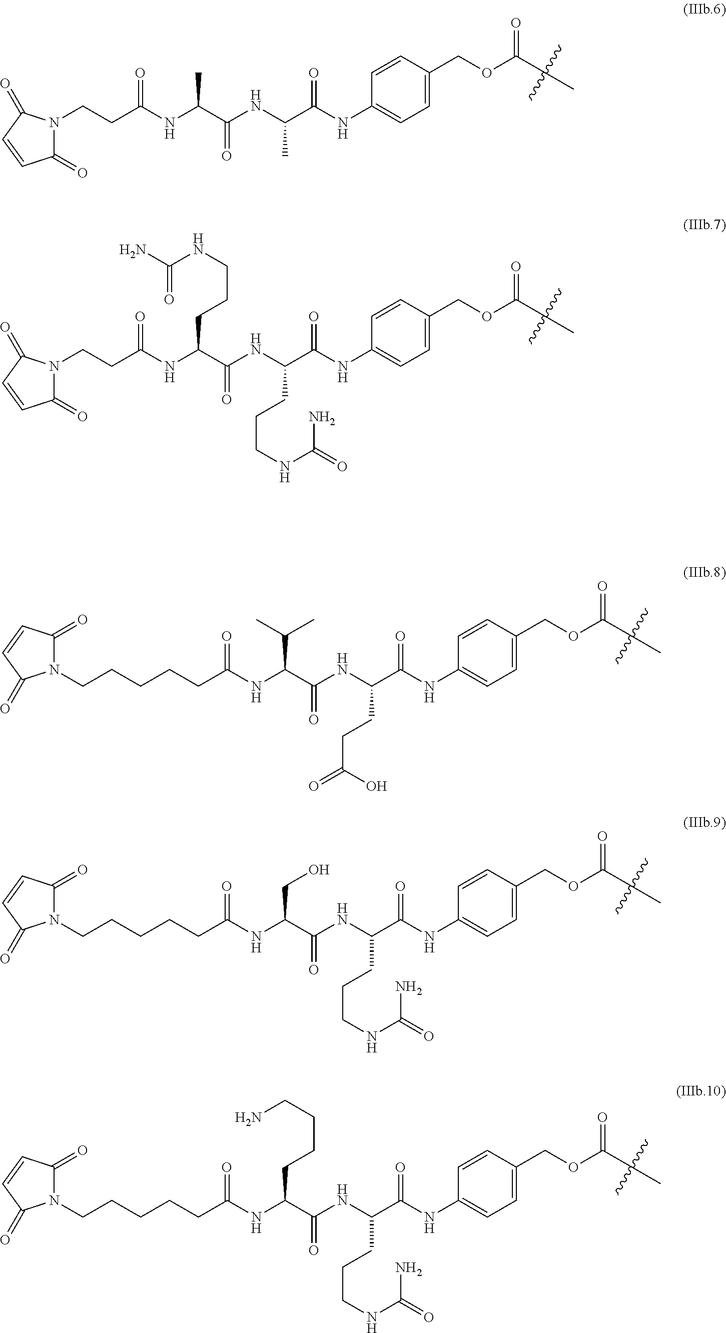

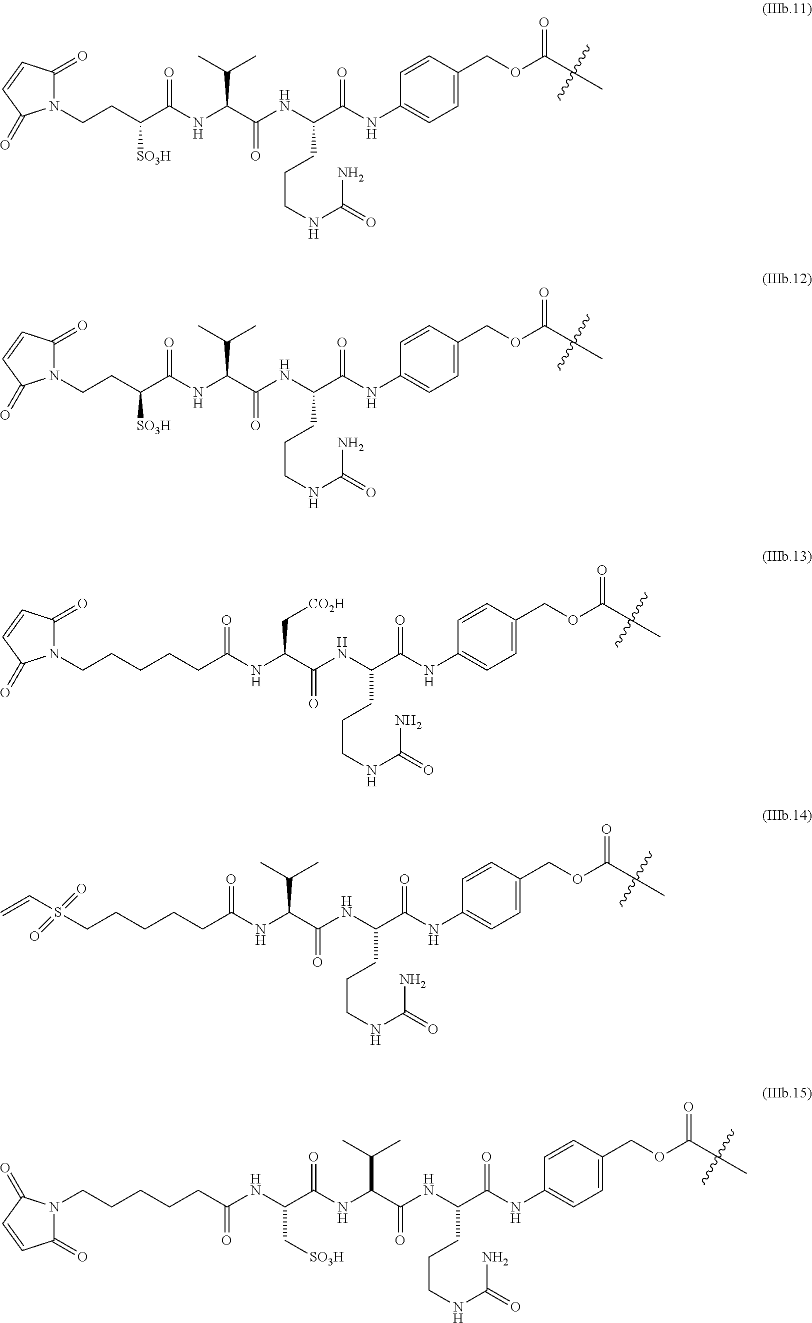

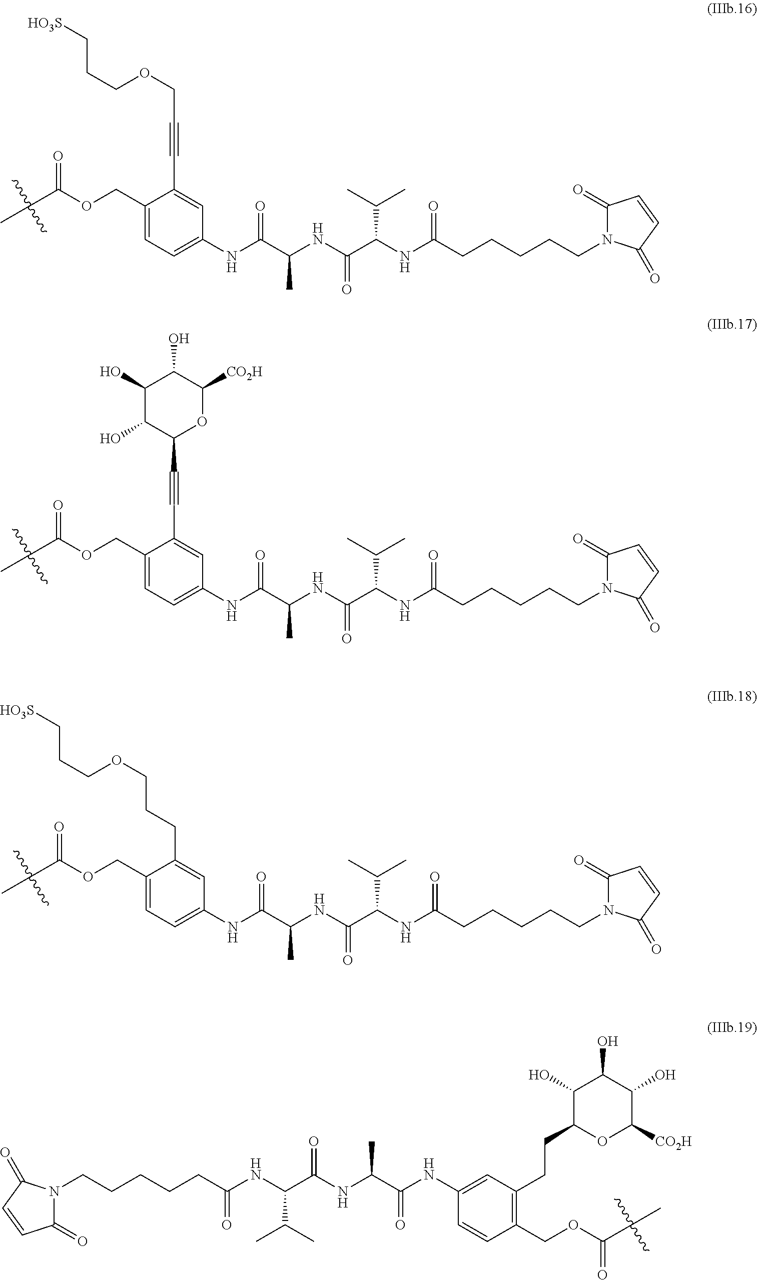

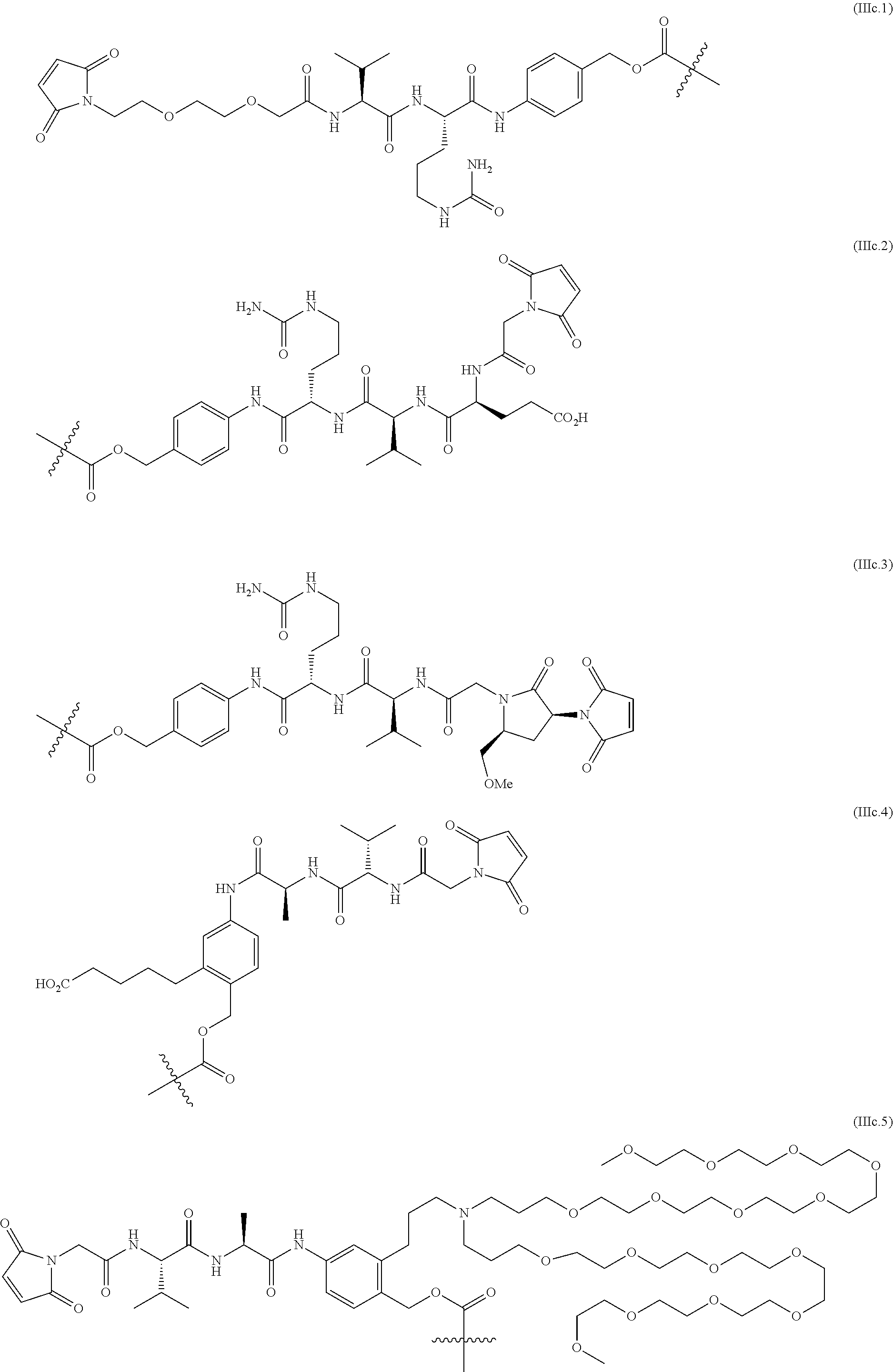

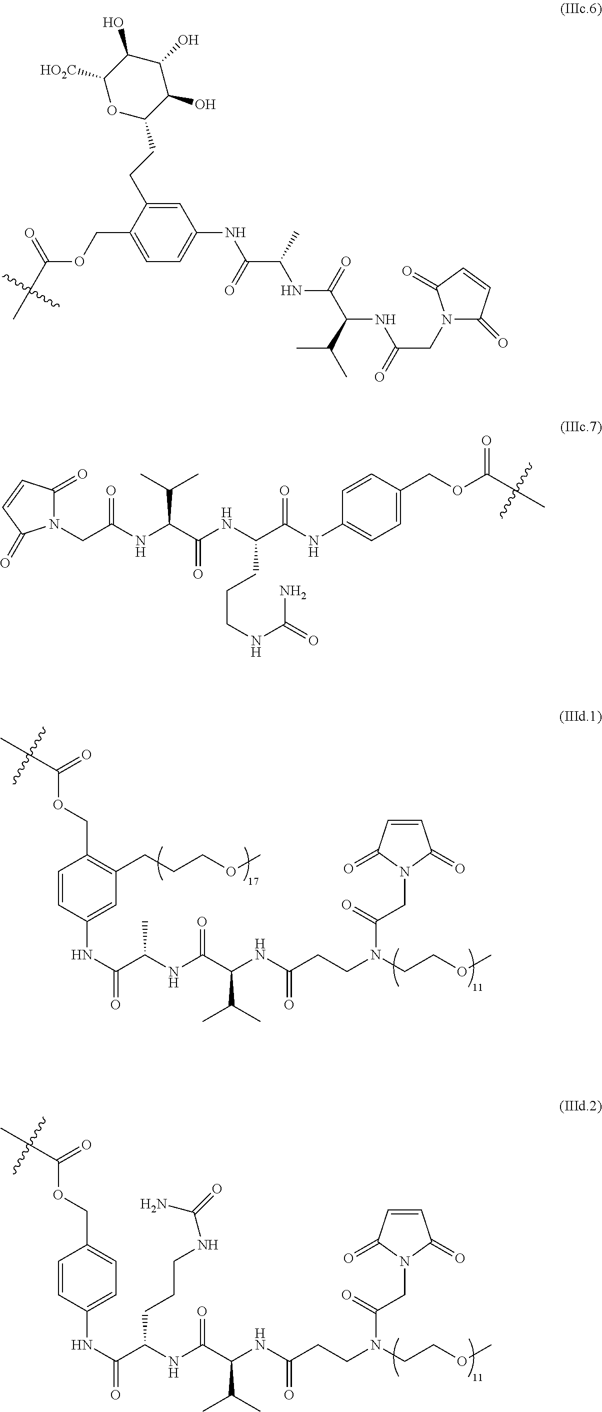

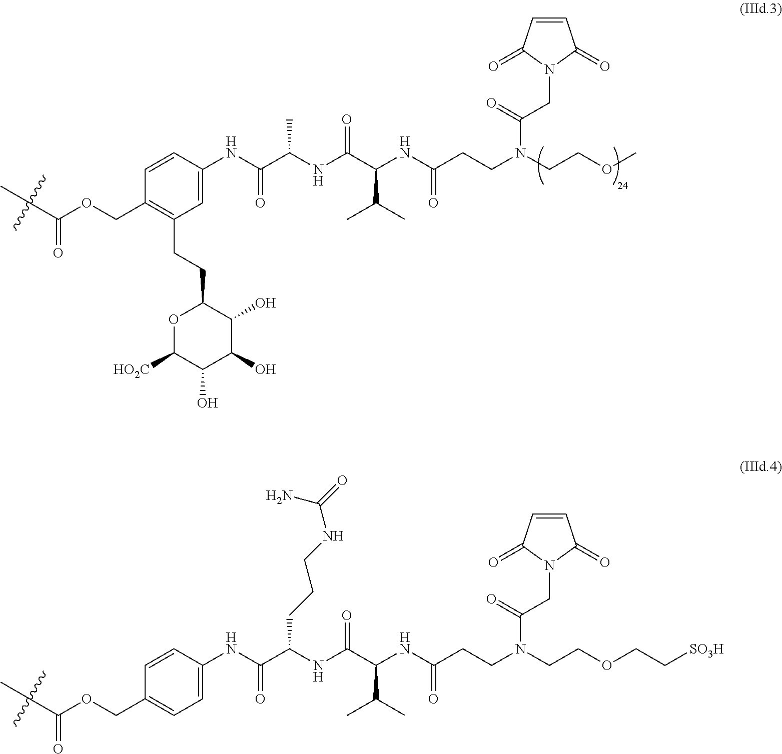

wherein [symbol] represents the point of attachment of the linker to an immune-modulatory compound or salt thereof.

The linker can contain an enzymatically cleavable sugar moiety, for example, a linker comprising structural formula (IVa), (IVb), (IVc), (IVd), or (IVe):

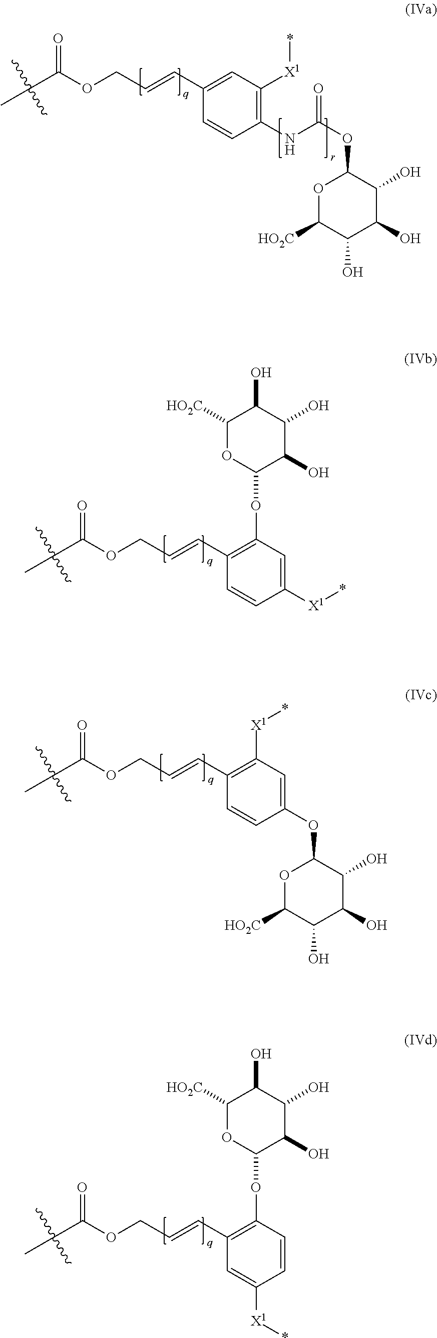

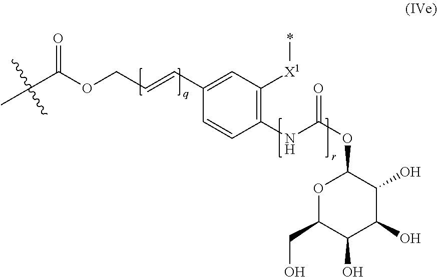

or a salt thereof, wherein: q is 0 or 1; r is 0 or 1; X¹ is CH₂, O, or NH; [symbol] represents the point of attachment of the linker to an immune-modulatory compound or salt thereof; and * represents the point of attachment to the remainder of the linker.

Exemplary embodiments of linkers according to structural formula (IVa) that may be included in the antibody construct immune-modulatory compound conjugates described herein can include the linkers illustrated below (as illustrated, the linkers include a group suitable for covalently linking the linker to an antibody construct):

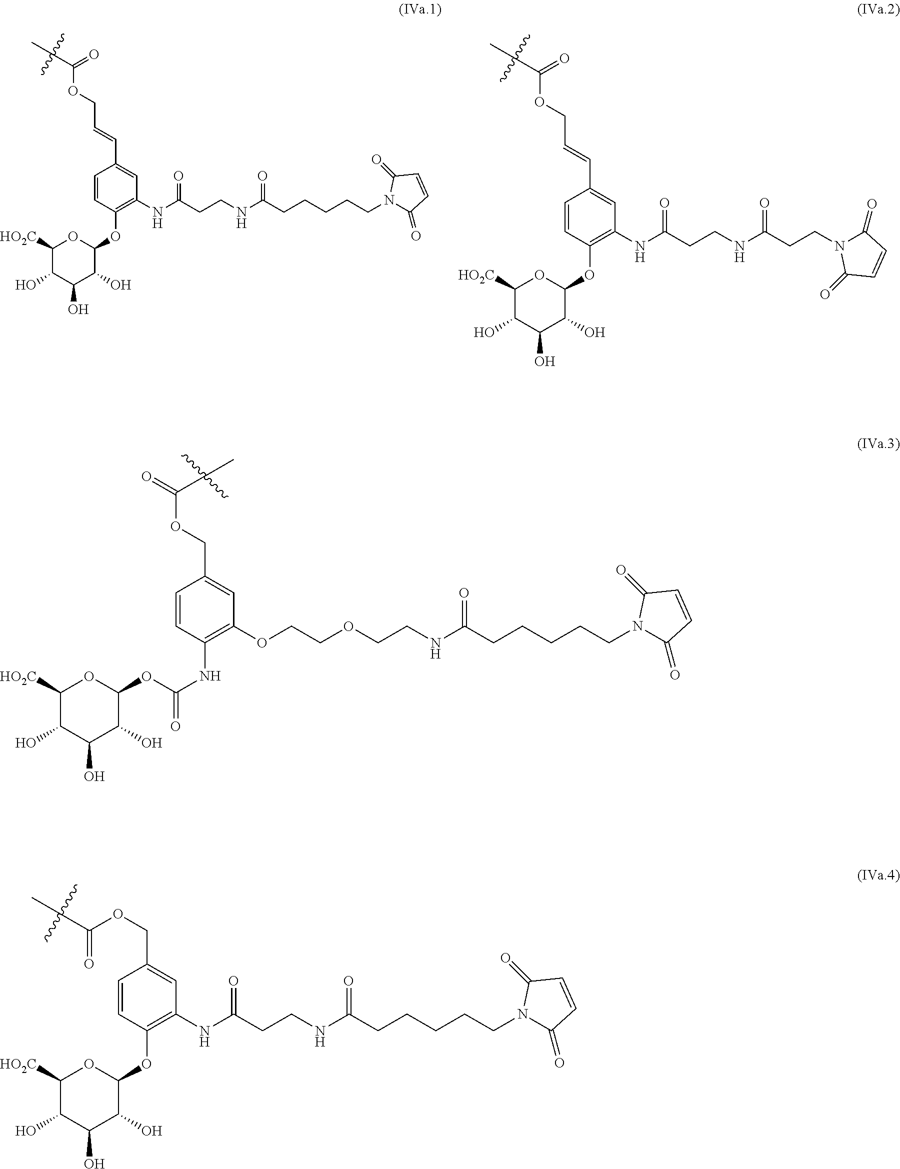

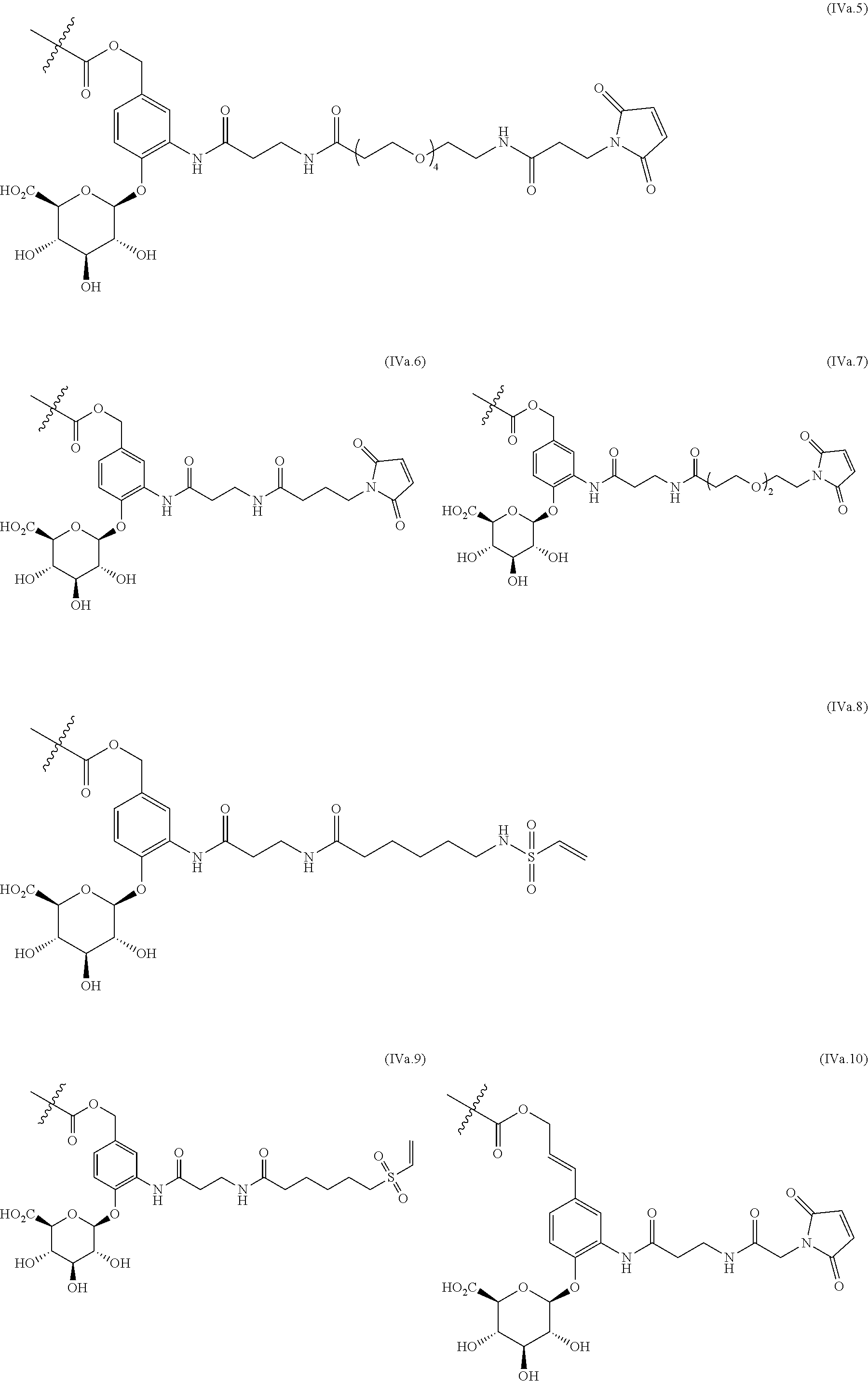

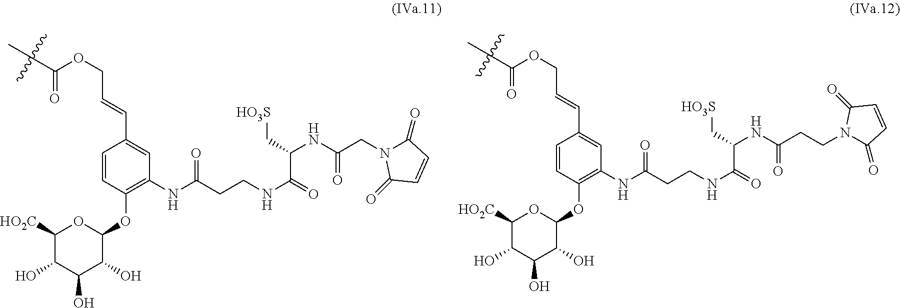

wherein [symbol] represents the point of attachment of the linker to an immune-modulatory compound or salt thereof.

Exemplary embodiments of linkers according to structural formula (IVb) that may be included in the conjugates described herein include the linkers illustrated below (as illustrated, the linkers include a group suitable for covalently linking the linker to an antibody construct):

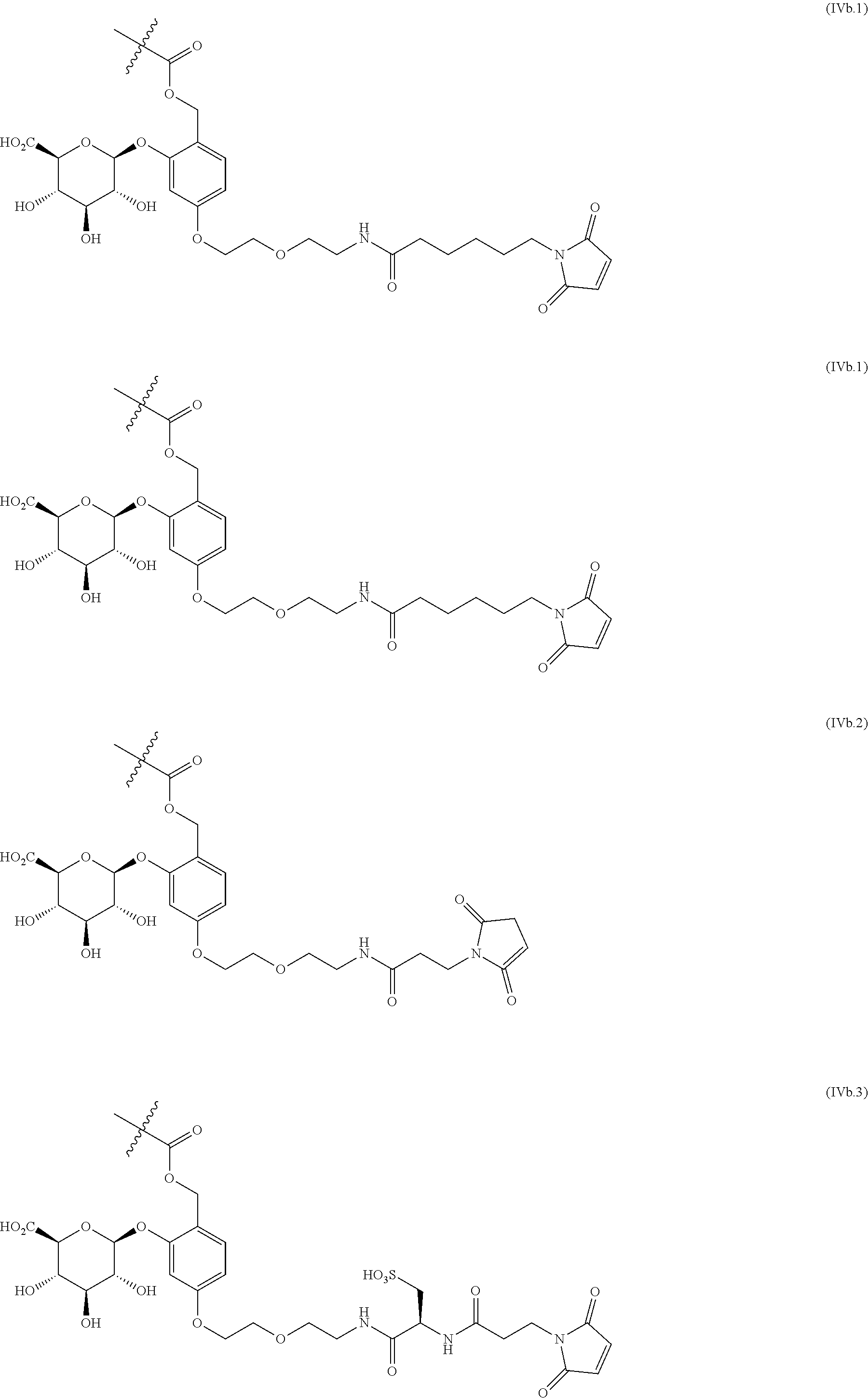

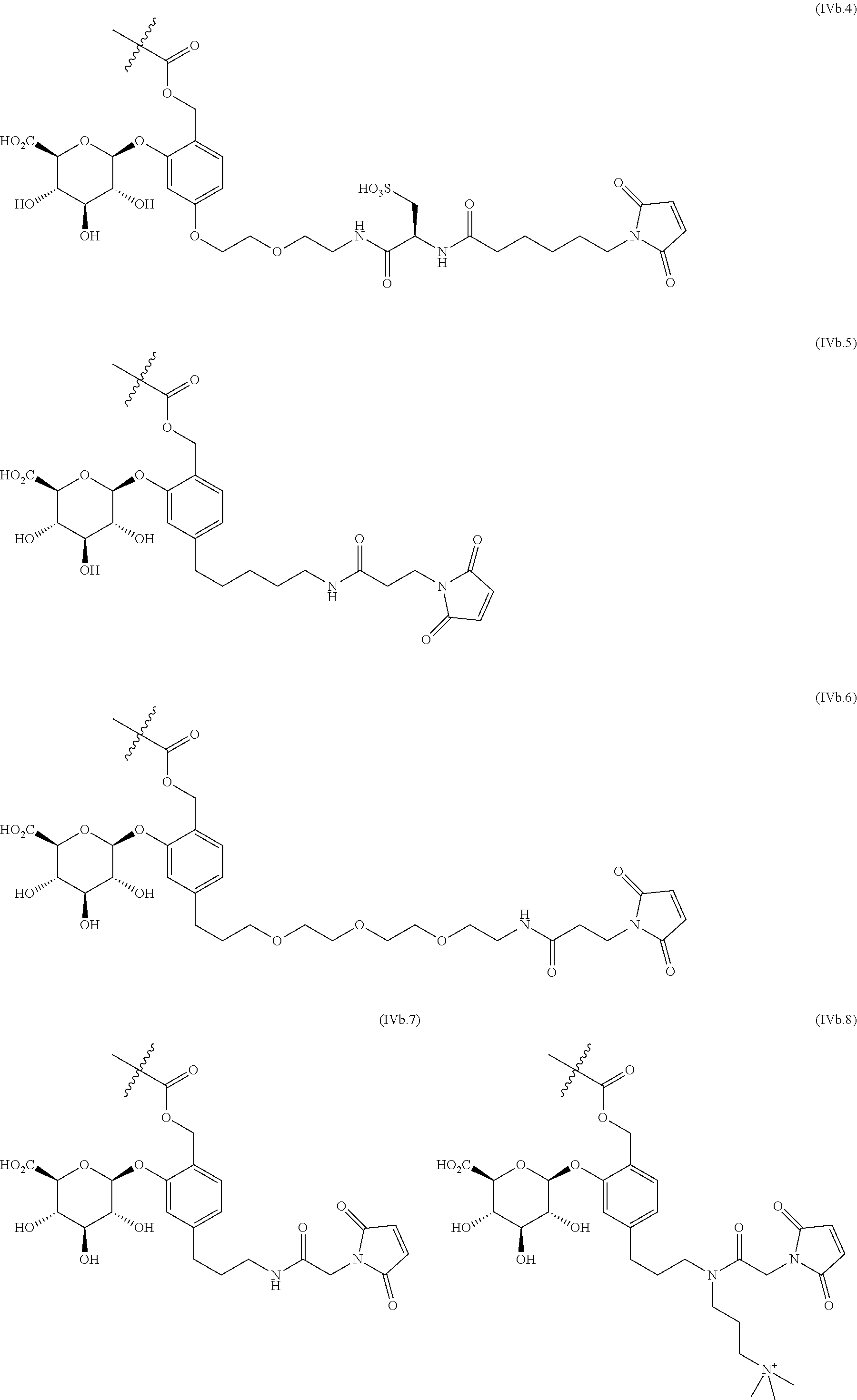

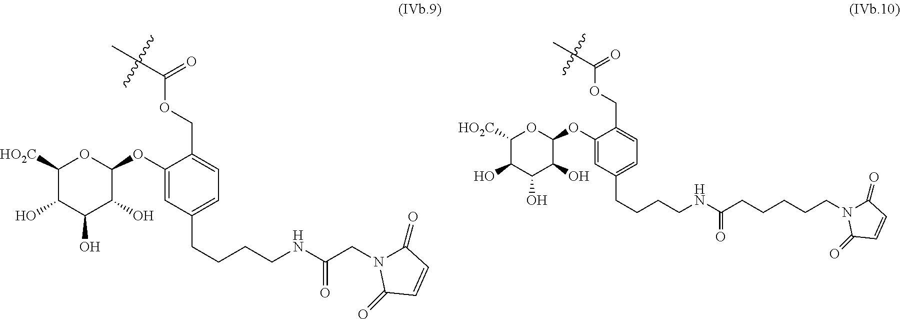

wherein [symbol] represents the point of attachment of the linker to an immune-modulatory compound or salt thereof.

Exemplary embodiments of linkers according to structural formula (IVc) that may be included in the conjugates described herein include the linkers illustrated below (as illustrated, the linkers include a group suitable for covalently linking the linker to an antibody construct):

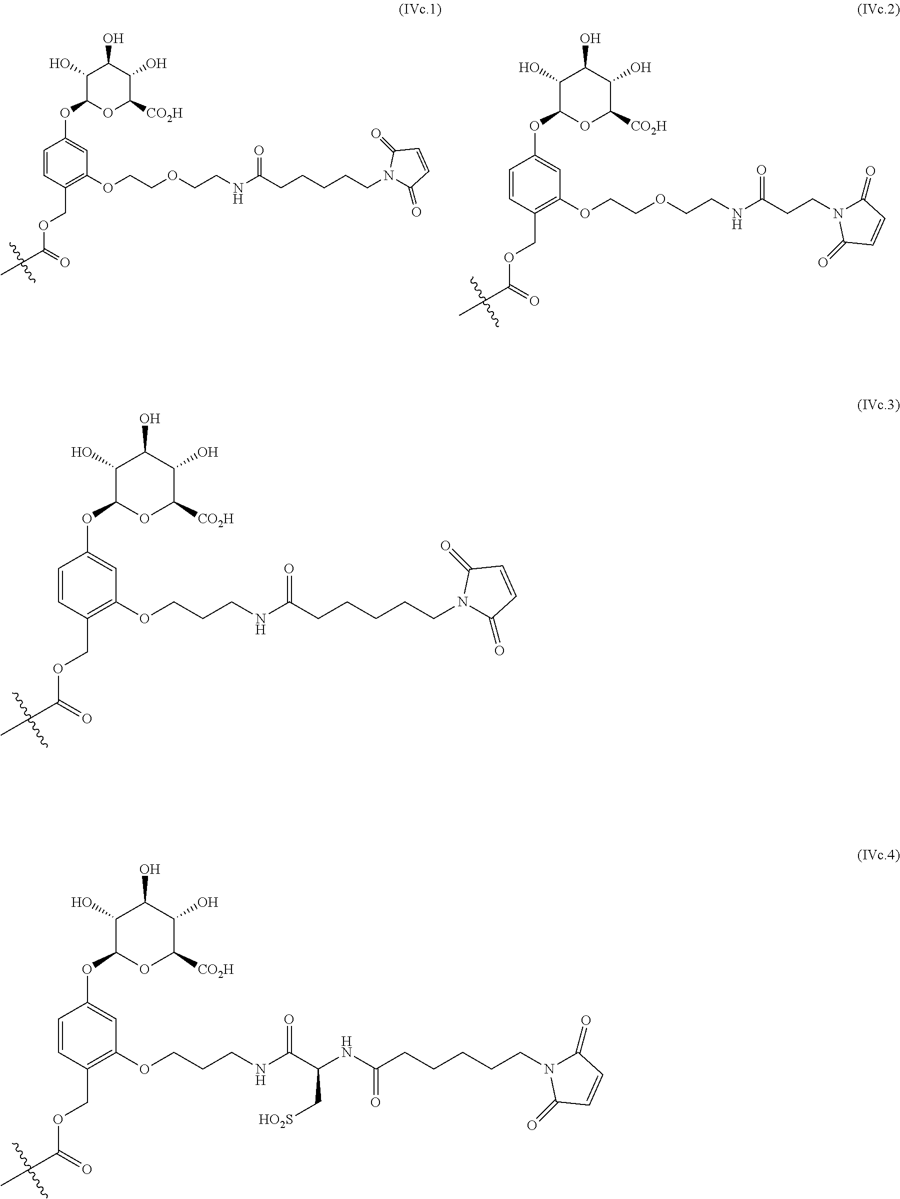

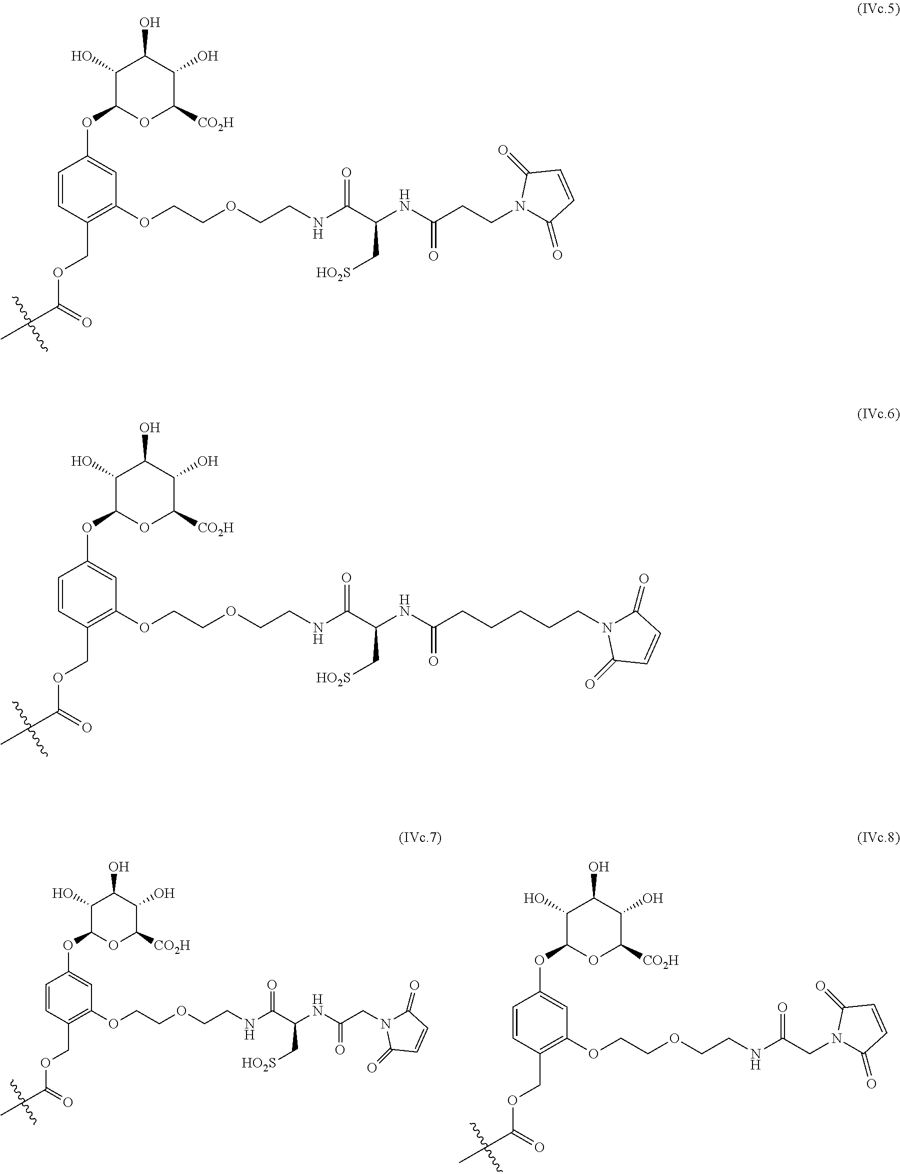

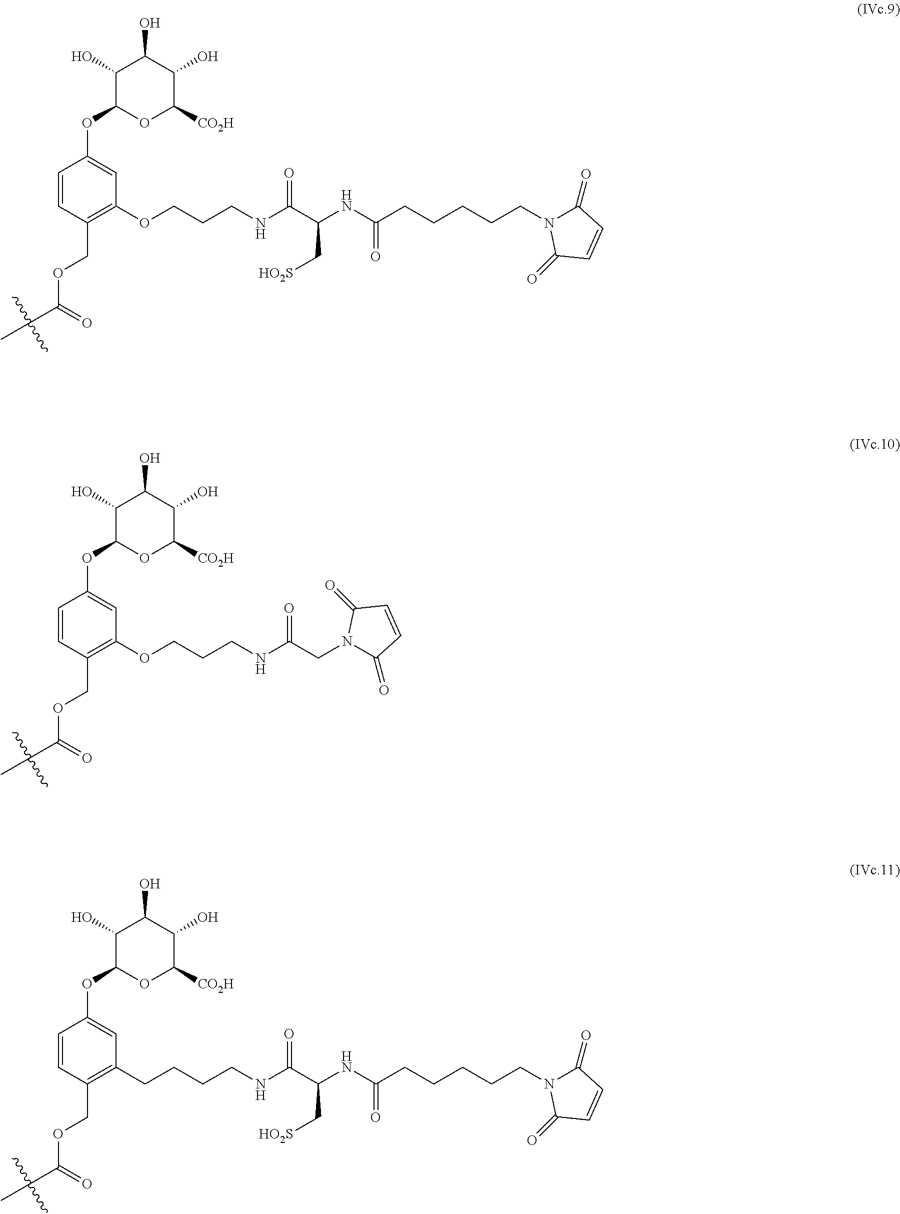

wherein [symbol] represents the point of attachment of the linker to an immune-modulatory compound or salt thereof.

Exemplary embodiments of linkers according to structural formula (IVd) that may be included in the conjugates described herein include the linkers illustrated below (as illustrated, the linkers include a group suitable for covalently linking the linker to an antibody):

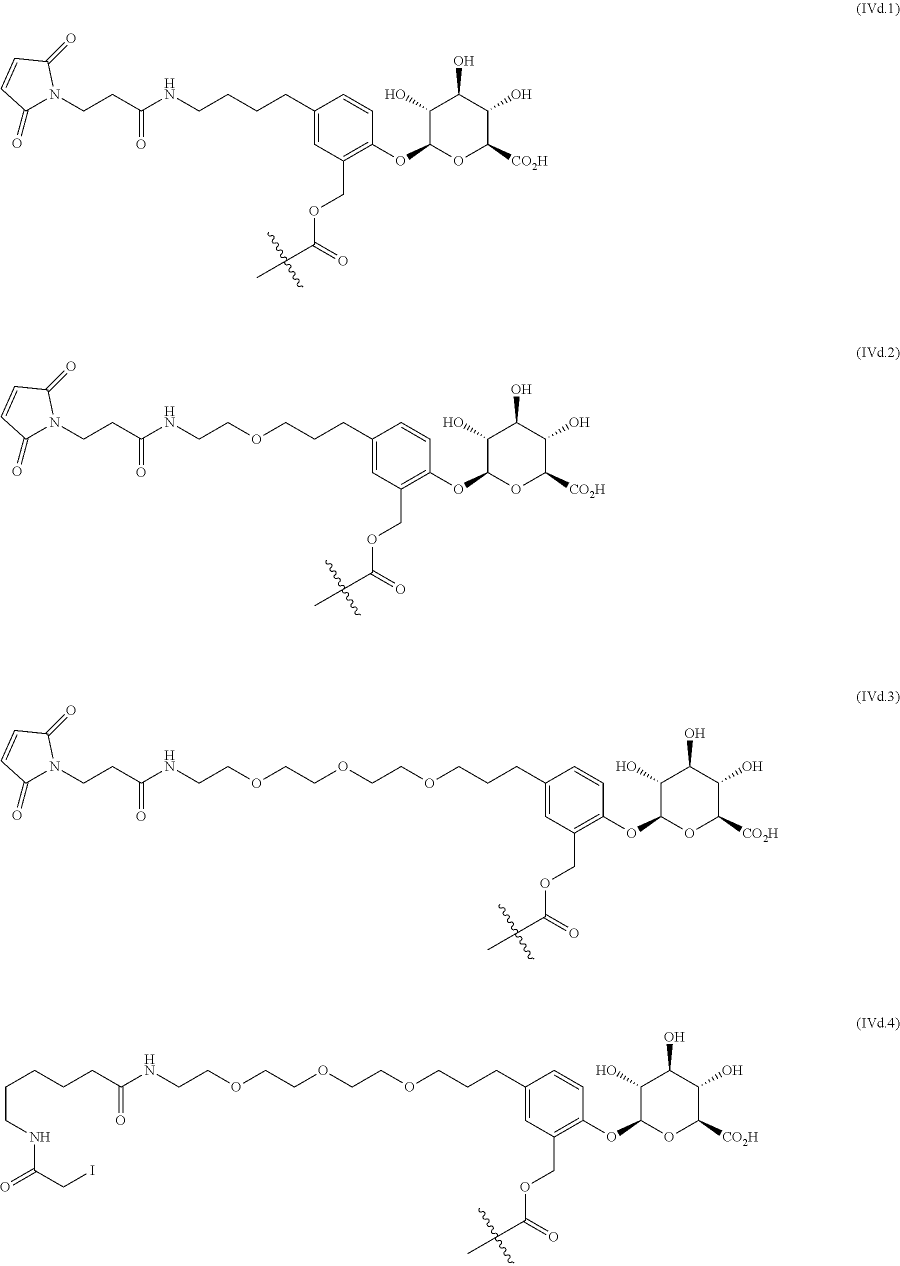

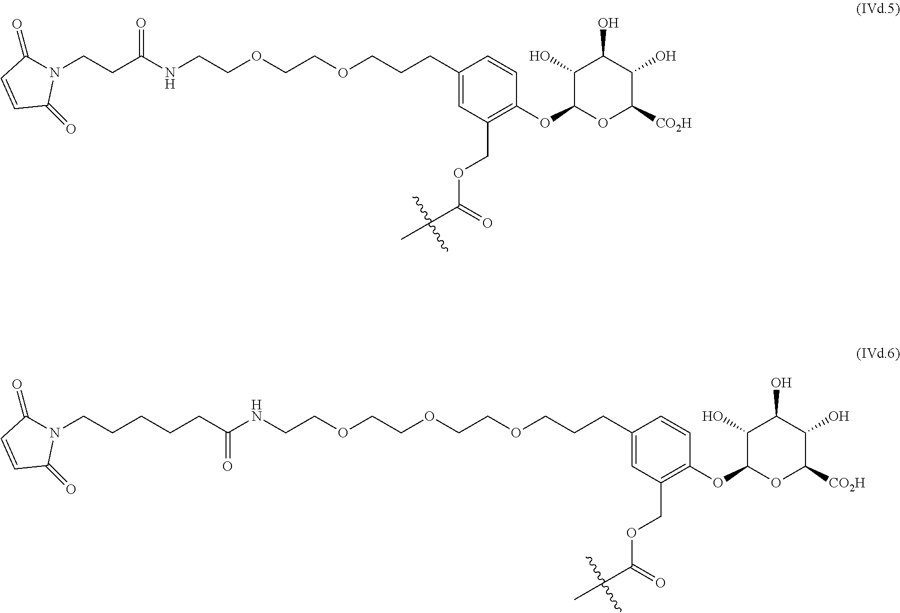

wherein [symbol] represents the point of attachment of the linker to an immune-modulatory compound or salt thereof.

Exemplary embodiments of linkers according to structural formula (IVe) that may be included in the conjugates described herein include the linkers illustrated below (as illustrated, the linkers include a group suitable for covalently linking the linker to an antibody construct):

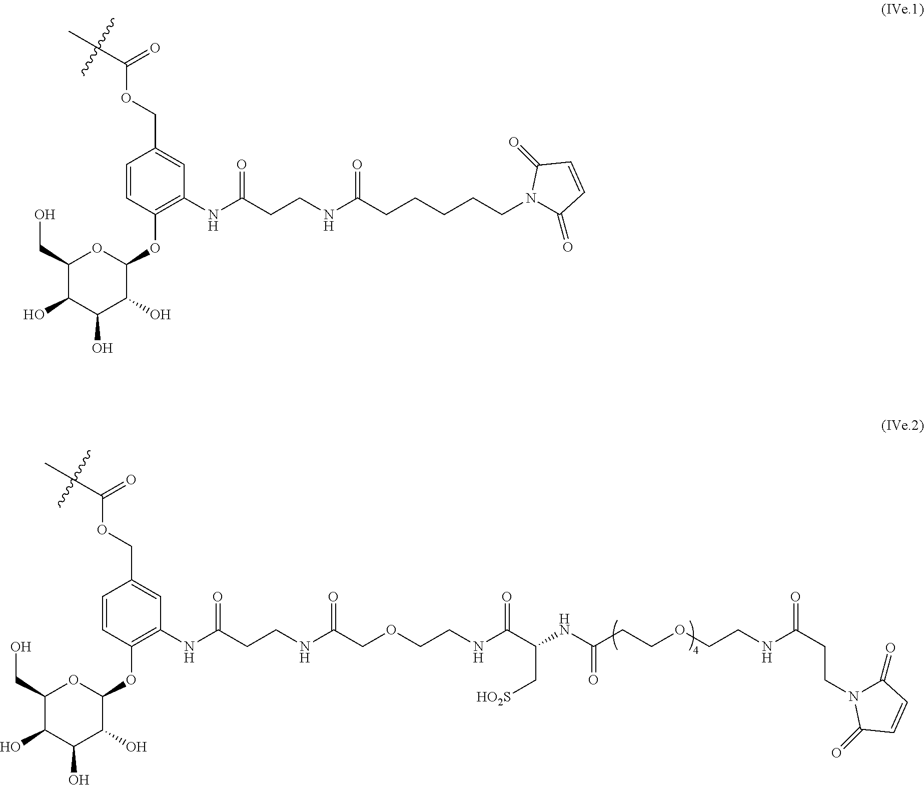

wherein [symbol] represents the point of attachment of the linker to an immune-modulatory compound or salt thereof.

Although cleavable linkers can provide certain advantages, the linkers included in the conjugates described herein need not be cleavable. For non-cleavable linkers, the immune-modulatory compound release may not depend on the differential properties between the plasma and some cytoplasmic compartments. The release of the immune-modulatory compound can occur after internalization of the antibody construct immune-modulatory compound conjugate via antigen-mediated endocytosis and delivery to lysosomal compartment, where the antibody construct can be degraded to the level of amino acids through intracellular proteolytic degradation. This process can release an immune-modulatory compound derivative, which is formed by the immune-modulatory compound, the linker, and the amino acid residue to which the linker was covalently attached. The immune-modulatory compound derivative from antibody construct immune-modulatory compound conjugate with non-cleavable linkers can be more hydrophilic and less membrane permeable, which can lead to less bystander effects and less nonspecific toxicities compared to antibody construct immune-modulatory compound conjugates with a cleavable linker. Antibody construct immune-modulatory compound conjugates with non-cleavable linkers can have greater stability in circulation than antibody construct immune-modulatory compound conjugates with cleavable linkers. Non-cleavable linkers can be alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker according to the formulations below:

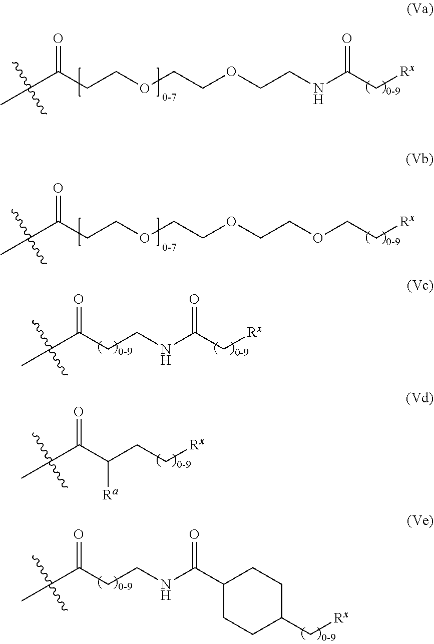

or salts thereof, wherein: R^{a} is selected from hydrogen, alkyl, sulfonate and methyl sulfonate; R^{x} is a moiety including a functional group capable of covalently linking the linker to an antibody construct; and [symbol] represents the point of attachment of the linker to an immune-modulatory compound or salt thereof.

Exemplary embodiments of linkers according to structural formula (Va)-(Ve) that may be included in the conjugates described herein include the linkers illustrated below (as illustrated, the linkers include a group suitable for covalently linking the linker to an antibody construct, and [symbol] represents the point of attachment to an immune-modulatory compound or salt thereof:

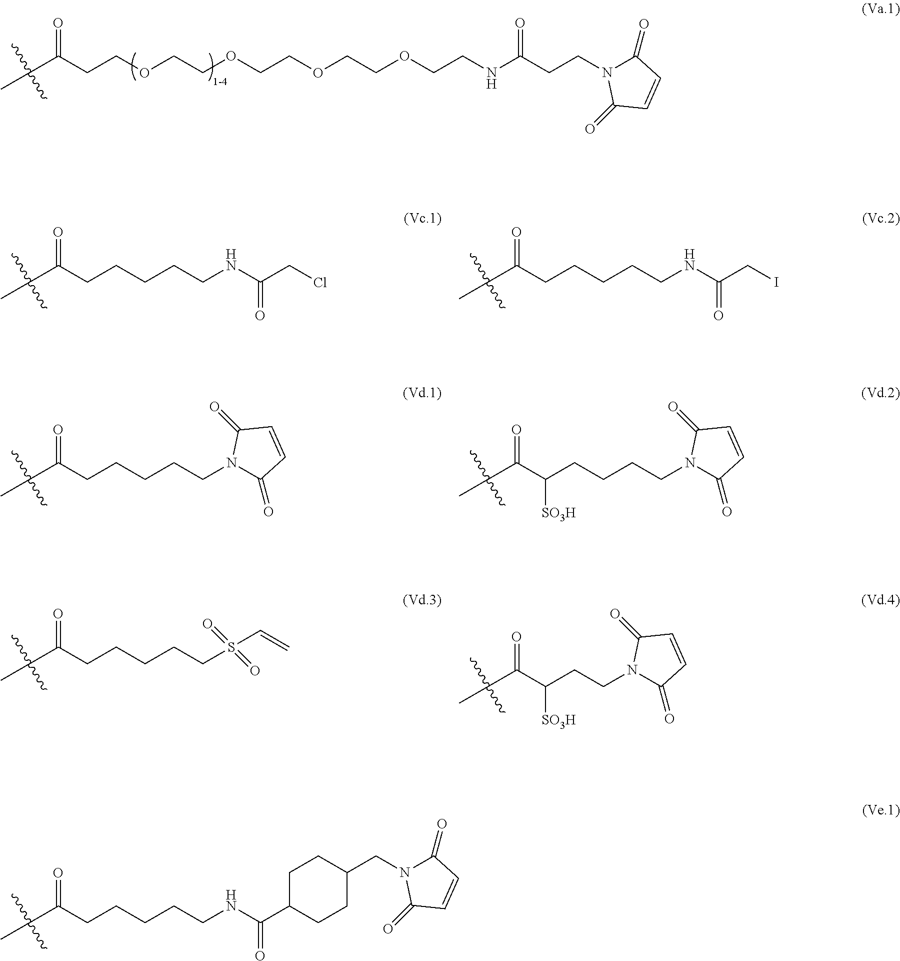

wherein [symbol] represents time point of attachment of the linker to an immune-modulatory compound or salt thereof.

Attachment groups that are used to attach the linkers to an antibody can be electrophilic in nature and include, for example, maleimide groups, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

One example of a “self-stabilizing” maleimide group that hydrolyzes spontaneously under antibody conjugation conditions to give a conjugate with improved stability is depicted in the schematic below. Thus, the maleimide attachment group is reacted with a sulfhydryl of an antibody to give an intermediate succinimide ring. The hydrolyzed form of the attachment group is resistant to deconjugation in the presence of plasma proteins.

### Normal System:

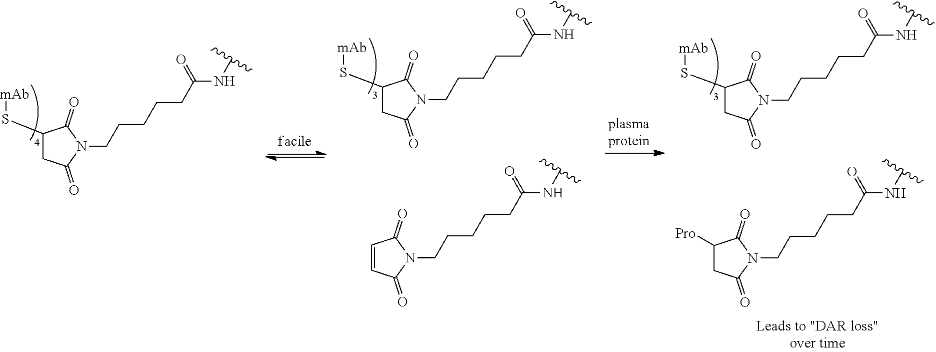

### Self-Stabilizing Attachment:

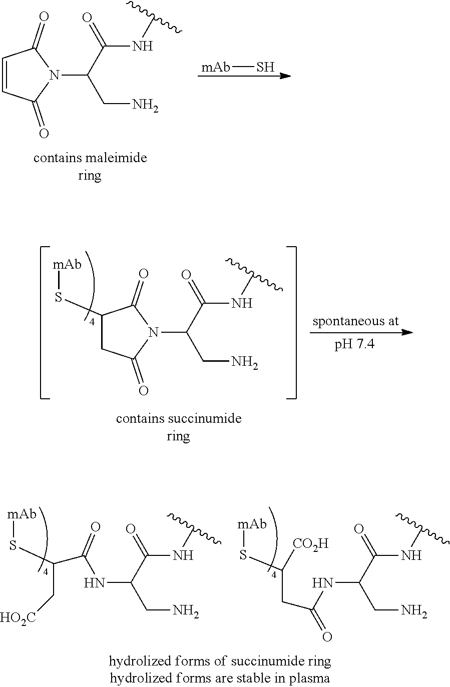

A method for bridging a pair of sulfhydryl groups derived from reduction of a native hinge disulfide bond has been disclosed and is depicted in the schematic below. An advantage of this methodology is the ability to synthesize homogenous DAR4 conjugates by full reduction of IgGs (to give 4 pairs of sulfhydryls) followed by reaction with 4 equivalents of the alkylating agent. Conjugates containing “bridged disulfides” are also claimed to have increased stability.

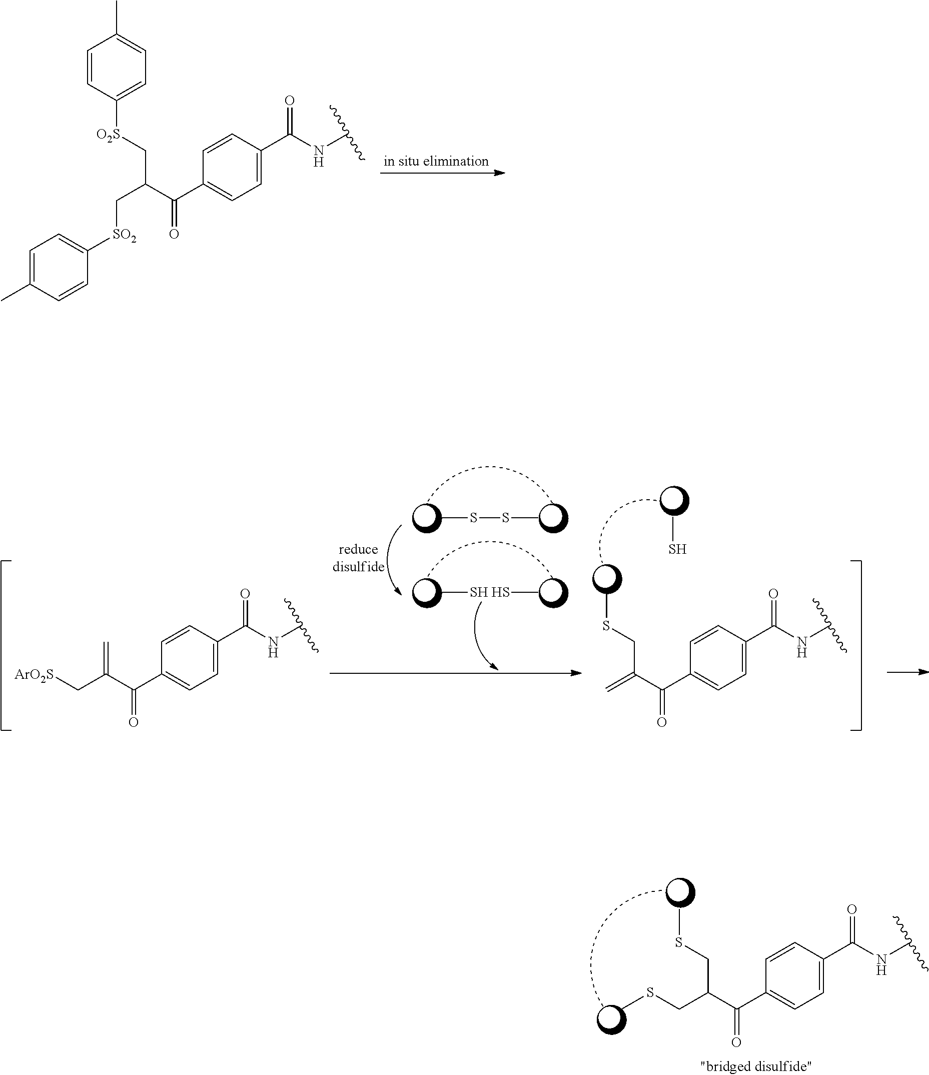

Similarly, as depicted below, a maleimide derivative that is capable of bridging a pair of sulfhydryl groups has been developed.

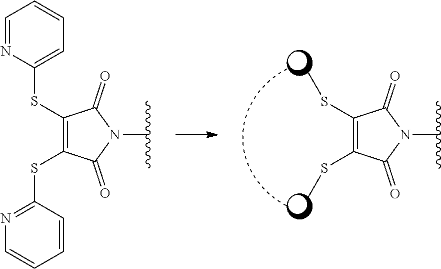 [SMILES: *N1C(=O)C(Sc2ccccn2)=C(Sc2ccccn2)C1=O ; *N1C(=O)C(SC)=C(SC)C1=O]

The attachment moiety can contain the following structural formulas (VIa), (VIb), or (VIc):

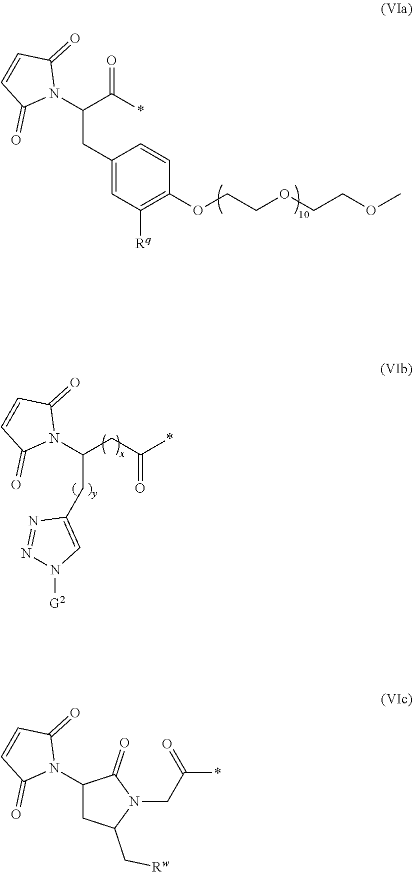

or salts thereof, wherein: R^{q} is H or —O—(CH₂CH₂O)₁₁—CH₃; x is 0 or 1; y is 0 or 1; G² is-CH₂CH₂CH₂SO₃H or —CH₂CH₂O—(CH₂CH₂O)₁₁—CH₃; R^{W} is-O—CH₂CH₂SO₃H or —NH(CO)—CH₂CH₂O—(CH₂CH₂O)₁₂—CH₃; and * represents the point of attachment to the remainder of the linker.

Exemplary embodiments of linkers according to structural formula (VIa) and (VIb) that can be included in the conjugates described herein can include the linkers illustrated below (as illustrated, the linkers can include a group suitable for covalently linking the linker to an antibody construct):

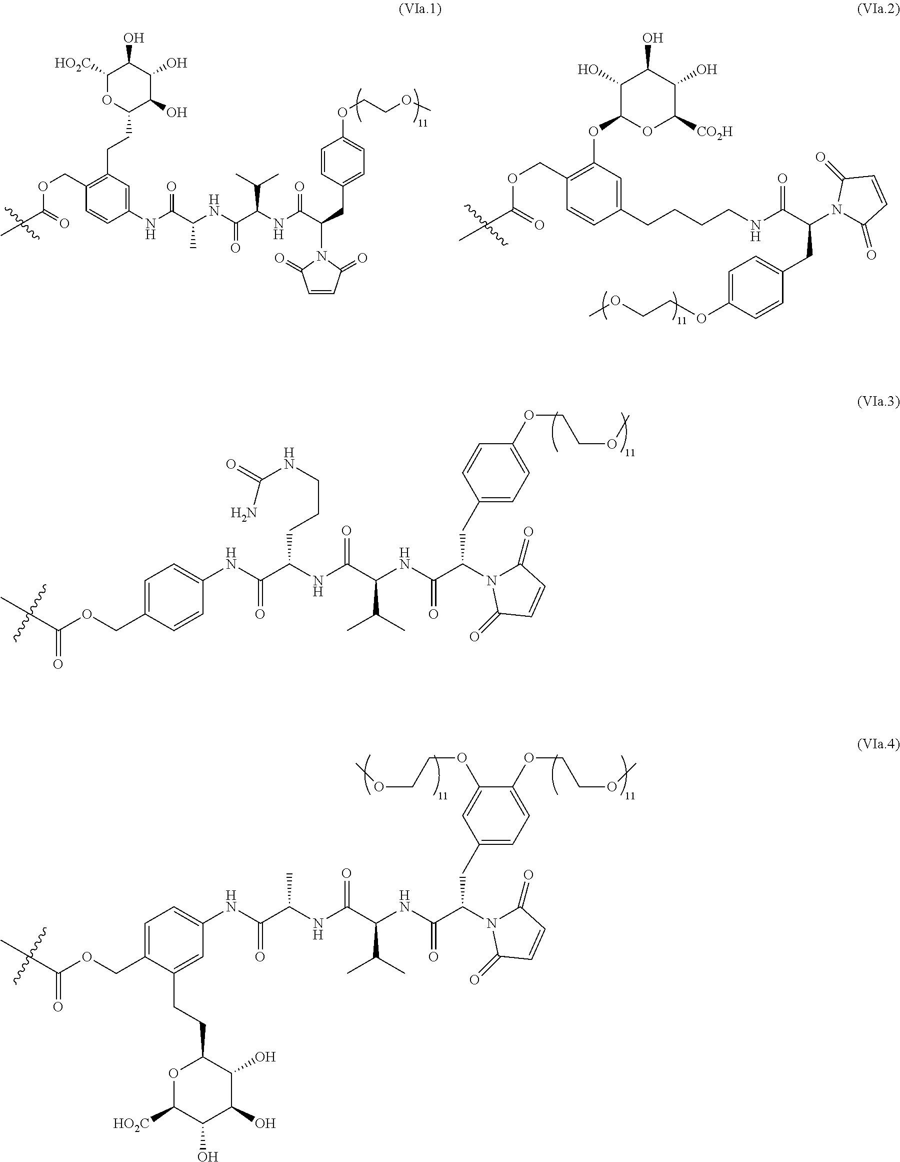

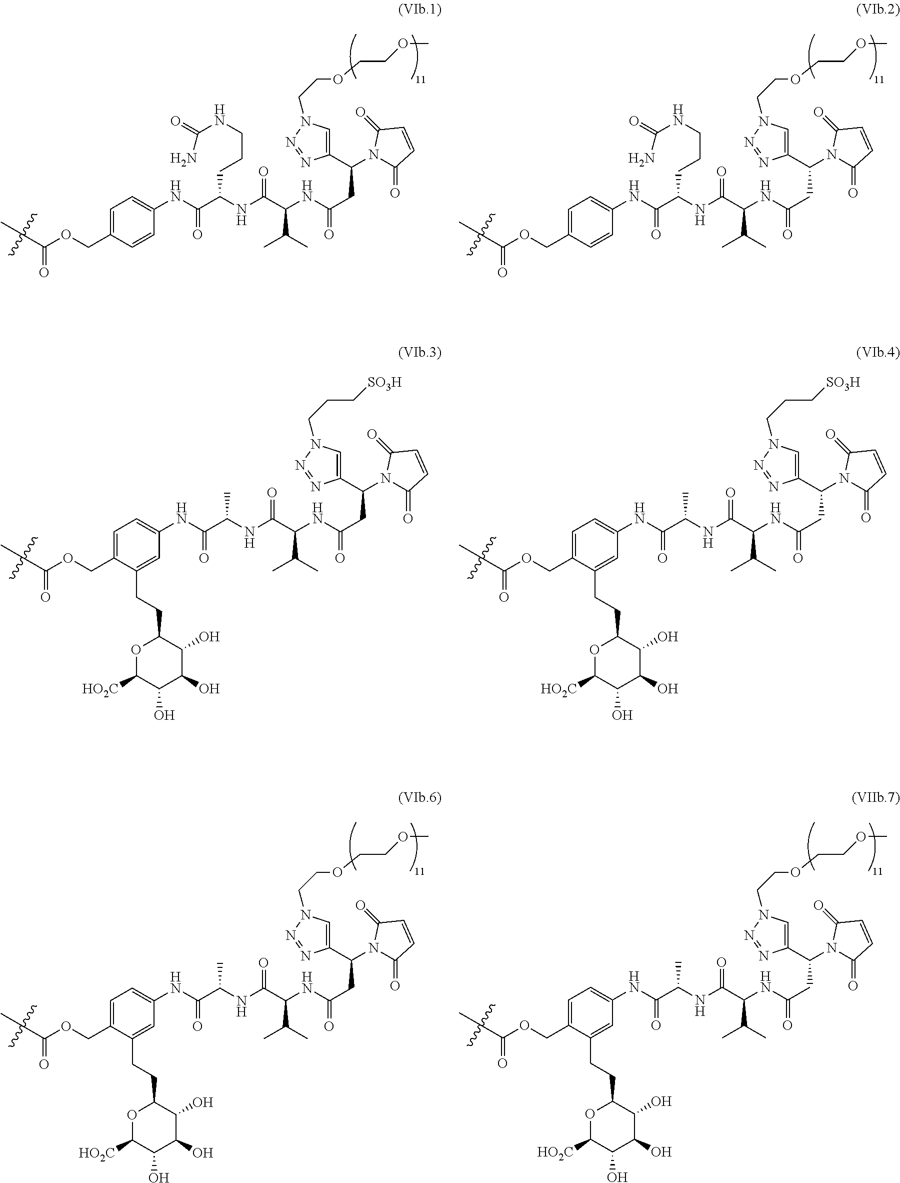

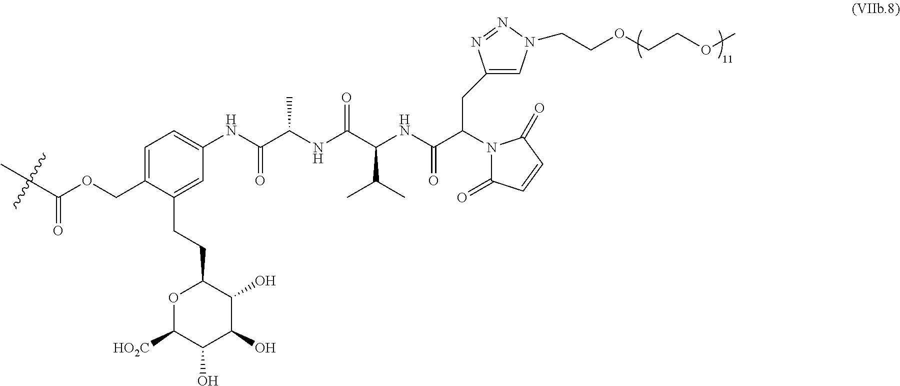

wherein [symbol] represents the point of attachment of the linker to an immune-modulatory compound or salt thereof.

Exemplary embodiments of linkers according to structural formula (VIc) that can be included in the antibody construct immune-modulatory compound conjugate described herein can include the linkers illustrated below (as illustrated, the linkers can include a group suitable for covalently linking the linker to an antibody construct):

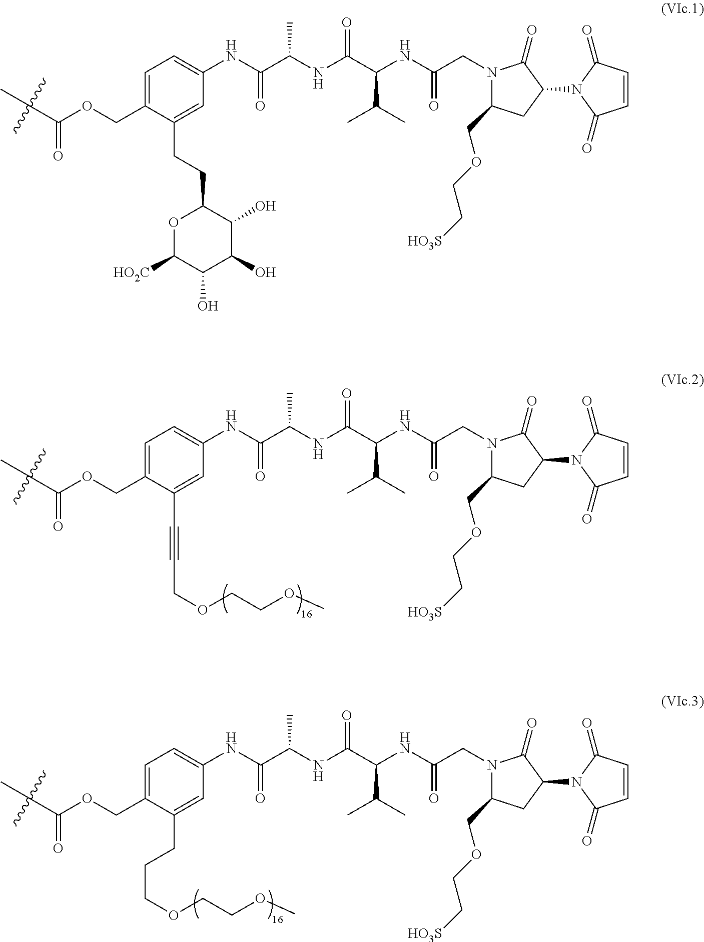

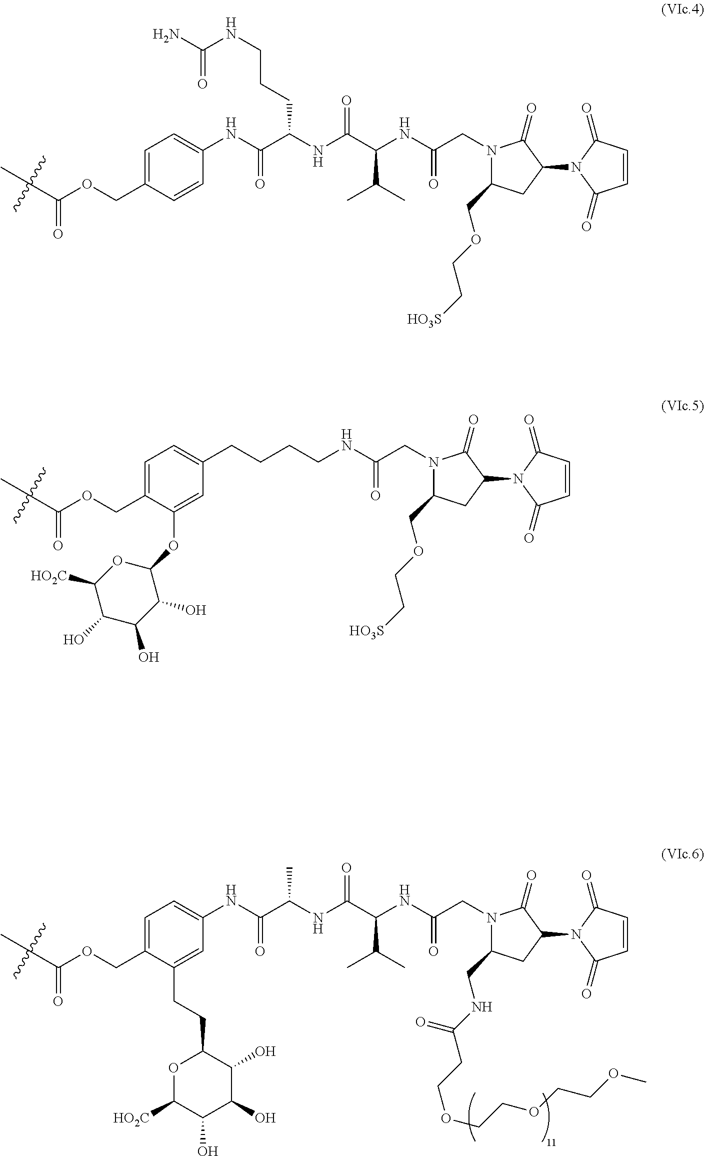

wherein [symbol] represents the point of attachment of the linker to an immune-modulatory compound or salt thereof.

As is known by skilled artisans, the linker selected for a particular conjugate may be influenced by a variety of factors, including but not limited to, the site of attachment to the antibody (e.g., lys, cys, or other amino acid residues), structural constraints of the drug pharmacophore and the lipophilicity of the drug. The specific linker selected for a conjugate should seek to balance these different factors for the specific antibody/drug combination.

For example, conjugates of cytotoxic compounds have been observed to effect killing of bystander antigen-negative cells present in the vicinity of the antigen-positive tumor cells. The mechanism of bystander cell killing by conjugates has indicated that metabolic products formed during intracellular processing of the conjugates may play a role. Neutral cytotoxic metabolites generated by metabolism of the conjugates in antigen-positive cells appear to play a role in bystander cell killing while charged metabolites may be prevented from diffusing across the membrane into the medium and therefore cannot affect bystander killing. In certain embodiments, the linker is selected to attenuate the bystander effect caused by a released immune-modulatory compound or derivative thereof caused by cellular metabolites of the conjugate. In certain embodiments, the linker is selected to increase the bystander effect of the immune-modulatory compound. In certain embodiments, the linker is selected to increase a bystander effect resulting from the same process, but as applied to an immune-modulatory compound metabolite. The increased bystander effect may be an increased effect on surrounding cells to treat fibrotic disease, autoimmune disease, or autoinflammatory disease.

The properties of the linker may also impact aggregation of the conjugate under conditions of use and/or storage. Typically, conjugates reported in the literature contain no more than 3-4 drug molecules per antibody molecule. Attempts to obtain higher drug-to-antibody ratios (“DAR”) often failed, particularly if both the drug and the linker were hydrophobic, due to aggregation of the conjugate. In many instances, DARs higher than 3-4 could be beneficial as a means of increasing potency. In instances where the immune-modulatory compound is hydrophobic in nature, it may be desirable to select linkers that are relatively hydrophilic as a means of reducing conjugate aggregation, especially in instances where DARs greater than 3-4 are desired. Thus, in certain embodiments, the linker incorporates chemical moieties that reduce aggregation of the conjugates during storage and/or use. A linker may incorporate polar or hydrophilic groups such as charged groups or groups that become charged under physiological pH to reduce the aggregation of the conjugates. For example, a linker may incorporate charged groups such as salts or groups that deprotonate, e.g., carboxylates, or protonate, e.g., amines, at physiological pH.

In particular embodiments, the aggregation of the conjugates during storage or use is less than about 40% as determined by size-exclusion chromatography (SEC). In particular embodiments, the aggregation of the conjugates during storage or use is less than 35%, such as less than about 30%, such as less than about 25%, such as less than about 20%, such as less than about 15%, such as less than about 10%, such as less than about 5%, such as less than about 4%, or even less, as determined by size-exclusion chromatography (SEC).

In certain embodiments, the ubiquitin ligase binding moiety or second moiety can be linked to the immune-modulatory compound or first moiety in the conjugate as described herein. The ubiquitin ligase binding moiety can be linked to the immune-modulatory compound via a spacer with a linear non-hydrogen atom number in the range of 1 to 20. The ubiquitin ligase binding moiety can be linked to the immune-modulatory compound via a spacer with a functional group such as ether, amide, alkane, alkene, alkyne, ketone, hydroxyl, carboxylic acid, thioether, sulfoxide, and sulfone. The ubiquitin ligase binding moiety can be linked to the immune-modulatory compound a linker comprising an aromatic, heteroaromatic, cyclic, bicyclic, and/or tricyclic moiety.

Linkers and linker covalent attachment sites of the linker to immune-modulatory compound can be cleavable or non-cleavable. A linker can be a non-cleavable linker attached to the immune-modulatory compound at site wherein the immune-modulatory compound may not lose target binding and may not lose immune-modulatory activity as determined by Kd measurement, by altered target protein function in a cell-based assay, or both. Linker length can be varied to optimize the activity of the immune-modulatory compound in the conjugate for its target protein. Such linkers can be short, flexible, rigid, hydrophilic, or hydrophobic. A linker can contain segments that have different characteristics, such as segments of flexibility or segments of rigidity. The linker can be chemically stable to extracellular environments. Non-limiting examples can be maleimidocaproyl linkers. A maleimidocaproyl linker can comprise N-maleimidomethylcyclohexane-1-carboxylate. A linker can be a combination of a maleimidocaproyl group and one or more polyethylene glycol molecules.

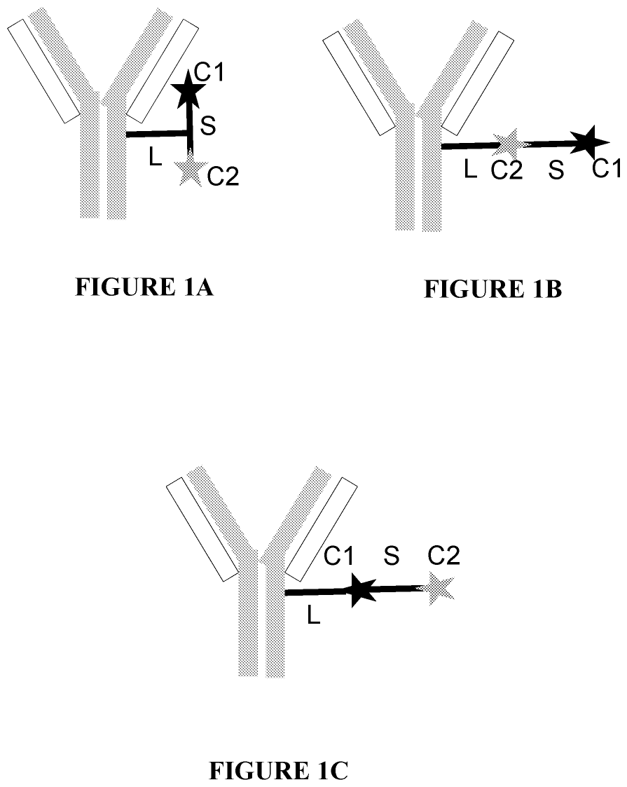

A linker (L) can comprise from 5 to 100 linear, non-hydrogen atoms that can be covalently attached to an antibody construct (e.g., an antibody) and can be: a) covalently attached to an immune-modulatory compound; b) covalently attached to an immune-modulatory compound 1 (C1) that can be covalently attached to a spacer (S) comprising from 5 to 100 linear, non-hydrogen atoms, in which the spacer can be covalently attached to a second compound (C2) (FIG. 1C); c) covalently attached to a second compound (C2), in which the second compound can be covalently attached to a spacer (S) comprising from 5 to 100 linear, non-hydrogen atoms, wherein the spacer can be covalently attached to an immune-modulatory compound (C1) (FIG. 1B); or d) covalently to a spacer, wherein the spacer can be covalently attached to a C1 and a C2 (FIG. 1A). In some embodiments of b)-d), C2 can be an E3 ubiquitin ligase binding moiety. The second compound has an activity such as a binding activity, an immune-modulatory activity or a different biological activity.

A linker (L) may comprise from 5 to 100 linear non-hydrogen atoms that may be covalently attached to an antibody construct (A) (such as an antibody) and may be:
i) covalently attached to an immune-modulatory compound (C₁) as in

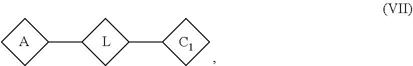

or
ii) covalently attached to an immune-modulatory compound (C1) which itself may be covalently attached to a spacer (S) comprising from 5 to 100 linear non-hydrogen atoms covalently attached to a second immune-modulatory compound (C₂) as in

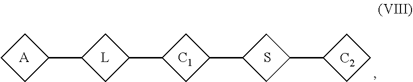

or
iii) covalently attached to an immune-modulatory compound (C₂) that may be covalently attached to a spacer (S) comprising from 5 to 100 linear non-hydrogen atoms covalently attached to a first immune-modulatory compound (C₁) as in

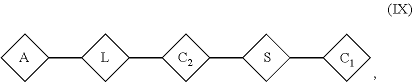

or
iv) covalently attached to a spacer (S) comprising from 5 to 100 linear non-hydrogen atoms covalently attached two immune-modulatory compounds (C₁ and C₂) as in

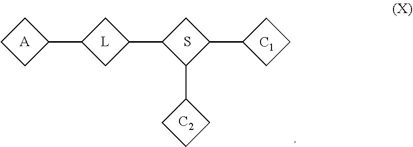

In some embodiments, C₂ is an E3 ubiquitin ligase binding moiety such that together C₁—S—C₂ may form a proteolysis-targeting chimera (PROTAC) complex (also referred to as a proteolysis targeting module or PTM).

In some embodiments, a protein targeting moiety, such as an immune-modulatory compound (IMC), is covalently attached to an E3 ubiquitin ligase binding moiety (ULM) through a spacer (S) and a linker (L) is covalently attached to the spacer (s), n is from 1-20 and z is from 1 to 20 as represented by the formula:

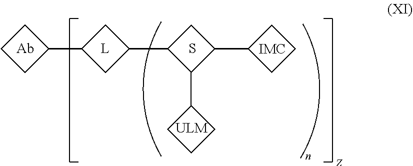

In some embodiments, L is a cleavable linker. The cleavable linker can be a peptide linker or other cleavable linker described above in the Section on Linkers. In some embodiments, L is a non-cleavable linker. In some embodiments, the Fc domain of the conjugate is an Fc null. In some embodiments, the Fc domain is a wild-type IgG that can bind to Fcγ receptors. In some embodiments, the Fc domain can bind to an Fc receptor, wherein the K_{d} for binding of the Fc domain of the conjugate to an Fc receptor is no greater than about 100 times the K_{d} for binding of a control antibody construct to the Fc receptor, wherein the control antibody construct is the unconjugated antibody construct. In some embodiments, the K_{d} for binding of the IMC of the conjugate to the protein active site is no greater than 100 times the K_{d} for binding of a control compound to the protein active site or wherein the IC50 of the IMC of the conjugate is no greater than 300-fold the IC50 of a control compound, wherein the control compound is the free IMC.

In some embodiments, a protein targeting moiety, such as an immune-modulatory compound (IMC), is covalently attached to an E3 ubiquitin ligase binding moiety (ULM) through a spacer (S) and a linker (L) is covalently attached to the protein targeting moiety, n is from 1-20 and z is from 1 to 20 as represented by the formula:

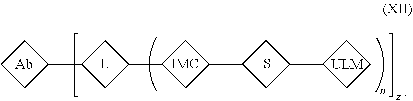

In some embodiments, L is a cleavable linker. The cleavable linker can be a peptide linker or other cleavable linker described above in the Section on Linkers. In some embodiments, L is a non-cleavable linker. In some embodiments, the Fc domain of the conjugate is an Fc null. In some embodiments, the Fc domain is a wild-type IgG that can bind to Fcγ receptors. In some embodiments, the Fc domain can bind to an Fc receptor, wherein the K_{d} for binding of the Fc domain of the conjugate to an Fc receptor is no greater than about 100 times the K_{d} for binding of a control antibody construct to the Fc receptor, wherein the control antibody construct is the unconjugated antibody construct. In some embodiments, the K_{d} for binding of the IMC of the conjugate to the protein active site is no greater than 100 times the K_{d} for binding of a control compound to the protein active site or wherein the IC50 of the IMC of the conjugate is no greater than 300-fold the IC50 of a control compound, wherein the control compound is the free IMC.

In some embodiments, a protein targeting moiety, such as an immune-modulatory compound (IMC), is covalently attached to an E3 ubiquitin ligase binding moiety (ULM) through a spacer (S) and linker L is covalently attached to the ubiquitin E3 ligase moiety (ULM), n is from 1-20 and z is from 1 to 20 as represented by the formula:

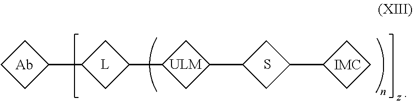

In some embodiments, L is a cleavable linker. The cleavable linker can be a peptide linker or other cleavable linker described above in the Section on Linkers. In some embodiments, L is a non-cleavable linker. In some embodiments, the Fc domain of the conjugate is an Fc null. In some embodiments, the Fc domain is a wild-type IgG that can bind to Fcγ receptors. In some embodiments, the Fc domain can bind to an Fc receptor, wherein the K_{d} for binding of the Fc domain of the conjugate to an Fc receptor is no greater than about 100 times the K_{d} for binding of a control antibody construct to the Fc receptor, wherein the control antibody construct is the unconjugated antibody construct. In some embodiments, the K_{d} for binding of the IMC of the conjugate to the protein active site is no greater than 100 times the K_{d} for binding of a control compound to the protein active site or wherein the IC50 of the IMC of the conjugate is no greater than 300-fold the IC50 of a control compound, wherein the control compound is the free IMC.

In certain embodiments, the E3 ubiquitin ligase binding moiety is linked to a protein targeting moiety, such as an immune-modulatory compound, in the conjugate as described herein, via a spacer. In certain embodiments, the E3 ubiquitin ligase binding moiety can be linked to the protein targeting moiety via a spacer having a linear non-hydrogen atom number in the range of 1 to 25 or 1 to 20. In certain embodiments, the spacer has 5 to 20 or 5 to 15 linear non-hydrogen atoms. The spacer is typically non-cleavable.

The E3 ubiquitin ligase binding moiety can be linked to the spacer of the protein targeting moiety with a functional group such as an ether, amide, alkane, alkene, alkyne, ketone, hydroxyl, carboxylic acid, thioether, sulfoxide, and sulfone. The E3 ubiquitin ligase binding moiety can be linked to the spacer of the protein targeting moiety via a spacer comprising an aromatic, heteroaromatic, cyclic, bicyclic, and/or tricyclic moiety.

Spacer length can be varied to optimize the activity of the protein targeting moiety for its target protein. In some embodiments, the spacer is non-cleavable and comprises segments of alkylene, alkenylene, alkynylene, —(CH₂O)—, —CH₂CH₂O)—, —(CH₂OCH₂)—, —C(O)—, —NH—, and —O—, having a length of from 1-25, 1-20, 1-15, 5-25, 5-20 or 5-15 linear non-hydrogen atoms. A spacer may be optionally substituted with C₁-C₈alkyl, C₂-C₈alkenyl, C₂-C₈alkynyl, —(CH₂O)ₙ₁H, —(CH₂CH₂O)ₙ₁H, —(CH₂O)ₙ₁CH₃, —C(O)OH or —NH₂, wherein n1 is from 1 to 8, and may further optionally comprise a reactive group, R^{x}, to form a functional group, such as an ether, amide, alkane, alkene, alkyne, ketone, hydroxyl, carboxylic acid, thioether, sulfoxide, and sulfone, forming an attachment to a linker (L). In some embodiments, the spacer is not unsubstituted. In some embodiments, the spacer is substituted with R^{x}.

A spacer may be a C₁₋₂₅alkylene or optionally substituted C₁₋₂₅ heteroalkylene, wherein the heteroalkylene is a C₁-2₄ alkylene chain interspersed with one or more groups independently selected from: —O—, —S—, —NH2-, and —C(O)NH—. The spacer may also be optionally substituted with a reactive group, R^{X}, that can form a functional group, such as an amide bond, an ester bond, an ether bond, a carbonate bond, a carbamate bond, or a thioether bond; such reactive groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups. In some embodiments, R^{x} can be —NH₂, —S, or a maleimide. In some embodiments, R^{x} is —NH₂. The spacer may also be optionally substituted with C₁-C₈alkyl, C₂-C₈alkenyl, C₂-C₈alkynyl, —(CH₂O)ₙ₁H, —(CH₂CH₂O)ₙ₁H, —(CH₂O)ₙ₁CH₃, —C(O)OH or —NH₂, wherein n1 is from 1 to 8. In some embodiments, the spacer is not unsubstituted. In some embodiments, the spacer is substituted with R^{x}.

In certain embodiments, the spacer (S) has the formula —C(O)N(R¹⁰⁰)R¹⁰¹C(O)N(R¹⁰⁰)—, —C(O)R¹⁰¹C(O)—, —C(O)R¹⁰¹N(R₁₀₀)—, —N(R₁₀₀)R¹⁰¹C(O)—, —N(R¹⁰⁰)C(O)R¹⁰¹C(O)—, —N(R¹⁰⁰)C(O)R¹⁰¹N(R₁₀₀)—, —N(R₁₀₀)R¹⁰¹C(O)N(R¹⁰⁰)—, —N(R¹⁰⁰)C(O)R¹⁰¹C(O)N(R¹⁰⁰)—, —N(R¹⁰⁰)C(O)R¹⁰¹N(R₁₀₀)C(O)—, and —C(O)N(R₁₀₀)R¹⁰¹C(O)N(R100)-; wherein each R¹⁰⁰ is independently selected from H or C₁-C₃ alkyl and R¹⁰¹ is —C₁-C₂₅alkylene-, —C₁-C₂₅alkenylene-, —C₁-C₂₅alkynlene-, —C₁-C₁₂alkylene(CH₂O)ₙC₁-C₁₅alkylene-, —C₁-C₁₂alkylene((CH₂OCH₂)ₙC₁-C₁₂alkylene-, —C₁-C₁₂alkylene(CH₂CH₂O)ₙC₁-C₁₂alkylene-, —C₁-C₁₂alkenylene-((CH₂O)ₙC₁-C₁₂alkylene-, —C₁-C₁₂alkenylene-(CH₂CH₂O)ₙC₁-C₁₂alkylene-, —C₁-C₁₂alkenylene-((CH₂OCH₂)ₙC₁-C₁₂alkylene-, —C₁-C₁₂alkylene-(CH₂O)ₙC₁-C₂₅alkenylene-, —C₁-C₁₂alkylene-(CH₂CH₂O)ₙC₁-C₂₅alkenylene-, —C₁-C₁₂alkylene-(CH₂OCH₂)ₙC₁-C₂₅alkenylene-, —C₁-C₁₂alkynylene-(CH₂O)ₙC₁-C₁₂alkylene-, —C₁-C₂₅alkynylene-(CH₂CH₂O)ₙC₁-C₁₂alkylene-, —C₁-C₁₂alkynylene-(CH₂OCH₂)ₙC₁-C₁₂alkylene-, —C₁-C₁₂alkynylene-(CH₂O)ₙC₁-C₂alkenylene-, —C₁-C₂alkynylene-(CH₂CH₂O)ₙC₁-C₁₂alkenylene-, —C₁-C₂alkynylene-(CH₂OCH₂)ₙC₁-C₂₅alkenylene-, —C₁-C₁₂alkynylene-(CH₂O)ₙC₁-C₂₅alkynylene-, —C₁-C₂₅alkynylene-(CH₂CH₂O)ₙC₁-C₂alkynylene-, —C₁-C₁₂alkynylene-(CH₂OCH₂)ₙC₁-C₁₂alkynylene-, in each case optionally substituted with a reactive moiety R^{x} for attachment to the linker (L), and n is 0 to 8. R^{X} can be a reactive group that can form an amide bond, an ester bond, an ether bond, a carbonate bond, a carbamate bond, or a thioether bond; such reactive groups can be, for example, amino groups; carboxyl groups; aldehyde groups; azide groups; alkyne and alkene groups; ketones; carbonates; carbonyl functionalities bonded to leaving groups such as cyano and succinimidyl and hydroxyl groups. In some embodiments, R^{x} can be —NH₂, —S or a maleimide. In some embodiments, R^{x} is —NH₂.

In certain embodiments, the spacer (S) comprises glutamate, a glycine-glutamate dipeptide, glycine-PEG1-glutamate, glycine-PEG2-glutamate, glycine-PEG3-glutamate, glycine-PEG4-glutamate or glycine-PEG5-glutamate, wherein the E3 ubquitin ligase binding moiety and the protein targeting moiety are attached to the spacer via amide bonds.

An E3 ubiquitin ligase binding moiety can bind to an E3 ubiquitin ligase, such as Von Hippel-Lindaue E3 ubiquitin ligase (VHL), cereblon, mouse double minute 2 homolog (MDM2), AMFR, APC/Cdc20, APC/Cdh1, C6orf157, Cb1, CBLL1, CHFR, CHIP, DTL (Cdt2), E6-AP, HACE1, HECTD1, HECTD2, HECTD3, HECW1, HECW2, HERC2, HERC3, HERC4, HERC5, HUWE1, HYD, ITCH, LNX1, mahogunin, MARCH-I, MARCH-II, MARCH-III, MARCH-IV, MARCH-VI, MARCH-VII, MARCH-VIII, MARCH-X, MEKK1, MIB1, MIB2, MycBP2, NEDD4, NEDD4L, Parkin, PELI1, Pirh2, PJA1, PJA2, RFFL, RFWD2, Rictor, RNF5, RNF8, RNF19, RNF190, RNF20, RNF34, RNF40, RNF125, RNF128, RNF138, RNF168, SCF/β-TrCP, SCF/FBW7, SCF/Skp2, SHPRH, SIAH1, SIAH2, SMURF1, SMURF2, TOPORS, TRAF6, TRAF7, TRIM63, UBE3B, UBE3C, UBR1, UBR2, UHRF2, WWP1, WWP2, or ZNRF1.

In other embodiments, an E3 ubiquitin ligase binding moiety can be selected from an E3 ubiquitin ligase selected from von Rippel-Lindau (VHL), cereblon, XIAP, E3A, MDM2, Anaphase-promoting complex (APC), UBR5 (EDDI), SOCS/BC-box/eloBC/CUL5/RING, LNXp80, CBX4, CBLLI, HACEI, HECTDI, HECTD2, HECTD3, HECWI, HECW2, HERCI, HERC2, HERC3, HERC4, HUWEI, ITCH, NEDD4, NEDD4L, PPIL2, PRPFI9, PIASI, PIAS2, PIAS3, PIAS4, RANBP2, RNF4, RBXI, SMURFI, SMURF2, STUB I, TOPORS, TRIPI2, UBE3A, UBE3B, UBE3C, UBE4A, UBE4B, UBOX5, UBR5, WWPI, WWP2, Parkin, A20/TNFAIP3, AMFR/gp78, ARA54, beta-TrCPI/BTRC, BRCAI, CBL, CHIP/STUB I, E6, E6AP/UBE3A, F-box protein I5/FBXOI5, FBXW7/Cdc4, GRAIL/RNFI28, HOIP/RNF3 I, cIAP-I/HIAP-2, cIAP-2/HIAP-I, cIAP (pan), ITCH/AIP4, KAPI, MARCH8, Mind Bomb I/MIBI, Mind Bomb 2/MIB2, MuRFI/TRIM63, NDFIPI, NEDD4, NleL, Parkin, RNF2, RNF4, RNF8, RNFI68, RNF43, SARTI, Skp2, SMURF2, TRAF-I, TRAF-2, TRAF-3, TRAF-4, TRAF-5, TRAF-6, TRIMS, TRIM2I, TRIM32, UBR5, and ZNRF3.

In further embodiments, an E3 ubiquitin ligase can be selected from the following types: HECT type, RING-type, PARKIN-finger type, RING-variant type, U-box type, A20-finger type, PIAS-finger type, PHD-finger type, Skp1-like type, Cullin-type, F-box type, SOCS-box type, BTB-type, DDB1-like type, and APC/cyclosome type.

An E3 ubiquitin ligase binding moiety can be a VHL binding moiety such as (S)-2-amino-N1-(4-(5-amino-6-((4-morpholinopyridin-3-yl)carbamoyl)pyrazin-2-yl)benzyl)-N5-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethyl)pentanediamide (Example 1) or a cereblon binding moiety such as 3-amino-6-(4-(2-((2S)-2-amino-6-(2-((2-(2,6-dioxopiperidin-3-yl)-1,3-dioxoisoindolin-4-yl)oxy)acetamido)hexanamido)ethyl)phenyl)-N-(4-morpholinopyridin-3-yl)pyrazine-2-carboxamide (Example 2).

In certain embodiments, the linker (L) is attached to the PTM at a reactive site R^{x} in the spacer. In certain embodiments, the linker (L) is attached to the PTM via an attachment site in the E3 ubiquitin ligase binding moiety. In certain embodiments, the linker (L) is attached to the PTM via an attachment site in the protein targeting moiety.

The linker (L) and/or covalent attachment site(s) of the linker (L) to the proteolysis targeting module can be cleavable or non-cleavable. In certain embodiments, the linker is cleavable. In certain embodiments, the linker is non-cleavable linker. In some embodiments, the linker is non-cleavable and is attached to the proteolysis targeting module at site wherein the protein targeting moiety can bind to its protein target, and, if active, does not lose immune-modulatory activity, as determined by Kd measurement, by altered target protein function in a cell-based assay, or both. Linker length can be varied to optimize the activity of the protein targeting moiety for its target protein. Such linkers can be short, flexible, rigid, hydrophilic, or hydrophobic. The linker can contain segments that have different characteristics, such as segments of flexibility or segments of rigidity. The linker can be chemically stable to extracellular environments. Non-limiting examples can be maleimidocaproyl linkers. A maleimidocaproyl linker can comprise N-maleimidomethylcyclohexane-1-carboxylate.

In some embodiments, the linker (L) is a cleavable linker and can be selected from the linkers of formulae IIa, IIb, IIc, IIIa, IIIb, IIIc, IIId, IVa, IVb, IVc, IVd and IVe and specific structures therein, as shown above.

A linker (L) can be a combination of a maleimidocaproyl group and one or more polyethylene glycol molecules.

A linker (L) may comprise from 5 to 100 linear non-hydrogen atoms that may be covalently attached to an antibody construct.

In some embodiments, the protein targeting moiety of the proteolysis targeting module can be an antagonist, such as a PI3K inhibitor, Calcineurin inhibitor, mTOR inhibitor, BTK inhibitor, JAK inhibitor, CRAC inhibitor, PARP1 antagonist, PPARg agonist, Kv1.3 antagonist, KCa3.1 antagonist, PP2A agonist, IRAK4 inhibitor, MYD88 inhibitor, BCL-2 antagonist, A2ar agonist, TLR7 antagonist, c-KIT kinase inhibitor, KCA3.1 agonist, TGFβR1 inhibitor, TGFβR2 inhibitor, ACC antagonist, ASK1 antagonist, GLI1 inhibitor, TNKS antagonist or TNIK antagonist, or any combination thereof.

In some embodiments, the protein targeting moiety binds to CSFR1, RON/MST1, PI3Kd, PI3Kg, PARP1, PD-L1, PP2A, A2ar, TYRO3, AXL, or MER. In certain embodiments, the protein targeting moiety is an antagonist or inhibitor of CSFR1, RON/MST1, PI3Kd, PI3Kg, PARP1, PD-L1, PP2A, A2ar, TYRO3, AXL, or MER.

In other embodiments, the protein targeting moiety can be a Pattern recognition receptor (PRR) agonist, such a PAMP molecule or a DAMP molecule. In some embodiments, the protein targeting moiety can be a Toll-like receptor agonist, a RIG-I agonist, a STING agonist, a GPCR agonist, an ion channel agonist, a membrane transporter agonist, or an ER protein agonist.

In certain embodiments, the antibody construct (such as an antibody) specifically binds to a first antigen selected from Cadherin 11, PDPN, Integrin α4β7, Integrin α2b1, MADCAM, Nephrin, Podocin, IFNAR1, BDCA, CD30, c-KIT, FAP, CD73, CD38, PDGFRB, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, and CD25. In some aspects, the first antigen is selected from Cadherin 11, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, LRRC15, and Cadherin11. In some embodiment, the antibody construct binds to a first antigen selected from Cadherin 11, PDPN, LRRC15, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, MMP14, GPX8, and F2RL2. In some embodiments, the antibody construct binds to a first antigen selected from Cadherin 11, FAP, TNFR2, or LRRC15. In some aspects, the antibody construct binds to a first antigen selected from FAP, and Cadherin 11. In some aspects, the antibody construct binds to a first antigen selected from LRRC15.

In certain embodiments, the antibody construct specifically binds to an antigen on a T cell, a B cell, a stellate cell, an endothelial cell, a tumor cell, an APC, a fibroblast cell, a fibrocyte cell, a myofibroblast, a synovial fibroblast, a podocyte, or a cell associated with the pathogenesis of fibrosis. In certain embodiments, the antibody construct specifically binds to an antigen on a T cell, an APC, and/or a B cell. In certain embodiments, the antibody construct specifically binds to an antigen selected from the group consisting of PD-1, GARP, CD25, PD-L1, or TNFR2. In certain embodiments, the antibody construct specifically binds to an antigen on a stellate cell, an endothelial cell, a fibroblast cell, a fibrocyte cell, a podocyte, or a cell associated with the pathogenesis of fibrosis. In certain embodiments, the antibody construct specifically binds to an antigen selected from the group consisting of PDGFRβ, integrin αvβ1, integrin αvβ3, integrin αvβ6, integrin αvβ8, Endosialin, FAP, ADAM12, LRRC15, MMP14, PDPN, CDH11, and F2RL2. In certain embodiments, the antibody construct specifically binds to an antigen antigen selected from the group consisting of FAP, ADAM12, LRRC15, MMP14, PDPN, CDH11, and F2RL2.

### Pharmaceutical Formulations

The conjugates and methods described herein may be considered useful as pharmaceutical compositions for administration to a subject in need thereof. Pharmaceutical compositions may comprise at least the conjugates described herein and one or more pharmaceutically acceptable carriers, diluents, excipients, stabilizers, dispersing agents, suspending agents, and/or thickening agents. The pharmaceutical composition may comprise the conjugate having an antibody construct, a linker and a PI3K inhibitor, Calcineurin inhibitor, mTOR inhibitor, BTK inhibitor, JAK inhibitor, CRAC inhibitor, PARP1 antagonist, PPARg agonist, Kv1.3 antagonist, KCa3.1 antagonist, PP2A agonist, IRAK4 inhibitor, MYD88 inhibitor, BCL-2 antagonist, A2ar agonist, TLR7 antagonist, c-KIT kinase inhibitor, KCA3.1 agonist, TGFβR1 inhibitor, TGFβR2 inhibitor, ACC antagonist, ASK1 antagonist, GLI1 inhibitor, TNKS antagonist, or TNIK antagonist. The pharmaceutical composition may comprise the conjugate having an antibody construct, a linker and a TGFβ1, TGFβR1, or TGFβR2 inhibitor. The pharmaceutical composition may comprise the conjugate having an antibody construct, a linker and TGFβR1, or TGFβR2 inhibitor. The pharmaceutical composition may comprise the conjugate having an antibody construct, a linker and a TGFβR1 inhibitor. The pharmaceutical composition may comprise the conjugate having an antibody construct, a linker and a TGFβR2 inhibitor. The pharmaceutical composition may comprise the conjugate having an antibody construct including a second antigen binding domain, a linker and a PI3K inhibitor, Calcineurin inhibitor, mTOR inhibitor, BTK inhibitor, JAK inhibitor, CRAC inhibitor, PARP1 antagonist, PPARg agonist, Kv1.3 antagonist, KCa3.1 antagonist, PP2A agonist, IRAK4 inhibitor, MYD88 inhibitor, BCL-2 antagonist, A2ar agonist, TLR7 antagonist, c-KIT kinase inhibitor, KCA3.1 agonist, TGFβR1 inhibitor, TGFβR2 inhibitor, ACC antagonist, ASK1 antagonist, GLI1 inhibitor, TNKS antagonist, or TNIK antagonist. The pharmaceutical composition may comprise the conjugate having an antibody construct including a second antigen binding domain, a linker and a TGFβ1, TGFβR1, or TGFβR2 inhibitor. The pharmaceutical composition may comprise the conjugate having an antibody construct including a second antigen binding domain, a linker and a TGFβR2 inhibitor. A pharmaceutical composition may comprise any conjugate described herein. The antibody construct may be an anti-LRRC15 antibody. The antibody construct may be an anti-FAP antibody. The antibody construct may be an anti-CDH11 antibody. The antibody construct may be an anti-TNFR2 antibody. The antibody construct may comprise a set or pair of sequences from TABLE 1 and/or TABLE 2, respectively, that confer antigen binding specificity for the desired antigen. A conjugate may comprise an antibody construct comprising a set or pair of sequences from TABLE 1 and/or TABLE 2, respectively, that confer antigen binding specificity for the desired antigen, a linker and a PI3K inhibitor, Calcineurin inhibitor, mTOR inhibitor, BTK inhibitor, JAK inhibitor, CRAC inhibitor, PARP1 antagonist, PPARg agonist, Kv1.3 antagonist, KCa3.1 antagonist, PP2A agonist, IRAK4 inhibitor, MYD88 inhibitor, BCL-2 antagonist, A2ar agonist, TLR7 antagonist, c-KIT kinase inhibitor, KCA3.1 agonist, TGFβR1 inhibitor, TGFβR2 inhibitor, ACC antagonist, ASK1 antagonist, GLI1 inhibitor, TNKS antagonist, or TNIK antagonist. A conjugate may comprise an antibody construct comprising an antigen binding domain(s) comprising a set or pair of sequences from TABLE 1 and/or TABLE 2, respectively, that confer antigen binding specificity for the desired antigen, a linker and a PI3K inhibitor, Calcineurin inhibitor, mTOR inhibitor, BTK inhibitor, JAK inhibitor, CRAC inhibitor, PARP1 antagonist, PPARg agonist, Kv1.3 antagonist, KCa3.1 antagonist, PP2A agonist, IRAK4 inhibitor, MYD88 inhibitor, BCL-2 antagonist, A2ar agonist, TLR7 antagonist, c-KIT kinase inhibitor, KCA3.1 agonist, TGFβR1 inhibitor, TGFβR2 inhibitor, ACC antagonist, ASK1 antagonist, GLI1 inhibitor, TNKS antagonist, or TNIK antagonist. A pharmaceutical composition may further optionally comprise buffers, antibiotics, steroids, carbohydrates, drugs (e.g., chemotherapy drugs), polypeptides, chelators, adjuvants, and/or preservatives.

Pharmaceutical compositions may be formulated using one or more physiologically-acceptable carriers comprising excipients and auxiliaries. A formulation may be modified depending upon the route of administration chosen. Pharmaceutical compositions comprising a conjugate as described herein may be manufactured, for example, by lyophilizing the conjugate, mixing, dissolving, emulsifying, encapsulating or entrapping the conjugate. The pharmaceutical compositions may also include the conjugates described herein in a free-base form or pharmaceutically-acceptable salt form.

Methods for formulation of the conjugates to form pharmaceutical compositions described herein may include formulating any of the conjugates with one or more inert, pharmaceutically-acceptable excipients or carriers to form a solid, semi-solid, or liquid composition. Solid compositions may include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the pharmaceutical compositions described herein may be lyophilized or in powder form for re-constitution with a suitable vehicle, e.g., sterile pyrogen-free water, before use.

Pharmaceutical compositions of the conjugates described herein may further comprise at least an active ingredient. The active ingredients may be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as described herein may often further comprise more than one active compound as necessary for the particular indication being treated. The active compounds may have complementary activities that do not adversely affect each other. For example, the composition may comprise a chemotherapeutic agent, cytotoxic agent, cytokine, growth-inhibitory agent, anti-hormonal agent, anti-angiogenic agent, and/or cardioprotectant. Such molecules may be present in combination in amounts that are effective for the purpose intended.

The compositions and formulations may be sterilized. Sterilization may be accomplished by filtration through sterile filtration.

The conjugates described herein may be formulated for administration as an injection. Non-limiting examples of formulations for injection may include a sterile suspension, solution or emulsion in oily or aqueous vehicles. Suitable oily vehicles may include, but are not limited to, lipophilic solvents or vehicles such as fatty oils or synthetic fatty acid esters, or liposomes. Aqueous injection suspensions may contain substances which increase the viscosity of the suspension. The suspension may also contain suitable stabilizers. Injections may be formulated for bolus injection or continuous infusion. Alternatively, the pharmaceutical compositions described herein may be lyophilized or in powder form for reconstitution with a suitable vehicle, e.g., sterile pyrogen-free water, before use.

For parenteral administration, the conjugates may be formulated in a unit dosage injectable form (e.g., solution, suspension, emulsion) in association with a pharmaceutically acceptable parenteral vehicle. Such vehicles may be inherently non-toxic, and non-therapeutic. Vehicles may be water, saline, Ringer's solution, dextrose solution, and 5% human serum albumin. Non-aqueous vehicles such as fixed oils and ethyl oleate may also be used. Liposomes may be used as carriers. The vehicle may contain minor amounts of additives such as substances that enhance isotonicity and chemical stability (e.g., buffers and preservatives).

Sustained-release preparations may also be prepared. Examples of sustained-release preparations may include semipermeable matrices of solid hydrophobic polymers that may contain the conjugate, and these matrices may be in the form of shaped articles (e.g., films or microcapsules). Examples of sustained-release matrices may include polyesters, hydrogels (e.g., poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations of the conjugates described herein may be prepared for storage by mixing a conjugate with a pharmaceutically acceptable carrier, excipient, and/or a stabilizer. This formulation may be a lyophilized formulation or an aqueous solution. Acceptable carriers, excipients, and/or stabilizers may be nontoxic to recipients at the dosages and concentrations used. Acceptable carriers, excipients, and/or stabilizers may include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

### Therapeutic Applications

The pharmaceutical compositions, conjugates and methods of the present disclosure may be useful for a plurality of different subjects including, but are not limited to, a mammal, human, non-human mammal, a domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

The compositions, conjugates and methods described herein may be useful as a therapeutic, for example, a treatment that may be administered to a subject in need thereof. A therapeutic effect of the present disclosure may be obtained in a subject by reduction, suppression, remission, or eradication of a disease state, including, but not limited to, a symptom thereof. A therapeutic effect in a subject having a disease or condition, or pre-disposed to have or is beginning to have the disease or condition, may be obtained by a reduction, a suppression, a prevention (e.g., of relapse), a remission, or an eradication of the condition or disease, or pre-condition or pre-disease state.

In practicing the methods described herein, therapeutically-effective amounts of the pharmaceutical compositions or conjugates described herein may be administered to a subject in need thereof, often for treating and/or preventing a condition or progression thereof. A pharmaceutical composition may affect the physiology of the subject, such as the immune system, inflammatory response, or other physiologic affect. A therapeutically-effective amount may vary widely depending on the severity of the disease, the age and relative health of the subject, the potency of the compounds used, and other factors.

Treat and/or treating may refer to any indicia of success in the treatment or amelioration of the disease or condition. Treating may include, for example, reducing, delaying or alleviating the severity of one or more symptoms of the disease or condition, or it may include reducing the frequency with which symptoms of a disease, defect, disorder, or adverse condition, and the like, are experienced by a patient. Treat may be used herein to refer to a method that results in some level of treatment or amelioration of the disease or condition, and may contemplate a range of results directed to that end, including but not restricted to prevention of the condition or prevent of relapse.

Prevent, preventing and the like may refer to the prevention of the disease or condition, e.g., progression of fibrosis, in the patient. For example, if an individual at risk of developing a fibrosis, autoimmune disease, or autoinflammatory disease is treated with the methods of the present disclosure and does not later develop fibrosis, autoimmune disease, or autoinflammatory disease, then the disease has been prevented, at least over a period of time, in that individual. Prevent, preventing and the like may also refer to preventing relapse in an individual already treated.

A therapeutically effective amount may be the amount of a composition or conjugate sufficient to provide a beneficial effect or to otherwise reduce a detrimental non-beneficial event to the individual to whom the composition or conjugate is administered. A therapeutically effective dose may be a dose that produces one or more desired or desirable (e.g., beneficial) effects for which it is administered, such administration occurring one or more times over a given period of time. An exact dose may depend on the purpose of the treatment, and may be ascertainable by one skilled in the art using known techniques.

The pharmaceutical compositions and conjugates described herein that may be used in therapy may be formulated and dosages established in a fashion consistent with good medical practice taking into account the disorder to be treated, the condition of the individual patient, the site of delivery of the composition or conjugate, the method of administration and other factors known to practitioners. The conjugates described herein may be prepared according to the description of preparation described herein.

Pharmaceutical compositions, that may be considered useful with the conjugates and methods described herein, may be administered to a subject in need thereof using a technique known to one of ordinary skill in the art which may be suitable as a therapy for the disease or condition affecting the subject. One of ordinary skill in the art would understand that the amount, duration and frequency of administration of a pharmaceutical composition described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods and compositions and conjugates described herein may be for administration to a subject in need thereof. Often, administration of the compositions and conjugates described herein may include routes of administration, non-limiting examples of administration routes include intravenous, intraarterial, subcutaneous, subdural, intramuscular, intracranial, intrasternal, intratumoral, or intraperitoneally. Additionally, a pharmaceutical composition may be administered to a subject by additional routes of administration, for example, by inhalation, oral, dermal, intranasal, or intrathecal administration.

Compositions and conjugates of the present disclosure may be administered to a subject in need thereof in a first administration, and in one or more additional administrations. The administrations may be administered to the subject in need thereof in cycles of, for example, 21 days, 14 days, 10 days, 7 days, 4 days, or 1 day after the first administration. The one or more additional administrations also may be administered to the subject in need thereof minutes, hours, days, weeks or months following the first administration. Any one of the additional administrations may be administered to the subject in need thereof less than 21 days, or less than 14 days, less than 10 days, less than 7 days, less than 4 days or less than 1 day after the first administration. The one or more administrations may occur more than once per day, more than once per week or more than once per month.

### Methods of Treatment

The compositions, conjugates, and methods provided herein may be useful for the treatment of a plurality of diseases, conditions, preventing a disease or a condition in a subject or other therapeutic applications for subjects in need thereof. Often the compositions, conjugates and methods provided herein may be useful for treatment of autoimmune diseases, inflammatory diseases, or fibrotic diseases and the like. The compositions, conjugates and methods provided herein may be useful in specifically targeting cells and/or tissues associated with fibrotic disease, autoimmune disease, or autoinflammatory disease. The compositions, conjugates, and methods provided herein may be useful in specifically targeting TGFβ1, TGFβR1, or TGFβR2. The compositions, conjugates, and methods provided herein may be useful in inhibiting TGFβR1 or TGFβR2. In one embodiment, the conjugates may serve as TGFβR1 inhibitors. In another embodiment, the conjugates of the present disclosure may serve as TGFβR2 inhibitors. A condition disclosed herein may be associated with expression of an antigen on the specific cells related to the disease described herein. Often, the antigen expressed by the cells may comprise an extracellular portion capable of recognition by the antibody construct of the conjugate. An antigen expressed by the cells may be an antigen that can be a recognized by an antibody construct described herein. An antibody construct of the conjugate or composition may recognize a fibrotic associated antigen, autoimmune associated antigen, or autoinflammatory associated antigen. For example, an antigen may be Cadherin 11, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, or any fragment thereof. An antigen may be Cadherin 11, LRRC15, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, or any fragment thereof. An antigen may be Cadherin 11, LRRC15, or FAP. An antigen may be Cadherin 11, TNFR2, or FAP. An antigen may be TNFR2.

As described herein, an antigen binding domain portion of the conjugate may be configured to recognize an antigen expressed by a disease cell, such as for example, a disease antigen. Often such antigens are known to those of ordinary skill in the art, or newly found to be associated with such a condition, to be commonly associated with, and/or specific to, such conditions. For example, a disease antigen is, but is not limited to, Cadherin 11, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, or any fragment thereof. A disease antigen may also be Cadherin 11, LRRC15, PDPN, Integrin α4β7, Integrin α2β1, MADCAM, Nephrin, Podocin, IFNAR1, BDCA2, CD30, c-KIT, FAP, CD73, CD38, PDGFRβ, Integrin αvβ1, Integrin αvβ3, Integrin αvβ8, GARP, Endosialin, CTGF, Integrin αvβ6, CD40, PD-1, TIM-3, TNFR2, DEC205, DCIR, CD86, CD45RB, CD45RO, MHC Class II, CD25, or any fragment thereof. A disease antigen also may be FAP, LRRC15, or Cadherin 11. A disease antigen may be Cadherin 11, TNFR2, or FAP. A disease antigen may be TNFR2.

Non-limiting examples of fibrosis or fibrotic diseases include adhesive capsulitis, arterial stiffness, arthrofibrosis, atrial fibrosis, cirrhosis, Crohn's disease, collagenous fibroma, cystic fibrosis, Desmoid-type fibromatosis, Dupuytren's contracture, elastofibroma, endomyocardial fibrosis, fibroma of tendon sheath, glial scar, idiopathic pulmonary fibrosis, keloid, mediastinal fibrosis, myelofibrosis, nuchal fibroma, nephrogenic systemic fibrosis, old myocardial infarction, Peyronie's disease, pulmonary fibrosis, progressive massive fibrosis, radiation-induced lung injury, retroperitoneal fibrosis, scar, and scleroderma/systemic sclerosis.

Non-limiting examples of diseases that can be treated using a method according to the disclosure include acute disseminated encephalomyelitis (ADEM), acute necrotizing hemorrhagic leukoencephalitis, Addison's disease, agammaglobulinemia, alopecia, amyloidosis, ankylosing spondylitis (AS), Anti-GBM/Anti-TBM nephritis, antiphospholipid syndrome (APS), arthritis, autoimmune angioedema, autoimmune aplastic anemia, autoimmune dysautonomia, autoimmune hemolytic anemia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenic purpura (ATP), autoimmune thyroid disease, autoimmune urticarial, avascular Necrosis (Osteonecrosis)\Back Pain, axonal and neuronal neuropathy (AMAN), Balo disease, Behcet's Disease, bursitis and other soft tissue diseases, Bullous pemphigoid, cardiomyopathy, carpal tunnel syndrome, Castleman disease (CD), celiac disease, Chagas disease, chronic fatigue syndrome, chronic inflammatory demyelinating polyneuropathy (CIDP), chronic recurrent multifocal osteomyelitis (CRMO), Churg-Strauss, Cicatricial pemphigoid/benign mucosal pemphigoid, Cogan's syndrome, cold agglutinin disease, congenital heart block, Coxsackie myocarditis, CREST syndrome, collagen vascular disease, CPDD (Calcium Pyrophosphate Dihydrate Crystal Deposition Disease), Crohn's Disease, demyelinating neuropathies, degenerative joint disease, dermatitis herpetiformis, dermatomyositis, Devic's disease (neuromyelitis optica), diabetes (Type I), discoid lupus, DISH (Diffuse Idiopathic Skeletal Hypertosis), Dressler's syndrome, endometriosis, eosinophilic esophagitis (EoE), eosinophilic fasciitis, erythema nodosum, essential mixed cryoglobulinemia, dupuytren, EDS (Ehlers-Danlos Syndrome), EMS (Eosinophilia-Myalgia Syndrome), Evans syndrome, experimental allergic encephalomyelitis, Felty's Syndrome, fibromyalgia, fibromyositis, fibrosing alveolitis, giant cell arteritis (temporal arteritis), giant cell myocarditis, glomerulonephritis, Goodpasture's syndrome, gout, granulomatosis with Polyangiitis, Graves' Disease, Guillain-Barrè syndrome, Hashimoto's thyroiditis, hemolytic anemia, Henoch-Schonlein purpura (HSP), herpes gestationis or pemphigoid gestationis (PG), hypogammalglobulinemia, idiopathic pulmonary fibrosis, idiopathic thrombocytopenic purpura, IgA Nephropathy, IgG4-related sclerosing disease, immunoregulatory lipoproteins, inclusion body myositis (IBM), infectious arthritis, inflammatory bowel disease, interstitial cystitis (IC), JH (Joint Hypermobility), joint inflammation, juvenile rheumatoid arthritis, juvenile arthritis—other types and related conditions, juvenile dermatomyositis, juvenile diabetes (Type 1 diabetes), juvenile idiopathic arthritis (JIA), juvenile myositis (JM), juvenile non-inflammatory disorders, juvenile psoriatic arthritis, juvenile scleroderma, juvenile spondyloarthropathy syndromes, juvenile systemic lupus erythematosis (SLE), juvenile vasculitis, Kawasaki disease, Ledderhose Disease (Dupuytren of the feet), Lambert-Eaton syndrome, leukocytoclastic vasculitis, lichen planus, lichen sclerosus, ligneous conjunctivitis, linear IgA disease (LAD), lupus, Discoid, lupus erythematosis, lyme Disease, Marfan Syndrome, MCTD (Mixed Connective Tissue Disease), Meniere's disease, microscopic polyangiitis (MPA), mixed connective tissue disease (MCTD), Mooren's ulcer, Mucha-Habermann disease, multiple sclerosis, myasthenia gravis, myocarditis, myofascial pain, narcolepsy, neuromyelitis optica, neutropenia, ocular cicatricial pemphigoid, optic neuritis, osteoarthritis, osteogenesis imperfecta, osteonecrosis (Avascular Necrosis), osteoporosis, Paget's Disease, palindromic rheumatism (PR), PANDAS (Pediatric Autoimmune Neuropsychiatric Disorders Associated with Streptococcus), paraneoplastic cerebellar degeneration (PCD), paroxysmal nocturnal hemoglobinuria (PNH), Parry Romberg syndrome, Parsonnage-Turner syndrome, pars planitis (peripheral uveitis), pemphigus, pemphigus/pemphigoid, peripheral neuropathy, perivenous encephalomyelitis, pernicious anemia, Peyronie's Disease, POEMS syndrome (polyneuropathy, organomegaly, endocrinopathy, monoclonal gammopathy, skin changes), PMR (polymyalgia rheumatica), polyarteritis nodossa, polyarthritis, polymyalgia rheumatic, polymyositis, postmyocardial infarction syndrome, postpericardiotomy syndrome, primary biliary cirrhosis, primary sclerosing cholangitis, progesterone dermatitis, pseudogout, Pseudoxanthoma Elasticum (PXE), psoriatic arthritis, psoriasis, pure red cell aplasia (PRCA), pyoderma gangrenosum, Raynaud's, reactive arthritis, Reiter's (Reactive Arthritis), relapsing polychondritis, retroperitoneal fibrosis, rheumatic fever, rheumatoid Arthritis, RLD (Restless Leg Syndrome), RSD (Reflex Sympathetic Dystrophy), Sarcoidosis, Schmidt syndrome, scleritis, Sjögren's Syndrome, soft tissue disease, sperm and testicular autoimmunity, spinal stenosis, stiff person syndrome (SPS), Still's Disease, subacute bacterial endocarditis (SBE), Susac's syndrome, sympathetic ophthalmia (SO), Takayasu's arteritis, temporal arteritis/Giant cell arteritis, thrombocytopenic purpura (TTP), Tolosa-Hunt syndrome (THS), transverse myelitis, temporal arteritis, TMJ (Tempero-Mandibular Joint) problems, thyroiditis, Type I, II, & III autoimmune polyglandular syndromes, ulcerative colitis, undifferentiated connective tissue disease (UCTD), undifferentiated spondylarthropathy, uveitis, Wegener's Granulomatosis, vasculitis, vesiculobullous dermatosis, and vitiligo.

In some embodiments, rheumatoid arthritis is treated using a conjugate described herein. In some embodiments, the antibody conjugate specifically binds to cadherin 11. In some embodiments, the antibody conjugate specifically binds to PDPN.

In some embodiments, inflammatory bowel disease, for example, Crohn's disease and ulcerative colitis, is treated using an antibody conjugate described herein. In some embodiments, the antibody conjugate binds to integrin α4β7. In some embodiments, the antibody conjugate binds to integrin α2β1. In some embodiments, the antibody conjugate binds to MADCAM.

In some embodiments, systemic lupus erythematosus is treated using an antibody conjugate described herein. In some embodiments, the antibody conjugate binds to nephrin. In some embodiments, the antibody conjugate binds to podocin. In some embodiments, the antibody conjugate binds to PDPN. In some embodiments, the antibody conjugate binds to IFNAR1. In some embodiments, the antibody conjugate binds to BDCA2. In some embodiments, the antibody conjugate binds to CD30.

In some embodiments, mastocytosis or uticaria pigmentosa is treated using an antibody conjugate described herein. In some embodiments, the antibody conjugate binds to c-KIT.

In some embodiments, multiple sclerosis, is treated using an antibody conjugate described herein.

In some embodiments, scleroderma or systemic sclerosis is treated using an antibody conjugate described herein.

In some embodiments, graft-versus-host-disease and transplant rejection is treated using an antibody conjugate described herein.

In some embodiments, asthma is treated using an antibody conjugate described herein.

In some embodiments, ankylosing spondylitis is treated using an antibody conjugate described herein.

In some embodiments, psoriasis is treated using an antibody conjugate described herein.

In some embodiments, type 1 diabetes is treated using an antibody conjugate described herein.

In some embodiments, fibrosis is treated using an antibody conjugate described herein. In some embodiments, the antibody conjugate specifically binds to FAP, LRRC15, or Cadherin 11. In some embodiments, the antibody conjugate specifically binds to FAP. In some embodiments, the antibody conjugate specifically binds to LRRC15. In some embodiments, the antibody conjugate specifically binds to TNFR2. In some embodiments, the antibody conjugate specifically binds to Cadherin 11.

The invention provides any therapeutic compound or conjugate disclosed herein for use in a method of treatment of the human or animal body by therapy. Therapy may be by any mechanism disclosed herein, such as by stimulation of the immune system. The invention provides any therapeutic compound or conjugate disclosed herein for use in stimulation of the immune system, vaccination or immunotherapy, including for example enhancing an immune response. The invention further provides any therapeutic compound or conjugate disclosed herein for prevention or treatment of any condition disclosed herein, for example cancer, autoimmune disease, inflammation, sepsis, allergy, asthma, graft rejection, graft-versus-host disease, immunodeficiency or infectious disease (typically caused by an infectious pathogen). The invention also provides any therapeutic compound or conjugate disclosed herein for obtaining any clinical outcome disclosed herein for any condition disclosed herein, such as reducing tumour cells in vivo. The invention also provides use of any therapeutic compound or conjugate disclosed herein in the manufacture of a medicament for preventing or treating any condition disclosed herein.

### General Schemes

### Synthesis of Immune-Stimulatory Compound-Linkers and Immune-Modulatory Compound-Linker Constructs

An construct of a linker and an immune-stimulatory compound or an immune-modulatory compound (denominated ISC) can be synthesized by various methods. For example, ISC-linker constructs can be synthesized as shown in Scheme B1.

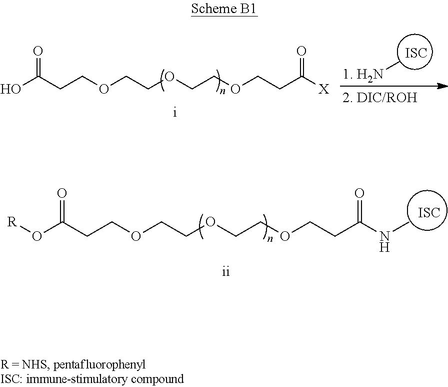

A PEGylated carboxylic acid (i) that has been activated for amide bond formation can be reacted with an appropriately substituted amine containing immune-stimulatory compound to afford an intermediate amide. Formation of an activated ester (ii) can be achieved by reaction the intermediate amide-containing carboxylic using a reagent such as N-hydroxysuccinimide or pentafluorophenol in the presence of a coupling agent such as diisopropylcarbodiimide (DIC) to provide compounds (ii).

An ISC-linker construct can be synthesized as shown in Scheme B2.

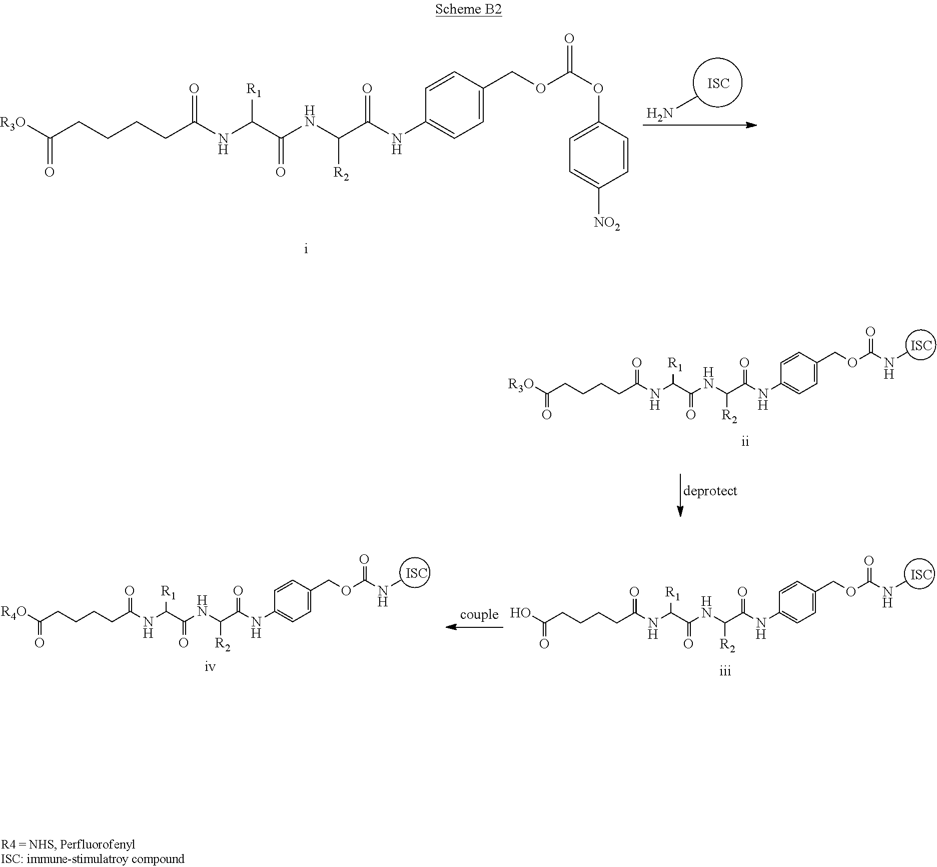

An activated carbonate such as (i) can be reacted with an appropriately substituted amine containing immune-stimulatory compound to afford carbamates (ii) which can be deprotected using standard methods based on the nature of the R₃ ester group. The resulting carboxylic acid (iii) can then by coupled with an activating agent such as N-hydroxysuccinimide or pentafluorophenol to provide compounds (iv).

A ISC-linker construct can be synthesized as shown in Scheme B3.

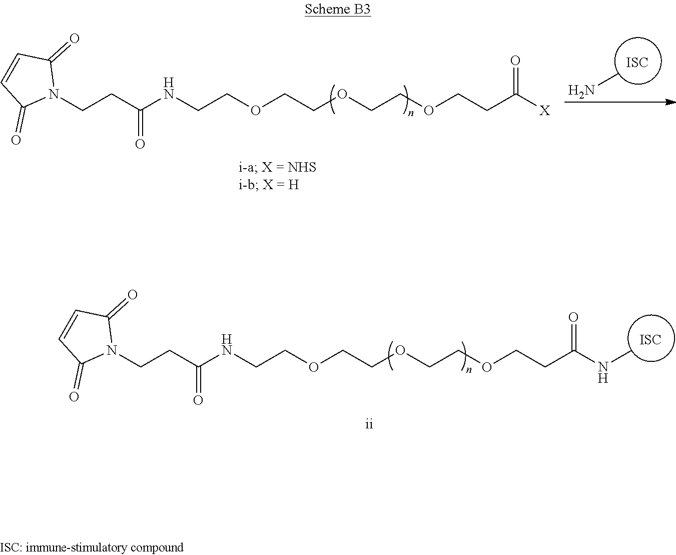

An activated carboxylic ester such as (i-a) can be reacted with an appropriately substituted amine containing immune-stimulatory compound to afford amides (ii). Alternatively, carboxylic acids of type (i-b) can be coupled to an appropriately substituted amine containing immune-stimulatory compound in the presence of an amide bond forming agent such as dicyclohexycarbodiimde (DCC) to provide the desired ISC.

An ISC-linker construct can be synthesized by various methods such as that shown in Scheme B4.

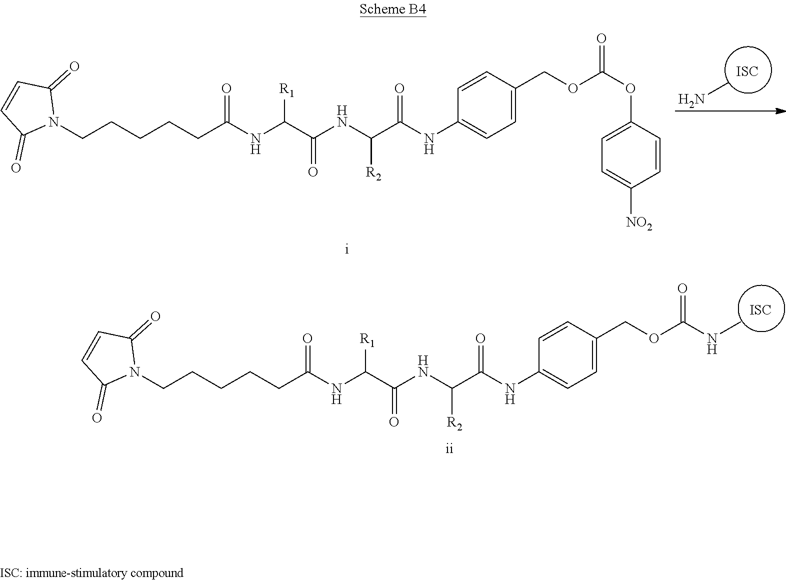

An activated carbonate such as: (i) can be reacted with an appropriately substituted amine containing immune-stimulatory compound to afford carbamates (ii) as the target ISC.

An ISC-linker construct can also be synthesized as shown in Scheme B5.

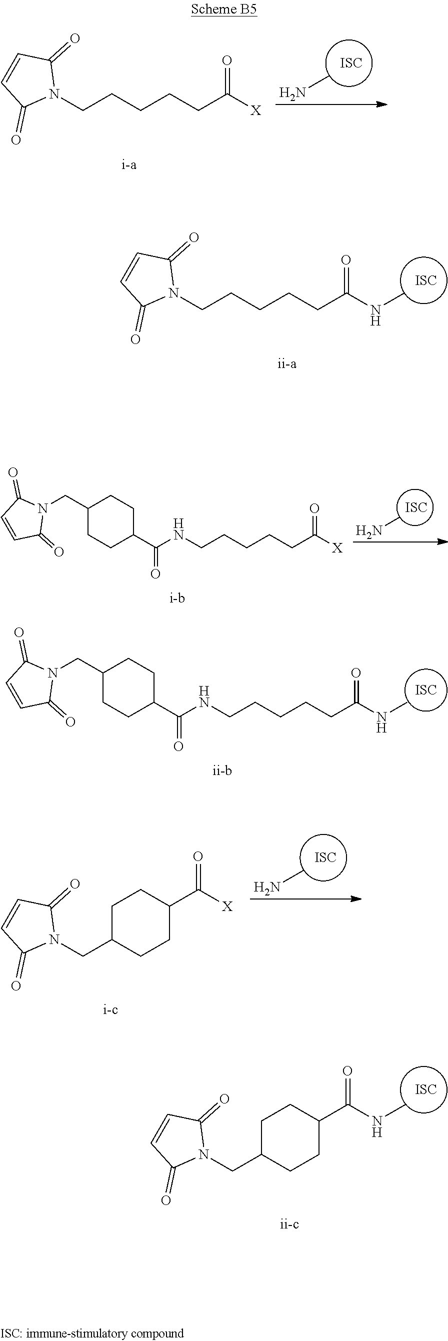

An activated carboxylic acid such as (i-a, i-b, i-c) can be reacted with an appropriately substituted amine containing immune-stimulatory compound to afford amides (ii-a, ii-b, ii-c) as the target linkered immune-stimulatory compounds.

### General Scheme for the Synthesis of Immune-Modulatory Conjugates Containing a PROTAC

An immune-modulatory conjugate containing a PROTAC (or PTM) as described herein can comprise an antibody construct Ab (such as an antibody) convalently attached via a linker (L) to a PROTAC, wherein the PROTAC comprises a ubiquitin E3 ligase binding group (E; also referred to as ULM), a spacer (S) and an immune-modulatory compound (K; also referred to as an IMC) (such as a kinase inhibitor). The general formula is: Ab-(L-(C₁—S—C₂))ₙ, wherein Ab is the antibody construct, C₁—S—C₂ is PROTAC or PTM, wherein, C₂ is an E3 ubiquitin ligase binding group (E or ULM) covalently bound to a spacer group (s) that is covalently bound to C₁, an immune-modulatory compound (E or IMC), and L is a linker covalently bonded to the antibody construct and to the PROTAC; and n has a value from about 1 to about 8.

In the following exemplary scheme, the immune-modulatory compound (E in this scheme) is a kinase inhibitor.

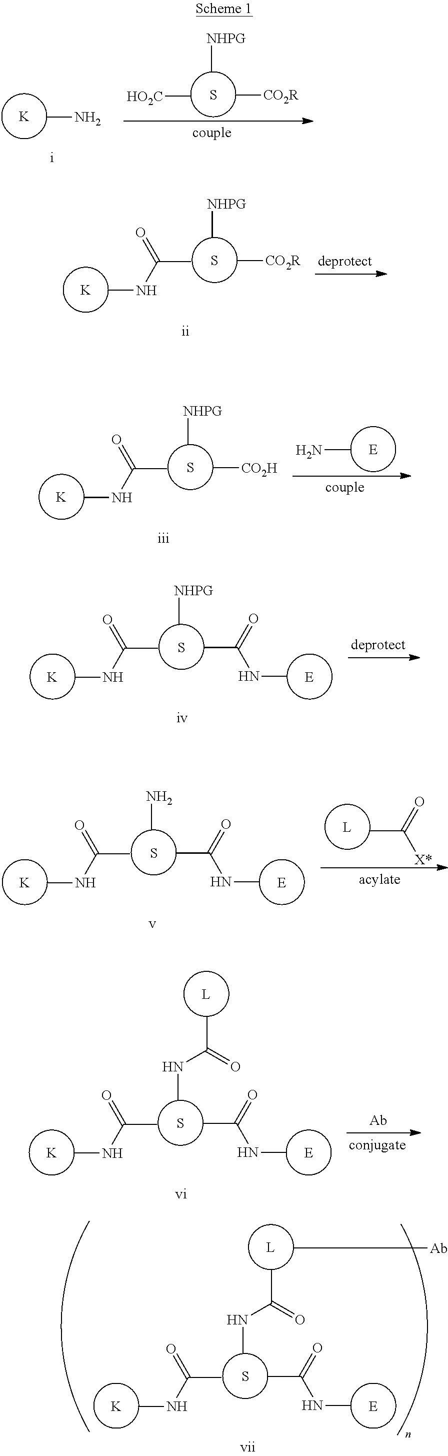

A kinase inhibitor containing a free amine functional group can be acylated with a multi-functional amino acid derivative such as aspartate or glutamate using standard amide bond coupling reactions such as HATU in DMF containing and amine base to provide intermediates (ii). Deprotection of compounds (ii) using known methods for the conversion of carboxylic esters to carboxylic acids, such as hydrogenation when R=Bn can provide compounds (iii) which can be coupled to an E3 ubiquitin ligase such as a group that binds VHL or cereblon to provide PROTACs (iv). Compounds that bind VHL may be hydroxyproline compounds such as those disclosed in WO 2013/106643, and other compounds described in US 2016/0045607, WO 2014/187777, US 2014/0356322, and U.S. Pat. No. 9,249,153. Compounds that bind to cereblon include thalidomide, lenalidomide, pomalidomide and analogs thereof. Other small molecule compounds that bind to cereblon are also known, e.g., the compounds disclosed as an in US 2016/0058872 and US2015/0291562. The amine protecting group can be converted to intermediates (v) using appropriate reagents such as TFA when PG=Boc. Acylation of amines (v) by activated linker reagents (X*═NHS) or by direct amide bond coupling can provide linked-PROTAC (L-C) compounds (vi) which can subsequently be conjugated to an antibody using known methods as described herein.

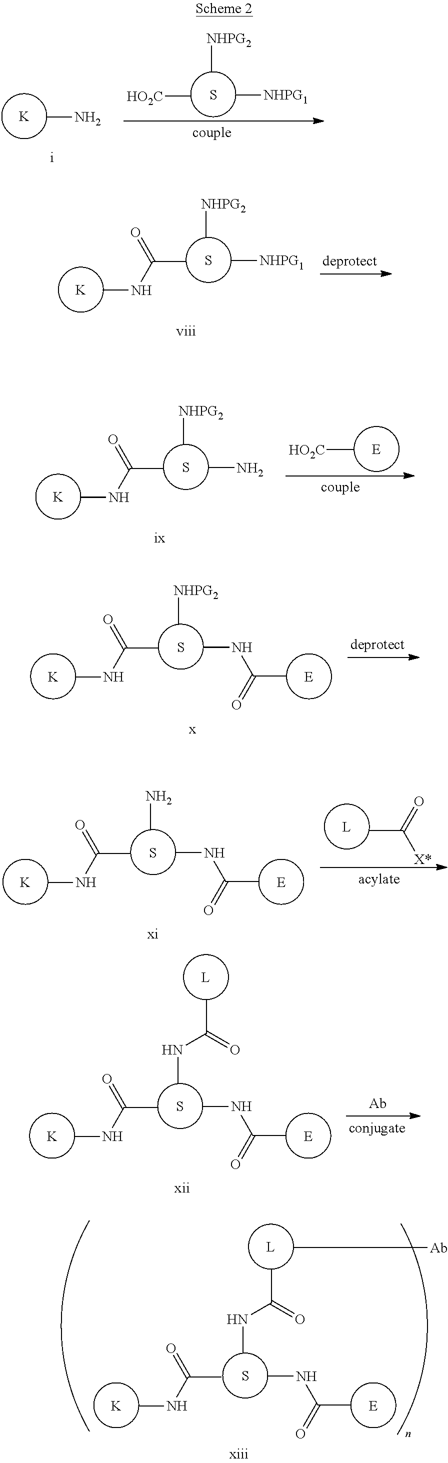

Alternatively, a kinase inhibitor containing a free amine functional group can be acylated with a multi-functional amino acid derivative such as lysine using standard amide bond coupling reactions such as HATU in DMF containing and amine base to provide intermediates (vii). Deprotection of compounds (vii) using known methods, such as hydrogenation when R=Cbz can provide compounds (ix) which can be coupled to an E3 ubiquitin ligase to provide PROTACs (x). The second amine protecting group (PG₂) can be converted to intermediates (v) using appropriate reagents such as TFA when PG=Boc. Acylation of amines (xi) by activated linker reagents (X*═NHS) or by direct amide bond coupling can provide linked-PROTAC compounds (xii), which can subsequently be conjugated to an antibody using known methods as described herein.

### FIGURE DESCRIPTIONS

FIG. 1A depicts an illustrative conjugate comprising an antibody construct, a linker (L), an immune-modulatory compound (C1; black star), a spacer (S), and a second compound (C2; gray star). The gray portion of the conjugate is the heavy chain of the antibody, and the white portion of the conjugate is the light chain of the antibody. The solid dark lines between the linker and the spacer, and the spacer and C1 and C2 denote covalent bonds.

FIG. 1B depicts an illustrative conjugate comprising an antibody construct, a linker (L), an immune-modulatory compound (C1; black star), a spacer (S), and a second compound (C2; gray star). The gray portion of the conjugate is the heavy chain of the antibody, and the white portion of the conjugate is the light chain of the antibody. The solid dark lines between the linker and C2, and the spacer and C1 denote covalent bonds.

FIG. 1C depicts an illustrative conjugate comprising an antibody construct, a linker (L), an immune-modulatory compound (C1; black star), a spacer (S), and a second compound (C2; gray star). The gray portion of the conjugate is the heavy chain of the antibody, and the white portion of the conjugate is the light chain of the antibody. The solid dark lines between the linker and C1, and the spacer and C2 denote covalent bonds.

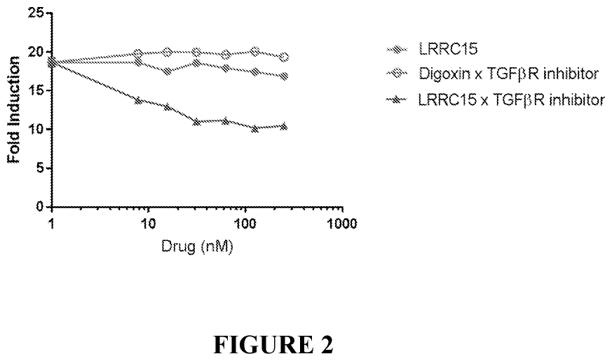

FIG. 2 shows the inhibition of the TGFβ/SMAD signaling pathway by an LRRC15 conjugate (LRRC15 antibody attached to a TGFβR inhibitor via a cleavable linker), as compared to the control antibody alone and an anti-digoxin conjugate (anti-digoxin antibody attached to the TGFβR inhibitor via a cleavable linker) control. The results show that the LRRC15-TGFβR inhibitor conjugate inhibited the TGFβ/SMAD signaling pathway following induction by TGFβ (darkest line; triangles), while the LRRC15 control antibody (middle line; closed circles) and anti-digoxin antibody-TGFβR inhibitor control conjugate (top lightest-gray line; open circles) did not significantly inhibit this signaling pathway. The y-axis is labeled as 0 to 25 in intervals of 5 for fold induction. The x-axis is labeled as a 1 to 1000 in logarithmic intervals for drug (nM).

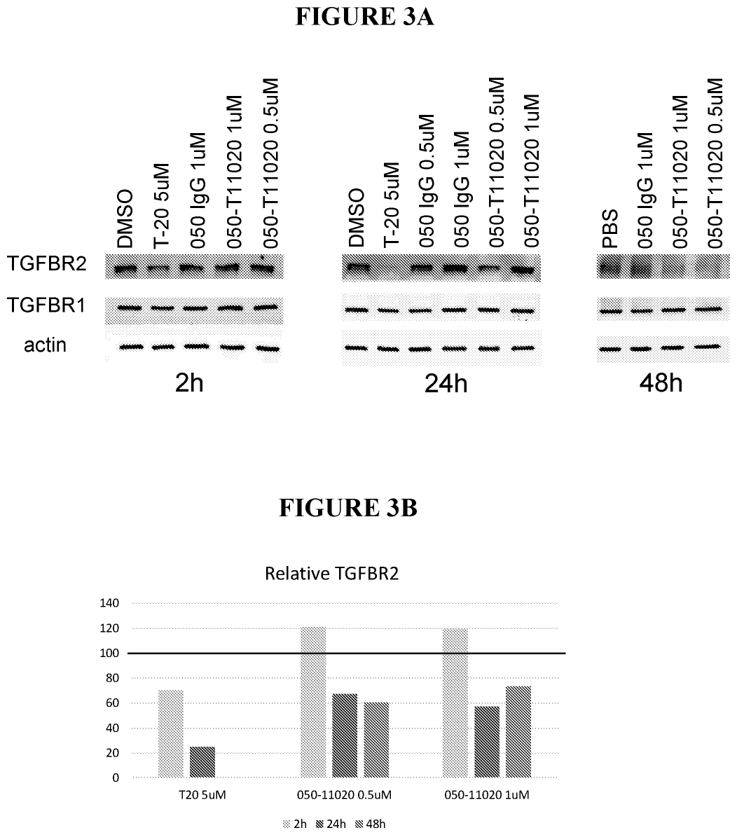

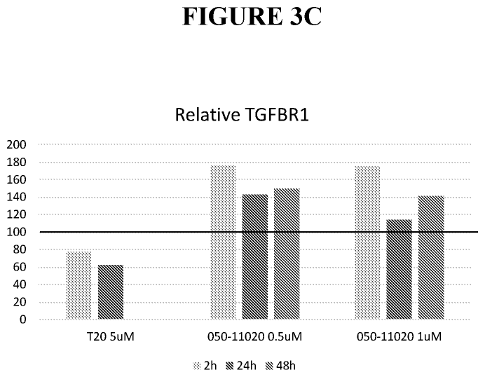

FIG. 3A shows the results of an assay for degradation of TFGβR2 by a TGFβR2-VHL PROTAC anti-HER2 antibody conjugate. Plasmid expressing HER2 was transfected into HEK293 cells, and the cells were treated with DMSO, PROTAC T-20, HER2 antibody (IgG1), or Her2 Antibody-Protac conjugate (050-T11020). Whole cell lysates were prepared from cells after 2 (left blot), 24 (middle blot), or 48 (right blot) hours incubation and quantitated with a BCA assay. Equal amounts of lysates were run on protein gels, transferred to PVDF, and TGFβR2 (top), TGFβR1 (middle), or control actin (bottom) were detected using commercially available reagents. At both tested concentrations of the conjugate, the level of target TGFβR2 was diminished at 24 and 48 hours of treatment as demonstrated by the diminished signal of TGFβR2 in the lanes containing 050-T11020. For the 2 hour blot, from left to right, the lanes represent DMSO; T-20 5 μM; 050 IgG 1 μM; 050-T11020 1 μM; and 050-T11020 0.5 μM. For the 24 hour blot, from left to right, the lanes represent DMSO; T-20 5 μM; 050 IgG 0.5 μM; 050 IgG 1 μM; 050-T11020 0.5 μM; and 050-T11020 1 μM. For the 48 hour blot, from left to right, the lanes represent PBS; 050 IgG 1 μM; 050-T11020 1 μM; and 050-T11020 0.5 μM.

FIG. 3B provides a quantification of the western blot data for TGFβR2 shown in FIG. 3A. To quantitate the amount of protein degradation, the signals on the Western blot were adjusted to actin loading control and data was presented as a percent of matched control on the y-axis, which is labeled from 0 to 140 in intervals of 20. A thick black line denotes 100 percent. The medium-gray bars at the left of each data set represent the data obtained at 2 hours of treatment. The darkest gray bars in the middle of each data set represent the data obtained at 24 hours. The lightest gray bars at the right of each data set represent the data obtained at 48 hours. On the x-axis, from the left to right, the data sets are T20 5 μM; 050-11020 0.5 μM; and 050-11020 1 μM.

FIG. 3C provides a quantification of the western blot data for TGFβR1. To quantitate the amount of protein degradation, the signals on the Western blot were adjusted to actin loading control and data was presented as a percent of matched control on the y-axis, which is labeled as 0 to 200 in intervals of 20. The medium-gray bars at the left of each data set represent the data obtained at 2 hours of treatment. The darkest gray bars in the middle of each data set represent the data obtained at 24 hours. The lightest gray bars on the right of each data set represent the data obtained at 48 hours. On the x-axis, from the left to right, the data sets are T20 5 μM; 050-11020 0.5 μM; and 050-11020 1 μM. Consistent with the western blot data, the amount of TGFβR1 protein remained fairly constant throughout the treatment period.

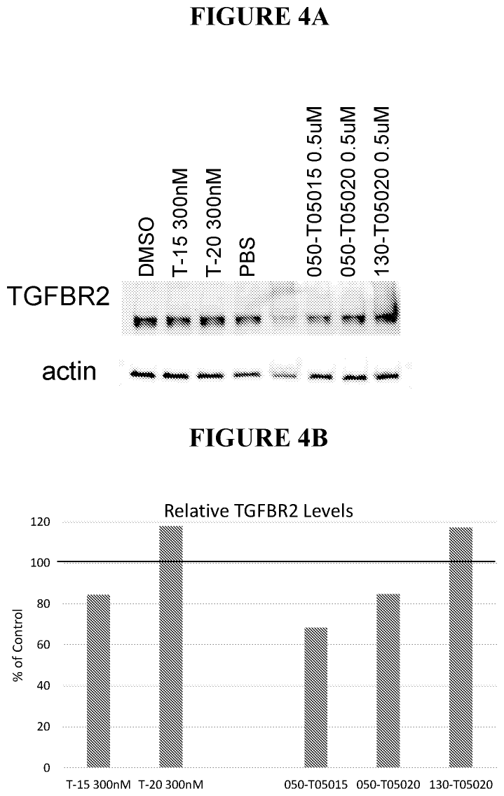

FIG. 4A and FIG. 4B show the results of an assay for antigen targeted degradation of TGFβR2 by an antibody conjugate with a PROTAC having VHL or Cereblon E3 binding moieties. BT474 cells were plated and treated the following day with either a PROTAC (T-15 or T-20), a conjugate of a HER2 antibody-TGFβR2-VHL binding PROTAC (050-T05020; T-20 PROTAC), a conjugate of a HER2 antibody-TGFβR2-Cereblon binding PROTAC (050-T05015; T-15 PROTAC), or a conjugate of a TROP2 antibody-TGFβR2-VHL binding PROTAC (130-T05020; T-20 PROTAC). Whole cell lysates were prepared 24 hours after treatment and quantitated with a BCA assay. Equal amounts of lysates were run on protein gels, transferred to PVDF, and TGFβR2 and actin were detected using commercially available reagents. FIG. 4A shows that HER2-antigen specific degradation was found with both the HER2 binding PROTAC conjugates, but not with the control TROP2-binding PROTAC conjugate, nor with the T-15 or T-20 PROTACS alone, as indicated by the retained signal of the TGFβR2 protein (top blot; actin control is bottom blot). The lanes, from left to right, represent DMSO; T-15 300 nM; T-20 300 nM; PBS; unlabeled; 050-T05015 0.5 mM; 050-T05020 0.5 μM; and 130-T05020 0.5 μM.

FIG. 4B provides a quantitation of TGFβR2 protein levels from FIG. 4A, and was determined by normalizing the TGFβR2 signals to actin loading control. The data are presented as a percent of vehicle control (100%) on the y-axis, which is labeled as 0 to 120 in intervals of 20. The x-axis, from left to right, represents T-15 300 nM; T-20 300 nM; 050-T05015; 050-T0520; and 130-T05020. The thick black line is at 100 percent of the y-axis.

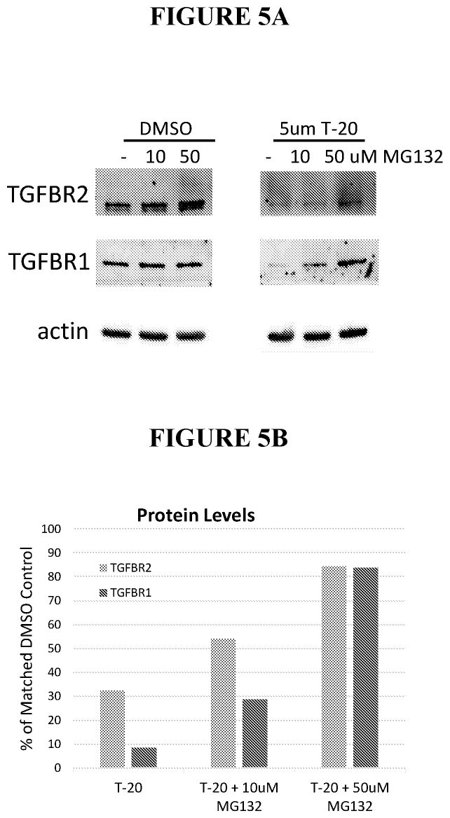

FIG. 5A and FIG. 5B show the results of an assay for cellular levels of TGFβR2 and TGFβR1 in the presence of a TGFβR2/TGFβR1-VHL PROTAC with or without the addition of a proteasome inhibitor. Normal human lung fibroblasts were treated with or without proteasome inhibitor MG-132 followed by the addition of DMSO or PROTAC T-20. Whole cell lysates were prepared and then quantitated with a BCA assay. Equal amounts of lysates were run on protein gels and transferred to PVDF membrane. TGFβR1, TGFβR2, and actin were detected using commercially available reagents. FIG. 5A provides the western blot results of the assay. The results demonstrate that the addition of the proteasome inhibitor protected TGFβR1 and TGFβR2 against degradation induced by T-20, as indicated by rescue of the TGFβR2 and TGFβR2 signals by addition of MG-132 in the presence of PROTAC T-20. TGFβR2 is the top row, TGFβR1 is the middle row, and actin is the bottom row. The left blots lanes represent, from left to right, MG132 concentrations of 0 (shown as -); 10; and 50 followed by addition of DMSO. The right blots represent, from left to right, MG132 concentrations of 0 (shown as -); 10; and 50 μM followed by addition of 5 μM T-20.

FIG. 5B provides quantification of the results of the FIG. 5A, and was obtained by adjusting the western signal to the actin loading control. The data are presented as a percent of the matched vehicle control on the y-axis, which is labeled from 0 to 100 at intervals of 10. The light gray bars represent the data for TGFβR2 and the dark gray bars represent the data for TGFβR1. The x-axis, from left to right, is labeled as T-20; T-20+10 μM MG132; and T-20+50 μM MG132.

### EXAMPLES

The following examples illustrate the various methods of making immune-modulatory compounds, linkers, linker-payloads (LPs) of immune-modulatory compounds and linkers, and conjugates described herein. It is understood that one skilled in the art may be able to make these compounds, LPs, and conjugates by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### Example 1

### Synthesis of Immune-Modulatory Compounds, Linker Payloads and Conjugates

A linker is linked with an immune-modulatory compound such as a PI3K inhibitor, Calcineurin inhibitor, mTOR inhibitor, BTK inhibitor, JAK inhibitor, CRAC inhibitor, PARP1 antagonist, PPARg agonist, Kv1.3 antagonist, KCa3.1 antagonist, PP2A agonist, IRAK4 inhibitor, MYD88 inhibitor, BCL-2 antagonist, A2ar agonist, TLR7 antagonist, c-KIT kinase inhibitor, KCA3.1 agonist, TGFβR1 inhibitor, TGFβR2 inhibitor, ACC antagonist, ASK1 antagonist, GLI1 inhibitor, TNKS antagonist, or TNIK antagonist. A linker linked to an immune-modulatory compound makes a linker-immune-modulatory compound (LP). Subsequently, a LP is conjugated to an antibody construct, such as an antibody, to form an antibody construct immune-modulatory compound conjugate or conjugate.

### Inhibitors of TGFβR2

### Example 1.1 Synthesis of (S)—N1-(4-(5-amino-6-((4-morpholinopyridin-3-yl)carbamoyl)pyrazin-2-yl)benzyl)-2-(6-(4-((2,5-dioxo-2,5-dihydro-1H-pyrrol-1-yl)methyl)-cyclohexane-1-carboxamido)hexanamido)-N5-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethyl)pentanediamide (Compound 1-1)

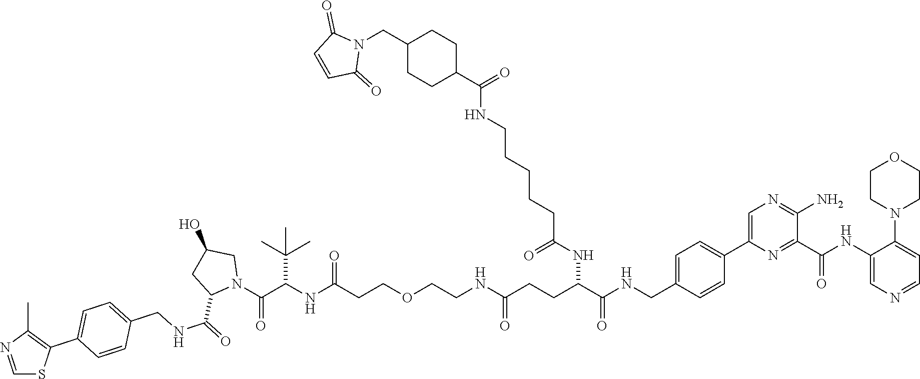 [SMILES: Cc1ncsc1-c1ccc(CNC(=O)[C@@H]2C[C@@H](O)CN2C(=O)[C@@H](NC(=O)CCOCCNC(=O)CC[C@H](NC(=O)CCCCCNC(=O)C2CCC(CN3C(=O)C=CC3=O)CC2)C(=O)NCc2ccc(-c3cnc(N)c(C(=O)Nc4cnccc4N4CCOCC4)n3)cc2)C(C)(C)C)cc1]

### Step A: Preparation of Int 1B-1

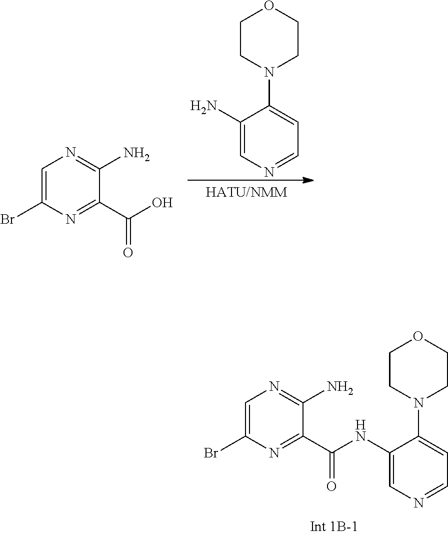 [SMILES: Nc1ncc(Br)nc1C(=O)O ; Nc1cnccc1N1CCOCC1 ; Nc1ncc(Br)nc1C(=O)Nc1cnccc1N1CCOCC1]

HATU (3.54 g, 9.36 mmol) was added to a solution containing 1.64 g (7.5 mmol) of 3-amino-6-bromopyrazine-2-carboxylic acid in 25 mL of DMF. The reaction was stirred for 5 minutes before adding 2.5 mL (22.5 mmol) of N-methylmorpholine and 1.68 g (9.36 mmol) of 4-morpholinopyridin-3-amine. The reaction mixture was stirred for 16 h then quenched with 10 mL of saturated NH₄Cl solution and then 10 mL of water. The mixture was extracted with EtOAc three times; the combined organics were washed with brine and then dried over Na₂SO₄. The solvent was then evaporated and the residue was chromatographed (0% to 20% CH₃OH/dichloromethane) to afford compound Int 1B-1 as a yellow solid.

### Step B: Preparation of Int 1B-2

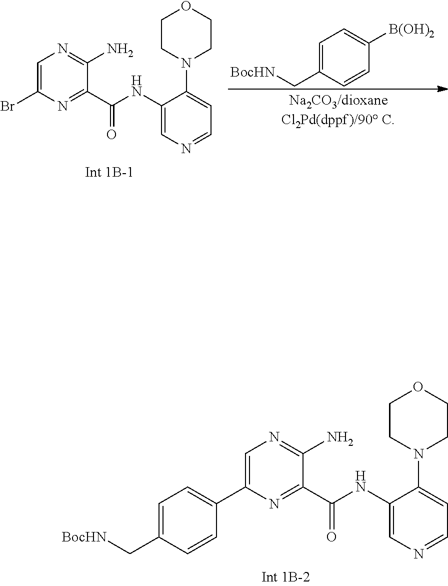 [SMILES: Nc1ncc(Br)nc1C(=O)Nc1cnccc1N1CCOCC1 ; CC(C)(C)OC(=O)NCc1ccc(B(O)O)cc1 ; CC(C)(C)OC(=O)NCc1ccc(-c2cnc(N)c(C(=O)Nc3cnccc3N3CCOCC3)n2)cc1]

A solution containing 1.5 g (4.0 mmol) of 3-amino-6-bromo-N-(4-morpholinopyridin-3-yl)pyrazine-2-carboxamide and 1.1 g (4.4 mmol) of (4-(2-(((tert-butoxy)carbonyl)-amino)methyl)phenyl)boronic acid in 25 mL of dioxane and 4.0 mL of 2N Na₂CO₃ (8.0 mmol) was degassed and back filled with nitrogen three times. 295 mg (0.4 mmol) of PdCl₂ (dppf) was added and the reaction vessel was degassed with nitrogen twice. The reaction mixture was then heated at 90° C. for 3 h then cooled and stirred overnight then filtered through a plug of Celite®. The filtrate was diluted with EtOAc, washed with water and then brine, and dried over Na₂SO₄. The solvent was then evaporated and the residue was chromatographed (0% to 20% MeOH/dichloromethane) to afford 1.3 g of compound Int 1B-2 as a white solid. LCMS (M+H)=506.

### Step C: Preparation of Int 1B-3

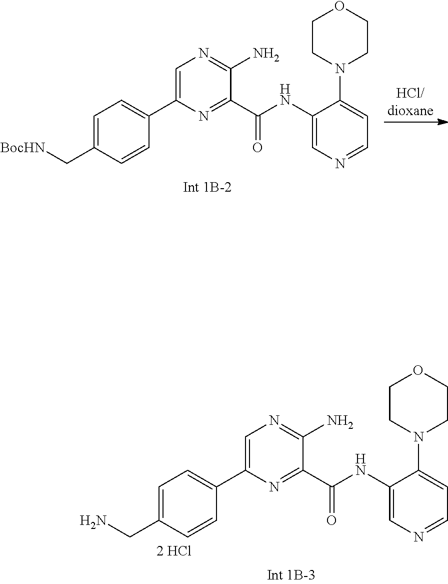 [SMILES: CC(C)(C)OC(=O)NCc1ccc(-c2cnc(N)c(C(=O)Nc3cnccc3N3CCOCC3)n2)cc1 ; NCc1ccc(-c2cnc(N)c(C(=O)Nc3cnccc3N3CCOCC3)n2)cc1]

A solution containing 1.2 g (2.4 mmol) of Int 1B-2 in 25 mL of EtOAc was added 10 mL of 4N HCl in dioxane at room temperature. The reaction was stirred for 3 h and the solvent was evaporated. The resulting solid was triturated three times with toluene to provide the desired amine salt which was used without purification. LCMS (M+H)=406.

### Step D: Preparation of Int 1B-4

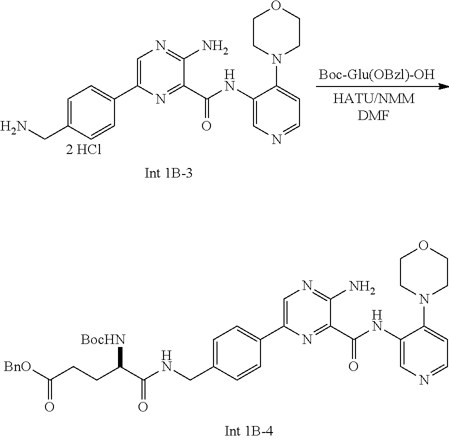 [SMILES: NCc1ccc(-c2cnc(N)c(C(=O)Nc3cnccc3N3CCOCC3)n2)cc1 ; CC(C)(C)OC(=O)N[C@H](CCC(=O)OCc1ccccc1)C(=O)NCc1ccc(-c2cnc(N)c(C(=O)Nc3cnccc3N3CCOCC3)n2)cc1]

To a solution containing 112 mg (0.276 mmol) of Int 1B-3 and 93 mg (0.276 mmol) of Boc-L-glutamic acid 5-benzyl ester in 2 mL of DMF was added 105 mg (0.276 mmol) of HATU and 0.06 mL (0.55 mmol) of N-methylmorpholine. The reaction mixture was stirred for 16 h then quenched with 1 mL of saturated NH₄Cl solution and 1 mL of water. The mixture was extracted with EtOAc three times; the combined organics were washed with brine and then dried over Na₂SO₄. The solvent was then evaporated and the residue was chromatographed (0% to 20% CH₃OH/dichloromethane) to afford 160 mg of compound Int 1B-4 as a yellow solid. LCMS (M+H)=725.

### Step E: Preparation of Int 1B-5

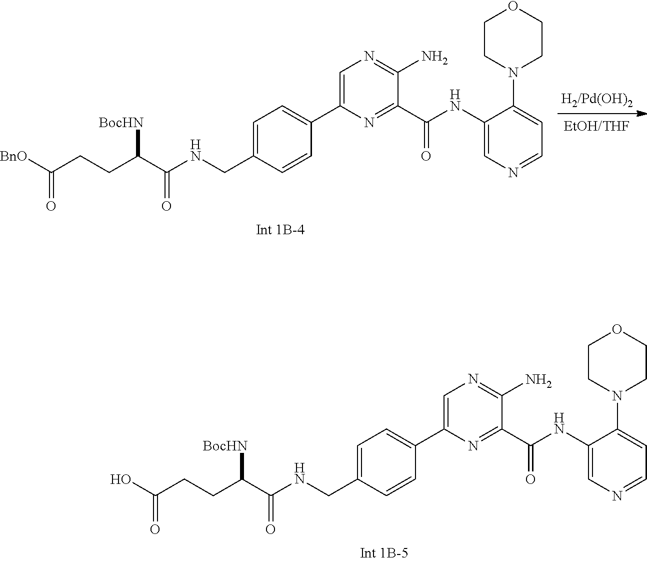 [SMILES: CC(C)(C)OC(=O)N[C@H](CCC(=O)OCc1ccccc1)C(=O)NCc1ccc(-c2cnc(N)c(C(=O)Nc3cnccc3N3CCOCC3)n2)cc1 ; CC(C)(C)OC(=O)N[C@H](CCC(=O)O)C(=O)NCc1ccc(-c2cnc(N)c(C(=O)Nc3cnccc3N3CCOCC3)n2)cc1]

A solution containing 100 mg (0.14 mmol) of Int 1B-4 in 20 mL of 1:1 THF-EtOH was degassed and back filled with nitrogen three times. 100 mg of 20% Pd(OH)₂ was added and the mixture was degassed two additional times. The reaction mixture was stirred for 16 h then filtered through Celite with EtOAc. Removal of the solvent and trituration with toluene afforded 75 mg of Int 1B-5 which was used directly in the next step. LCMS (M+H)=635.

### Step F: Preparation of Int 1B-6

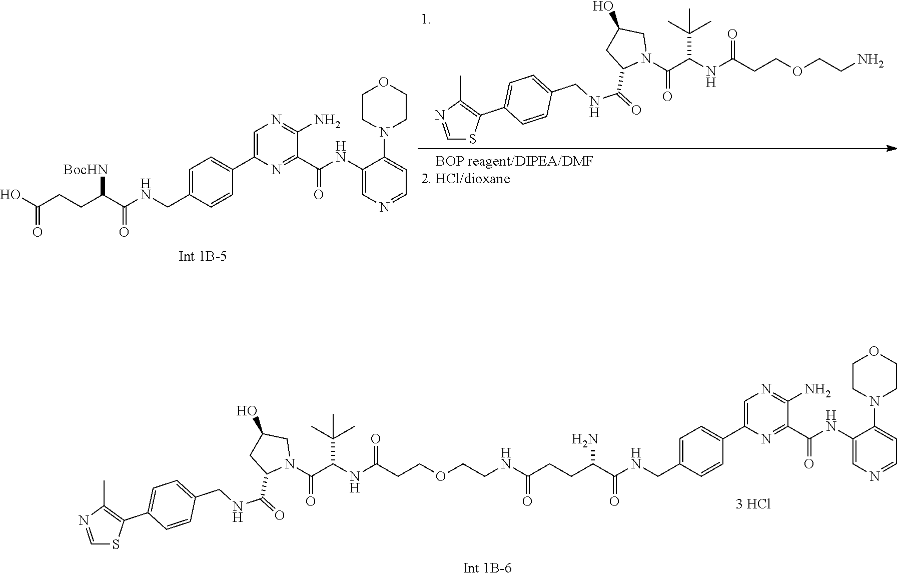

To a solution containing 75 mg (0.12 mmol) of Int 1B-5 and 82 mg (0.15 mmol) of (2S,4R)-1-((S)-2-(3-(2-aminoethoxy)propanamido)-3,3-dimethylbutanoyl)-4-hydroxy-N-(4-(4-methylthiazol-5-yl)benzyl)pyrrolidine-2-carboxamide in 1.0 mL of DMF was added 66 mg (0.15 mmol) of BOP reagent and 0.026 mL (0.24 mmol) of diisopropylethylamine. The reaction mixture was stirred for 16 h then quenched with 1 mL of saturated NaHCO₃ solution and 1 mL of water. The mixture was extracted with EtOAc three times; the combined organic extracts were washed with brine and then dried over Na₂SO₄. The solvent was then evaporated and the residue was chromatographed (0% to 20% CH₃OH/dichloromethane) to afford 58 mg of the desired compound as a yellow solid which was immediately dissolved in 5 mL of EtOAc then treated with 1 mL of 4 N HCl in dioxane at room temperature and the reaction was stirred for 3 h. The solvent was removed under reduced pressure and the residue was azeotroped three times with toluene then stirred with ether and filtered to afford 43 mg of (S)-2-amino-N¹-(4-(5-amino-6-((4-morpholinopyridin-3-yl)carbamoyl)pyrazin-2-yl)benzyl)-N5-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)-ethyl)pentanediamide trihydrochloride as bright yellow crystalline solid. LCMS (M+H)=1062.

Int 1B-6 is PROTAC T-015

### Step G: Preparation of Compound 1-1

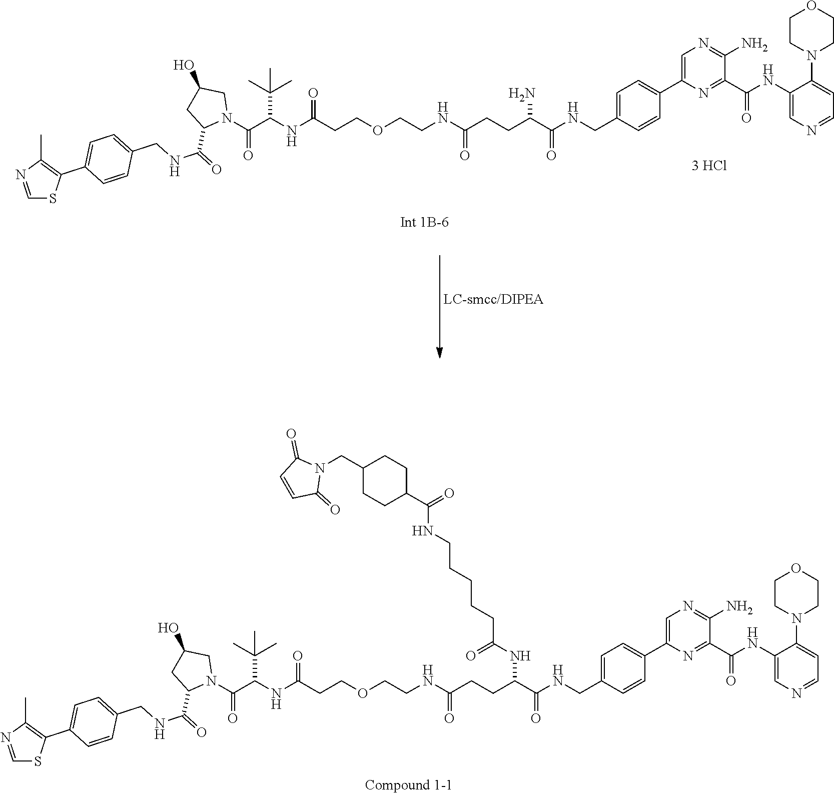 [SMILES: Cc1ncsc1-c1ccc(CNC(=O)[C@@H]2C[C@@H](O)CN2C(=O)[C@@H](NC(=O)CCOCCNC(=O)CC[C@H](N)C(=O)NCc2ccc(-c3cnc(N)c(C(=O)Nc4cnccc4N4CCOCC4)n3)cc2)C(C)(C)C)cc1 ; Cc1ncsc1-c1ccc(CNC(=O)[C@@H]2C[C@@H](O)CN2C(=O)[C@@H](NC(=O)CCOCCNC(=O)CC[C@H](NC(=O)CCCCCNC(=O)C2CCC(CN3C(=O)C=CC3=O)CC2)C(=O)NCc2ccc(-c3cnc(N)c(C(=O)Nc4cnccc4N4CCOCC4)n3)cc2)C(C)(C)C)cc1]

A solution containing 43 mg (0.037 mmol) of (S)-2-amino-N¹-(4-(5-amino-6-((4-morpholinopyridin-3-yl)carbamoyl)pyrazin-2-yl)benzyl)-N5-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethyl)pentanediamide trihydrochloride was combined with (16 mg, 0.037 mmol) of LC-smcc (succinimidyl 4-(N-maleimidomethyl)cyclohexane-1-carboxy-(6-amidocaproate)) in 1.5 mL of DCM and DIPEA (0.064 mL, 0.36 mmol). After stirring overnight, the reaction became cloudy and LCMS indicated the presence of product. The reaction was concentrated then taken up in a minimum amount of THF and water. The mixture was neutralized with saturated NaHCO₃ and the mixture was chromatographed (30 g, C18, H₂O to CH₃CN, liquid load) to provide Compound 1-1 (31.8, mg) as a yellow solid after lyophilization from CH₃CN/H₂O. ¹H NMR (CD₃OD) δ 9.46 (s, 1H), 8.84 (s, 1H), 8.78 (s, 1H), 8.26 (d, J=8.5 Hz, 1H), 8.03 (d, J=8.5 Hz, 2H), 7.44 (d, J=8.5 Hz, 2H), 7.41 (d, J=8.4 Hz, 2H), 7.35 (d, J=8.4 Hz, 2H), 7.26 (d, J=5.5 Hz, 1H), 6.78 (s, 2H), 4.66 (s, 1H), 4.59 (m, 2H), 4.46 (t, J=7.0 Hz, 4H), 4.37 (m, 2H), 3.88 (d, J=11.5 Hz, 1H), 3.81-3.70 (m, 5H), 3.69 (t, J=5.5 Hz, 2H), 3.55-3.49 (m, 3H), 3.11 (t, J=11.5 Hz, 2H), 3.10-3.01 (m, 5H), 2.50 (t, J=15.0 Hz, 2H), 2.33 (s, 3H), 2.35-2.22 (m, 6H), 2.11-2.01 (m, 4H), 1.94 (m, 1H), 1.76-1.58 (m, 8H), 1.50-1.25 (m, 8H), 1.11 (s, 9H), 1.05-0.95 (m, 4H). LCMS (M+H)=1395.6.

The following compounds in TABLE 3, TABLE 4, and TABLE 5 were prepared in an analogous manner to that described for the synthesis of Compound 1-1 by substituting the appropriate aryl boronic acid in step B and E3 ligase ligand/spacer group in step E.

TABLE 3
Exemplary Compounds

| Compound | 1-2 |
|---|---|
| Structure | 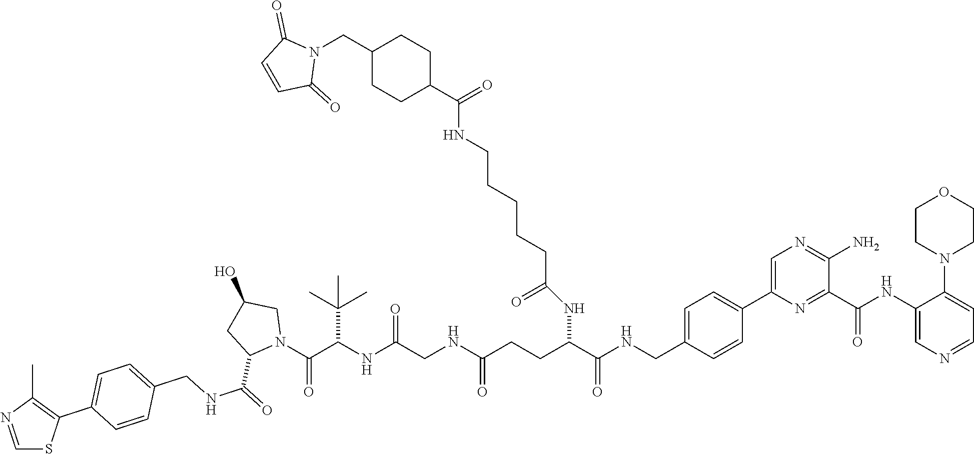 [SMILES: Cc1ncsc1-c1ccc(CNC(=O)[C@@H]2C[C@@H](O)CN2C(=O)[C@@H](NC(=O)CNC(=O)CC[C@H](NC(=O)CCCCCNC(=O)C2CCC(CN3C(=O)C=CC3=O)CC2)C(=O)NCc2ccc(-c3cnc(N)c(C(=O)Nc4cnccc4N4CCOCC4)n3)cc2)C(C)(C)C)cc1] |
| IUPAC | (S)-N1-(4-(5-amino-6-((4- |
| Name | morpholinopyridin-3- |
| | yl)carbamoyl)pyrazin-2-yl)benzyl)- |
| | 2-(6-(4-((2,5-dioxo-2,5-dihydro- |
| | 1H-pyrrol-1- |
| | yl)methyl)cyclohexane-1- |
| | carboxamido)hexanamido)-N5-(2- |
| | (((S)-1-((2S,4R)-4-hydroxy-2-((4- |
| | (4-methylthiazol-5- |
| | yl)benzyl)carbamoyl)pyrrolidin-1- |
| | yl)-3,3-dimethyl-1-oxobutan-2- |
| | yl)amino)-2- |
| | oxoethyl)pentanediamide |
| ArB(OH)2 | 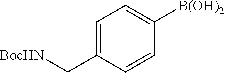 [SMILES: CC(C)(C)OC(=O)NCc1ccc(B(O)O)cc1] |
| E3 Ligand | 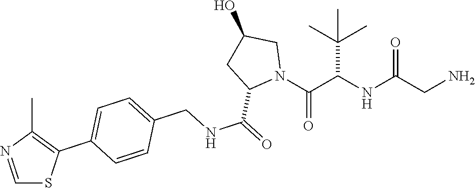 [SMILES: Cc1ncsc1-c1ccc(CNC(=O)[C@@H]2C[C@@H](O)CN2C(=O)[C@@H](NC(=O)CN)C(C)(C)C)cc1] |
| M + 1 | 1336 |
| Compound | 1-3 |
| Structure | 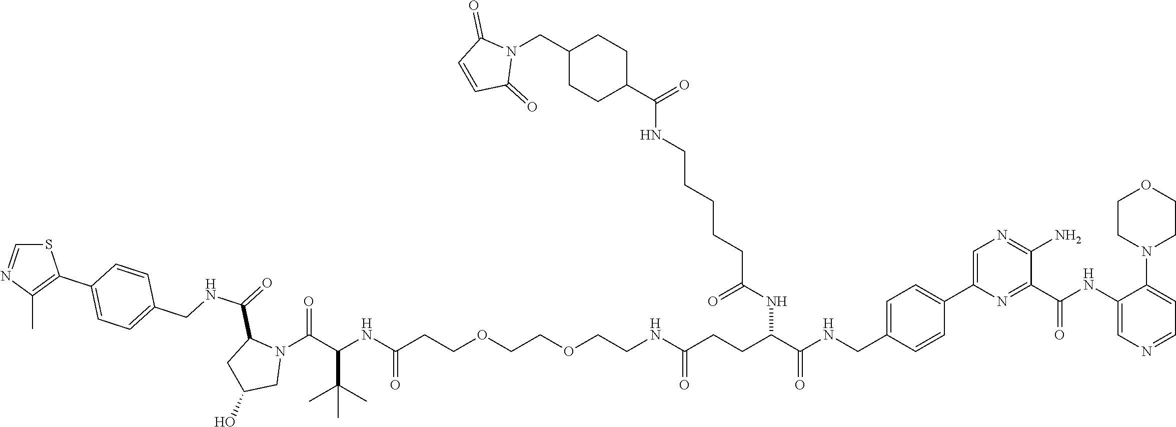 [SMILES: Cc1ncsc1-c1ccc(CNC(=O)[C@@H]2C[C@@H](O)CN2C(=O)[C@@H](NC(=O)CCOCCOCCNC(=O)CC[C@H](NC(=O)CCCCCNC(=O)C2CCC(CN3C(=O)C=CC3=O)CC2)C(=O)NCc2ccc(-c3cnc(N)c(C(=O)Nc4cnccc4N4CCOCC4)n3)cc2)C(C)(C)C)cc1] |
| IUPAC | (S)-N1-(4-(5-amino-6-((4- |
| Name | morpholinopyridin-3- |
| | yl)carbamoyl)pyrazin-2-yl)benzyl)-2- |
| | (6-(4-((2,5-dioxo-2,5-dihydro-1H- |
| | pyrrol-1-yl)methyl)cyclohexane-1- |
| | carboxamido)hexanamido)-N5-(2-(2-(3- |
| | (((S)-1-((2S,4R)-4-hydroxy-2-((4-(4- |
| | methylthiazol-5- |
| | yl)benzyl)carbamoyl)pyrrolidin-1-yl)- |
| | 3,3-dimethyl-1-oxobutan-2-yl)amino)- |
| | 3- |
| | oxopropoxy)ethoxy)ethyl)pentanediamide |
| ArB(OH)2 | 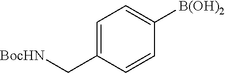 [SMILES: CC(C)(C)OC(=O)NCc1ccc(B(O)O)cc1] |
| E3 Ligand | 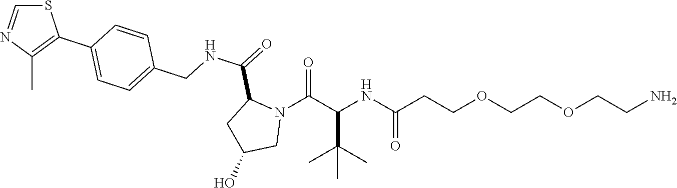 [SMILES: Cc1ncsc1-c1ccc(CNC(=O)[C@@H]2C[C@@H](O)CN2C(=O)[C@@H](NC(=O)CCOCCOCCN)C(C)(C)C)cc1] |
| M + 1 | 1438 |

TABLE 4
Exemplary Compounds

| Compound | 1-4 |
|---|---|
| Structure | 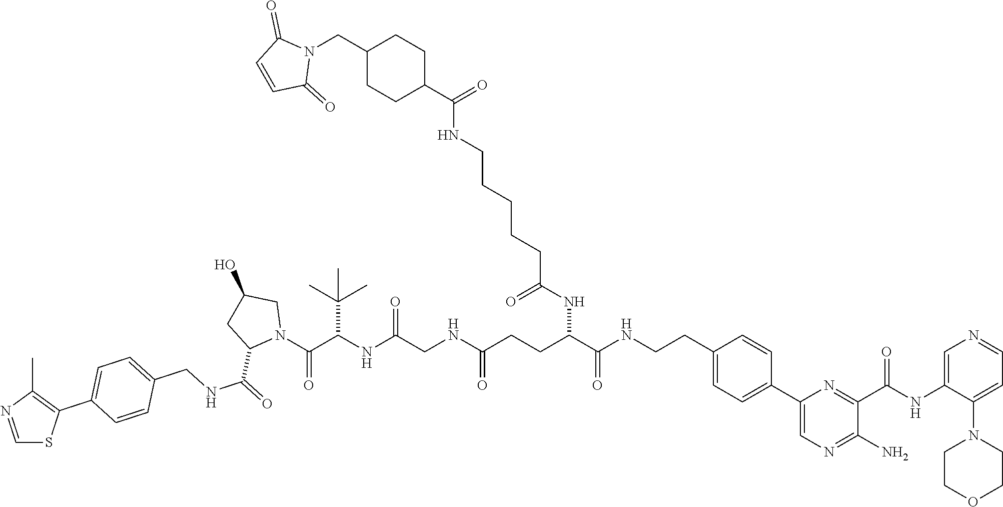 [SMILES: Cc1ncsc1-c1ccc(CNC(=O)[C@@H]2C[C@@H](O)CN2C(=O)[C@@H](NC(=O)CNC(=O)CC[C@H](NC(=O)CCCCCNC(=O)C2CCC(CN3C(=O)C=CC3=O)CC2)C(=O)NCCc2ccc(-c3cnc(N)c(C(=O)Nc4cnccc4N4CCOCC4)n3)cc2)C(C)(C)C)cc1] |
| IUPAC | (S)-N1-(4-(5-amino-6-((4- |
| Name | morpholinopyridin-3- |
| | yl)carbamoyl)pyrazin-2- |
| | yl)phenethyl)-2-(6-(4-((2,5-dioxo- |
| | 2,5-dihydro-1H-pyrrol-1- |
| | yl)methyl)cyclohexane-1- |
| | carboxamido)hexanamido)-N5-(2- |
| | (((S)-1-((2S,4R)-4-hydroxy-2-((4- |
| | (4-methylthiazol-5- |
| | yl)benzyl)carbamoyl)pyrrolidin-1- |
| | yl)-3,3-dimethyl-1-oxobutan-2- |
| | yl)amino)-2- |
| | oxoethyl)pentanediamide |
| ArB(OH)₂ | 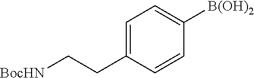 [SMILES: CC(C)(C)OC(=O)NCCc1ccc(B(O)O)cc1] |
| E3 Ligand | 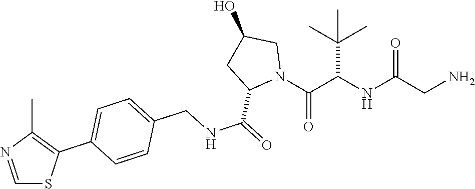 [SMILES: Cc1ncsc1-c1ccc(CNC(=O)[C@@H]2C[C@@H](O)CN2C(=O)[C@@H](NC(=O)CN)C(C)(C)C)cc1] |
| M + 1 | 1350 |
| Compound | 1-5 |
| Structure | 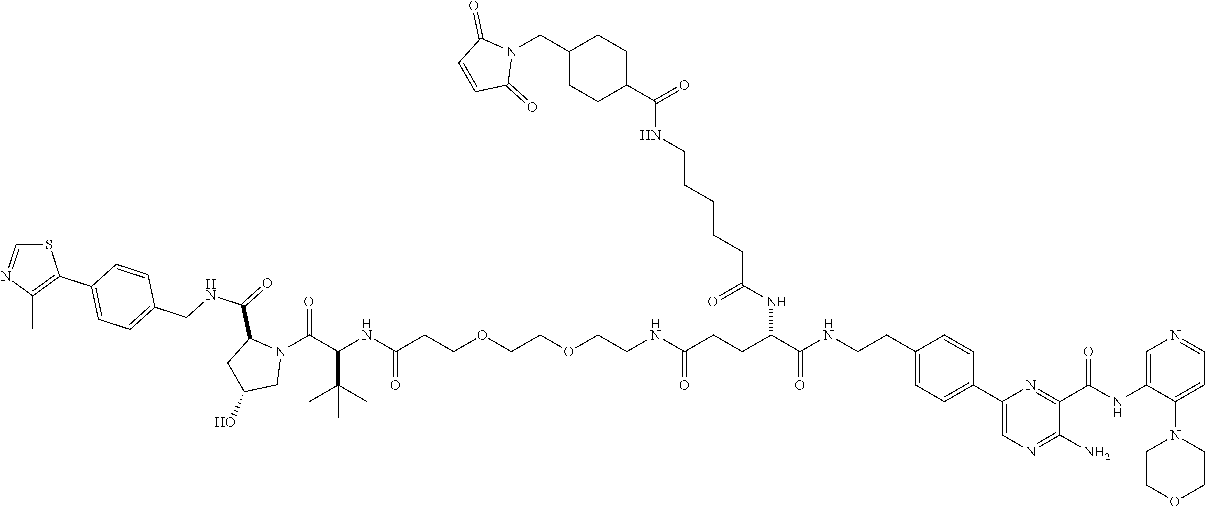 [SMILES: Cc1ncsc1-c1ccc(CNC(=O)[C@@H]2C[C@@H](O)CN2C(=O)[C@@H](NC(=O)CCOCCOCCNC(=O)CC[C@H](NC(=O)CCCCCNC(=O)C2CCC(CN3C(=O)C=CC3=O)CC2)C(=O)NCCc2ccc(-c3cnc(N)c(C(=O)Nc4cnccc4N4CCOCC4)n3)cc2)C(C)(C)C)cc1] |
| IUPAC | (S)-N1-(4-(5-amino-6-((4- |
| Name | morpholinopyridin-3- |
| | yl)carbamoyl)pyrazin-2-yl)phenethyl)- |
| | 2-(6-(4-((2,5-dioxo-2,5-dihydro-1H- |
| | pyrrol-1-yl)methyl)cyclohexane-1- |
| | carboxamido)hexanamido)-N5-(2-(2-(3- |
| | (((S)-1-((2S,4R)-4-hydroxy-2-((4-(4- |
| | methylthiazol-5- |
| | yl)benzyl)carbamoyl)pyrrolidin-1-yl)- |
| | 3,3-dimethyl-1-oxobutan-2-yl)amino)- |
| | 3- |
| | oxopropoxy)ethoxy)ethyl)pentanediamide |
| ArB(OH)₂ | 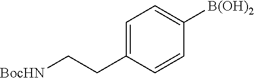 [SMILES: CC(C)(C)OC(=O)NCCc1ccc(B(O)O)cc1] |
| E3 Ligand | 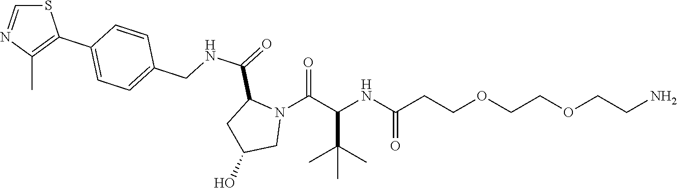 [SMILES: Cc1ncsc1-c1ccc(CNC(=O)[C@@H]2C[C@@H](O)CN2C(=O)[C@@H](NC(=O)CCOCCOCCN)C(C)(C)C)cc1] |
| M + 1 | 1452 |

TABLE 5
Exemplary Compounds

| Compound | 1-6 |
|---|---|
| Structure | 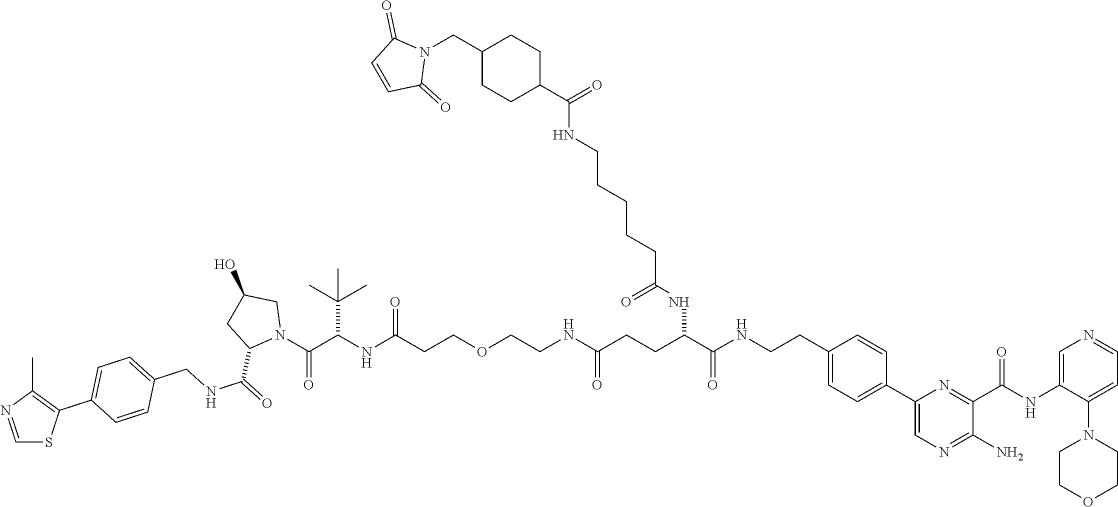 [SMILES: Cc1ncsc1-c1ccc(CNC(=O)[C@@H]2C[C@@H](O)CN2C(=O)[C@@H](NC(=O)CCOCCNC(=O)CC[C@H](NC(=O)CCCCCNC(=O)C2CCC(CN3C(=O)C=CC3=O)CC2)C(=O)NCCc2ccc(-c3cnc(N)c(C(=O)Nc4cnccc4N4CCOCC4)n3)cc2)C(C)(C)C)cc1] |
| IUPAC | (S)-N1-(4-(5-amino-6-((4- |
| Name | morpholinopyridin-3- |
| | yl)carbamoyl)pyrazin-2-yl)phenethyl)- |
| | 2-(6-(4-((2,5-dioxo-2,5-dihydro-1H- |
| | pyrrol-1-yl)methyl)cyclohexane-1- |
| | carboxamido)hexanamido)-N5-(2-(3- |
| | (((S)-1-((2S,4R)-4-hydroxy-2-((4-(4- |
| | methylthiazol-5- |
| | yl)benzyl)carbamoyl)pyrrolidin-1-yl)- |
| | 3,3-dimethyl-1-oxobutan-2-yl)amino)- |
| | 3-oxopropoxy)ethyl)pentanediamide |
| ArB(OH)₂ | 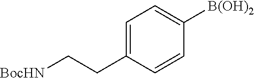 [SMILES: CC(C)(C)OC(=O)NCCc1ccc(B(O)O)cc1] |
| E3 Ligand | 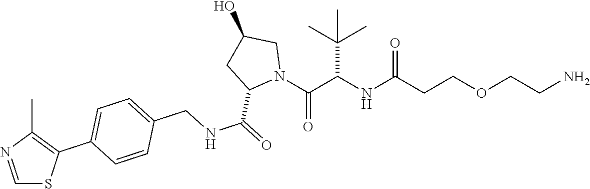 [SMILES: Cc1ncsc1-c1ccc(CNC(=O)[C@@H]2C[C@@H](O)CN2C(=O)[C@@H](NC(=O)CCOCCN)C(C)(C)C)cc1] |
| M + 1 | 1408 |

### Example 1.2. Synthesis of 3-amino-6-(4-(2-((2S)-2-(6-(4-((2,5-dioxo-2,5-dihydro-1H-pyrrol-1-yl)methyl)cyclohexane-1-carboxamido)hexanamido)-6-(2-((2-(2,6-dioxopiperidin-3-yl)-1,3-dioxoisoindolin-4-yl)oxy)acetamido)hexanamido)ethyl)phenyl)-N-(4-morpholinopyridin-3-yl)pyrazine-2-carboxamide (Compound 2-1)

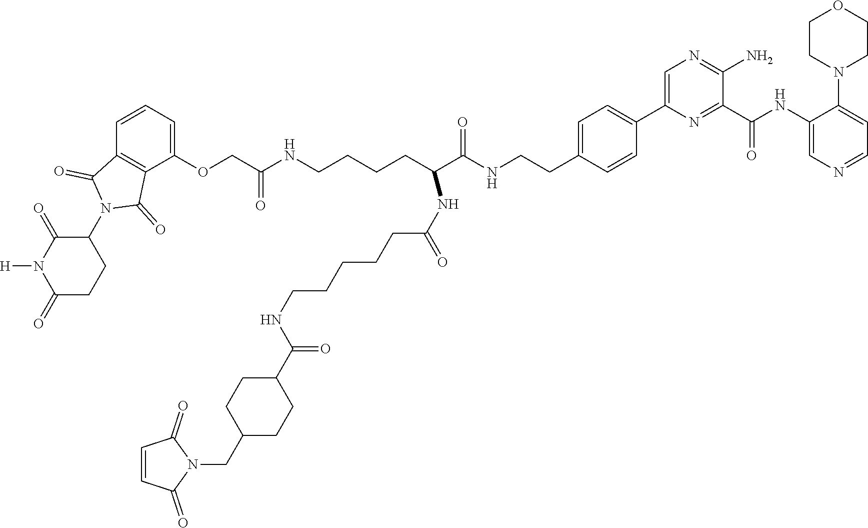 [SMILES: Nc1ncc(-c2ccc(CCNC(=O)[C@H](CCCCNC(=O)COc3cccc4c3C(=O)N(C3CCC(=O)NC3=O)C4=O)NC(=O)CCCCCNC(=O)C3CCC(CN4C(=O)C=CC4=O)CC3)cc2)nc1C(=O)Nc1cnccc1N1CCOCC1]

### Step A: Preparation of Int 7B-1

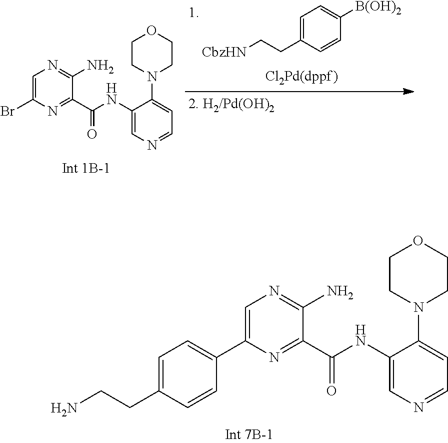 [SMILES: Nc1ncc(Br)nc1C(=O)Nc1cnccc1N1CCOCC1 ; O=C(NCCc1ccc(B(O)O)cc1)OCc1ccccc1 ; NCCc1ccc(-c2cnc(N)c(C(=O)Nc3cnccc3N3CCOCC3)n2)cc1]

A solution containing 3.0 g (8.0 mmol) of 3-amino-6-bromo-N-(4-morpholinopyridin-3-yl)pyrazine-2-carboxamide and 2.6 g (8.8 mmol) of (4-(2-(((tert-butoxy)carbonyl)-amino)ethyl)phenyl)boronic acid in 50 mL of dioxane and 8 mL of 2N Na₂CO₃ (16.0 mmol) was degassed and back filled with nitrogen three times. 600 mg (0.8 mmol) of PdCl₂ (dppf) was added and the reaction vessel was degassed with nitrogen twice. The reaction mixture was then heated at 90° C. for 3 h then cooled and stirred overnight then filtered through a plug of Celite®. The filtrate was diluted with EtOAc, washed with water and then brine, and dried over Na₂SO₄. The solvent was then evaporated and the residue was chromatographed (0% to 20% MeOH/dichloromethane) to afford 2.5 g of compound Int 1.2a as a brown solid. The material was dissolved in 100 mL of 1:1 THF:EtOH was degassed and back filled with nitrogen three times. 500 mg of 20% Pd(OH)₂ was added and the mixture was degassed two additional times. The reaction mixture was stirred for 16 h then filtered through Celite with EtOAc. Removal of the solvent afforded 2.0 g of Int 7B-1 which was used directly in the next step. LCMS (M+H)=420.

### Step B: Preparation of Int 7B-2

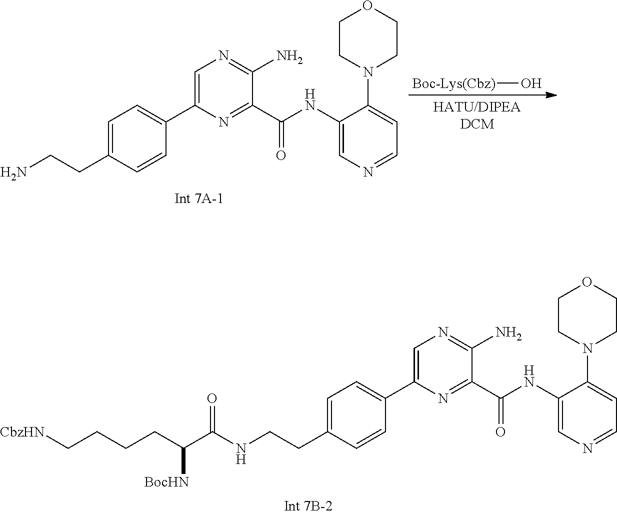

To a solution containing 228 mg (0.60 mmol) of Boc-L-Lys(Z)—OH in 5 mL of DMF was added 228 mg (0.60 mmol) of HATU and the reaction was stirred for 5 minutes before the addition of 210 mg (0.50 mmol) of Int 7B-1 and 121 mg (1.2 mmol) of N-methylmorpholine. The reaction mixture was stirred for 3 h then quenched with 5 mL of saturated NaHCO₃ solution and 2 mL of water. The mixture was extracted with EtOAc three times; the combined organics were washed with brine and then dried over Na₂SO₄. The solvent was then evaporated and the residue was chromatographed (0% to 20% CH₃OH/dichloromethane) to afford 190 mg of compound Int 7B-2 as a yellow solid. LCMS (M+H)=782.

### Step C: Preparation of Int 7B-3

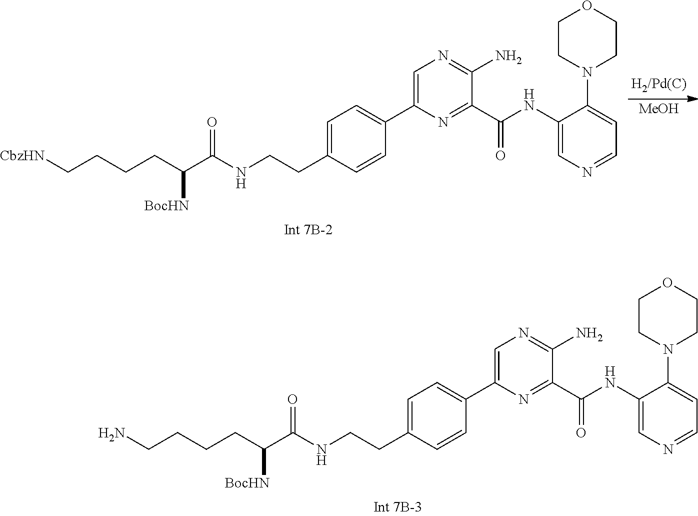 [SMILES: "MeOH" ; CC(C)(C)OC(=O)N[C@@H](CCCCNC(=O)OCc1ccccc1)C(=O)NCCc1ccc(-c2cnc(N)c(C(=O)Nc3cnccc3N3CCOCC3)n2)cc1 ; CC(C)(C)OC(=O)N[C@@H](CCCCN)C(=O)NCCc1ccc(-c2cnc(N)c(C(=O)Nc3cnccc3N3CCOCC3)n2)cc1]

A solution containing 164 mg (0.21 mmol) of Int 7B-2 in 10 mL of methanol was degassed three times while back filling with nitrogen before the addition of 50 mg of 5% Pd on carbon. A balloon of hydrogen was added and the reaction was stirred for 3 h then filtered through Celite with EtOAc. Removal of the solvent afforded 40 mg of Int 7B-3 as a yellow solid. ¹H NMR (CD₃OD) δ 9.48 (s, 1H), 8.77 (s, 1H), 8.28 (d, J=5.6 Hz, 1H), 8.02 (d, J=8.0 Hz, 2H), 7.40 (d, J=8.0 Hz, 2H), 7.28 (d, J=5.2 Hz, 1H), 3.98-3.79 (m, 5H), 3.51 (m, 2H), 3.04 (t, J=4.8 Hz, 4H), 2.90 (t, J=5.1 Hz, 2H), 2.62 (t, J=7.2 Hz, 2H), 1.68 (m, 1H), 1.51 (s, 9H), 1.44-1.22 (m, 5H). LCMS (M+H)=648.3.

### Step D: Preparation of Int 7B-4

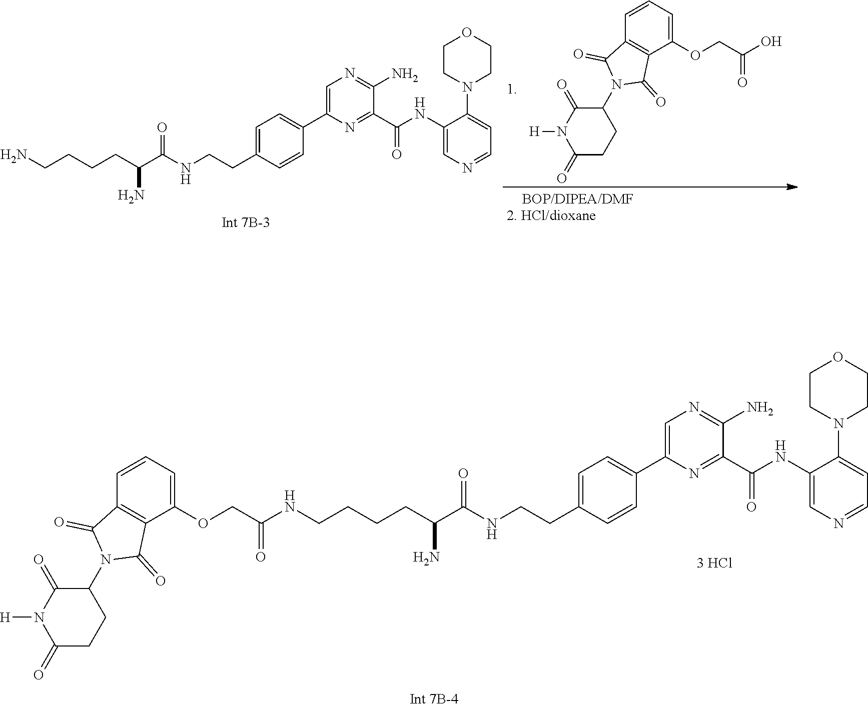 [SMILES: NCCCC[C@H](N)C(=O)NCCc1ccc(-c2cnc(N)c(C(=O)Nc3cnccc3N3CCOCC3)n2)cc1 ; O=C(O)COc1cccc2c1C(=O)N(C1CCC(=O)NC1=O)C2=O ; Nc1ncc(-c2ccc(CCNC(=O)[C@@H](N)CCCCNC(=O)COc3cccc4c3C(=O)N(C3CCC(=O)NC3=O)C4=O)cc2)nc1C(=O)Nc1cnccc1N1CCOCC1]

To a solution containing 58 mg (0.09 mmol) of Int 7B-3 and 30 mg (0.09 mmol) of 2-((2-(2,6-dioxopiperidin-3-yl)-1,3-dioxoisoindolin-4-yl)oxy)acetic acid in 1 mL of DMF was added 48 mg (0.11 mmol) of BOP reagent and 0.047 mL (0.27 mmol) of diisopropylethylamine. The reaction mixture was stirred for 16 h then quenched with 1 mL of saturated NaHCO₃ solution and 1 mL of water. The mixture was extracted with EtOAc three times and the combined organic extracts were washed with brine and then dried over Na₂SO₄. The solvent was then evaporated and the residue was chromatographed (0% to 20% CH₃OH/dichloromethane) to afford 60 mg of the desired compound which was immediately dissolved in 4 mL of EtOAc and 1 mL of methanol then treated with 2 mL of 4 N HCl in dioxane at room temperature and the reaction was stirred for 2 h. The solvent was removed under reduced pressure and the yellow solid was evaporated three times from diethyl ether to afford 49 mg of Int 7B-4 as bright yellow crystalline solid. LCMS (M+H)=862.

Int 7B-4 is PROTAC T-20.

### Step E: Preparation of Compound 2-1

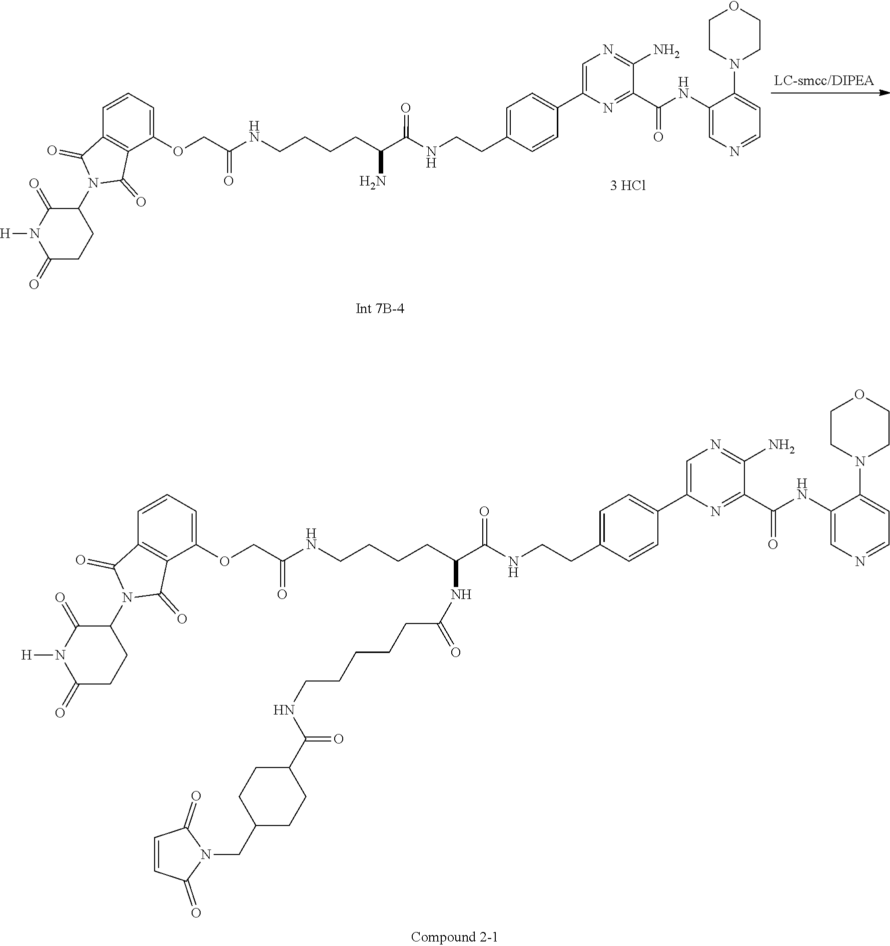 [SMILES: Nc1ncc(-c2ccc(CCNC(=O)[C@@H](N)CCCCNC(=O)COc3cccc4c3C(=O)N(C3CCC(=O)NC3=O)C4=O)cc2)nc1C(=O)Nc1cnccc1N1CCOCC1 ; Nc1ncc(-c2ccc(CCNC(=O)[C@H](CCCCNC(=O)COc3cccc4c3C(=O)N(C3CCC(=O)NC3=O)C4=O)NC(=O)CCCCCNC(=O)C3CCC(CN4C(=O)C=CC4=O)CC3)cc2)nc1C(=O)Nc1cnccc1N1CCOCC1]

To a solution containing 43 mg (0.05 mmol) of (S)-2-amino-N¹-(4-(5-amino-6-((4-morpholinopyridin-3-yl)carbamoyl)pyrazin-2-yl)benzyl)-N5-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethyl)pentanediamide trihydrochloride as bright yellow crystalline solid which was combined with (32 mg, 0.07 mmol) of LC-smcc (succinimidyl 4-(N-maleimidomethyl)cyclohexane-1-carboxy-(6-amidocaproate)) in 3 mL of DCM and DIPEA (0.13 mL, 0.7 mmol). After stirring overnight, the reaction became cloudy and LCMS indicated the presence of product. The reaction was concentrated then taken up in a minimum amount of THF and water. The mixture was neutralized with saturated NaHCO₃ and the mixture was chromatographed (30 g, C18, H₂O to CH₃CN, liquid load) to provide Compound 2-1 as a yellow solid after lyophilization from CH₃CN/H₂O. LCMS (M+H)=1194.3.

### Example 2

### Synthesis of Intermediates for Conjugation to Antibodies

### TRAF2 And NCK Interacting Kinase (TNIK) Inhibitors

### Example 2.1 Synthesis of 4-((S)-2-((S)-2-(6-(2,5-dioxo-2,5-dihydro-1H-pyrrol-1-yl)hexanamido)-3-methylbutanamido)-5-ureidopentanamido)benzyl((6-((8-(((1s,4s)-4-hydroxy-cyclohexyl)oxy)quinazolin-2-yl)amino)-1H-benzo[d]imidazol-4-yl)methyl)carbamate (Compound 2.1)

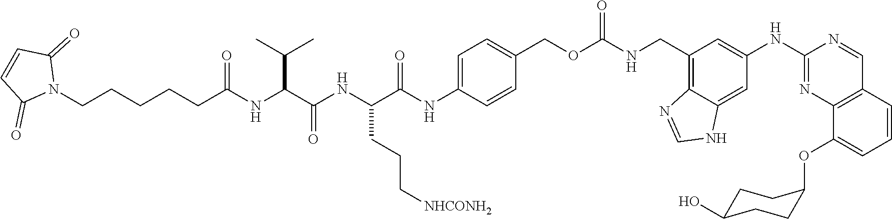

### Step A: Preparation of Int 2.1a

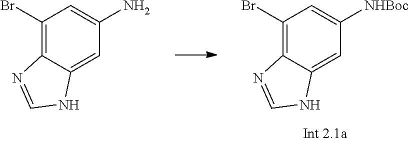 [SMILES: Nc1cc(Br)c2nc[nH]c2c1 ; CC(C)(C)OC(=O)Nc1cc(Br)c2nc[nH]c2c1]

4-bromo-1H-benzo[d]imidazol-6-amine (903 mg, 4.26 mmol) was dissolved in a mixture of 15 mL THF, 8 mL of H₂O and 20 mL of MeOH. Solid NaHCO₃ (716 mg, 8.52 mmol) was added and the mixture was stirred for 15 min before adding 1.4 g (6.39 mmol) of Boc₂O. The reaction mixture was concentrated and covered with MeOH to give a fine dark suspension. Silica gel was then added and the mix was concentrated to dryness. Silica gel column chromatography (ISCO 125 g, DCM to 20% MeOH/DCM) provided the desired material (1.1 g) as a yellow solid. ¹H NMR (CDCl₃) δ 8.48 (s, 1H), 8.41 (s, 1H), 8.14 (s, 1H), 7.54 (s, 1H), 1.69 (s, 9H).

### Step B: Preparation of Int 2.1b

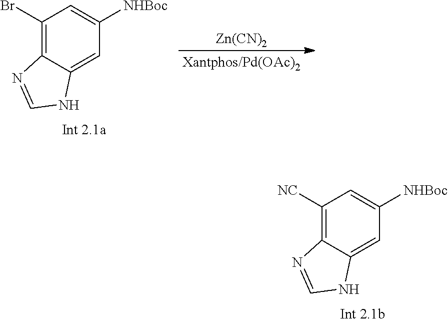

To a mixture containing Int 2.1a (0.979 g, 2.37 mmol) in 25 mL of DMF was added 4,5-bis(diphenylphosphino)-9,9-dimethylxanthene (0.137 g, 0.237 mmol) and Zn(CN)₂ (0.418 g, 3.56 mmol). The mixture was purged with N₂ for 10 minutes. Palladium(II) acetate (0.053 g, 0.237 mmol) was then added and the mixture purged with N₂ for 10 min then heated to 80° C. After 5 h the reaction was cooled and diluted with EtOAc and filtered through Celite. Chromatography (24 g Gold silica, DCM to 20% MeOH/DCM) gave tert-butyl (4-cyano-1H-benzo[d]imidazol-6-yl)carbamate gave the desired product as a pale pink solid which was used directly in the next step.

### Step C: Preparation of Int 2.1c

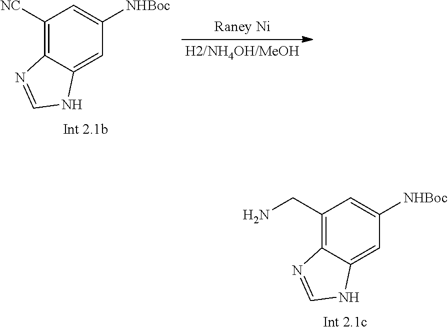 [SMILES: CC(C)(C)OC(=O)Nc1cc(C#N)c2nc[nH]c2c1 ; CC(C)(C)OC(=O)Nc1cc(CN)c2nc[nH]c2c1]

H Cube: 22 mL of 30% concentrated NH₄OH was diluted to 200 mL with MeOH. Half of this solution was used to prime and wash the H-cube lines and 2N NH₄OH in MeOH (88 ml) was used to dissolve the sample. Used 70×4 mm Ra—Ni column, 60° C., 10 psi, 1 ml/min, 0.026 molar in NH₄OH/MeOH for 4 h (recirculate) on the H-cube instrument when LCMS showed product with some SM remained. The sample was concentrated and placed under high vacuum for 16 h. Chromatography (40 g silica, Gold, DCM to 80:18:2 DCM:MeOH:NH₄OH) gave a partial separation and 440 mg of the desired product as a white solid which was used directly in the next step without additional purification. LCMS (M+H)=263.

### Step D: Preparation of Int 2.1d

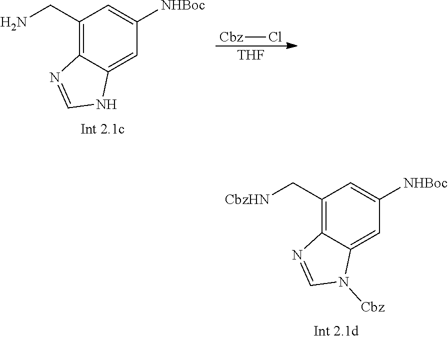 [SMILES: CC(C)(C)OC(=O)Nc1cc(CN)c2nc[nH]c2c1 ; O=C(Cl)OCc1ccccc1 ; CC(C)(C)OC(=O)Nc1cc(CNC(=O)OCc2ccccc2)c2ncn(C(=O)OCc3ccccc3)c2c1 ; "THF"]

To an ice-cold mixture of Int 2.1c and 3 mL saturated NaHCO₃ in 17 mL of THF was added benzyl chloroformate (0.29 mL, 2.0 mmol) dropwise. The reaction mixture was stirred for 3 h, then concentrated, covered with EtOAc and filtered through Na₂SO₄, concentrated with silica gel and dry loaded onto a 24 g silica Gold cartridge. Elution with 100% heptanes to 100% EtOAc gave 450 mg of Int 2.1d as a white solid. ¹H NMR (CDCl₃) δ 8.52 (s, 1H), 8.29 (s, 1H), 7.6-7.3 (m, 11H), 6.60 (bs, 1H), 5.55 (s, 2H), 5.25 (s, 2H), 4.61 (s, 2H), 1.56 (s, 9H).

### Step E: Preparation of Int 2.1e

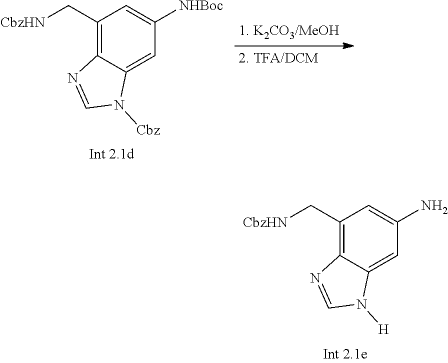 [SMILES: CC(C)(C)OC(=O)Nc1cc(CNC(=O)OCc2ccccc2)c2ncn(C(=O)OCc3ccccc3)c2c1 ; Nc1cc(CNC(=O)OCc2ccccc2)c2nc[nH]c2c1]

To a suspension of benzyl 4-((((benzyloxy)carbonyl)amino)methyl)-6-((tert-butoxycarbonyl)amino)-1H-benzo[d]imidazole-1-carboxylate (161 mg, 0.303 mmol) in 10 mL of MeOH was added K₂CO₃ (84 mg, 0.606 mmol). The reaction was stirred at room temperature for 1 h when TLC showed the reaction to be complete. Chromatography (4 g silica, Gold, DCM to 20% MeOH/DCM) gave the mon-deprotected compound (141.8, mg) as a white solid. This material was dissolved in 9 mL of DCM and treated with 1 mL of TFA. The reaction was stirred at room temperature for 1 h then concentrated. The residue taken up in DCM and treated with 1 mL of Et₃N. The reaction was concentrated and chromatographed (4 g silica gel, Gold, DCM to 80:18:2 DCM:MeOH:NH₄OH) to give 115 mg of Int 2.1e as a pale yellow semi-solid. ¹H NMR (CDCl₃) δ 7.70 (s, 1H), 7.25 (m, 5H), 6.72 (s, 1H), 6.42 (s, 1H), 5.87 (bs, 1H), 5.05 (s, 2H), 4.44 (s, 2H).

### Step F: Preparation of Int 2.1f

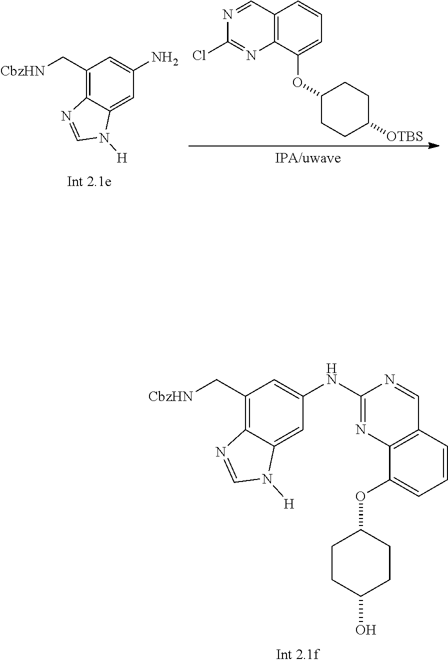

A mixture containing 56 mg (0.188 mmol) of Int 2.1e and 96 mg (0.244 mmol) of 8-(((1s,4s)-4-((tert-butyldimethylsilyl)oxy)cyclohexyl)oxy)-2-chloroquinazoline in 2 mL of isopropanol was heated in a microwave tube for 2 h at 150° C. The reaction was cooled and 0.5 mL of water was added. Tetrabutylammonium fluoride (564 μl, 0.564 mmol) was added and the mixture was stirred for 2 h. The solvent was concentrated then partitioned between EtOAc and NaHCO₃. The EtOAc was washed with water then brine and dried (Na₂SO₄), filtered and concentrated. The material was adsorbed onto silica gel using DCM then concentrated. Chromatography (24 g silica gel, Gold, DCM to 10% MeOH/DCM) to give 41 mg of Int 2.1f as a yellow solid. ¹H NMR (CD₃OD) δ 9.12 (s, 1H), 9.05 (bs, 1H), 8.06 (s, 1H), 7.41-7.06 (m, 9H), 5.13 (s, 2H), 4.89 (s, 1H), 4.71 (bs, 2H), 3.86 (bs, 1H), 2.20-2.07 (m, 4H), 1.89-1.80 (m, 2H), 1.73 (t, J=12 Hz, 2H). LCMS (M+H)=539.6.

### Step G: Preparation of Compound 2.1g

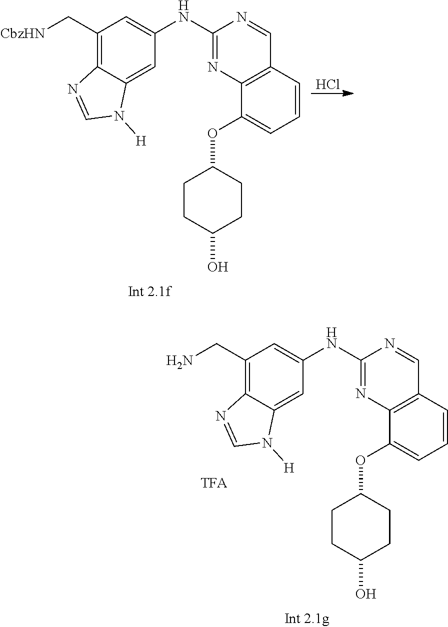

A mixture containing benzyl ((6-((8-(((1s,4s)-4-hydroxycyclohexyl)oxy)quinazolin-2-yl)amino)-1H-benzo[d]imidazole-4-yl)methyl)carbamate (181 mg, 0.336 mmol) was combined with water (2 ml) and 4 N HCl in dioxane (2 ml) in a microwave tube then heated in a microwave for 2 h at 100° C. The solvents were removed under reduced pressure and saturated NaHCO₃ was added to make the free-base. This mixture was loaded onto a 100 g C18 column using a minimum of MeOH to finish the loading. Elution with H₂O to CH₃CN (TFA modifier) gave 121 mg of Int 2.1g as yellow solid after co-evaporation with DCM and heptane. ¹H NMR (D₂O) δ 9.12 (s, 1H), 8.97 (s, 1H), 8.53 (s, 1H), 7.48 (d, J=2.0 Hz, 1H), 7.42 (dd, J=2.0, 8.0 Hz, 1H), 7.34 (d, J=8.0 Hz, 1H), 7.30 (m, 3H), 4.70 (s, 1H), 4.44 (s, 2H), 3.80 (m, 1H), 1.94 (m, 2H), 1.67 (m, 6H). LCMS (M+H)=405.3.

### Step H: Preparation of Compound 2.1

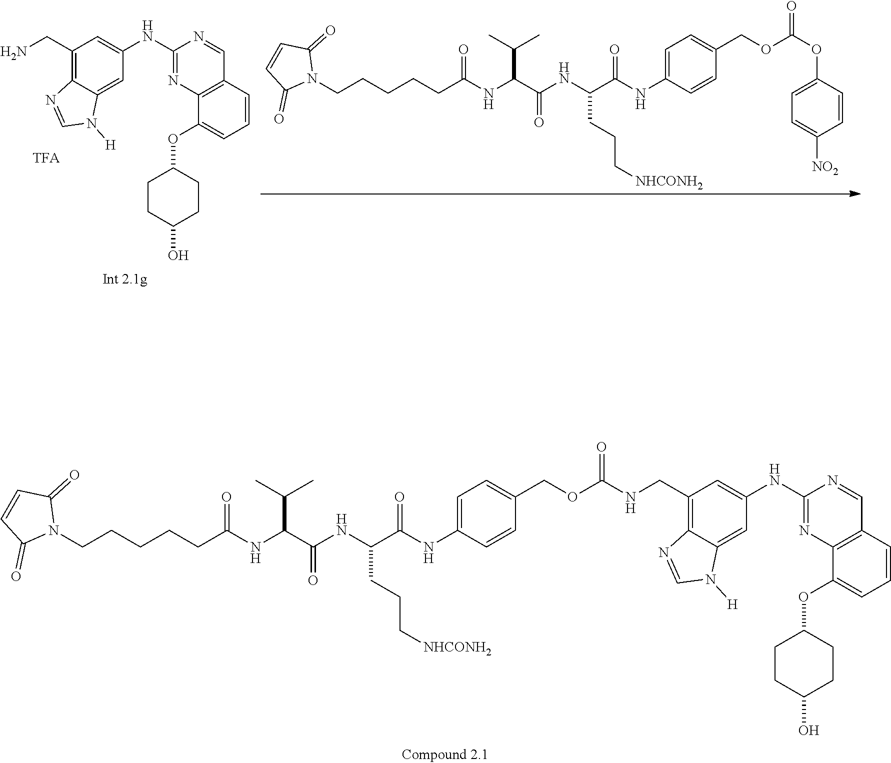

To a solution containing 4-((S)-2-((S)-2-(6-(2,5-dioxo-2,5-dihydro-1H-pyrrol-1-yl)hexanamido)-3-methylbutanamido)-5-ureidopentanamido)benzyl (4-nitrophenyl) carbonate (45.6 mg, 0.062 mmol) in 1 mL of DMF was added 618 uL of a 0.1 M solution of (1s,4s)-4-((2-((4-(aminomethyl)-1H-benzo[d]imidazol-6-yl)amino)quinazolin-8-yl)oxy)cyclohexan-1-ol (618 μL, 0.062 mmol) and N,N-diisopropylethylamine (21.53 μl, 0.124 mmol). The reaction was stirred for 16 h then concentrated. The residue was chromatographed (30 g C18, H₂O to CH₃CN, liquid loaded using a mixture of THF, saturated NaHCO₃ (aq) and H₂O) to give 25 mg of Compound 2.1 as a yellow solid. ¹H NMR (CD₃OD) δ 9.12 (s, 1H), 9.07 (bs, 1H), 8.06 (s, 1H), 7.56 (d, J=8.0 Hz, 2H), 7.40 (d, J=8.0 Hz, 1H), 7.33-7.22 (m, 4H), 7.12 (bs, 1H), 6.76 (s, 2H), 5.08 (s, 2H), 4.89 (m, 1H), 4.82 (s, 2H), 4.72 (m, 2H), 4.49 (m, 1H), 4.15 (d, J=7.5 Hz, 1H), 3.86 (bs, 1H), 3.44 (m, 3H), 3.22-3.08 (m, 4H), 2.26 (t, J=7.5 Hz, 2H), 2.18-2.03 (m, 6H), 1.89 (m, 3H), 1.73 (m, 3H), 1.65-1.51 (m, 7H), 1.30 (3H), 0.96 (m, 6H). LCMS (M+H)=1003.9.

### TGFβR2 Inhibitors

### Example 2.2 The Following Compounds are Prepared in a Manner Similar to that Described for Compound 2.1

Compound 2.3 LP of 42-O-(aminoethyl) Rapamycin

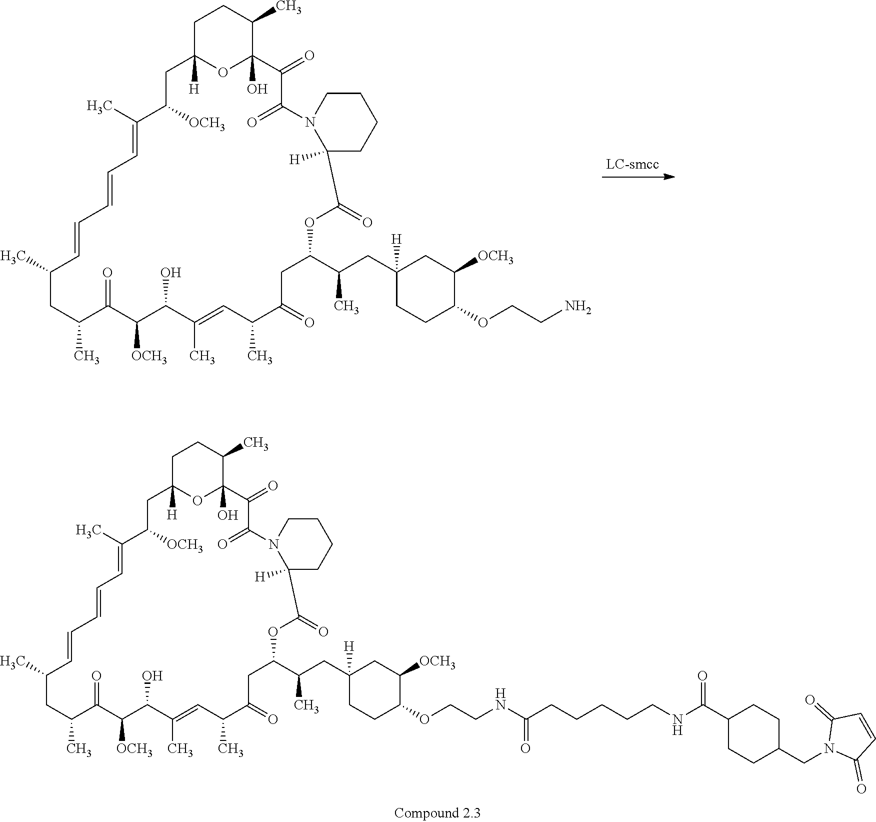 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCN)[C@H](OC)C2)CC(=O)[C@H](C)/C=C(\C)[C@@H](O)[C@@H](OC)C(=O)[C@H](C)C[C@H](C)/C=C/C=C/C=C/1C ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCNC(=O)CCCCCNC(=O)C3CCC(CN4C(=O)C=CC4=O)CC3)[C@H](OC)C2)CC(=O)[C@H](C)/C=C(\C)[C@@H](O)[C@@H](OC)C(=O)[C@H](C)C[C@H](C)/C=C/C=C/C=C/1C]

### Example 3

### Generation of Antibody-TGFb Inhibitor Conjugates Through Partial Reduction of Native Intrachain Disulfide Bonds of Non-Engineered Antibodies

The mAb (3-8 mg/mL in PBS) was exchanged into HEPES (100 mM, pH 7.0, 1 mM DTPA) via molecular weight cut-off centrifugal filtration (Millipore, 30 kDa). The resultant mAb solution was transferred to a tared 50 mL conical tube. The mAb concentration was determined to be 3-8 mg/mL by A₂₈₀. To the mAb solution was added TCEP (2.0-4.0 equivalents, 1 mM stock) at room temperature and the resultant mixture was incubated at 37° C. for 30-90 minutes, with gentle shaking. Upon being cooled to room temperature, a stir bar was added to the reaction tube. With stirring, the linker-payload from Examples 1 and 2 (5-10 equivalents, 10 mM DMSO) was added dropwise. The resultant reaction mixture was allowed to stir at ambient temperature for 30-60 minutes, at which point N-ethyl maleimide (3.0 equivalents, 100 mM DMA) was added. After an additional 15 minutes of stirring, N-acetylcysteine (6.0-11.0 equivalents, 50 mM HEPES) was added. The crude ADC was then exchanged into PBS and purified by preparative SEC (e.g. HiLoad 26/600, Superdex 200 pg) using PBS as the mobile phase. The pure fractions were concentrated via molecular weight cut-off centrifugal filtration (Millipore, 30 kDa), sterile filtered, and transferred to 15 mL conical tubes. Drug-antibody construct ratios (molar ratios) were determined by methods described in Example 4 below.

### Example 4

### General Procedure for the Determination of the Drug-Antibody Construct Ratios

### Hydrophobic Interaction Chromatography

10 μL of a 6 mg/mL solution of a conjugate is injected into an HPLC system set-up with a TOSOH TSKgel Butyl-NPR™ hydrophobic interaction chromatography (HIC) column (2.5 μM particle size, 4.6 mm×35 mm) attached. Then, over the course of 18 minutes, a method is run in which the mobile phase gradient is run from 100% mobile phase A to 100% mobile phase B over the course of 12 minutes, followed by a six-minute re-equilibration at 100% mobile phase A. The flow rate is 0.8 mL/min and the detector is set at 280 nM. Mobile phase A is 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

### Mass Spectrometry

One microgram of antibody conjugate (antibody construct immune-modulatory compound conjugate) is injected into an LC/MS such as an Agilent 6550 iFunnel Q-TOF equipped with an Agilent Dual Jet Stream ESI source coupled with Agilent 1290 Infinity UHPLC system. Raw data is obtained and is deconvoluted with software such as Agilent MassHunter Qualitative Analysis Software with BioConfirm using the Maximum Entropy deconvolution algorithm. The average mass of intact antibody conjugate is calculated by the software, which used top peak height at 25% for the calculation. This data is then imported into another program to calculate the molar ratio of the antibody conjugate such as Agilent molar ratio calculator.

### Example 5

### TGFβ Reporter Assay

Materials and General Procedures.

TGFβ/SMAD Signaling Pathway SBE reporter cell line was obtained from BPS Bioscience. Cells were passed, expanded, and stored in liquid nitrogen as per the supplier's instructions with the exception that growth media is changed to DMEM-C with Geneticin (DMEM supplemented with 10% fetal bovine serum, 1×NEAA, 1 mM Pyruvate, 2 mM glutamine, 50 μg/mL penicillin, 50 U/mL streptomycin and 400 μg/mL of Geneticin). The assay media was MEM supplemented with 0.5% fetal bovine serum, 1×NEAA, 1 mM Pyruvate, 50 μg/mL penicillin and 50 U/mL streptomycin.

General Procedure for In Vitro Small Molecule Screening.

Test samples (at desired concentrations diluted in assay media) were added to a 96-well assay plate, 20 μL per well. Reporter cells were harvested from the tissue culture flasks by incubation in small quantity of PBS at 37° C. for two minutes after the media in the flask is removed and cells rinsed with PBS. Cells were counted and diluted in the assay media at approximately 0.5×10⁶ cells/mL and then 80 μL/well of cells were added to the assay plate containing the 20 μL/well of test samples (or media only) and incubated for approximately 5-6 hours at 37° C. in a 5% CO₂ humidified incubator. After that time, 15 μL of TGFβ diluted to 12 ng/mL in the assay media was added to the plate. Controls included TGFβ titration (from 50 to 0 ng/mL) without inhibitors, and media only (without cells, inhibitor or TGFβ). Plates were incubated at 37° C. in a 5% CO₂ humidified incubator for 18 h. Luciferase substrate solution is subsequently added at 100 μL per well, incubated in the dark at room temperature for 15 min, and luminescence is measured using a luminometer.

### Example 6

### Determination of K_{d} Values

K_{d} is measured using surface plasmon resonance assays using a BIACORE®-2000 or a BIACORE®-3000 (BIAcore, Inc., Piscataway, N.J.) at 25° C. with immobilized antigen CM5 chips at 10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/mL (0.2 μM) before injection at a flow rate of 5 μL/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μL/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, e.g., Chen et al., J. Mol. Biol. 293:865-881 (1999). If the on-rate exceeds 106 M-1 s−1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### Example 7

### A FAP-TGFβR2 Targeted Conjugate Inhibited a TGFβ Signal

To examine inhibition of TGFβ-mediated effects on fibroblast function, FAP-expressing GM05389 lung fibroblasts were cultured overnight in the absence of serum. The following day, cells were treated for 1 hour with titrating concentrations of a conjugate of FAP antibody and a TGFβR2 immune-modulatory compound LP (FAP-TGFβR2 conjugate). Cells were then stimulated with 10 ng/mL TGFβ and then cultured for an additional 72 hours. Supernatants were harvested and soluble collagen concentration was determined using either the COL1A1 AlphaLISA or the colorimetric Sircol Collagen Assay. For some conditions, fibroblast proliferation was quantified using the CellTiter-Glo Luminescent Cell Viability Assay. For analysis of a-SMA (smooth muscle actin) induction, GM05389 cells were fixed using 4% PFA, permeabilized, and stained intracellularly using a PE-conjugated anti-a-SMA antibody. Flow cytometric analysis was then used to determine the level a-SMA expression. The MFI was lower for the conjugate-treated cells, indicating lower TGFβR2 mediated signaling.

### Example 8

### A FAP-TGFbR2 Conjugate Increases TGFbR2 Degradation in Treated Cells

Targeted TGFβR2 degradation in fibroblasts is demonstrated for a FAP-TGFβR2 conjugate comprised of an immune-modulatory compound comprised in part of a binding moiety for the E3 ubiquitin ligase Cereblon as follows. FAP-expressing GM05389 lung fibroblasts are cultured overnight in the absence of serum in 6-well tissue culture plates. Cells are then treated for 24 hours with titrating concentrations of 2 FAP-TGFβR2 conjugates, one where the immune-modulatory compound contains an E3 ubiquitin binding moiety and one that lacks the E3 ubiquitin ligase binding moiety. An additional control is treatment with a conjugate with a binding domain that does not recognize an antigen on GM05389 cells. After the incubation, media is removed by aspiration, the cells are washed with warm PBS buffer removed by aspiration, and the cells are lysed by addition of 100 μl of 1×Cell Lysis Buffer (Cell Signaling Technologies, Inc.) containing a protease inhibitor cocktail, incubation on ice, are placed into a plate shaker for 2 minutes and are collected after homogenization by up and down pipetting. Aliquots are removed and lysate protein concentration is determined by BCA using a standard procedure using BSA to generate a standard curve. The remainder of the lysates are subjected to SDS-PAGE gel electrophoresis and western blot analysis as follows: 1) the lysates are prepared for SDS PAGE using a standard 4×SDS sample buffer and heating at 100 C, 2) equal lysate protein amounts are added for each lysate to a lane and are separated by electrophoresis, 3) the separated proteins are transferred to Immobilon-FL PVDF membranes (Millipore, Inc.), 4) a specific anti-TGFβR2 mouse antibody is added to the membranes in standard blocking buffer, followed by incubation and then washes; 5) an HRP-labeled anti-mouse IgG antibody (Cell Signaling Technologies, Inc.) is added, incubated with the membrane followed by washes; and 6) an HRP enzyme assay kit is used to generate a chemiluminescence signal that was quantitated by a BioRad Chemi Lab reader. The diminished level of chemiluminescence found with increasing levels of the added FAP-TGFβR2 conjugate containing the E3 ubiquitin binding moiety compared to the signal for control lanes is demonstrated by the conjugate lowered TGFβR2 levels in the treated cells.

### Example 9

### An LRRC15-TGFβR2 Inhibitor Conjugate Inhibited the TGFβ/SMAD Signaling Pathway

To demonstrate that a conjugate of an LRRC15 antibody attached to a TGFβR2 inhibitor via a linker was active and able to inhibit the TGFβ/SMAD signaling pathway, following TGFβ induction, an assay was performed using a reporter cell line.

Materials and general procedures: a parental TGFβ/SMAD signaling pathway reporter cell line was maintained in DMEM supplemented with 10% fetal bovine serum, 1×NEAA, 1 mM Pyruvate, 2 mM glutamine, 50 μg/mL penicillin, 50 U/mL streptomycin and 400 ug/mL of Geneticin. The reporter cell line was transiently transfected with a vector encoding LRRC15 reporter by plating the reporter cells in 6 well plates and the following day transfecting them using Lipofectamine 3000 per manufacturer's instructions. Twenty four hours post-transfection, transfected LRRC15 reporter cells and control reporter cells (not expressing LRRC15) were harvested from the tissue culture flasks by incubation in small quantity of Versene at room temperature for three to five minutes after the media in the flask was removed and cells rinsed with PBS. Cells were counted and diluted in assay media at ˜0.8×10⁶ cells/mL, then 50 μL/well were added to 96-well assay plate. (Assay media was MEM supplemented with 0.5% fetal bovine serum, 1×NEAA, 1 mM Pyruvate, 50 μg/mL penicillin and 50 U/mL streptomycin.) A volume of 50 μL/well of test samples (at desired concentrations diluted in assay media) were added to an assay plate containing the cells, and incubated for 5-6 hours at 37° C. in a 5% CO₂ humidified incubator. After that time, 15 μL of TGFβ diluted to 12.5 ng/mL in the assay media was added to the plate. Controls included LRRC15 antibody alone, anti-digoxin-TGFβR2 inhibitor conjugate, TGFβ titration, cells with TGFβ treatment only, and media only (without cells, inhibitor or TGFβ). Plates were incubated at 37° C. in a 5% CO₂ humidified incubator for 24 hrs. Luciferase substrate solution was subsequently added at 75 μL per well, incubated in dark with shaking at room temperature for 10 min, and luminescence was measured using a luminometer.

Referring to FIG. 2, the results show that the LRRC15-TGFβR2 inhibitor conjugate was able to reduce signaling by the TGFβ/SMAD signaling pathway. In the figure, the X-axis shows concentration of conjugate or antibody added.

### Example 10

### A DEC205-Rapamycin Conjugate Lowers Dendritic Cell Stimulation of an Allo MLR Reaction

To demonstrate that a DEC-205-rapamycin conjugate can lower the ability of dendritic cells to stimulate T cells, human dendritic cells are treated with a DEC205-rapamycin conjugate and then are evaluated for their ability to activate allogeneic T cells in the context of a mixed lymphocyte reaction (MLR). Dendritic cells are differentiated from monocytes isolated from peripheral blood by culturing in RPMI-1640 medium supplemented with 10% (v/v) heat-inactivated fetal calf serum (FCS), 2 mM L-glutamine, 50 U/ml penicillin and 50 μg/mL streptomycin (Invitrogen) supplemented with 1000 U/mL recombinant human (rh) GM-CSF (R&D Systems) and 25 ng/mL rh IL-4 (R&D Systems) for 5 days. Fresh medium containing rhGM-CSF and rhIL-4 is added on Day 3 (0.5 volume). Immature DC are harvested and are extensively washed with RPMI and then are re-plated in 24 well-plates in complete RPMI media. The DEC205-rapamycin conjugate is added to dendritic cells over a range of concentrations from 200 nM to 0.1 nM and dendritic cells and the conjugate are incubated at 37° C. for 24 hours. Control treatments, such as untreated, DEC-205 mAb, and rapamycin small molecule, are included in the experiment. After 24 hours, dendritic cells are harvested, are washed and are replated at a 1:1 ratio with CFSE-labeled T cells in a 96-well plate. After a period of 3-5 days, T cell proliferation are assessed by CFSE dilution on a flow cytometer (Becton Dickenson, Fortessa). Decreasing levels of T cell proliferation are observed with increasing concentrations of the DEC205-rapamycin conjugate.

### Example 11

### General Procedure for Determining Protein Degradation by Conjugates Containing Proteolysis Targeting Modules

Proteolysis targeting modules (PTMs) and immune-modulatory compounds are prepared as described above. Conjugates of PTMs and antibody constructs s are prepared as described in Example 3 for interchain disulfide conjugation. The average DAR is about 4.

Cells are plated, allowed to adhere, and treated with vehicle, an inhibitor, a PROTAC or a conjugate in the presence or absence of proteasome inhibitor, such as MG-132. After treatment, media is aspirated and cells are rinsed with ice cold PBS. Ice cold lysis buffer (20 mM TrisHCl pH 7.5, 150 mM NaCl, 1% Triton X-100, 2 mM EDTA and 10% glycerol) containing phosphatase and protease inhibitors is added to wells and cells are removed from the plate using a cell scraper. Lysates are transferred to a 1.5 ml tube and rocked for one hour at 4° C. with vortexing every ˜15 minutes. Tubes are spun at 8500×g for 10 minutes and supernatants are drawn into an insulin needle twice. Cell lysates are frozen at ˜80° C. Protein concentration is determined using a BCA assay and equal amounts of samples are boiled with reducing loading buffer. The samples are then subjected to electrophoresis on 4-20% polyacrylamide gels which are then transferred to PVDF membranes. Blocking and staining are done in 5% w/v soy milk PBS with 0.05% Tween 20 and washing using PBS with 0.05% Tween. For PROTACs and conjugates thereof targeting TGFβR2, blots are incubated overnight with rocking at 4° C. with 1:200 primary anti-TGFβR2 antibody (Santa Cruz, sc-17791). For PROTACs and conjugates thereof targeting TGFβR1, blots are incubated overnight with rocking at 4° C. with 1:3000 primary anti-TGFβR1 antibody (R&D, MAB5871). Loading controls are detected with 1:15000 diluted primary antibody incubation at room temperature for 1 hour with rocking (Tubulin—Abcam, ab7291; Actin—Abcam, ab8224). Secondary antibodies are diluted 1:10000 and blots are incubated for 1 hour at room temperature with rocking (Jackson ImmunoResearch, 115-035-003 or 112-035-003). ECL reagent is used to detect the signal and blots are imaged using the ChemiDoc MP (Biorad). Analysis of densitometry is done using the ImageLab software and signals are adjusted based on loading control.

### Example 12

### Degradation of TFGβR2 by a TGFβR2-VHL PROTAC Conjugated to an Anti-HER2 Antibody

Protac T-20 was prepared as described in Example 1. Pertuzumab was used as the Her2 antibody. Her2 Antibody-Protac conjugate (050-T11020; Compound 2-1 (Example 1)) was prepared by attachment of a maleimidomethylcyclohexane-1-carboxylate linker to T-20 to form a linker-T-20 construct (T11020) followed by conjugation of T11020) to the Her2 antibody generally following the protocol in Example 3 for interchain cysteine conjugation. The average drug loading was about 4.

Plasmid expressing HER2 was transfected into HEK293 cells using commercially available materials and conditions. Twenty four hours after transfection, cells were treated with DMSO, PROTAC T-20, HER2 antibody (IgG1), or Her2 Antibody-Protac conjugate (050-T11020). Whole cell lysates were prepared from cells after 2, 24, or 48 hours incubation and quantitated with a BCA assay. Equal amounts of lysates were run on protein gels, transferred to PVDF, and TGFβR2 was detected using commercially available reagents. To quantitate the amount of protein degradation, the signals on the Western blot were adjusted to actin loading control and data is presented as a percent of matched control. Referring to FIG. 3A, FIG. 3B, and FIG. 3C, at both tested concentrations, 0.5 uM and 1 uM of conjugate, the level of target TGFβR2 was diminished at 24 and 48 hours of treatment, while TGFβR2.

### Example 13

### Antigen Targeted Degradation of TGFβR2 by Antibody Conjugates Having VHL and Cereblon E3 Binding Moieties

A HER2 antigen positive, TROP2 antigen negative cell line BT474 was used to demonstrate antigen specific delivery of PROTAC conjugates.

Protac T-15 and T-20 were prepared as described above in Example 1. Pertuzumab was used as the Her2 antibody. Sacituzumab was used as the Trop2 antibody. Her2 Antibody-Protac conjugates (050-T05015 and 050-T05020) were prepared by attachment of an MC-VC-PAB linker to T-15 or T-20 to form T05015 and T05020 constructs, respectively, followed by conjugation to the Her2 antibody generally following the protocol in Example 3 for interchain cysteine conjugation. The average drug loading was about 4. Trop2-Protac conjugate (130-T05020) was similarly prepared.

BT474 cells were plated and treated the following day with either a small molecule (T-15 or T-20), a conjugate of a HER2 antibody TGFβR2-VHL binding PROTAC (050-T05020), a conjugate of a HER2 antibody TGFβR2-Cereblon binding PROTAC (050-T05015) or a conjugate of a TROP2 antibody TGFβR2-VHL binding PROTAC (130-T05020). Whole cell lysates were prepared 24 hours after treatment and quantitated with a BCA assay. Equal amounts of lysates were run on protein gels, transferred to PVDF, and TGFβR2 and actin were detected using commercially available reagents. Quantitation of protein bands was performed and Western signal was adjusted to actin loading control and data is presented as a percent of vehicle control. Referring to FIG. 4A and FIG. 4B, HER2-antigen specific degradation was found with both the HER2 binding PROTAC conjugates, but not with the control TROP2-binding PROTAC conjugates.

### Example 14

### Lowered Cellular Level of TGFβR2 and TGFβR1 by a TGFβR2/TGFβR1-VHL PROTAC is Proteasome Inhibitor Sensitive

Normal human lung fibroblasts were treated with or without proteasome inhibitor MG-132 followed by the addition of DMSO or T-20. Whole cell lysates were prepared and then quantitated with a BCA assay. Equal amounts of lysates were run on protein gels and transferred to PVDF membrane. TGFβR1, TGFβR2, and actin were detected using commercially available reagents. Western signal was adjusted to actin loading control and data is presented as a percent of the matched vehicle control. Referring to FIG. 5A and FIG. 5B, addition of the proteasome inhibitor protected TGFβR1 and TGFβR2 against degradation induced by T-20.

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

1. A conjugate represented by formula (VII): 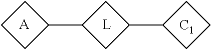
wherein C₁ comprises an immune-modulatory compound that is a TGFβR inhibitor; A is an antibody construct comprising an antigen binding domain and an Fc domain, wherein the antigen binding domain specifically binds to LRRC15; and L is a linker; and
optionally wherein a molar ratio of immune-modulatory compound to antibody construct is less than 8.

2.-13. (canceled)

14. The conjugate of claim 1, wherein:
(a) a Kd of the conjugate for binding of the Fc domain to an Fcγ receptor is no greater than 2 times, 5 times, or 10 times a Kd of the unconjugated antibody construct for binding of the Fc domain to the Fcγ receptor; or
(b) a Kd of the conjugate for binding of the Fc domain to an FcRn receptor is no greater than about 2 times, 5 times, or 10 times a Kd for the unconjugated antibody construct for binding of the Fc domain to the FcRn receptor; or
(c) a Kd of the conjugate for binding of the Fc domain to an Fcγ receptor is greater than 100 times a Kd of the unconjugated antibody construct for binding to the Fcγ receptor, and wherein a Kd of the conjugate for binding of the Fc domain to an FcRn receptor is no greater than about 2 times, 5 times, or 10 times a Kd of the unconjugated antibody construct for binding of the Fc domain to the FcRn receptor; or
(d) the Fc domain is an Fc null.

15.-26. (canceled)

27. The conjugate of claim 1, wherein the immune-modulatory compound lowers fibrogenic activity of a stellate cell or a myofibroblast.

28.-29. (canceled)

30. The conjugate of claim 1, wherein the immune-modulatory compound is an inhibitor of TGFβRI, TGFβRII, or both.

31.-48. (canceled)

49. The conjugate of claim 1, wherein the Fc domain comprises at least one amino acid residue change selected from a group consisting of:
a) N297A, N297G, N297Q or N297G as in the EU index of Kabat numbering and relative to SEQ ID NO: 437;
b) K322A/L234A/L235A as in the EU index of Kabat numbering and relative to SEQ ID NO: 437;
c) L234F/L235E/P33 IS N296A as in the EU index of Kabat numbering and relative to SEQ ID NO: 437; and
d) P329G/L234A/L235A as in the EU index of Kabat numbering and relative to SEQ ID NO: 437.

50. The conjugate of claim 1, wherein the Fc domain comprises an IgG4 Fc domain comprising S228P/L235E/P329G as in Kabat numbering.

51.-54. (canceled)

55. The conjugate of claim 1, wherein the first antigen binding domain comprises a set of CDRs having:
(a) HCDR1 comprising an amino acid sequence of SEQ ID NO: 440, HCDR2 comprising an amino acid sequence of SEQ ID NO: 441, HCDR3 comprising an amino acid sequence of SEQ ID NO: 442, LCDR1 comprising an amino acid sequence of SEQ ID NO: 443, LCDR2 comprising an amino acid sequence of SEQ ID NO: 444, and LCDR3 comprising an amino acid sequence of SEQ ID NO: 445;
(b) HCDR1 comprising an amino acid sequence of SEQ ID NO: 446, HCDR2 comprising an amino acid sequence of SEQ ID NO: 447, HCDR3 comprising an amino acid sequence of SEQ ID NO: 448, LCDR1 comprising an amino acid sequence of SEQ ID NO: 449, LCDR2 comprising an amino acid sequence of SEQ ID NO: 450, and LCDR3 comprising an amino acid sequence of SEQ ID NO: 451;
(c) HCDR1 comprising an amino acid sequence of SEQ ID NO: 452, HCDR2 comprising an amino acid sequence of SEQ ID NO: 453, HCDR3 comprising an amino acid sequence of SEQ ID NO: 454, LCDR1 comprising an amino acid sequence of SEQ ID NO: 455, LCDR2 comprising an amino acid sequence of SEQ ID NO: 456, and LCDR3 comprising an amino acid sequence of SEQ ID NO: 457;
(d) HCDR1 comprising an amino acid sequence of SEQ ID NO: 458, HCDR2 comprising an amino acid sequence of SEQ ID NO: 459, HCDR3 comprising an amino acid sequence of SEQ ID NO: 460, LCDR1 comprising an amino acid sequence of SEQ ID NO: 461, LCDR2 comprising an amino acid sequence of SEQ ID NO: 462, and LCDR3 comprising an amino acid sequence of SEQ ID NO: 463;
(e) HCDR1 comprising an amino acid sequence of SEQ ID NO: 464, HCDR2 comprising an amino acid sequence of SEQ ID NO: 465, HCDR3 comprising an amino acid sequence of SEQ ID NO: 466, LCDR1 comprising an amino acid sequence of SEQ ID NO: 467, LCDR2 comprising an amino acid sequence of SEQ ID NO: 468, and LCDR3 comprising an amino acid sequence of SEQ ID NO: 469;
(f) HCDR1 comprising an amino acid sequence of SEQ ID NO: 470, HCDR2 comprising an amino acid sequence of SEQ ID NO: 471, HCDR3 comprising an amino acid sequence of SEQ ID NO: 472, LCDR1 comprising an amino acid sequence of SEQ ID NO: 472, LCDR2 comprising an amino acid sequence of SEQ ID NO: 474, and LCDR3 comprising an amino acid sequence of SEQ ID NO: 475; or
(g) HCDR1 comprising an amino acid sequence of SEQ ID NO: 476, HCDR2 comprising an amino acid sequence of SEQ ID NO: 477, HCDR3 comprising an amino acid sequence of SEQ ID NO: 478, LCDR1 comprising an amino acid sequence of SEQ ID NO: 479, LCDR2 comprising an amino acid sequence of SEQ ID NO: 480, and LCDR3 comprising an amino acid sequence of SEQ ID NO: 481.

56.-59. (canceled)

60. The conjugate of claim 55, wherein the first antigen binding domain comprises:
(a) a VH sequence having at least 80% sequence identity to an amino acid sequence of SEQ ID NO: 482, and a VL sequence having at least 80% sequence identity to an amino acid sequence of SEQ ID NO: 483;
(b) a VH sequence having at least 80% sequence identity to an amino acid sequence of SEQ ID NO: 484, and a VL sequence having at least 80% sequence identity to an amino acid sequence of SEQ ID NO: 485;
(c) a VH sequence having at least 80% sequence identity to an amino acid sequence of SEQ ID NO: 486, and a VL sequence having at least 80% sequence identity to an amino acid sequence of SEQ ID NO: 487;
(d) a VH sequence having at least 80% sequence identity to an amino acid sequence of SEQ ID NO: 488, and a VL sequence having at least 80% sequence identity to an amino acid sequence of SEQ ID NO: 489; or
(e) a VH sequence having at least 80% sequence identity to an amino acid sequence of SEQ ID NO: 490, and a VL sequence having at least 80% sequence identity to an amino acid sequence of SEQ ID NO: 491.

61.-64. (canceled)

65. The conjugate of claim 1, wherein the antigen is expressed by stellate cells, podocytes, or myofibroblasts.

66. (canceled)

67. A pharmaceutical composition comprising the conjugate of claim 1 and a pharmaceutically acceptable carrier.

68. A method of treating a fibrotic disease in a subject in need thereof, comprising administering a therapeutically effective dose of the pharmaceutical composition of claim 67.

69. (canceled)

70. The method of claim 68, wherein the conjugate is administered intravenously, cutaneously, subcutaneously, or injected at a site of affliction.

71.-86. (canceled)

87. The method of claim 68, wherein the fibrotic disease is selected from the group consisting of adhesive capsulitis, arterial stiffness, arthrofibrosis, atrial fibrosis, cirrhosis, Crohn's disease, collagenous fibroma, cystic fibrosis, Desmoid-type fibromatosis, Dupuytren's contracture, elasto fibroma, endomyocardial fibrosis, fibroma of tendon sheath, glial scar, idiopathic pulmonary fibrosis, keloid, mediastinal fibrosis, myelofibrosis, nuchal fibroma, nephrogenic systemic fibrosis, old myocardial infarction, Peyronie's disease, pulmonary fibrosis, progressive massive fibrosis, radiation-induced lung injury, retroperitoneal fibrosis, scar, and scleroderma/systemic sclerosis.

88. (canceled)

89. The method of claim 68, wherein the antibody construct is an antibody.

90. (canceled)

91. The method of claim 68, wherein the antibody comprises heavy and light chain variable regions having amino acid sequences selected from the pairs of heavy and light variable region sequences set forth in SEQ ID NOS:482-491.

92. (canceled)

93. The conjugate of claim 1, wherein the antibody construct is an antibody.

94. The conjugate of claim 1, wherein L is represented by the formula: 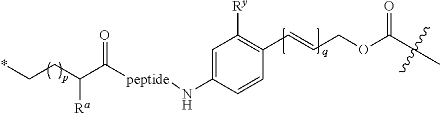
wherein peptide represents a peptide cleavable by a lysosomal enzyme; R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)—[(C₁₋₄ alkylene)-G¹]₂; p is an integer ranging from 0 to 5; q is 0 or 1; [symbol] represents the point of attachment of the linker to an immune-modulatory compound or salt thereof; and * represents the point of attachment to the remainder of the linker.

95. The conjugate of claim 94, wherein the cleavable peptide comprises a dipeptide selected from Val-Cit, Val-Ala, and Phe-Lys.

96. The conjugate of claim 1, wherein L is attached to the antibody construct at a lysine or cysteine residue.
